SEQUENCE LISTING (2043 sequences)

SEQ ID NO: 1 - protein, 1244 aa, synthetic construct
  source 1..1244: note = ART1
METFSGFTNLYPLSKTLRFRLIPVGETLKHFIDSGILEEDQHRAESYVKVKAIIDDYHRAYIENSLSGFELPLESTKFNSLEEYYLYHNIRNKTEEIQNLSSKVRTNLRKQVVAQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAIVNFWNTIHDTVLAEGGLKTIIASLGSYGLEGIFLKNDLQLTDISQKATGSWGKISSEIKQKIEVMNPQKKKESYETYQERIDKIFKSYKSFSLAFINECLRGEYKIEDYFLKLGAVNSSSLQKENHFSHILNTYTDVKEVIGLYSESTDTKLIQDNDSIQKIKQFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 2 - protein, 1197 aa, synthetic construct
  source 1..1197: note = ART2
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 3 - protein, 1252 aa, synthetic construct
  source 1..1252: note = ART3
MIDLKQFIGIYPVSKTLRFELRPVGKTQEWIEKNRVLEGDEQKAADYPVVKKLIDDYHKVCIHDSLNHVHFDWEPLKDAIEIFQKTKSDEAKKRLEAEQAMMRKKIAAAIKDFKHFKELTAATPSDLITSVLPEFSDDGSLKSFRGFATYFSGFQENRNNIYSQEAISTGVPYRLVHDNFPKFLSDLEVFERIKSTCPEVINQASAELQPFLEGVMIDDIFSLDFYNSLLTQNGIDFFNQVIGGVSEKDKQKYRGINEFSNLYRQQHKEIAASKKAMTMIPLFKQILSDRDTLSYIPAQIRTEDELVSSITQFYDHITHFEHDGKTINVLSEIVALLGKLDTYDPNGICITARKLTDISQKVYGKWSVIEEKMKEKAIQQYGDISVAKNKKKVDAFLSRKAYSLSDLCFDEEISFSRYYSELPQTLNAISGYWLQFNEWCKSDEKQKFLNNQTGTEVVKSLLDAMMELFHKCSVLVMPEEYEVDKSFYNEFLPLYEELDTLFLLYNKVRNYLTQKPSDVKKFKLNFESPSLASGWDQNKEMKNNAILLFKDGKSYLGVLNAKNKAKIKDAKGDVSSSSYKKMIYKLLSDPSKDLPHKIFAKGNLDFYKPSEYILEGRELGKYKKGPNFDKKFLHDFIDFYKAAISIDPDWSKFNFQYSPTESYDDIGMFFSEIKKQAYKIRFTDISEAQVNEWVDNGQLYLFQLYNKDYAEGAHGRKNLHTLYWENLFTDENLSNLVLKLNGQAELFCRPQSIKKPVSHKIGSKMLNRRDKSGMPIPESIYRSLYQYYNGKKKESELTVAEKQYIDQVIVKDVTHEIIKDRRYTRQEYFFHVPLTFNANADGNEYINEHVLNYLKDNPDVNIIGIDRGERHLIYLTLINQRGEILKQKTFNVVNSYNYQAKLEQREKERDEARKSWDSVGKIKDLKEGFLSAVIHEITNMMIENNAIVVLEDLNFGFKRGRFKVERQVYQKFEKMLIDKLNYLSFKDREAGEEGGILRGYQMAQKFISFQRLGKQSGFLFYIPAAYTSKIDPVSGFVNHFNFSDITNAEKRKDFLMKMDRIEMKNGNIEFTFDYRKFKTFQTDYQNVWTVSTFGKRIVMRIDEKGYKKMVDYEPTNDIIKAFKNKGILLSEGSDLKALIAEIEANATNAGFYSTLLYAFQKTLQMRNSNAVTEEDYILSPVAKDGHQFCSTDEANKGKDAQGNWVSKLPVDADANGAYHIALKGLYLLRNPETKKIENEKWLQFMVEKPYLE

SEQ ID NO: 4 - protein, 1287 aa, synthetic construct
  source 1..1287: note = ART4
MSYNREKMEEKELGKNQNFQEFIGVSPLQKTLRNELIPTETTKKNIAQLDLLTEDEVRAQNREKLKEMMDDYYRDVIDSTLRGELLIDWSYLFSCMRNHLSENSKESKRELERTQDSVRSQIHDKFAERADFKDMFGASIITKLLPTYIKQNSKYSERYDESVKIMKLYGKFTTSLTDYFETRKNIFSKEKISSAVGYRIVEENAEIFLQNQNAYDRICKIAGLDLHGLDNEITAYVDGKTLKEVCSDEGFAKVITQGGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 5 - protein, 1295 aa, synthetic construct
  source 1..1295: note = ART5
MSAVFKIKESTMKDFTHQYSLSKTLRFELKPVGETAERIEDFKNQGLKSIVEEDRQRAEDYKKMKRILDDYHKEFIEEVLNDDIFTANEMESAFEVYRKYMASKNDDKLKKEITEIFTDLRKKIAKAFENKSKEYCLYKGDFSKLINEKKTGKDKGPGKLWYWLKAKADAGVNEFGDGQTFEQAEEALAKFNNFSTYFTGFNQNRDNIYTDAEQQTAISYRVINENMTRYFDNCIRYSSIENKYPELVKQLEPLSGKFAPGNYKDYLSQTAIDIYNEAVGHKSDDINAKGINQFINEYRQRNSIKGRELPIMSVLYKQILSDINKDLIIDKFENAGELLDAVKTLHRELTDKKILLKIKQTLNEFLTEDNSEDIYIKSGTDLTAVSNAIWGEWSVIPKALEMYAENITDMNAKAREKWLKREAYHLKTVQEAIEAYLKDNEEFETRNISEYFTNFKSGENDLIQVVQSAYAKMESIFGIEDFHKDRRPVTESGEPGEGFRQVELVREYLDSLINVEHFIKPLHMFRSGKPIELEDCNSNFYDPLNEAYKELDVVFGIYNKVRNYVTQKPYSKDKFKINFQNSTLLDGWDVNKESANSSVLLLKNGKYYLGVMKQGASNILNYRPEPSDSKNKINAKKQLSEIALAGATDDYYEKMIYKLLPDPAKMLPKVFFSAKNIEFYNPSQEIIYIRENGLFKKDAGDKESLKKWIGFMKTSLLKHPEWGSYFNFEFEPAEDYQDISIFYKQVAEQGYSVTFDKIKTSYIEEKVASGELYLFEIYNKDFSPHSKGRPNLHTMYWKSLFEKENLQNLVTKLNGEAEVFFRQHSIKRNEKVVHRANRPIQNKNPLTEKKQSIFEYDLVKDRRFTKDKFFLHCPITLNFKEAGPGRFNDKVNKYIAGNPDIRIIGIDRGERHLLYYSLIDQSGRIVEQGTLNQITSTLNSGGREIPKTTDYRGLLDTKEKERDKARKSWSMIENIKELKSGYLSHIVHKLAKLMVKNNAVVVLEDLNFGFKRGRFKVEKQVYQKFEKALIEKLNYLVFKDARPAEPGHYLNAYQLTAPLESFKKLGKQSGFIYYVPAWNTSKIDPVTGFVNQFYIEKNSMQYLKNFFGKFDSIRFNPDKNYFEFGFDYKNFHNKAAKSKWTICTHGDKRSWYNRKQRKLEIHNVTENLASLLSGKGINFADGGSIKDKILSVDDASFFKSLAFNFKLTAQLRHTFEDNGEEIDCIISPVAAADGTFFCSETAKKLNMELPHDADANGAYNIARKGLMVLRQIRESGKPKPISNADWLDFAQQNED

SEQ ID NO: 6 - protein, 1245 aa, synthetic construct
  source 1..1245: note = ART6
MQERKKISHLTHRNSVQKTIRMQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 7 - protein, 1281 aa, synthetic construct
  source 1..1281: note = ART7
MNILKENYMKEIKELTGLYSLTKTIGVELKPVGKTQELIEAKKLIEQDDQRAEDYKIVKDIIDRYHKDFIDKCLNCVKIKKDDLEKYVSLAENSNRDAEDFDKIKTKMRNQITEAFRKNSLFTNLFKKNLIKEYLPAFVSEEEKSVVNKFSKFTTYFDAFNDNRKNLYSGDAKSGTIAYRLIHENLPMFLDNIASFNAISGIGVNEYFSSIETEFTDTLEGKRLTEFFQIDFFNNTLTQKKIGNYNYIVGAVNKAVNLYKQQHKTVRVPLLKPLYKMILSDRVTPSWLPERFESDEEMLTAIKAAYESLREVLVGDNDESLRNLLLNIEHYDLEHIYIANDSGLTSISQKIFGCYDTYTLAIKDQLQRDYPATKKQREAPDLYDERIDKLYKKVGSFSIAYLNRLVDAKGHFTINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 8 - protein, 1293 aa, synthetic construct
  source 1..1293: note = ART8
MAKENIFNELTGKYQLSKTLRLELKPVGNTQQMLKDEDVFEKDRIIREKYRETRPHFDRLHREFIEQALKNQKLSDLGKYFQCLAKLQNNKKDKEAQEEFKRISQNLRKEVNDLFKIDPLFGEGVFALLKEKYGEKDDAFLREQDGQYVLDENKKKISIFDSWKGFTGYFTKFQETRKNFYKDDGTATAVATRIIDQNLKRFCENIQIFKSIQKKVDFKEVEDNFSVDLEDIFSLGFYSSCFLQEGIDVYNKILGGEPKTTGEKLRGLNELINRYRQDHKGEKLPFFKMLDKQILSEKEKFIESIEDDEELLKTLKEFYSSAEEKTTVLKELFNDFIKNNENYDLSEIYISREALNTISHRWVSAATLPEFEKSVYEVMKKDKPSGLSFDKDDNSYKFPDFIALSYIKGSFEKLSGEKLWKDGYFRDETRNGDKGFLIGNESLWTQFIKIFEFEFNSLFEAKNTERSVGYYHFKKDFEKIITNDFSVNPEDKVIIREFADNVLAIYQMAKYFAIEKKRKWMDQYDTGDFYNHPDFGYKTKFYDNAYEKIVKARMLLQSYLTKKPFSTDKWKLNFECGYLLNGWSSSFNTYGSLLFRTGNEYYLGVVNGSALRTEKIKRLTGNITEANSCHKMVYDFQKPDNKNVPRIFIRSKGDKFAPAVSELNLPVDSILEIYDKGLFKTENKNSPFFKPSLKKLIDYFKLGFSRHASYKHYQFKWKDSSEYKNISEFYNDTIRSCYQIKWEELNFEEVKKLTNSKDLFLFQIYNKDFSEKSTGNKNLHSIYFDGLFLDNNINAQDGVILKLSGGGEIFFRPKTDVKKLGSRTDTKGKLVIKNKRYSQDKIFLHFPIELNYSNTQESNFNKLVRNFLADNPDINIIGVDRGEKHLIYYAGIDQKGNTLKDKDDKDVLGSLNEINGVNYYKLLEERAKAREKARQDWQNIQGIKDLKMGYISLVVRKLADLIIEYNAILVLEDLNMRFKQIHGGIEKSVYQQLEKALIEKLNFLVNKGEKDPERAGHLLRAYQLTAPFSTFKDMGKQTGVLFYTQASYTSKTCPQCGFRPNIKLHFDNLENAKKMLEKINIVYKDNHFEIGYKVSDFTKTEKTSRGNILYGDRQGKDTFVISSKAAIRYKWFARNIKNNELNRGESLKEHTEKGVTIQYDITECLKILYEKNGIDHSGDITKQSIRSELPAKFYKDLLFYLYLLTNTRSSISGTEIDYINCPDCGFHSEKGFNGCIFNGDANGAYNIARKGMLILKKINQYKDQHHTMDKMGWGDLFIGIEEWDKYTQVVSRS

SEQ ID NO: 9 - protein, 1273 aa, synthetic construct
  source 1..1273: note = ART9
MKEIKELTGLYSLTKTIGVELKPVGKTQELIEAKKLIEQDDQRAEDYKIVKDIIDRYHKDFIDKCLNCVKIKKDDLEKYVSLAENSNRDAEDFDKIKTKMRNQITEAFRKNSLFTNLFKKNLIKEYLPAFVSEEEKSVVNKFSKFTTYFDAFNDNRKNLYSGDAKSGTIAYRLIHENLPMFLDNIASFNAISGIGVNEYFSSIETEFTDTLEGKRLTEFFQIDFFNNTLTQKKIGNYNYIVGAVNKAVNLYKQQHKTVRVPLLKPLYKMILSDRVTPSWLPERFESDEEMLTAIKAAYESLREVLVGDNDESLRNLLLNIEHYDLEHIYIANDSGLTSISQKIFGCYDTYTLAIKDQLQRDYPATKKQREAPDLYDERIDKLYKKVGSFSIAYLNRLVDAKGHFTINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 10 - protein, 1300 aa, synthetic construct
  source 1..1300: note = ART10
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 11 - protein, 1205 aa, synthetic construct
  source 1..1205: note = ART11
MYYQGLTKLYPISKTIRNELIPVGKTLEHIRMNNILEADIQRKSDYERVKKLMDDYHKQLINESLQDVHLSYVEEAADLYLNASKDKDIVDKFSKCQDKLRKEIVNLLKSHENFPKIGNKEIIKLLQSLSDTEKDYNALDSFSKFYTYFTSYNEVRKNLYSDEEKSSTAAYRLINENLPKFLDNIKAYSIAKSAGVRAKELTEEEQDCLFMTETFERTLTQDGIDNYNELIGKLNFAINLYNQQNNKLKGFRKVPKMKELYKQILSEREASFVDEFVDDEALLTNVESFSAHIKEFLESDSLSRFAEVLEESGGEMVYIKNDTSKTTFSNIVFGSWNVIDERLAEEYDSANSKKKKDEKYYDKRHKELKKNKSYSVEKIVSLSTETEDVIGKYIEKLQADIIAIKETREVFEKVVLKEHDKNKSLRKNTKAIEAIKSFLDTIKDFERDIKLISGSEHEMEKNLAVYAEQENILSSIRNVDSLYNMSRNYLTQKPFSTEKFKLNFNRATLLNGWDKNKETDNLGILLVKEGKYYLGIMNTKANKSFVNPPKPKTDNVYHKVNYKLLPGPNKMLPKVFFAKSNLEYYKPSEDLLAKYQAGTHKKGENFSLEDCHSLISFFKDSLEKHPDWSEFGFKFSDTKKYDDLSGFYREVEKQGYKITYTDIDVEYIDSLVEKDELYLFQIYNKDFSPYSKGNYNLHTLYLTMLFDERNLRNVVYKLNGEAEVFYRPASIGKDELIIHKSGEEIKNKNPKRAIDKPTSTFEYDIVKDRRYTKDKFMLHIPVTMNFGVDETRRFNEVVNDAIRGDDKVRVIGIDRGERNLLYVVVVDSDGTILEQISLNSIINNEYSIETDYHKLLDEKEGDRDRARKNWTTIENIKELKEGYLSQVVNVIAKLVLKYDAIICLEDLNFGFKRGRQKVEKQVYQKFEKMLIDKLNYLVIDKSRSQENPEEVGHVLNALQLTSKFTSFKELGKQTGIIYYVPAYLTSKIDPTTGFANLFYVKYESVEKSKDFFNRFDSICFNKVAGYFEFSFDYKNFTDRACGMRSKWKVCTNGERIIKYRNEEKNSSFDDKVIVLTEEFKKLFNEYGIAFNDCMDLTDAINAIDDASFFRKLTKLFQQTLQMRNSSADGSRDYIISPVENDNGEFFNSEKCDKSKPKDADANGAFNIARKGLWVLEQLYNSSSGEKLNLAMTNAEWLEYAQQHTI

SEQ ID NO: 12 - protein, 1306 aa, synthetic construct
  source 1..1306: note = ART12
MAKNFEDFKRLYPLSKTLRFEAKPIGATLDNIVKSGLLEEDEHRAASYVKVKKLIDEYHKVFIDRVLDNGCLPLDDKGDNNSLAEYYESYVSKAQDEDAIKKFKEIQQNLLSIIAKKLTDDKAYANLFGNKLIESYKDKADKTKLIDSDLIQFINTAESTQLVSMSQDEAKELVKEFWGFTTYFEGFFKNRKNMYTPEEKSTGIAYRLINENLPKFIDNMEAFKKAIARPEIQANMEELYSNFSEYLNVESIQEMFLLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKHVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNRPLTITKEVFDLNNVLYGKYKKFQKGYLTATGDNVGYTHAVNVWIKFCMDFLDSYDSTCIYDFSSLKPESYLSLDSFYQDVNLLLYKLSFTDVSASFIDQLVEEGKMYLFQIYNKDFSEYSKGTPNMHTLYWKALFDERNLADVVYKLNGQAEMFYRKKSIENTHPTHPANHPILNKNKDNKKKESLFEYDLIKDRRYTVDKFMFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISTQTDKAFFTGLLHILKLTLQMRNSITGTETDYLVSPVADENGIFYDSRSCGDQLPENADANGAYNIARKGLMLVEQIKDAEDLDNVKFDISNKAWLNFAQQKPYKNG

SEQ ID NO: 13 - protein, 1306 aa, synthetic construct
  source 1..1306: note = ART13
MAKNFEDFKRLYSLSKTLRFEAKPIGATLDNIVKSGLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKRVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNKPLTITKEVFDLNNVLYGKYKKFQKGYLTATGDNVGYTHAVNVWIKFCMDFLNSYDSTCIYDFSSLKPESYLSLDAFYQDANLLLYKLSFARASVSYINQLVEEGKMYLFQIYNKDFSEYSKGTPNMHTLYWKALFDERNLADVVYKLNGQAEMFYRKKSIENTHPTHPANHPILNKNKDNKKKESLFDYDLIKDRRYTVDKFMFHVPITMNFKSVGSENINQDVKAYLRHADDMHIIGIDRGERHLLYLVVIDLQGNIKEQYSLNEIVNEYNGNTYHTNYHDLLDVREEERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISAQTDKAFFTGLLHILKLTLQMRNSITGTETDYLVSPVADENGIFYDSRSCGNQLPENADANGAYNIARKGLMLIEQIKNAEDLNNVKFDISNKAWLNFAQQKPYKNG

SEQ ID NO: 14 - protein, 1274 aa, synthetic construct
  source 1..1274: note = ART14
MAKNFEDFKRLYSLSKTLRFEAKPIGATLDNIVKSDLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINDYINLYNQKHKDDKLPKLKALFKQILSDRNAISWLPEEFNSDQEVLNAIKDCYERLSENVLGDKVLKSMLGSLADYSLDGIFIRNDLQLTDISQKMFGNWSVIQNAIMQNIKHVAPARKHKESEEEYENRIAGIFKKADSFSISYIDACLNETDPNNAYFVENYFATLGAVDTPTMQRENLFALVQNAYTEITALLHSDYPTEKNLAQDKANVAKIKALLDAIKSLQHFVKPLLGKGDESDKDERFYGELASLWAELDTMTPLYNMIRNYMTRKPYSQKKIKLNFENPQLLGGWDANKEKDYATIILRRNGLYYLAIMNKDSKKLLGKAMPSDGECYEKMVYKLLPGANKMLPKVFFAKSRMEDFKPSKELVEKYYNGTHKKGKNFNIQDCHNLIDYFKQSIDKHEDWSKFGFKFSDTSTYEDLSGFYREVEQQGYKLSFARVSVSYINQLVEEGKMYLFQIYNKDFSEYSKGTPNMHTLYWKALFDERNLADVVYKLNGQAEMFYRKKSIENTHPTHPANHPILNKNKDNKKKESLFGYDLIKDRRYTVDKFLFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVKYHAIVVLEDLNMGFMRGRQKVEKQVYQKFEKMLIEKLNYLVDKKADASVSGGLLNAYQLTSKFDSFQKLGKQSGFLFYIPAWNTSKIDPVTGFVNLLDTRYQNVEKAKSFFSKFDAIRYNKDKEWFEFNLDYDKFGKKAEGTRTKWTLCTRGMRIDTFRNKEKNSQWDNQEVDLTAEMKSLLEHYYIDIHSNLKDAISAQTDKAFFTGLLHILKLTLQMRNSITGTETDYLVSPVVDENGIFYDSRSCGDELPENADANGAYNIARKGLMMIEQIKDAKDLDNLKFDISNKAWLNFAQQKPYKNG

SEQ ID NO: 15 - protein, 1251 aa, synthetic construct
  source 1..1251: note = ART15
MLFQDFTHLYPLSKTVRFELKPIGRTLEHIHAKNFLSQDETMADMYQKVKVILDDYHRDFIADMMGEVKLTKLAEFYDVYLKFRKNPKDDELQKQLKDLQAVLRKESVKPIGNGGKYKAGHDRLFGAKLFKDGKELGDLAKFVIAQEGKSSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIKHHTARHDDESVQAGKLGQYFKHGLAGVDNPIQKIHNNHSTIKGFLERERPAGERALPKIKSGKNPEMTQLRQLKELLDNALNVAHFAKLLMTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMIYKLLPGPNKMLPRVFFAKSNLDYYNPSAELLDKYAQGTHKKGDNFNLKDCHALIDFFKAGINKHPEWQNFGFKFSPTSSYRDLSDFYREVEPQGYQVKFVDINADYIDELVEQGQLYLFQIYNKDFSPKAHGKPNLHTLYFRALFSEDNLANPIYKLNGEAQIFYRKASLGMNETTIHRAGEILENKNPDNPKERVFTYDIIKDRRYTQDKFMLHVPITMNFGVQGMTIKEFNKKVNQSIRQYDDVNVIGIDRGERHLLYLTVINSKGEILEQRSLNDITTASANGTQMTTPYHKILDKREIERLNARVGWGEIETIKELKSGYLSHVVHQVSQLMLKYNAIVVLEDLNFGFKRGRFKVEKQIYQNFENALIKKLNHLELKDKADDEIGSYKNALQLTNNFTDLKNIGKQTGFLFYVPAWNTSKIDPETGFVDLLKPRYENIAQSQAFFGKFDKICYNADKDYFEFHIDYAKFTDKAKNSRQTWTICSHGDKRYVYDKTANQNKGATKGINVNDELKSLFARYHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 16 - protein, 1251 aa, synthetic construct
  source 1..1251: note = ART16
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGPNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 17 - protein, 1251 aa, synthetic construct
  source 1..1251: note = ART17
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGSNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 18 - protein, 1257 aa, synthetic construct
  source 1..1257: note = ART18
MKYTDFTGIYPVSKTLRFELIPQGSTVENMKREGILNNDMHRADSYKEMKKLIDEYHKVFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFHIVAKVPEMQEHFKTIADELRSHLQVGDDIDKMFNLQFFNKVLTQSQLAVYNAVIGGKSEGNKKIQGINEYVNLYNQQHKKARLPMLKLLYKQILSDRVAISWLQDEFDNDQDMLDTIEAFYNKLDSNETGVLGEGKLKQILMGLDGYNLDGVFLRNDLQLSEVSQRLCGGWNIIKDAMISDLKRSVQKKKKETGADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDSFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHTLLEEKEAGNKKARQDWQTIESIKELKEGYLSQVIHKISMLMQRYHAIVVLEDLNGSFMRSRQKVEKQVYQKFEHMLINKLNYLVNKQYDAAEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 19 - protein, 1245 aa, synthetic construct
  source 1..1245: note = ART19
METFSGFTNLYPLSKTLRFRLIPVGETLKYFIGSGILEEDQHRAESYVKVKAIIDDYHRAYIENSLSGFELPLESTGKFNSLEEYYLYHNIRNKTEEIQNLSSKVRTNLRKQVVAQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAIVNFWNTIHDTVLAEGGLKTIIASLGSYGLEGIFLKNDLQLTDISQKATGSWGKISSEIKQKIEVMNPQKKKESYETYQERIDKIFKSYKSFSLAFINECLRGEYKIEDYFLKLGAVNSSSLQKENHFSHILNTYTDVKEVIGFYSESTDTKLIRDNGSIQKIKLFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 20 - protein, 1245 aa, synthetic construct
  source 1..1245: note = ART20
METFSGFTNLYPLSKTLRFRLIPVGETLKHFIDSGILEEDQHRAESYVKVKAIIDDYHRAYIENSLSGFELPLESTGKFNSLEEYYLYHNIRNKTEEIQNLSSKVRTNLRKQVVVQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAMVNFWNTIHDTVLAEGGLKTIIASLGSYGLEGIFLKNDLQLTDISQKATGSWSKISSEIKQKIEVMNPQKKKESYESYQERIDKLFKSYKSFSLAFINECLRGEYKIEDYFLKLGAVNSSSLQKENHFSHILNAYTDVKEAIGFYSESTDTKLIQDNDSIQKIKQFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDGNCYEKMEYKLLPGANKMLPKVFFSKSRINEFMPNERLLSNYEKGTHKKSGICFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLCSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNALGKQSGFLFYIPAWNTSKMDPVTGFVNLFDVKYESVDKAKSFFSKFDSMRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 21 - protein, 1245 aa, synthetic construct
  source 1..1245: note = ART21
METFSGFTNLYPLSKTLRFRLIPVGETLKHFIGSGILEEDQHRAESYVKVKAIIDDYHRTYIENSLSGFELPLESTGKFNSLEEYYLYHNIRNKTEEIQNLSSKVRTNLRKQVVTQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLLEKFENDSQVVGAMVNFWNTIHDTVLAEGGLKTIIASLGSYGLEGIFLKNDLQLTDISQKATGSWSKISSEIKQKIEAMNPQKKKESYESYQERIDKLFKSYKSFSLAFVNECLRGEYKIEDYFLKLGAVNSSLLQKENHFSHILNTYTDVKEVIGFYSESTDTKLIQDNDSIQKIKQFLDAVKDLQAYVKPLLGNSDETGKDERFYGDLIEYWSLLDLITPLYNMVRNYVTQKPYSVDKIKINFQNPTLLNGWDLNKEMDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLNKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKANGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYDEFTKKAEGTKTDWTVCSYGNRIITFRNPDKNSQWDNKEINLTENIKLLFERFGIDLSSNLKDEIMERTEKEFFIELISLFKLVLQMRNSWTGTDIDYLVSPVCNENGEFFDSRNVDETLPQNADANGAYNIARKGMILLDKIKKNNGEKKLTLSITNREWLSFAQGCCKNG

SEQ ID NO: 22 - protein, 1261 aa, synthetic construct
  source 1..1261: note = ART22
MLFQDFTHLYPLSKTVRFELKPIGKTLEHIHAKNFLSQDKTMADMYQKVKAILDDYHRDFIADMMGEVKLTKLAEFCDVYLKFRKNPKDDGLQKQLKDLQAVLRKEIVKPIGNGGKYKVGYDRLFGAKLFKDGKELGDLAKFVIAQESESSPKLPQIAHFEKFSTYFTGFHDNRKNMYSSDDKHTAIAYRLIHENLPRFIDNLQILATIKQKHSALYDQIASELTASGLDVSLASHLGGYHKLLTQEGITAYNRIIGEVNSYTNKHNQICHKSERIAKLRPLHKQILSDGMGVSFLPSKFADDSEMCQAVNEFYRHYADVFAKVQSLFDRFDDYQKDGIYVEHKNLNELSKRAFGDFGFLKRFLEEYYADVIDPEFNEKFAKTEPDSDEQKKLAGEKDKFVKGVHSLASLEQVIEYYTAGYDDESVQADKLGQYFKHRLAGVDNPIQKIHNSHSTIKGFLERERPAGERALPKIKSDKSPEMTQLRQLKELLDNALNVVHFAKLVSTETVLDTRSDKFYGEFRPLYVELAKITTLYNKVRDYLSQKPFSTEKYKLNFGNPTLLNGWDLNKEKDNFGVILQKDGCYYLALLDKAHKKVFDNAPNTGKSVYQKMVYKQIANARRDLACLLIINGKVVRKTKGLDDLREKYLPYDIYKIYQSESYKVLSPNFNHQDLVKYIDYNKILASGYFEYFDFRFKESSEYKSYKEFLDDVDNCGYKISFCNINADYIDELVEQGQLYLFQIYNKDFSPKAHGKPNLHTLYFKALFSEDNLANPIYKLNGEAQIFYRKASLDMNETTIHRAGEVLENKNPDNPKQRQFVYDIIKDKRYTQDKFMLHVPITMNFGVQGMTIEGFNKKVNQSIQQYDDVNVIGIDRGERHLLYLTVINSKGEILEQRSLNDIITTSANGTQMTTPYHKILNKKKEGRLQARKDWGEIETIKELKAGYLSHVVHQISQLMLKYNAIVVLEDLNFGFKRGRLKVENQVYQNFENALIKKLNHLVLKDKTDDEIGSYKNALQLTNNFTDLKSIGKQTGFLFYVPARNTSKIDPETGFVDLLKPRYENITQSQAFFGKFDKICYNTDKGYFEFHIDYAKFTDEAKNSRQTWVICSHGDKRYVYNKTANQNKGATKGINVNDELKSLFACHHINDKQPNLVMDICQNNDKEFHKSLMYLLKALLALRYSNANSDEDFILSPVANDEGVFFNSALADDTQPQNADANGAYHIALKGLWVLEQIKNSDDLDKVDLEIKDDEWRNFAQNR

SEQ ID NO: 23 - protein, 1275 aa, synthetic construct
  source 1..1275: note = ART23
MGKNQNFQEFIGVSPLQKTLRNELIPTETTKKNITQLDLLTEDEIRAQNREKLKEMMDDYYRDVIDSTLHAGIAVDWSYLFSCMRNHLRENSKESKRELERTQDSIRSQIYNKFAERADFKDMFGASIITKLLPTYIKQNPEYSERYDESMEILKLYGKFTTSLTDYFETRKNIFSKEKISSAVGYRIVEENAEIFLQNQNAYDRICKIAGLDLHGLDNEITAYVDGKTLKEVCSDEGFAKAITQEGIDRYNEAIGAVNQYMNLLCQKNKALKPGQFKMKRLHKQILCKGTTSFDIPKKFENDKQVYDAVNSFTEIVMKNNDLKRLLNITQNVNDYDMNKIYVAADAYSTISQFISKKWNLIEECLLDYYSDNLPGKGNAKENKVKKAVKEETYRSVSQLNELIEKYYVEKTGQSVWKVESYISRLAETITLELCHEIENDEKHNLIEDDDKISKIKELLDMYMDAFHIIKVFRVNEVLNFDETFYSEMDEIYQDMQEIVPLYNHVRNYVTQKPYKQEKYRLYFNTPTLANGWSKNKEYDNNAIILMRDDKYYLGILNAKKKPSKQTMAGKEDCLEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 24 - protein, 1261 aa, synthetic construct
  source 1..1261: note = ART24
MNTSLFSSFTRQYPVTKTLRFELKPMGATLGHIQQKGFLHKDEELAKIYKKIKELLDEYHRAFIADTLGDAQLVGLDDFYADYQALKQDSKNSHLKDKLTKTQDNLRKQITKNFEKTPQLKERYKRLFTKELFKAGKDKGDLEKWLINHDSEPNKAEKISWIHQFENFTTYFQGFYENRKNMYSDEVKHTAIAYRLIHENLPRFVDNIQVLSKIKSDYPDLYHELNHLDSRTIDFADFKFDDMLQMDFYHHLLIQSGITAYNTLLGGKVLEGGKKLQGINELINLYGQKHKIKIAKLKPLHKQILSDGQSVSFLPKKFDNDYELCQTVNHFYREYVAIFDELVVLFQKFYDYDKDNIYINHQQLNQLSHELFADFRLLSRALDFYYCQIIDGDFNNKINNAKSQNAKEKLLKEKERYTKSNHSINELQKAINHYASHHEDTEVKVISDYFSATNIRNMIDGIHHHFSTIKGFLEKDNNQGESYLPKQKNSNDVKNLKLFLDGVLRLIHFIKPLALKSDDTLEKEEHFYGEFMPLYDKLVMFTLLYNKVRDYISQKPYNDEKIKLNFGNSTLLNGWDVNKEKDNFGVILCKEGLYYLAILDKSHKKVFDNAPKATSSHTYQKMVYKLLPGPNKMLPKVFFAKSNIGYYQPSAQLLENYEKGTHKKGSNFSLTDCHHLIDFFKSSIAKHPEWKEFGFRFSDTHTYQDLSDFYKEIEPQSYKVKFIDIDADYIDDLVEKGQLYLFQLYNKDFSKQSYGKPNLHTLYFKSLFSDDNLKNPIYKLNGEAEIFYRRASLSVSDTTIHQAGEILTPKNPNNTHNRTLSYDVIKNKRYTTDKFFLHIPITMNFGIENTGFKAFNHQVNTTLKNADKKDVHIIGIDRGERHLLYVSVIDGDGRIVEQRTLNDIVSISNNGMSMSTPYHQILDNREKERLAARTDWGDIKNIKELKAGYLSHVVHEVVQMMLKYNAMIVLEDLNFGFKHGRFKVEKQVYQNFENALIKKLNYLVLKNADNHQLGSVRKALQLTNNFTDIKSIGKQTGFIFYVPAWNTSKIDPTTGFVDLLKPRYENMAQAQSFISRFKKIAYNHQLDYFEFEFDYADFYQKTIDKKRIWTLCTYGDVRYYYDHKTKETKTVNITKELKSLLDKHDLSYQNGHNLVDELANSHDKSLLSGVMYLLKVLLALRYSHAQKNEDFILSPVMNKDGVFFDSRFADDVLPNNADANGAYHIALKGLWVLNQIQSADNMDKIDLSISNEQWLHFTQSR

SEQ ID NO: 25 - protein, 1288 aa, synthetic construct
  source 1..1288: note = ART25
MVGNKISNSFDSFTGINALSKTLRNELIPSDYTKRHIAESDFIAADTNKNEDQYVAKEMMDDYYRDFISKVLDNLHDIEWKNLFELMHKAKIDKSDATSKELIKIQDMLRKKIGKKFSQDPEYKVMLSAGMITKILPKYILEKYETDREDRLEAIKRFYGFTVYFKEFWASRQNVFSDKAIASSISYRIIHENAKIYMDNLDAYNRIKQIACEEIEKIEEEAYDFLQGDQLDVVYTEEAYGRFISQSGIDLYNNICGVINAHMNLYCQSKKCSRSKFKMQKLHKQILCKAETGFEIPLGFQDDAQVINAINSFNALIKEKNIISRLRTIGKSISLYDVNKIYISSKAFENVSVYIDHKWDVIASSLYKYFSEIVKGNKDNREEKIQKEIKKVKSCSLGDLQRLVNSYYKIDSTCLEHEVTEFVTKIIDEIDNFQITDFKFNDKISLIQNEQIVMDIKTYLDKYMSIYHWMKSFVIDELVDKDMEFYSELDELNEDMSEIVNLYNKVRNYVTQKPYSQEKIKLNFGSPTLADGWSKSKEFDNNAIILIRDEKIYLAIFNPRNKPAKTVISGHDVCNSETDYKKMNYYLLPGASKTLPHVFIKSRLWNESHGIPDEILRGYELGKHLKSSVNFDVEFCWKLIDYYKECISCYPNYKAYNFKFADTESYNDISEFYREVECQGYKIDWTYISSEDVEQLDRDGQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKKLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDENGRFYDSENYIFNEETELPKDADANGAYCIALKGLYNVIAIKNNWKEGEKFNRKLLSLNNYNWFDFIQNRRF

SEQ ID NO: 26 - protein, 1876 aa, synthetic construct
  source 1..1876: note = ART26
MVGNKISNSFDSFTGINALSKTLRNELIPSDYTKRHIAESDFIAADTNKNEDQYVAKEMMDDYYRDFISKVLDNLHDIEWKNLFELMHKAKIDKSDATSKELIKIQDMLRKKIGKKFSQDPEYKVMLSAGMITKILPKYILEKYETDREDRLEAIKRFYGFTVYFKEFWASRQNVFSDKAIASSISYRIIHENAKIYMDNLDAYNRIKQIACEEIEKIEEEAYDFLQGDQLDVVYTEEAYGRFISQSGIDLYNNICGVINAHMNLYCQSKKCSRSKFKMQKLHKQILCKAETGFEIPLGFQDDAQVINAINSFNALIKEKNIISRLRTIGKSISLYDVNKIYISSKAFENVSVYIDHKWDVIASSLYKYFSEIVKGNKDNREEKIQKEIKKVKSCSLGDLQRLVNSYYKIDSTCLEHEVTEFVTKIIDEIDNFQITDFKFNDKISLIQNEQIVMDIKTYLDKYMSIYHWMKSFVIDELVDKDMEFYSELDELNEDMSEIVNLYNKVRNYVTQKPYSQEKIKLNFGSPTLADGWSKSKEFDNNAIILIRDEKIYLAIFNPRNKPAKTVISGHDVCNSETDYKKMNYYLLPGASKTLPHVFIKSRLWNESHGIPDEILRGYELGKHLKSSVNFDVEFCWKLIDYYKECISCYPNYKAYNFKFADTESYNDISEFYREVECQGYKIDWTYISSEDVEQLDRDGQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKKLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDENGRFYDSENYIFNEETELPKDADANGAYCIALKGLYNVIAIKNNWKEGEKFNRKLLSLNNYNWFDFIQNRRFQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKRLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDEKGRFYDSENYIFNEETELPKDADANGAYCIALKGLYNVIAIKNNWKEGEKFNRKLLSLNNYNWFDFIQNRRF

SEQ ID NO: 27 - protein, 1245 aa, synthetic construct
  source 1..1245: note = ART27
MQEHKKISHLTHRNSVQKTIRMQLNPVGKTMDYFQAKQILENDEKLKEDYQKIKEIADRFYRNLNEDVLSKTGLDKLKDYAEIYYHCNTDADRKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAVDDLDTTYSGLCGTNLYDVFTVDYFNFLLPQSGITEYNKIIGGYTTSDGTKVKGINEYINLYNQQVSKRYKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYKEAAPLFNESYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIRKNGTFKKGDKFSLDDCHKLIDFYKESFKKYPNWLIYNFKFKKTNEYNDISEFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKRASTFPYDLIKDKRYTKWQFSLHFPITMNFKAPDRAMINDDVRNLLKSCNNNFIIGIDRGERNLLYVSIIDSNGAIIYQHSLNIIGNKFKGKTYETNYREKLETREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGMQSGFIFYVRPDFTSKIDPVTGFVNLLYPRYENIDKAKDMISRFDDIRYNAGEDFFEFDIDYDKFPKTASDYRKKWTICTNGERIEAFRNPASNNEWSYRTIILAEKFKELFDNNSINYRDSDNLKAEILSQTKGKFFEDFFKLLRLTLQMRNSNPETGEDRILSPVKDKNGNFYDSSKYDEKSNLPCDADANGAYNIARKGLWIVEQFKKSDNVSTVEPVIHNDKWLKFVQENDMANN

SEQ ID NO: 28 - protein, 1271 aa, synthetic construct
  source 1..1271: note = ART28
MKNLANFTNLYSLQKTLRFELKPIGKTLDWIIKKDLLKQDEILAEDYKIVKKIIDRYHKDFIDLAFESAYLQKKSSDSFTAIMEASIQSYSELYFIKEKSDRDKKAMEEISGIMRKEIVECFTGKYSEVVKKKFGNLFKKELIKEDLLNFCEPDELPIIQKFADFTTYFTGFHENRENMYSNEEKATAIANRLIRENLPRYLDNLRIIRSIQGRYKDFGWKDLESNLKRIDKNLQYSDFLTENGFVYTFSQKGIDRYNLILGGQSVESGEKIQGLNELINLYRQKNQLDRRQLPNLKELYKQILSDRTRHSFVPEKFSSDKALLRSLLDFHKEVIQNKNLFEEKQVSLLQAIRETLTDLKSFDLDRIYLTNDTSLTQISNFVFGDWSKVKTILAIYFDENIANPKDRQRQSNSYLKAKENWLKKNYYSIHELNEAISVYGKHSDEELPNTKIEDYFSGLQTKDETKKPIDVLDAIVSKYADLESLLTKEYPEDKNLKSDKGSIEKIKNYLDSIKLLQNFLKPLKPKKVQDEKDLGFYNDLELYLESLESANSLYNKVRNYLTGKEYSDEKIKLNFKNSTLLDGWDENKETSNLSVIFRDTNNYYLGILDKQNNRIFESIPEIQSGEETIQKMVYKLLPGANNMLPKVFFSEKGLLKFNPSDEITSLYSEGRFKKGDKFSINSLHTLIDFYKKSLAVHEDWSVFNFKFDETSHYEDISQFYRQVESQGYKITFKPISKKYIDTLVEDGKLYLFQIYNKDFSQNKKGGGKPNLHTIYFKSLFEKENLKDVIVKLNGQAEVFFRKKSIHYDENITRYGHHSELLKGRFSYPILKDKRFTEDKFQFHFPITLNFKSGEIKQFNARVNSYLKHNKDVKIIGIDRGERHLLYLSLIDQDGKILRQESLNLIKNDQNFKAINYQEKLHKKEIERDQARKSWGSIENIKELKEGYLSQVVHTISKLMVEHNAIVVLEDLNFGFKRGRQKVERQVYQKFEKMLIEKLNFLVFKDKEMDEPGGILKAYQLTDNFVSFEKMGKQTGFVFYVPAWNTSKIDPKTGFVNFLHLNYENVNQAKELIGKFDQIRYNQDRDWFEFQVTTDQFFTKENAPDTRTWIICSTPTKRFYSKRTVNGSVSTIEIDVNQKLKELFNDCNYQDGEDLVDRILEKDSKDFFSKLIAYLRILTSLRQNNGEQGFEERDFILSPVVGSDGKFFNSLDASSQEPKDADANGAYHIALKGLMNLHVINETDDESLGKPSWKISNKDWLNFVWQRPSLKA

SEQ ID NO: 29 - protein, 1245 aa, synthetic construct
  source 1..1245: note = ART29
MQEHKKISHLTHRNSVQKTIRMQLNPVGKTMDYFQAKQILENDEKLKENYQKIKEIADRFYRNLNEDVLSKTRLDKLKDYTDIYYHCNTDADRKRLDECASELRKEIVKNFKNRDEYNKLFNKKMIEIVLPKHLKNEDEKEVVTSFKNFTTYFTGFFTNRKNMYSDGEESTAIAYRCINENLPKHLDNVKAFEKAISKLSKNAIDDLDTTYSGLCGTNLYDVFTVDYFNFLLPQSGITEYNKIIGGYTTNDGTKVKGINEYINLYNQQVSKRDKIPNLKILYKQILSESEKVSFIPPKFEDDNELLSAVSEFYANDETFDGMPLKKAIDETKLLFGNLDNPSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIYKSGTFKTGDKFSLDDCHKLIDFYKESFKKYPNWLIYNFKFKKTNEYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKKTSTFPYDLIKDKRYTKWQFSLHFPITMNFKAPDKAMINDDVRNLLKSCNNNFIIGIDRGERNLLYVSVIDSNGAIIYQHSLNIIGNKFKEKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGMQSGFIFYVRPDFTSKIDPVTGFVNLLYPQYENIDKAKDMISRFDEIRYNAGEDFFEFDIDYDEFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDQWLKFVQENDMANN

SEQ ID NO: 30 - protein, 1245 aa, synthetic construct
  source 1..1245: note = ART30
MQEHKKISHLTHRNSVQKTIRMQLNPVGKTMDYFQAKQILENDEKLKEDYQKIKEIADRFYRNLNEDVLSKTGLDKLKDYADIYYHCNTDADRKRLNECASELRKEIVKNFKNRDEYNKLFNKKMIEIVLPKHLKNEDEKEVVASFKNFTTYFTGFFTNRKNMYSDGEESTAIAYRCINENLPKHLDNVKVFEKAISKLSKNAIDDLGATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYKEAAPLFSENYDNEKGLKNDDKSISLIKNFLDAIKEIEKFIKPLSETNITGEKNDLFYSQFTPLLDNISRIDILYDKVRNYVTQKPFSTDKIKLNFGNSQLLNGWDKDKEREYGAVLLCKDEKYYLAIIDKSNNSILENIDFQDCNESDYYEKIVYKLLTKINGNLPRVFFSEKRKKLLSPSDEILKIYKSGTFKKGDKFSLDDCHKLIDFYKESFKKYPNWLIYNFKFKNTNEYNDISEFYNDVASQGYNISKMKIPTTFIDKLVDEGKIYLFQLYNKDFSPHSKGTPNLHTLYFKMLFDERNLEDVVYKLNGEAEMFYRPASIKYDKPTHPKNTPIKNKNTLNDKKASTFPYDLIKDKRYTKWQFSLHFPITMNFKAPDKAMINDDVRNLLKSCNNNFIIGIDRGERNLLYVSVIDSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 31 - protein, 1244 aa, synthetic construct
  source 1..1244: note = ART31
MQERKKISHLTHRNSVKKTIRMQLNPVGKTMDYFQAKQILENDEKLKENYQKIKEIADRFYRNLNEDVLSKTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDAYSGLCGTNLYDVFTVDYFNFLLPQSGITEYNKIIGGYTTNDGTKVKGINEYINLYNQQVSKRDKIPNLQILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIRKKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCKDEKYYLAIIDKSNNRILENIDFQDCDESDCYEKIIYKLLPTPNKMLPKVFFAKKHKKLLSPSDEILKIYKNGTFKKGDKFSLDDCHKLIDFYKESFKKYPKWLIYNFKFKKTNGYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKRASTFPYDLIKDKRYTKWQFSLHFPITMNFKDPDKAMINDDVRNLLKSCNNNFIIGIDRGERNLLYVSVINSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYEKIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 32 - protein, 1175 aa, synthetic construct
  source 1..1175: note = ART32
KTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLQILYKQILSESEKVSFIPPKFEDDNELLSAVSEFYANDETFDEMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENNEIREKSIVDYYKTSLMQLTDNLSDKYNEVAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCRDEKYYLAIIDKSNNRILENIDFQDCDESDCYEKIIYKLLPTPNKMLPKVFFAKKHKKLLSPSDEILKIRKNGTFKKGDKFSLDDCHKLIDFYKESFKKYPNWLIYNFKFKKTNEYNDIREFYNDVALQGYNISKMKIPTSFIDKLVDEGKIYLFQLYNKDFSPHSKGTPNLHTLYFKMLFDERNLEDVVYKLNGEAKMFYRPASIKYDKPTHPKNTPIKNKNTLNDKKASTFPYDLIKDKRYTKWQFSLHFSITMNFKAPDKAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVEPVIHNDKWLKFVQENDMANN

SEQ ID NO: 33 - protein, 1278 aa, synthetic construct
  source 1..1278: note = ART33
MSININKFSDECRKIDFFTDLYNIQKTLRFSLIPIGATADNFEFKGRLSKEKDLLDSAKRIKEYISKYLADESDICLSQPVKLKHLDEYYELYITKDRDEQKFKSVEEKLRKELADLLKEILKRLNKKILSDYLPEYLEDDEKALEDIANLSSFSTYFNSYYDNCKNMYTDKEQSTAIPYRCINDNLPKFIDNMKAYEKALEELKPSDLEELRNNFKGVYDTTVDDMFTLDYFNCVLSQSGIDSYNAIIGNDKVKGINEYINLHNQTAEQGHKVPNLKRLYKQIGSQKKTISFLPSKFESDNELLKAVYDFYNTGDAEKNFTALKDTITEFEKIFDNLSEYNLDGVFVRNDISLTNLSQSMFNDWSVFRNLWNDQYDKVNNPEKAKDIDKYNDKRHKVYKKSESFSINQLQELIATTLEEDINSKKITDYFSCDFHRVTTEVENKYQLVKDLLSSDYPKNKNLKTSEEDVALIKDFLDSVKSLESFVKILTGTGKESGKDELFYGSFTKWFDQLRYIDKLYDKVRNYITEKPYSLDKIKLSFDNPQFLGGWQHSKETDYSAQLFMKDGLYYLGVMDKETKREFKTQYNTPENDSDTMVKIEYNQIPNPGRVIQNLMLVDGKIVKKNGRKNADGVNAVLEELKNQYLPENINRIRKTESYKTTSNNFNKDDLKAYLEYYIARTKEYYCKYNFVFKSADEYGSFNEFVDDVNNQAYQITKVKVSEKQLLSLVEQGKLYLFKIYNKDFSEYSKGKKNLHTMYFQMLFDDRNLENLVYKLQGGAEMFYRPASIKKDSEFKHDANVEIIKRTCEDKVNDKDNPTDDEKAKYYSKFDYDIVKNKRFTKDQFSLHLTLAMNCNQPDHYWLNNDVRELLKKSNKNHIIGIDRGERNLIYVTIINSDGVIVDQINFNIIENSYNGKKYKTDYQKKLNQREEDRQKARKTWKTIETIKELKDGYISQVVHQICKLIVQYDAIVVMENLNGGFKRGRTKVEKQVYQKFETMLINKLNYYVDKGTDYKECGGLLKAYQLTNKFETFERIGKQSGIIFYVDPYLTSKIDPVTGFANLLYPKYETIPKTHNFISNIDDIRYNQSEDYFEFDIDYDKFPQGSYNYRKKWTICSYGNRIKYYKDSRNKTASVVVDITEKFKETFTNAGIDFVNDNIKEKLLLVNSKELLKSFMDTLKLTVQLRNSEINSDVDYIISPIKDRNGNFYYSENYKKSNNEVPSQPQDGDANGAYNIARKGLMIINKLKKADDVTNNELLKISKKEWLEFAQKGDLGE

SEQ ID NO: 34 - protein, 1562 aa, synthetic construct
  source 1..1562: note = ART34
MKATSIWDNFTRKYSVSKTLRFELRPVGKTEENIVKKEIIDAEWISGKNIPKGTDADRARDYKIVKKLLNQLHILFINQALSSENVKEFEKEDKKSKTFVAWSDLLATHFDNWIQYTRDKSNSTVLKSLEKSKKDLYSKLGKLLNSKANAWKAEFISYHKIKSPDNIKIRLSASNVQILFGNTSDPIQLLKYQIELDNIKFLKDDGSEYTTKELADLLSTFEKFGTYFSGFNQNRANVYDIDGEISTSIAYRLFNQNIEFFFQNIKRWEQFTSSIGHKEAKENLKLVQWDIQSKLKELDMEIVQPRFNLKFEKLLTPQSFIYLLNQEGIDAFNTVLGGIPAEVKAEKKQGVNELINLTRQKLNEDKRKFPSLQIMYKQIMSERKTNFIDQYEDDVEMLKEIQEFSNDWNEKKKRHSASSKEIKESAIAYIQREFHETFDSLEERATVKEDFYLSEKSIQNLSIDIFGGYNTIHNLWYTEVEGMLKSGERPLTRVEKEKLKKQEYISFAQIERLISKHSQQYLDSTPKEANDRSLFKEKWKKTFKNGFKVSEYTNLKLNELISEGETFQKIDQETGKETTIKIPGLFESYENAILVESIKNQSLGTNKKESVPSIKEYLDSCLRLSKFIESFLVNSKDLKEDQSLDGCSDFQNTLTQWLNEEFDVFILYNKVRNHVTKKPGNTDKIKINFDNATLLDGWDVDKEAANFGFLLKKADNYYLGIADSSFNQDLKYFNEGERLDEIEKNRKNLEKEESKNISKIDQEKVKKYKEVIDDLKAISNLNKGRYSKAFYKQSKFTTLIPKCTTQLNEVIEHFKKFDTDYRIENKKFAKPFIITKEVFLLNNTVYDTATKKFTLKIGEDEDTKGLKKFQIGYYRATDDKKGYESALRNWITFCIEFTKSYKSCLNYNYSSLKSVSEYKSLDEFYKDLNGIGYTIDFVDISEEYINKKINEGKLYLFQIYNKDFSEKSKGKENLHTTYWKLLFDSKNLEDVVIKLNGQAEVFFRPASIHEKEKITHFKNQEIQNKNPNAVKKTSKFEYDIIKDNRFTKNKFLFHCPITLNFKADGNPYVNNEVQENIAKNPNVNIIGIDRGEKHLLYFTVINQQGQILDAGSLNSIKSEYKDKNQQSVSFETPYHKILDKKESERKEARESWQEIENIKELKAGYLSHVVHQLSNLIVKYNAIVVLEDLNKGFKRGRFKVEKQVYQKFEKSLIEKLNYLVFKDRKESNEPGHHLNAYQLTNKFLSFERLGKQSGVLFYATASYTSKVDPVTGFMQNIYDPYHKEKTREFYKNFTKIVYNGNYFEFNYDLNSVKPDSEEKRYRTNWTVCSCVIRSEYDSNSKTQKTYNVNDQLVKLFEDAKIKIENGNDLKSTILEQDDKFIRDLHFYFIAIQKMRVVDSKIEKGEDSNDYIQSPVYPFYCSKEIQPNKKGFYELPSNGDSNGAYNIARKGIVILDKIRLRVQIEKLFEDGTKIDWQKLPNLISKVKDKKLLMTVFEEWAELTHQGEVQQGDLLGKKMSKKGEQFAEFIKGLNVTKEDWEIYTQNEKVVQKQIKTWKLFSNST

SEQ ID NO: 35 - protein, 1742 aa, synthetic construct
  source 1..1742: note = ART35
MKAINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNGMKAINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 36 - protein, 1205 aa, synthetic construct
  source 1..1205: note = ART11*
MYYQGLTKLYPISKTIRNELIPVGKTLEHIRMNNILEADIQRKSDYERVKKLMDDYHKQLINESLQDVHLSYVEEAADLYLNASKDKDIVDKFSKCQDKLRKEIVNLLKSHENFPKIGNKEIIKLLQSLSDTEKDYNALDSFSKFYTYFTSYNEVRKNLYSDEEKSSTAAYRLINENLPKFLDNIKAYSIAKSAGVRAKELTEEEQDCLFMTETFERTLTQDGIDNYNELIGKLNFAINLYNQQNNKLKGFRKVPKMKELYKQILSEREASFVDEFVDDEALLTNVESFSAHIKEFLESDSLSRFAEVLEESGGEMVYIKNDTSKTTFSNIVFGSWNVIDERLAEEYDSANSKKKKDEKYYDKRHKELKKNKSYSVEKIVSLSTETEDVIGKYIEKLQADIIAIKETREVFEKVVLKEHDKNKSLRKNTKAIEAIKSFLDTIKDFERDIKLISGSEHEMEKNLAVYAEQENILSSIRNVDSLYNMSRNYLTQKPFSTEKFKLNFNRATLLNGWDKNKETDNLGILLVKEGKYYLGIMNTKANKSFVNPPKPKTDNVYHKVNYKLLPGPNKMLPKVFFAKSNLEYYKPSEDLLAKYQAGTHKKGENFSLEDCHSLISFFKDSLEKHPDWSEFGFKFSDTKKYDDLSGFYREVEKQGYKITYTDIDVEYIDSLVEKDELYFFQIYNKDFSPYSKGNYNLHTLYLTMLFDERNLRNVVYKLNGEAEVFYRPASIGKDELIIHKSGEEIKNKNPKRAIDKPTSTFEYDIVKDRRYTKDKFMLHIPVTMNFGVDETRRFNEVVNDAIRGDDKVRVIGIDRGERNLLYVVVVDSDGTILEQISLNSIINNEYSIETDYHKLLDEKEGDRDRARKNWTTIENIKELKEGYLSQVVNVIAKLVLKYDAIICLEDLNFGFKRGRQKVEKQVYQKFEKMLIDKLNYLVIDKSRSQENPEEVGHVLNALQLTSKFTSFKELGKQTGIIYYVPAYLTSKIDPTTGFANLFYVKYESVEKSKDFFNRFDSICFNKVAGYFEFSFDYKNFTDRACGMRSKWKVCTNGERIIKYRNEEKNSSFDDKVIVLTEEFKKLFNEYGIAFNDCMDLTDAINAIDDASFFRKLTKLFQQTLQMRNSSADGSRDYIISPVENDNGEFFNSEKCDKSKPKDADANGAFNIARKGLWVLEQLYNSSSGEKLNLAMTNAEWLEYAQQHTI

SEQ ID NO: 37 - protein, 1263 aa, synthetic construct
  source 1..1263: note = MAD7
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 38 - protein, 1334 aa, synthetic construct
  source 1..1334: note = MAD2
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 39 - protein, 10 aa, synthetic construct
  source 1..10: note = peptide motif
YLFQIYNKDF

SEQ ID NO: 40 - protein, 7 aa, synthetic construct
  source 1..7: note = NLS of SV40 large T-antigen
PKKKRKV

SEQ ID NO: 41 - protein, 16 aa, synthetic construct
  source 1..16: note = nucleoplasmin bipartite NLS
KRPAATKKAGQAKKKK

SEQ ID NO: 42 - protein, 9 aa, synthetic construct
  source 1..9: note = c-myc NLS
PAAKRVKLD

SEQ ID NO: 43 - protein, 11 aa, synthetic construct
  source 1..11: note = c-myc NLS
RQRRNELKRSP

SEQ ID NO: 44 - protein, 38 aa, synthetic construct
  source 1..38: note = hRNPA1 M9 NLS
NQSSNFGPMKGGNFGGRSSGPYGGGGQYFAKPRNQGGY

SEQ ID NO: 45 - protein, 42 aa, synthetic construct
  source 1..42: note = importin-a IBB domain NLS
RMRIZFKNKGKDTAELRRRRVEVSVELRKAKKDEQILKRRNV

SEQ ID NO: 46 - protein, 8 aa, synthetic construct
  source 1..8: note = myoma T protein NLS
VSRKRPRP

SEQ ID NO: 47 - protein, 8 aa, synthetic construct
  source 1..8: note = myoma T protein NLS
PPKKARED

SEQ ID NO: 48 - protein, 8 aa, synthetic construct
  source 1..8: note = human p53 NLS
PQPKKKPL

SEQ ID NO: 49 - protein, 12 aa, synthetic construct
  source 1..12: note = mouse c-abl IV NLS
SALIKKKKKMAP

SEQ ID NO: 50 - protein, 5 aa, synthetic construct
  source 1..5: note = influenza virus NS1 NLS
DRLRR

SEQ ID NO: 51 - protein, 7 aa, synthetic construct
  source 1..7: note = influenza virus NS1 NLS
PKQKKRK

SEQ ID NO: 52 - protein, 10 aa, synthetic construct
  source 1..10: note = hepatitis virus delta antigen NLS
RKLKKKIKKL

SEQ ID NO: 53 - protein, 10 aa, synthetic construct
  source 1..10: note = mouse Mx1 protein NLS
REKKKFLKRR

SEQ ID NO: 54 - protein, 20 aa, synthetic construct
  source 1..20: note = human poly(ADP-ribose) polymerase NLS
KRKGDEVDGVDEVAKKKSKK

SEQ ID NO: 55 - protein, 17 aa, synthetic construct
  source 1..17: note = human glucocorticoid receptor NLS
RKCLQAGMNLEARKTKK

SEQ ID NO: 56 - protein, 9 aa, synthetic construct
  source 1..9: note = synthetic NLS motif
PAAKKKKLD

SEQ ID NO: 57 - RNA, 19 nt, synthetic construct
  source 1..19: mol_type = other RNA; note = scaffold sequence
taatttctactcttgtaga

SEQ ID NO: 58 - RNA, 15 nt, synthetic construct
  source 1..15: mol_type = other RNA; note = scaffold sequence
atctacaacagtaga

SEQ ID NO: 59 - RNA, 15 nt, synthetic construct
  source 1..15: mol_type = other RNA; note = scaffold sequence
atctacaaaagtaga

SEQ ID NO: 60 - RNA, 20 nt, synthetic construct
  source 1..20: mol_type = other RNA; note = scaffold sequence
ggaatttctactcttgtaga

SEQ ID NO: 61 - RNA, 19 nt, synthetic construct
  source 1..19: mol_type = other RNA; note = scaffold sequence
taattcccactcttgtggg

SEQ ID NO: 62 - RNA, 15 nt, synthetic construct
  source 1..15: mol_type = other RNA; note = scaffold sequence
atctacaagagtaga

SEQ ID NO: 63 - RNA, 15 nt, synthetic construct
  source 1..15: mol_type = other RNA; note = scaffold sequence
atctacactagtaga

SEQ ID NO: 64 - RNA, 20 nt, synthetic construct
  source 1..20: mol_type = other RNA; note = scaffold sequence
taatttctactaagtgtaga

SEQ ID NO: 65 - RNA, 21 nt, synthetic construct
  source 1..21: mol_type = other RNA; note = scaffold sequence
taattttctacttgttgtaga

SEQ ID NO: 66 - RNA, 18 nt, synthetic construct
  source 1..18: mol_type = other RNA; note = scaffold sequence
aatttctactgttgtaga

SEQ ID NO: 67 - RNA, 19 nt, synthetic construct
  source 1..19: mol_type = other RNA; note = scaffold sequence
gaatttctactgttgtaga

SEQ ID NO: 68 - RNA, 36 nt, synthetic construct
  source 1..36: mol_type = other RNA; note = scaffold sequence
gtctaaaggtaccaccaaatttctactgttgtagat

SEQ ID NO: 69 - RNA, 36 nt, synthetic construct
  source 1..36: mol_type = other RNA; note = scaffold sequence
gcttagaacctttaaataatttctactattgtagat

SEQ ID NO: 70 - RNA, 10 nt, synthetic construct
  source 1..10: mol_type = other RNA; note = modulator sequence
taatttctac

SEQ ID NO: 71 - skipped

SEQ ID NO: 72 - RNA, 11 nt, synthetic construct
  source 1..11: mol_type = other RNA; note = modulator sequence
ggaatttctac

SEQ ID NO: 73 - RNA, 10 nt, synthetic construct
  source 1..10: mol_type = other RNA; note = modulator sequence
taattcccac

SEQ ID NO: 74 - RNA, 12 nt, synthetic construct
  source 1..12: mol_type = other RNA; note = modulator sequence
taattttctact

SEQ ID NO: 75 - skipped

SEQ ID NO: 76 - RNA, 10 nt, synthetic construct
  source 1..10: mol_type = other RNA; note = modulator sequence
gaatttctac

SEQ ID NO: 77 - RNA, 10 nt, synthetic construct
  source 1..10: mol_type = other RNA; note = modulator sequence
aaatttctac

SEQ ID NO: 78 - DNA, 508 bp, synthetic construct
  source 1..508: mol_type = other DNA; note = CMV
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 79 - DNA, 81 bp, synthetic construct
  source 1..81: mol_type = other DNA; note = SCP
gtacttatataagggggtgggggcgcgttcgtcctcagtcgcgatcgaacactcgagccgagcagacgtgcctacggaccg

SEQ ID NO: 80 - DNA, 385 bp, synthetic construct
  source 1..385: mol_type = other DNA; note = CMVe-SCP
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtacttatataagggggtgggggcgcgttcgtcctcagtcgcgatcgaacactcgagccgagcagacgtgcctacggaccg

SEQ ID NO: 81 - DNA, 947 bp, synthetic construct
  source 1..947: mol_type = other DNA; note = CMVmax
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggg

SEQ ID NO: 82 - DNA, 195 bp, synthetic construct
  source 1..195: mol_type = other DNA; note = JET
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 83 - DNA, 1723 bp, synthetic construct
  source 1..1723: mol_type = other DNA; note = CAG
atctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 84 - DNA, 511 bp, synthetic construct
  source 1..511: mol_type = other DNA; note = PGK
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 85 - DNA, 1195 bp, synthetic construct
  source 1..1195: mol_type = other DNA; note = EF-1a
gaattcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacatcatttt

SEQ ID NO: 86-2019 - skipped (1934 entries)

SEQ ID NO: 2020 - DNA, 1000 bp, synthetic construct
  source 1..1000: mol_type = other DNA
gcctcccaaagtgctgagattatgggcatgagccaccgcacctggccctgacaagaacctttgagttaggtataatggttcaccccaatttatagataatgaacccaagtcacaggggaagtgaagtcagttgcctaaggtcagacagcagtaaatggttctctgaccctaactccactgcctccctctcataaaaacactgggtggttacagtgggcccacctggagaagtcaagctattctctccatctcaagaacattaatttaatcatcctttttaccatataagataacatcttcacaggttctgaggatgagaatgttgacatctttgggtggtcgttattcagcctatcacaggtatccagggaagaaaaaaggaatttccaaaaagagaaaatacgaacattgggaaggctaattacagatggtgactactgaagggttagtcagaagcataatggaggcagtgatgagatgacagcacagatgcatgactctagtcccagcaactcctaaaaggtaaagaaatgtatcctgccaccctcagcttctttggggtgtcctcataaaagagaggcagtaaagcagaatcagagtcagatagagaggttgtaagaagagaagcagaggtgagtaagctgtgtttcaaacccagagtcaaggctcttgcccctctgcggtgctgccgaagcccagggtgggtggggactgacatgcaactcaggtactgtgtggcagactttgtgccttggcatgaaactatgcctgcccacaggaaggggcaccattttctcattagctcaaagagacttctgctggccaattcctgtcttctcaatactgcagctctccagagacaacactgttctctattctcctgtaagtgaggcagagcctggcagtaccctctatgccacctctcactagtacaggttagcactcagggtggcccactggtgtgtgtctcagctgctggtgtgcgtgctggtgcaggtac

SEQ ID NO: 2021 - DNA, 1000 bp, synthetic construct
  source 1..1000: mol_type = other DNA
tgggctgagggttgtggctggatctctttgcattgccacatccacaacagaattttgagaagtccgagaattctaaattggagcctgaccttcttcataatagtatatttgtcaaggtaggaggataaaacattttattgaacagtttgctaagctgatttaaaattttccagcatttagctatatggtatatggacctccacatgtatgatttcatttatattaaatgtccagaatagacaaatctataatgacaataaagagattagtagttgccagaggctgggaggaggggagaaacaatgagtgattgcggacgggtgtggggtttctttctggggcgataacagtgtcctggaattcaatagtgataatggatgcacactgtgaatatactaaaagccactcacactttaaaagtgtgggttttatggtaatttgaatgatatatcaagctatcaccaaaaatacacaatgggagttcagaaatgccaccccaaactatgatgatttgacatgctcattactttgaactgatgtcacttggggaaaaacagatgcaggcagagactttctctgagatctgcttatctgcctaagacagatcaagggatcctccaaaaggaactcaattgtcatgaatcccctcccctggaaccttatcaaccagggacaatgaacttagatcagagagggggagactggaggttgacatcatgcctagacagccacctcttcttctgagggctgctccaagagaacctttattacttgagaggcttctcatttgcataacaagaaacctttgttcaccatacacttcctcccctcatattctcataactggtgtcaccaccacccacgcagaagtccaaagcctctattcccttctgtacctcagggtgctatataagcttcaatcatctgacccttctttgaatctcatattttgtgggcttgcatgggtatgtacataattaaaaatggatttcctcttgtt

SEQ ID NO: 2022 - DNA, 1000 bp, synthetic construct
  source 1..1000: mol_type = other DNA
atttacacacatgccacagacagaaacattttaatagacctttgcttatggaaaagtaaagcaaaaatgtaattctagaagggagaaattttagtcaattagaaaataagatggtcaggcattgtagctctcatgtgtaatcccagtgctttggaaggttgaggcgagaggattgcttgagaccaggagtttgcgaccagcctagacaacatagctggtcatataaaaaaacttcaaaaaaattagctagctgtagagctttctgcctatatttccagctactcaaggatgaggcaaaagaatcccttaagcccaggaggttgaggttgcagtgaactgtaattgcaccaccacactctagcctgggtaacagagcaaggtcccatctcctaaaaaaaaaagaaaggaaaataaaaagaaaataaactattctccataataatgtagacagcaatcctcactgtgaaccagaaggaacctcggcaaattttttagacatcaatgggatttcactatcagctgagagtgttccctttttagcatggcaagctgtttcctgaagcaatagagagaagcaagaccaaggaaaaatctagaaagagcctctctgtagaaaagcagagcaatgatctctaatcacaatgctatcaaatattccaggctaaattttcctttatagcattaaaattttcctcacatccacaagattccaatagttttcttaatgccatagcctggtgtctattctgccttgtggattcccataatgcaaaatgccattaaaaaaggaacagaccatgagaagtgggcctccgaagcacatgaagcttggtatcatcagaaagataaggggcaacagtcaggaataattgttgggacatttaataagtccctggaaattcctagaaacataatttttttttgagtctaagatgctatcattttaaggtgcaccattattttatttgctacaatgtagaaaacaataacactgccaatt

SEQ ID NO: 2023 - DNA, 1000 bp, synthetic construct
  source 1..1000: mol_type = other DNA
tgattaggtaaaatatcagagacacaaatcaggttaaattgattttttattgtaattacatttaaaattttagaattcatcagtaggtatgaacaaacatatacatacatatatataatttatattataagtttattatttatactatacattataaaaataactgagagataaactttcgtttatccttaatgctaaaataattcatttaccttggagagatcagaactctgtccatttcccctacataaaaactagagagtactattgctttctctttctcgggcttactctggtctcatagaatatgcattttcattttttttcaacagaatatccgtggatagctaaaatttctgcttcctttgtcaacatttgtatttccccagtggacatttctgcaaaatttattttcatttctttgttaccagagaaactctgttggtcaagttcaatagcatcctcagcataatttcagaaggaaattacagggagcaattgaagtccatcactttcttggaggggaaatattaacaccctcacctcttgctcccaatattaggtggtaggcaggagtgagttactcattttctgaaggagcagtaactctttggacccctcgagtcacttggtaaataaactctagcactgccccgaagagtgcctcagagatttcaaggaataaatgctttaaaggtaggaaaatgctaagaaacaccatcatataagtgagttatttccaattttattttaaatacagccatatattattacatacagccacacattattaaataatgtattaatacattattattaaatacagccatatatatgtatatatgtgtgtgtgtatatatatacatatatatgtaagtatgtagctgctataccctcctgaagcaatgaatgtagctgctataccctccagaagcaatgataccctccagaggtgataacagatacaagtaacaaccacactctctggttttgacaacca

SEQ ID NO: 2024 - DNA, 1000 bp, synthetic construct
  source 1..1000: mol_type = other DNA
cagagagcttccaaggcattatcccatccaaagggtaaagaggctgggatatttatcgactagctcccattcttcactggctgtaacttgtccacgtctcacagctgtaactcccttgtattccctacctatctggtgtgaggaccaagcttgtgtctgtggatagagaaagccctaaagcagaaagtctaggtgcttgcacaaaaagatcatctgcacagaatgatgatcaagagatgtgagtggggcaccacaacatttacctcaggaatctctgttcaggactcagctttggtctcaaaccttgggaagcttatacactgaggcagtgttaggatctcttttctctgccttcctgtgcttttaagtgtatttcactgtttttgatcccttgtctgccccttatatttgactatcaggctcttgaaggtctattacacttactcattgtttttaccccctgttcctatctcagtgcccaacacagagctgacagttaatatatgttggttggatgcatgtgtgggtatcttatctttttatcctttaaaagacctcacacgtagatgaaaattttaaaatcattaattcaatcatcaattcaattcaatcatctttttatcctttaaaagacctcacacattgatgaaaattttaaaatcattaattcaattgaagaggccttgtgattgacatgagtataaattggaccattattaacttcaaactaattctactatgccagaaaccatgcctgaagtattaaaacatcacgttaaaaaacaaaagacaaaaaaaaaacttatctaaaaaattacattaaataaaatagaccaaaggtaaatcttactcaagttttcaggaaaaaaaaattgttttctatactcttttctcacctattcttccttgtcacagagaagcaattattatattagactttcctttttcaatgtgtagatgacatcatatgatttaaattttttatgtatttctcttgcaa

SEQ ID NO: 2025 - DNA, 1000 bp, synthetic construct
  source 1..1000: mol_type = other DNA
atcagcagcagaggctgcagaacagcggatattagtgaaaagcaaatgttgctgtctgatcgttcctgtggaagttttgtctcagaggagtacccggccgtgtgaggtgtcagtctgcccctactcgggggtgcctcccagttaggctactcaggggtcagggacccacttgaggaggcagtctgtctgttctcagatctcaagctgtgtgctgggagaaccactactctcttcaaagctgtcagacagggacatttaagtctgcagaggtttctgctgccttttgttgggctatgccctgcccccagaggtggagtctacagagacaggcaggccttgagctgcagtgggctccacccagttcgagcttcctggctgctttgtttacctacaatggtgggctcccctcccccagccttgctgctgccttgcagtttgatctcagactgctgtgctagcaatgagcgaggctccatgggcgtaggaccctccgagccaggtgggatacaatcttctagtttgctgtttgctaggaccattggaaaagcacagtattagggtgggagtgacccgattttccaggtgctgtctgtcacccctttccttggctaggaaagggaattccctgaccccttgcgcttcctgggtgaggtgatgccttgccctgcttcggctcatgctcagtgcactgcacccactgtcttgcacccactgtccgacaatccccagtgtgatgaacccggtacctcagttggaaatgcagaaatcattcatcttctgagtcactcacgctgggagctgtagactggagctgttcctattcggccatctacatgttctttcttccctcatcatcacttctttacttcttttatttcacttctggctttctgtcctcccacgctgaggaagactgatttggtggacatgtatttattctgctgagtaccagttgatgtggaagtagttgttttatagtcaacatgtttttatgactaat

SEQ ID NO: 2026 - DNA, 1000 bp, synthetic construct
  source 1..1000: mol_type = other DNA
gagtgatgtctaatcacaatctgtgataggtatttgctttaaggtgcatctaataacatgacagtgattttcatctcatataaccttcattaactctggttccctgctaagataaagccttccctataagccaactgagaatactgtagtcagaatttacaggtacttcccattgtggttgttcaccttatttgtgccagtttttcttcttctttattcataccttttgccatgtgaatttgcatttcttctgggttggagtcaagtatatatttatcctttttacctttgactctgaggctggccaaaggaataaggtggatgtgacaaggtacaatttctgagcctagcccttagaggccttccatgtttccacttgttctcttgcacttgcgacgttgctgtcaaaagaacatgcaatggctagctagcagcctgtgcacctgcagtgagaaccagagccacccagttgctgcagcctgagaccaagctgctcagctaagcatagcttagatcaccattgagttctgaggtggtttgtcatacagcaatggcaatcagatatatccacacaaatataattttagtttatatttttgttactgcagttctcatcttattctgaggatacgtgacaaaataattctttcaaaaatattgatgctgtgccagattactattttgaatgaattattagacaaatacttcatatgtatcttattatgtgggtttacacattatttatcttattgatttaacttcaaaactaaactttagtttagctcttgggccctatctgggaaagggtcatcttttaatcaccattaaatcactgaagtcatcagtttattcaaagtactctgcacaaaattagcattctttagtggttgtgaaataaatagactttaaacttatcattaatattcccaatggtactatgggggaggcaaaattttctatcttcttagtggtttttttttttttggctagggctaaggat

SEQ ID NO: 2027 - DNA, 1000 bp, synthetic construct
  source 1..1000: mol_type = other DNA
acgcacctgagaaatgtgttaaggattaagatgctagtgctagatgtttgattttctgaatcgaaccactattggtgagatccagaagctcaaagacatgatatacccaccttcaaataatgtttatgtaggtaatctattctcaggatttatagacactgctgttaagacctattgtcattggggtaaaaaaaaatccttattatattatacaaattattatatactattatattatagaaattatatttctattaaatagcttgtgtagaaagtaaccatatatagttagaaaaacactgatctcaagaacaggattttagatttgactctgacaatttctgttcggtcttgtataaatgtatcaatttagatttagggctttattttctaatccataaaatgtgtagcatacttctgctagctatacatttactgaagttattattttaaactatttttattttcatttttttgttttgagttataatcataattaatggattcaagtgacagagaaaagaaagtaattagtcatcttttttcagaatacagtctttgttctgaaggtatttcgtatgaatcaagtttcaaatcttcagataaattttcaccttgccaatgtgctttctgctctaaatcattcctgaattttgctatgatttttctttcttataaaatcttgacactaaattgtcaggagatatacatatatgtatatatgtaaaatatatatatcatatataaatatatataaattttgagttaaagtactattacagtattcaattctaccagtaattctaatagtatgaaaataaagtcaccagttgaagtaagacctactgacaccttctattatatttcgataattctatttgaaactaattatatagtaggacattttcattgttttcagtattaactggcactcatgtagatattgcaggccaaattttacctctaccttttggaattttctggggtagacttgagaatt

SEQ ID NO: 2028 - DNA, 1000 bp, synthetic construct
  source 1..1000: mol_type = other DNA
tacatgtgtaaacagttttagcgtagatttcctcgcacttttaaattttggattcttaatttccctgtcccccctgccccccccccaaaaaaaacctgctaacgtttaaacgaacacagtttgggaaatctgcgttaagtccttcgtgggagtggggttgctcagctcacagtaggccacgaacctgaattttctcttgtctgctgcccccttttgatagatggagggaagagcaggcttccagtgcaatggacagaagagggagcctgcaagttggtaacagagtctattagggaaagagagagtcacttgaatcctcagagctgctcctgtcaactgctttgtgcagtttttgtgacttattagctgcttgtttgcactctatctacgcctgcccaggtgtgtttgggccctagagcgaagggagcacaggcgttcatttagaaacttatccctccgtccaaatattggatgcttaccatgtgcctggtgcaatgcagggtgatacaaagaggaagataagtgaggcattcttatcgaaggaccagacactcttccagcctgactatattcattacactcgtgcctgacctttctttgactctaagattcttcctttctaaatgtgaatcttaaagactgaagtctttgatctaagactgctttcttatcacatcacatccaacaaccaacttttcacagcttcccagatcccaaattctgtttagcaaggacacttggatttttttgttttttgttataaatgacctcttcaggttcatattttcactatgtccagaattcttattttattctgttttgtgctgacattggaggcagagtctgtgtcacagaatacaccactaggggttaccctggacatggaagggtattcactcggggaagaaattttaatggaatttttaatatctagagctgtcattatcctgtgatggttcacaagaaatggaacacttaaaaatttctacagaaaaaaagg

SEQ ID NO: 2029 - DNA, 1000 bp, synthetic construct
  source 1..1000: mol_type = other DNA
gccacaaatttgttttctgtatctgtagatttgcatttttttccgaacatctcatatgaatagaatcacaaaatttgtgtattttgtgccaaacttctttcacttagcatactgatttcaaaattgatccaacttatagcatatatcagtactttattcctttttagggcaaagaaatcttccattacacggataccccacattttatttctctacccatcgcttgctgggcatgagttgtttgtgacaaatattcatatacatattcttgtgtggacatatgttttcgcttctcttgggtatatatctaggagtaggattgctgggtcatatggtaagtctctatttaatggtttagactcagtactttgttttctgcctttccacagctcagtttcataaagaggcaggagccttttgttcagggctccttggcagtaaggtaatttcttcttctgcattgtatccagctgacccttgctcagtgctgttctttgggggaaagatggaatgctgggaagccagcacctcttattccttctagctaacacttttacagtgacggatataatagatatcttcaactagtattgttgaattatctccctgatgctgtccaattttgcttcatatattttggggctctgttattaggtatgcatatatagtcattattgttatatctttgtggtggtgtggcctttttattattttagcacttttatatctttacctctaataacgtttttaaaaattgaacgttgattttgtctgatgttagtacaaccacttcagcttctttgtagttgctgtttgcatgacatatctttctccattcttttactttcaatctatttgtatctctgggtctaaaatgtgtagatagcacatagttgaatcttttaaaaaatacattttacaatctctgatttttattggaatgtttaatccatccacatttaatgttacgattgatggagctggacttatttctgccata

SEQ ID NO: 2030 - DNA, 1000 bp, synthetic construct
  source 1..1000: mol_type = other DNA
aacacagagctaaaaccaagtaagaggcgattctccaaaagcacttcctcagcaaacagcatatctattgtgtgtgggttctttaattggctgagaactgaatttcacctttggcattaaagagaagtgtttatttttactgtcttcactgttttaatgtttaaacaaaatctaaatactgaggtgaactctatcataaaacaagtgaaacggcaacataggttgatccagaaagaagcaaattccagcatggcgggcactacatgtttcagctcatcagttatctgaatcttatggctctaaagatggatggatgagaatacataggcagaagcttcctggtgaggctggtatgattctgttgtcctatcttcaacactatccttctaccttcagggttgctgttgtaggttttatttctttggcttctgttgccagtaatggaaaaggaccacatggaagactgtatttatgtacatcatgtccaaacagaatatcctataatagtgaatcttggaagaaagcttgagagatgtggcccagcgcggtggctcacacctgtaatcccagcactttgggagactgaggtgggctgatcacgaggtcaggagttcgagaccagtgtgaccaacatggtgaaaccccatctctactaaaaagacaaaaattagccgggcctggtggtgttgcacccgtaatcccagctacccaggaggctgaggcaggagaattgcttgaacccaggaggcagaggttgcagtgagccaagatcgtaccactgcactccagccctccagcctggacaacagagcaagactctgtctcaaaagaaaaaaaaaataccagtttgagagatgtatgtgaggactgattaccgaaagcgaaagggtttagtacatctcatgagaacagagcagtcacaagtgatataaaccaaactcccttggaaatttgtaatctatcaacttctttatttaaagagaataggaggtttactgtg

SEQ ID NO: 2031 - DNA, 1000 bp, synthetic construct
  source 1..1000: mol_type = other DNA
actcccactcctactaattacagcttgtgtgtccttcagtcattcacttcccttcacatgaccagcccagcagaaatgaactaccaggaacatgagctcagagcgatgggctggccacctgccaagcacctctgaatggaaagagcagaattttgcattgcctgccatgccacgtggagcaggccctgggtggctctttaggggatgggtgtggactcccacaacaaaaccaagggccatattcaaagttaaaagctctgccatagatggtatttgttgaggctgtgtgtggtagctcatgcatgtatgcccaacactttaggaggctgaggtgggaagatcacttgaggctgggagttcaagtctagcctaggcaagatagtgagatcccttctctaaaaaagataaaatattaactgggcatcatggacgtgcctgtagccccagctactggggaggctgaggcaggaggatggcttgagtccaggagtttgagactgcagtgagctgtgattgcaccattgctccctagcccgggtgacagaacaagacttttatttctttaaaaaaaaaaaaaaaaaagaaggtgtttactgcagttgctttattaaaaaaaaagtaaatgaatgttctgactgttctacttttgaaaataagtggcaaggaattagaactgtatctttcagcaacaaaatgtacactgtggttccatgtcacagccaggaatggagtcagatgtctcagaccagaatcacagctctgccacctcctgtgacatggacttgctaagctaccttgactctctggagctcactatgcccatcaataacaagaaataaataaatccgtcctgtaaggttgtcaggagaaacaaatgaggcactatatgtggaagttcctggaatagtgaccagcacagaggacgtctcaaagaaagatttgctgaaccccaaaagacaggaggactggaggaacaacaaagagacaggaaagctagcat

SEQ ID NO: 2032 - DNA, 1000 bp, synthetic construct
  source 1..1000: mol_type = other DNA
aattcatagcccagccaaggaacttagaagagtagagggaagtcatttttcactcccctacaagaacattctgctgtaaagaggagctagaaataatttttgttttaaattcaaccaaacatagggataattctgaaatttggaaccaaaagaattataagtacactactggtgaatttgtgcttatctgaaatctacacatgtagctgtctttatgtatctctgtatatcgatgtttttctatatatataatcagtgaagtaagatatctagtcattcatttactcaccaagtgattgcagtggggtgacagggacagtggggggtgtggtggcgggttgccagagcatgaggagtatgcaatagaatctaagaaatcatacctacctggccaggcacagttgctcatgcctgtaatcccagcactttgggaggcagaggcaggcggatcacttgaggtcaggagttccagaccagcctggccaacatggtgaaatcccatctctactaaaaatacaaaaaatacaaaaaattagctgggtgtggtggcacatgcctgtaatcctggctactctggaggctgaggcaggagaatggcttgaacctgggaggcagaggctgcagtgagctgaaattgtactactgcactccagcctgggtgacagagtgagactccatctcaaaaaaaaaaaaaaaaaaaaaaaatcagacctgccttccatgagctcatggtatacttgaatctccataggctagttattcaggagggtatgtaatgtaactcaacaatgcacaattacttaaattcgctcaggagaattacctcattttgcccaacttgttactgtgaaaaaaaaaaaagaaagaaaatttcaggaccttccaaatttattatgccaaagggaaaagtcaagccctggaaaccaagtcatgtaacacggctgtttttcttctctggtgcatgactgttgcttcctgatctttttgttgatgttataca

SEQ ID NO: 2033 - DNA, 1000 bp, synthetic construct
  source 1..1000: mol_type = other DNA
catataaattaaatatttatgttatattgaaggaatacttttagacttgtttaaacacaaatctttaaaaattacatatcactcttgcatgtacataaaaaatgaaaatataggcaattaaattaagagaggtctacagtgtctttacatcaagtctgactctactgagtccctttttgactcagagtcattaatatattgtttttttccagtaataatgtagtgatgcagcctgtcttcaaagactgctctactattgactcagattttctcccaagccattgatactagttttgaagctgatgctttttaaatcttgctgtcagacttacgggaaggttttcatacaacagggctcatattctttcctcaaattatccttacatgtaaatgttcagaatgtcgagatgatacataggccagttatgccactgtgaatatctaccaaggtcacatgtgtaatgaacaaagacagctatttctgctgctggctggcagtgatttgcaagattttgttgactgtaggacatatcctacttcaatgatgttaaaatgtgaacaaatatgcacttcagactttgtaaaatgtagcacagcacttacagagcacactaggcttctggcactcgcataaaatgaagacttggagttttagctgagtactaaaggaggaccatcctcccaccgaaggatgaagaatttaaggatatgtaagttgagctgtacttatgttcatctgtgatttttacaagtcacttattgctacatgtatcctttaaatatgcgttgtccttcctcctaaaatggtttcaccataataagtgaaatgtcagcttgtcacattaaattataaattataaattaccatcaccttagtcctctacatatccttcaacttcattatgacactgtccttcagagataaggaacagaaaggctttaatgaaaacttcagctaatgtaataattagggaaggatgagctaattaagaaacataca

SEQ ID NO: 2034 - DNA, 1000 bp, synthetic construct
  source 1..1000: mol_type = other DNA
caaagtctccctagagggcaaaattgtccccattgaagaccactgggttagatagaaacttacatctcacacatggagagtccaggctggcatggtcgctctgctgtgcactgggagcccaggttcctcctcgctttgcaaattgtacaagctgccctcatcacctggatgcctacatctcacttaagagtctcagttctaggagggcacagacaatggtgtactggtaaacagactctgttaaaaaaaaaaaaaaaaaaaaccaacacaatcaggaacattttttaaaagcccagatttgtagtgtttgcagattcttatgttttaaatactcctgccatggctgatgtgaaactaccaacagtttaacaactggcttactaaatttctgaatatttaccatttgtcccttgtaagacagtattagtgggctgcagtatatcaacagagaaagggaaggaaaagatacaaccttttgttgaaggacaaaatgacatttcacttttcttcagccccactggccaaaacttagtcccatgttcaccttagctgcaggggaggctgaaatgcagtgtttattctaaacaaccatgtatccagccacaataccaggggaatttatcaccaagagaaagagagagagaatatctagtgcttgaaaattatcagtctctgccacaattttatttaaaaaataaccagaaaaatgagagtgaattttatctgagaggatcttagaaatctcagcatcgagaaggtaataaataaagagagataagtcacagacttcctgcgacagtcaagaattccccatgcagatgacaccccaggagatgccgggtgattgttcttacaatttcttcagttgaaggtaaatgtggcactagccatttattcttttagctcacgttgtttgaagtgcatcgcctatgtacttcaccctttggactcactagaaaacaaagagaattttggaattagaagaggcttaataatgtta

SEQ ID NO: 2035 - DNA, 1000 bp, synthetic construct
  source 1..1000: mol_type = other DNA
aaatataaataaaacatttcttttggaaattttataattcaagctaatttaaaattatgtaaacctctatctttcatgtaatcttcttccttcttttaaaacaacatttttttggtggtcatctgttcgggagaaaatgaaattttctgtggataagcagatattcttcacggagaaagctaacattctgcattcctctattttaaaagtggaaaacatagtcctgttatttgtatttagatgtatttctcaccaaagagtgccaggctggattacagaagatctatattctgatcttgtcctttttctttgcaagcctgaggaattgtccagacacagaattccctagatccccagatttctcacctataatatgaagggttgaaagagaggtctcaatcggctttgaattttctgttctatacttctgcaccaccactgtagcactgacaattgcatgaaaatattaagctctattatgttttcagtactatccttagcttctttaaaaaattagtctagctgtgtttgtaaataaatgatgtcactggaaaaatggtttcataccattgttgtcaatagttgaatgtggcttgccctcaggaacaatgcattcttcaataatatggaggatggaaggtgtataaggactcagatagctattattctcatttgcccatgatcctttcatatccccgcctctggtttagcattctctttcttccaggggaatttctcccccattccatgcattctagtagaattttttatcacagtagattgtcctgccctgccacagaaatgggcatttgacacagtggccacaaagattggtctaagcagtaggcctgtgacccaaggtaggccaattagagttttctgtagaattttttagattcaaagtgtatgtgtgtgggggggatgactcttcttgaattttatattaggatgcatgccagaaattgttgaaaggtctttaatgtaccatgtacaggaagct

SEQ ID NO: 2036 - DNA, 1000 bp, synthetic construct
  source 1..1000: mol_type = other DNA
cacctataagaggaaatatacttatgtctaggtggactccaatgtgtctgtttactgatacttatttattcattattttcaagtaaaatgtagaagtgaataacttaagagaataactatttttatgagagaaaaatacccactttcttttttattactttgttcctctagaggttcatgaataatatattgaacatgtgaggagtgaggcctgtctagctcttttcctaacatcttccactcctgtggcctcttattaggtacctttctcagtgaagatatacaataagaattttgcatgcttattgggaatttatctgtgaaaaatcactcaaatgtcattaagtcttttctgataaaccttaatcatccaacaaccagagtttttcttaaaatagctgttgctctagaagaataccatagaatgaagttgcttcctagcatggcagtcaaggatcctggttccaagtatgagctctgaagaagatagactatgttcaccgcttactatagctgagtgcccttggacaattcatttaaactgcccctaattttcttccatcatctgtaaaatgaatgtaataatagctcttaatgagtattaaattagataataagggcactggcatttattaagaacttaataaatgttagcttttgttatttcacatttttccttgatcactcctaccaggaataaaattctgggagggtataagtaggtagtgaagtgctaactggtctggttaattgttagagttctgttaaaaaaaagttatttgaaaaaagtattttggagctaggatctaatttattaatatatctggattttctttttcaattttggtgtccattattcacataagtaattgtggttttgctatattttttcctcctgaaaaattatggctatacaactaactttattgtatactgaattttggaattttttaggatttgatgttcttactggggagaggattttgaattatttaaccac

SEQ ID NO: 2037 - DNA, 1000 bp, synthetic construct
  source 1..1000: mol_type = other DNA
aacaagaggaaagcatacaaatttatttaatacatgttttatgtggcacaggagccctcataaagtaataaaaaatccccaaacacagttagagctgaacatttatatactaatctggacaaaacatttatatactgcgtggacaaagagcagtaaattgtgaaaatggaacaaggcaagggggcttagactacagtagttaatcatcaagaagtgacaaaaaaaaataagggttagttaataagatttgtttaagcagatttctcccagctttagctctctgtctctggtgatcagaatgcactccttccttcagactcagtgagcacatattccacacggaagatttcttccctagcttttaggaaatccagagaaccctttttgtatctgttgtttttttttttttttaaatgtcttgtctttaactcaaaacaatttatgtgccaggatgacatatctttggataatgtgttctgaactccttcagtacatacgtatataaattaaagcaaatattttttatgataagctggcataatagtttcataatttaatcactgatttaaaaatttaattaaaattattttttaatattttgtgtaataatttttgaggagtatcttttgtgcttaatgagtggcagatgacacccatgttcttagcagcatcattcacaatagctaaaagataggaacaactgcgtattgatggatgaatggataagcaaaatgaggtatatacatataagggaatattcttcatccttaaaaaggaaggaaattctgacatatgctacaacaaggttgaacctctaaggacattatgctaaatgaaataaaccagtctcaaaaagacaaatactatgtgattccagatacataaggcacctagagacaaactgatagagacagaaagtagaatgagtgattaccaggggttgtgagaggaaaaaagagagggttgtttgatacagagtttcagttttgcaagataaaag

SEQ ID NO: 2038 - DNA, 1000 bp, synthetic construct
  source 1..1000: mol_type = other DNA
aacaggagaaaagcgtacaagtttattaaatagaagttttgcagccgggcgcgctggctcacgcttgtaatcctggcactttgggaggccgaggcgggcagatcacgaggtcaggagatcgagaccacggtgaaaccccgtctctactaaaaatacaacaaattagccaggcgtggtagcgaggcaggagaatggtgtgaacccgggaggcggagcttgcctctgcactccagatcatgccactgcactccagcctgggtgacagaccaagactccgtctcaaaaaaaaaaaaaaaaaaaaaaaaaaaaaagaagttttgcatgacatgggaaccctcataaaaaaagtgaagtcccaaaaaagtggcaaaatctaaatgcttttatattatgttgacgaaagaggggcaattgtggaaaagtaactaaattatgagggttaggctaacagaagataaaaattattttaacaagttctgtttgtataaaattttctcaatttcagctacccatccttgatgattagaatgttgcattccttctggtatacaaggaacatcttccatatgggggttttatcttctgctttcagaaaaaaaaaaataactctgtgtgtggtaggatgaaggtgtcagaacattcttcttgcacctgctggttgctatctttttaaactgcatttgtttcaaaacaatccttatgacaaaggggtgtattttggggtggcatattctgtcacccgtcaatatcaaagtggtttttgagtttgtgctcatcttctttccttattctgttccttgtaaggtaagactaaatataatggaatttgccgtcacatgtctcttatatgtagtgagttttaacaggcattccaggaaatgccatatggtcttttagcttggaaattattttagaaaacgataaaatctttagtgtgaagttatttcccagatatgtatcgctaaaattatcattacaggtgctctaggtaatatgtttg

SEQ ID NO: 2039 - DNA, 1000 bp, synthetic construct
  source 1..1000: mol_type = other DNA
ttagtacttccatccctttcctggctgctctaactttacaggtacttgtaagtggcaattaagcactttttcctaattccagagtcttgccccacttcagagcaacatagagtggcctagacaggctgaggtactttgccgcttcagtcatcattaatctatggtatttactgatgagaagtaaagtggtagaagaaaaaaaaattttctgttatcctgggcacttggaaatgaatgtattctcacaatctgttctcaaaacaacttactgattctggggttctggaagctctgatgtgcaggtgagccttttaaattcctcactgttggagctcctatctaggactcactggctggatgaaaacggttctttttattgctttctgaatgtctgctagacaggcgtaagcaacaccttatatctgccttctgaaaaaggtaaaagaactgggacccatccaccatgctggacagctcggcagtggcagtggcctcccccagaccctgttccgagtgctccaccaacaaactcaccagcagtcagagtctagcctctccccaaacttcaccttcatcacaattcattttaagcccttccacaacccaatcaactctagatctacttaatggataataatttgatctcatgcaaactgcactttcctcttctcagaatgatccttctaccccttaattaaacatttgagagtgaaagaagagaaaattcgggttcaaagattggtaagtctaagaaacctaaggaaaaggagttagtaaacatgttaatcaaagagtgagcacttttcggaagcgcaacattcagatacctttcttgattggattccagaagactatttctgggaagaggagatttgcatttttctaaagtcttctacccacagcctaaccaccctagggctttgaaatattttttttctgatgtgcagtcataattgaataaataaaatgattcctgatcatttcttctcttcagctttat

SEQ ID NO: 2040 - DNA, 1000 bp, synthetic construct
  source 1..1000: mol_type = other DNA
tattcctgtatttctattgtacttttttgcattaagaaacattttccaatgtaacattttaatagatttttcactatttgttgagttatttttgagtggttgtacttgagcttgccatctatgtcttaacttcagatttgtactaacttaattccagggagatatagaagcattattcctacatagctctatatcaacccccttttcctgtggtattattgttatacaaggtacaccatatatgttacaaatacaattatttatagttataattattactttaaatatatcatttatgtctattaaagaagctgagagcagagaggagataaagtatatatttatagaatttgttatattaagcttcttatttgtcattctgattctctttgttctggtggacttgagtaaatatgtgatgttatttcattatgcacacacagctttgctccttgtcattttatttatgctgtctttctcaagtatattgcatttaaatacattataggaccaacaattcaaatatatttatgttgtgttatacaattgctttttaaaatcagttaagatagatgggatatgcactgatagtatggtttttaaaattatactttaagttctgggttacatatgcagaacatgctgtttggttacataggtatacacgtgccatggtggtttgctgcacccatcaacccaccacctacattaggtatttctcctaatgttatctgtcctctggcctccaaccccccgacatgccccagtgtgtgatgttcccctccctgtgtccatgtgttctcattgttcaactcccacttatgagtgagaacatgtggtgtttggttttctgatcttgtgatagtttcctgagaatgatggtttccagcttcatccatgtccctgaaaaagatatgaactcatcctagacaataattcaaacacacacacacacacacacacacacacacacacacacacgcaaatggcactagtatct

SEQ ID NO: 2041 - DNA, 1000 bp, synthetic construct
  source 1..1000: mol_type = other DNA
tccagaaaacataacaattcagaacatatatttaatccctcctcaatccagatccttgttgaaacaatgaaagagtacaatatactgccatgaaaagtactgagaaaagtctacagatagtgacatggaagaaaagaaaaaatattaaatagatcaaactagttatataatttgtatctcatttctgtaaaataaatttaacatttataagtgtattagtttgttctcacattgctataataaaatacctgagactgggtaattaaaaaaaaaaacagatttaattggcacacagttctataggctgtacagagaaaacagtggcttctgcttctggggaggtttcaggaaacttccaatcatgatggaagccgaaggggaagcagacacatcttacgtggccagagcaggagcacaagtgtgaagggaagtgtctgttcatattcttcactcactttttaatggggttgtttgtttttttcttagaaatttaagttccttgtagattctggatattaggcctttgtcagatggatagattgcaaaaatgttctcccattctgcaggttgcctgttcactttgatgatagtttcttttgctgagcagaagctctttagtttaattttgcagggacatggatgaagctggaaaccattatcttcagtagactaactgttaacaggaacagaaaaccaaaaacaaacaaaagcatgaagagggaagtgtcacccacatgagaactcactattgtgatgacaacaccaaggggaatggtgttaaaccatgagaaccggcccccatgatccaatcacttcccaccaggccccacctccaatactggatattacaattcaacaagagatttgggcaggaatacagatccaaaccatatcagtaaatataataaatatatattaataaatatgtaaatatatgtatgcaagttaacaaatgaaccagttggtatgtaagtatgtatataaaggaccatagcagtta

SEQ ID NO: 2042 - DNA, 1045 bp, synthetic construct
  source 1..1045: mol_type = other DNA
ctgaatactagaggagcaagtacaacaaatggaaaatgggatcaagtatgagtgagagttgctaagatgcctggtagggatgcaaaggggtagagagcctggggagagagggtgagggagggaagcactggtttctcaagcaaaagctaaaatttttctattaagatttaacctgatgctacactttggtggtgcagcaagggtctcaaatggtataaaactcaggtgatcatgctttatgtctgtctctagaaaaatgctccaaaaatgataagtagtgataatccgcagtctcgttgcataaaatcagccccaggtgaatgactaagctccatttccctaccccacccttattacaataacctcgacaccaactctagtccgtgggaagataaactaatcggagtcgcccctcaaatcttacagctgctcactcccctgcagggcaacgcccagggaccaagttagccccttaagcctaggcaaaagaatcccgcccataatcgagaagcgactcgacatggaggcgatgacgagatcacgcgaggaggaaaggagggagggcttcttccaggcccagggcggtccttacaagacgggaggcagcagagaactcccataaaggtattgcggcactcccctccccctgcccagaagggtgcggccttctctccacctcctccaccgcagctccctcaggattgcagctcgcgccggtttttggagaacaagcgcctcccacccacaaaccagccggaccgacccccgctcctcccccacccccacgagtgcctgtagcaggtcgggcttgtctcgcccttcaggcggtgggaacccggggcggagccgcggccgccgccatccagaagtctcggccggcagcccgcccccgcctccagcgcgcgcttcctgccacgttgcgcaggggcgcggggccagacactgcggcgctcggcctcggggaggaccgtaccaacgcccgcctccccgccacccccgcgccccgcgcagtggtttcgctcatgtgagactcgagccagtagca

SEQ ID NO: 2043 - DNA, 1020 bp, synthetic construct
  source 1..1020: mol_type = other DNA
gccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaaccca

(19) United States
(12) Patent Application Publication
(10) Pub. No.: US 2025/0308637 A1
(43) Pub. Date: Oct. 2, 2025

(54) SYSTEMS AND METHODS FOR ASSESSING RISK OF GENOME EDITING EVENTS

(71) Applicants: Josiah Seaman, Boulder, CO (US); Jonathan Rubin, Boulder, CO (US); Gargi Datta, Boulder, CO (US); ARTISAN DEVELOPMENT LABS, INC., Boulder, CO (US)

(72) Inventors: Josiah Seaman, Boulder, CO (US); Jonathan Rubin, Boulder, CO (US); Gargi Datta, Boulder, CO (US); Nicholas Eion Timmins, Boulder, CO (US); Jamie Kershner, Boulder, CO (US); Panos Chrysanthopoulos, Boulder, CO (US); Calley Hirsch, Boulder, CO (US); Jonathan Leff, Boulder, CO (US); Elizabeth Hutton, Boulder, CO (US); Daniel Munson, Boulder, CO (US); Patrick Bedford, Boulder, CO (US)

(73) Assignee: Celyntra Therapeutics SA, Mont-Saint-Guibert (BE)

(21) Appl. No.: 18/866,571

(22) PCT Filed: May 22, 2023
(86) PCT No.: PCT/US2023/023161

§ 371 Date: Nov. 18, 2024

Related U.S. Application Data
(60) Provisional application No. 63/344,509, filed on May 20, 2022.

(51) Int. Cl. G16B 35/00; G16B 20/00; G16B 40/20

(52) U.S. Cl. CPC G16B 35/00; G16B 20/00; G16B 40/20

(57) ABSTRACT

CRISPR-Cas-based genome editing technologies demonstrate great potential as tools to facilitate gene therapy for hereditary diseases, as well as therapies that are not amenable to conventional gene therapy. However, CRISPR-Cas-based genome editing technologies may demonstrate off-target genome editing that may affect their therapeutic efficacy or other aspects. Provided herein are systems and methods to assess the hazard levels of unintended genome editing events.

### REFERENCE TO RELATED APPLICATIONS

This application claims the benefit of and priority to U.S. Provisional Patent Application No. 63/344,509, filed May 20, 2022, the disclosure of which is hereby incorporated by reference in its entirety for all purposes.

### BACKGROUND

Genome editing technologies have great potential as tools to facilitate gene therapy for hereditary diseases, by the destruction or repair of the responsible genes. It can also be used to develop therapies that are not amenable to conventional gene therapy, for instance, the universalization of allogeneic therapeutic cells such as universal chimeric antigen receptor (CAR) T cells. The genome editing technologies currently in clinical trials include zinc-finger nuclease (ZFN), transcription activator-like effector nuclease (TALEN), and CRISPR/Cas system. Each of these genome editing tools specifically binds to target DNA sequences and introduces double-strand break (DSB) at the specific target site, followed by genome editing using the DNA-repair mechanism of cells. However, this type of genome editing mechanism has specific safety issues that differ from conventional gene therapy, with one of the most important issues being off-target genome editing. Therefore, there is a need for systems and methods to assess the safety issues of unintended genome editing events with a regulatory lens for human gene therapy technologies. Provided herein are systems and methods for assessing the risk of unintended genome editing for guide nucleic acids, e.g., guide RNAs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic representation showing the structure of an exemplary single guide Type V-A CRISPR system. FIG. 1B is a schematic representation showing the structure of an exemplary dual guide Type V-A CRISPR system.

FIGS. 2A-C show a series of schematic representation showing incorporation of a protecting group (e.g., a protective nucleotide sequence or a chemical modification) (FIG. 2A), a donor template-recruiting sequence (FIG. 2B), and an editing enhancer (FIG. 2C) into a Type V-A CRISPR-Cas system. These additional elements are shown in the context of a dual guide Type V-A CRISPR system, but it is understood that they can also be present in other CRISPR systems, including a single guide Type V-A CRISPR system, a single guide Type II CRISPR system, or a dual guide Type II CRISPR system.

FIG. 3 shows a schematic of a Type V-A nucleic acid guide nuclease comprising a dual guide nucleic acid.

FIG. 4 shows an exemplary risk-based, decision making algorithm.

FIG. 5 shows results from assessing in silico data, categorizing risks associated with severity levels; and relative risk scores for three gRNAs comprising spacer sequences complementary to TRAC, B2M, CIITA targets.

FIG. 6 shows an exemplary risk profile for a gRNA comprising a spacer sequence complementary to target sequence in a CIITA gene.

FIG. 7 shows an exemplary risk profile for a gRNA comprising a spacer sequence complementary to target sequence in a TRAC gene.

FIG. 8 shows an exemplary risk profile for a gRNA comprising a spacer sequence complementary to target sequence in a B2M gene.

FIG. 9 shows an exemplary process for evelauting gNAs

FIG. 10 shows results of evaluation of TRAC gRNAs by Amplicon-seq.

FIG. 11 shows the number of off-target sites of high, moderate, or low hazard level for three different TRAC gRNAs, where the off-target sites are called by CasOFFinder and queried with various databases.

FIG. 12 shows the number of off-target sites of high, moderate, or low hazard level for three different TRAC gRNAs, where the off-target sites are called by Digenome-Seq as modified by Mantis and queried with various databases.

FIG. 13 shows validation of all off-target sites categorized as high or moderate hazard by rhAmp-seq for TRAC43 gRNA

### DETAILED DESCRIPTION

I. Systems and methods for identifying potential off-target sites and calculating risk thereof
II. Engineered non-naturally-occurring CRISPR-cas systems
III. Compositions and methods for targeting, editing, and/or modifying genomic DNA
IV. Examples
V. Embodiments
VI. Equivalents

### I. SYSTEMS AND METHODS FOR IDENTIFYING POTENTIAL OFF-TARGET SITES AND CALCULATING RISK THEREOF

Genome editing technologies can result in unintended, off-target edits. In certain cases, those unintended edits are innocuous, displaying little no to phenotypic change. In other cases, the edits can cause detrimental phenotypes to the host ranging from minor to severe. Therefore, there is a need to develop systems and methods to assess the impact of off-target sites and to help guide the selection of guide nucleic acids comprising spacer sequences comprising minimal off-target affects and/or spacer sequences comprising acceptable off-target site risk profiles, also referred to herein as hazard levels or the like.

In particular, many therapeutics or other cell-based products are, or can be, produced by CRISPR methods that utilize a CRISPR nuclease complexed with a compatible guide nucleic acid (gNA) (CRISPR complex) that comprises a spacer sequence that is partially or completely complementary to a target nucleotide sequence (target sequence) in a target polynucleotide (e.g., gene or, in some cases, intergenic DNA) in a cell into which the CRISPR complex, and/or one or more polynucleotides coding for one or more components of the complex, is introduced. The intended result includes at least a strand break at or near the target site, in some case followed by insertion of an exogenous gene or other polynucleotide at the site of the strand break. The cell is thus modified to have a desired function, and populations of the modified cell or its progeny can be used in a therapeutic. An example is chimeric antigen receptor (CAR)-T cells, in which modified T cells are produced that express a CAR targeted to cells associated with a pathology, e.g., cancer; the CAR-T cells are then introduced into an individual suffering from the pathology with the intention of destroying or rendering inactive the cells associated with the pathology. However, off-target sites for the gNA can also be affected in off-target events and the resulting change or changes in cells in which these events have occurred can present one or more hazards, also referred to herein as risks, when the cells are used in therapy, and/or that cause effects that render the affected cells less suitable to a process involved in producing a therapeutic or other cell-based product (e.g, effects on growth or proliferation). An “off-target event,” as that term is used herein, includes one or more effects in a cell caused by binding of a nuclease and its associated gNA to an off-target site in a polynucleotide that alter the polynucleotide or a set of polynucleotides in the cell. Examples include insertions, deletions, translocations, and the like, as detailed further herein. A “hazard,” as that term is used herein, includes unintended effects, or potential unintended effects, in the desired use or uses of the product, or in the method of making the product. A hazard can be assigned a hazard level, where the hazard level can be based, at least in part, on one or more likely deleterious effects of the hazard. A hazard level can be applied to a particular off-target site (e.g., high, medium, or low; or a numerical indicator of hazard, sometimes in combination with frequency and/or assay performance, as described in more detail below) or a particular gNA (usually based on combining hazard levels for off-target sites for the gNA). Hazard levels for a particular gNA can be modified at one or more stages in the process; e.g., on the basis of cell-based assays and/or other information. For example, a hazard level for a gNA determined on the basis of in silico determination of potential off-target sites for the gNA can be produced at one stage of a method, and a hazard level for the gNA determined on the basis of in vitro determination of off-target sites may be used in another stage, usually subsequent to the in silico stage.

Typically, a polynucleotide, e.g., gene, to be targeted in a CRISPR method may have dozens or even hundreds of potential target sequences, generally determined by proximity to a PAM for the nuclease used in the CRISPR method, for which spacer sequences can be produced, each of which is potentially useful in modifying the polynucleotide, and each of which will have different potential off-target sites. Although it is possible to test all potential gNAs, with all potential spacer sequences for a given polynucleotide, in cell-based assays to determine likely deleterious effects and thus determine which spacer or spacers are likely to have the least unintended effects for cells ultimately used in therapy and/or least effect on a process of producing a therapeutic comprising the cells, the number of potential spacer sequences renders the process prohibitively cumbersome, inefficient, and highly costly. There is thus a need for methods and compositions that can be used to efficiently and rapidly reduce the number of potential gNAs, e.g., to be evaluated in cell-based assays and/or other assays or by other methods, in a way that eliminates those whose potential off-target effects are deemed to be at a hazard level that is likely too hazardous to use in producing cells ultimately used in therapy and/or that are not suitable for a process to produce a therapeutic. This reduction can be based, at least in part, on preliminary hazard levels determining for the prospective gNAs that are based on a process that comprises combining hazard levels for each potential off-target site for the gNA and, in some cases, on other information regarding the gNA. The resulting subset of potential gNAs with their respective spacer sequences can then be used, e.g., in cell-based or other assays to obtain an overall hazard level for each gNA. One or more reports can be generated at one or more stages of the process, e.g., to be evaluated by a user or users who may, in some cases, manually alter a selection of gNAs either included or not included in the report, to be used in further stages of the process. It can desirable to generate a recommendation for use of one or gNAs in a CRISPR process to produce a product that will be used in one or more processes, e.g., therapy The recommendation can be based on overall hazard levels as well as, in some cases, mitigating information for particular aspects of the analysis, such as the product to be produced, the process for producing it, and/or the intended therapy. The process can be iterative, so that results obtained at one stage help determine input for another stage. A result of using the methods and compositions can be, e.g, a recommendation to a user of one or more spacer sequences for gNAs to be employed by the user in a process, e.g., development of a therapeutic.

Certain methods and compositions provided herein can be used in selecting one or more gNAs to be used in CRISPR methods of modifying target polynucleotides, e.g., genomic

DNA, where the gNA or gNAs each comprise a spacer sequence partially or completely complementary to a target sequence in the target polynucleotide. One or more potential off-target sites for a given gNA are evaluated by determining a hazard level for each potential off-target site; typically, a specific gNA will have a plurality of potential off-target sites, and the hazard levels for its potential off-target sites may be combined to determine a hazard level for the gNA. A plurality of gNAs, each of which targets a different target sequence in a target polynucleotide and each of which has a plurality of potential off-target sites, can be evaluated and ranked based, at least in part, on the hazard level of each gNA. In certain cases, a hazard levels for a plurality of gNAs for a given target polynucleotide are used, generally in combination with other information, such as efficiency of genetic modification for each of the spacers, to determine a subset of the plurality of gNAs that is then subjected to further evaluation. Efficiency of modification can be based, e.g., on a determination of frequency of INDELS in a population of cells into which each gNA, or one or more polynucleotides coding therefor, and its compatible CRISPR nuclease, or one or more polynucleotides coding therefor, and/or frequency of one or more desired editing effects in the cells (e.g., lack of expression of a protein for which the targeted polynucleotide codes and/or expression of a protein the sequence of which has been introduced into the polynucleotide), and/or one or more other desired effects. gNAs that pass one or more levels of evaluation may be further subjected to cell-based testing and an overall hazard level for each gNA may be determined based, at least in part, on the results of the cell-based testing. Cell-based testing can include sequencing, e.g., to validate potential off-target sites as actual off-target sites, often including increasing the resolution of the off-target site, e.g., a greater resolution of the genomic position of the off-target site. Other cell-based testing can provide information for a given gNA regarding translocations; insertions; expression levels of products associated with pathology, growth, proliferation, and/or viability; and/or other characteristics. In some cases, evaluation of gNA for potential use in a CRISPR process that is directed at producing a product, e.g., a cell-based product, that will be used for a particular purpose, can include factors that can modulate (e.g., mitigate) one or more effects of one or more events for an off-target site for a gNA.

Any suitable method may be used to determine potential off-target sites to be evaluated for a given spacer sequence, e.g., in silico, in vitro, or cell-based methods. An “in vitro” method, as that term is used herein, include a method for evaluating potential off-target sites in DNA that is not within a cell, e.g., that has been removed from a cell. “Cell-based methods,” as that term is used herein, include methods using intact cells.

Any suitable method may be used to evaluate a hazard level for a particular off-target site. In certain embodiments, one or more databases are queried with a genomic location for an off-target site, and the information that results from the queries may be used to assign a hazard level to the site. The databases may be any suitable databases, such as databases that include information regarding cancer, disease, biological function, protein coding, regulatory elements, and/or functional non-coding regions. The hazard level can be a numerical score, a discrete classification (e.g., high hazard, moderate hazard, low hazard), or any other suitable measure.

### Determining Spacer Sequences and Off-Target or Potential Off-Target Sites

A polynucleotide, e.g., gene, to be targeted for modification in a CRISPR method can be evaluated for target sequences that can be used to target a CRISPR nuclease complexed with a gNA comprising a spacer sequence partially or completely complementary to the target polynucleotide by means well-known in the art. A target polynucleotide may have dozens or even hundreds of potential target sequences, generally determined by proximity to a PAM for the nuclease used in the CRISPR method, for which spacer sequences can be produced, each of which is potentially useful in gNAs modifying the polynucleotide, and each of which will have different potential off-target sites. Allowable homology for a PAM sequence can be used to widen or narrow the selection of potential target sequences. In certain embodiments, the nuclease is a Type V CRISPR nuclease, such as a Type VA nuclease. In certain embodiments, the nuclease comprises an amino acid sequence at least 60, 70, 80, 90, 95, 98, 99% identical and/or not more than 70, 80, 85, 86, 87, 88, 89, 89.5, 88.6, 88.7, 88.8, 88.9, 90, 95, 98, 99% identical, or 100% identical, in some cases preferably 95-100% identical to SEQ ID NO: 37, more preferably 98-100%, or even 100% identical, in other cases 60-88.9%, preferably 70-88.9%, more preferably 80-88.9%, even more preferably 85-88.9% identical. Thus, a plurality of spacer sequences corresponding to a plurality of potentially useful gNAs may be determined for a given target polynucleotide. In certain embodiments at least 20, 40, 50, 60, 70, 80, 90, 95, or 99% and/or not more than 40, 50, 60, 70, 80, 90, 95, 99, or 100%, or exactly 100%, preferably 40-100%, more preferably 60-100%, even more preferably 80-100%, still more preferably 90-100% of target sequences as determined above can be provided to a method as described herein, e.g., a computer-implemented method, to evaluate gNAs corresponding to spacer sequences that are partially or completely complementary to the target sequences, e.g., at least 70, 80, 90, 95, or 99% and/or not more than 90, 95, 99, or 100%, or exactly 100%, complementary to the target sequences, preferably 70-100%, more preferably 80-100%, even more preferably 90-100%, sill more preferably 95-100%, and in certain cases 100%, complementary to the target sequences. The gNAs can be evaluated in a method that comprises determining a plurality of potential off-target sites for each of the gNAs and determining a hazard level for each of the plurality of potential off-target sites for each gNA. In certain cases, a hazard level for an off-target site is determined in a method that comprises querying one or more databases with a genomic location of the off-target site, such as one or more of the databases described below (Functional Categories and Databases). Hazard levels thus determined for each off-target site for each gNA can be combined to determine a hazard level for each gNA. Further information can be provided to the method from in vitro and/or cell-based testing of one or more of the gNAs in combination with its compatible nuclease, and hazard levels for the one or more gNAs may be modified based on the further information; for example, a plurality of potential off-target sites for each of a plurality of gNAs may be determined by in silico methods and a hazard level for each potential off-target is the determined based on querying one or more databases with a genomic location of the potential off-target site, then the hazard levels for the potential off-target sites combined to produce a hazard level for each gNA. This information can be used, often in combination with other information, e.g., information about editing efficiency of each gNA, to select a subset of the plurality of gNAs for in vitro and/or cell-based testing, e.g., in vitro testing. The in vitro testing can provide information indicating one or a plurality of off-target sites for each gNA which can then be used in a second determination of hazard level for the gNA. This information can be used to select a further subset of the gNAs which are then subjected to cell-based testing, and a third determination of hazard level for each gNA determined based, at least in part, on results of the cell-based testing. In certain cases, cell-based testing includes one or more cell-based assays as described herein.

Genomes and/or Cells Used to Determine Potential Off-Target Sites

Potential off-target sites can be determined in silico, in vitro, in cell-based methods, or a combination of these. In silico methods require a genomic sequence or part of a genomic sequence to be used. The genomic sequence may be any suitable genomic sequence. In general, a genomic sequence that is similar or identical to the genomic sequence of the cells in which a CRISPR method will be used to produce a product is preferable. Thus, in some cases, CRISPR methods will be used to modify cells removed from an individual, e.g., a mammal, for example, a human, and those modified cells or progeny thereof will be reintroduced into the individual. In this case, the genome of the individual may be used for in silico determinations of potential off-target sites. In some cases, CRISPR methods will be used to modify cells that are allogeneic to cells of an individual into which the CRISPR-modified cells will be introduced but that have been or will be modified to reduce or eliminate immunogenicity in the individual. In this case, the genome of the allogeneic cells may be used for in silico determinations of potential off-target sites. However, more typically, a genome will be used that is more generalized, e.g., for CRISPR methods that will be used to produce cells to be introduced into humans, a human genome may be used, such as one of those known in the art. In vitro methods utilize DNA that has been removed from a cell, and the cell from which the DNA has been removed may be any suitable cell, preferably a cell that is the same type or similar type to cells that will used in a final product or in producing a final product. For example, if the final product will be a T-cell, then in vitro methods for determining potential off-target sites may utilize DNA from T-cells, e.g., T-cells of the same type as will be used in the product or in producing the product. In some cases, the final product may be derived from a stem cell, such as an iPSC, and DNA for in vitro methods to determine potential off-target sites will be removed from the stem cell, e.g., iPSC.

### In Silico Methods

In embodiments in which an in silico method is used, any suitable in silico method may be used; in some cases the in silico method may depend on the type of CRISPR nuclease to be used. Exemplary in silico methods include CasOFFinder, CRISPick, CRISPOR, E-CRISP, GUIDES, RGEN Cas-Designer, RGEN Cas-Offinder, CHOPCHOP, CRISPRitz, DeepCpf1, FlashFry, CRISPR Scan (gRNAs), CRISPRseek, Off-Spotter, CCTop, CINDEL, GT-Scan, GT-Scan2, GT-Scan TUSCAN, True Design (ThermoFisher), CRISPR Design Tool (Horizon Discovery), IDT CRISPR-Cas9 guide RNA design checker, IDT Predesigned Alt-R® CRISPR-Cas9 guide RNA, IDT Custom Alt-R® CRISPR-Cas9 guide RNA, DeskgenSynthego, CRISPR-DT, CROP-IT, DeepCRISPR, Elevation, CRISPR-OFF, uCRISPR, and MIT. Choice of an in silico method may depend, in some cases, on the type of CRISPR nuclease to be used. For convenience, in silico methods will be described herein for CasOFFinder. CasOFFinder is an off-target prediction program that uses sequence homology to predict the location of off-target cut sites for both Cas9 and Cas12a nucleases. The program allows the user to select the number of allowable mismatches and whether to allow DNA or RNA bulges. Any suitable number of allowable mismatches may be used, although more than four allowable mismatches can produce a large number of potential off-target sites; in certain cases more than four allowable mismatches, such as 5 or such as 6 mismatches, may be allowed at one stage of the method, and 4 or fewer mismatches, such as 4, 3, 2, or 1 mismatches, for example 4 mismatches are allowed at one or more later stages.

### In Vitro Methods

In embodiments in which an in vitro method is used to determine potential off-target sites, any suitable in vitro method may be used. Exemplary in vitro methods include Digenome-seq, GUIDE-seq, CIRCLE-seq, GUIDE-Tag, RGEN-seq, and INDUCE-seq. For convenience, in vitro methods will be described herein for Digenonome-Seq. Digenome-Seq is an unbiased, cell-free off-target site assay which examines the susceptibility of purified cell-free DNA to be cleaved at all genomic locations. This assay has been demonstrated with Cas12a nucleases and involves incubation of purified genomic DNA with an RNP, followed by whole genome sequencing.

In certain embodiments, data generated in vitro by a method that produces a plurality of signals related to potential off-target sites can be processed by a method to eliminate false positive off-target sites, so that information used in methods to determine hazard levels of off-target sites does not include the likely false-positive sites. For example, the method can evaluate scores of flanking bases to call a peak in signal, as opposed to evaluating the cleavage score of each base individually. Additionally or alternatively, the read coverage of adjacent bases within each scoring window is also included in peak assessment. This size of the scoring window itself is adapted to individual nuclease signatures. Additionally or alternatively, the position of adjacent PAMs is considered.

An exemplary method for processing the plurality of signals that can be used with, e.g., Digenome-Seq, is the Mantis software tool. The Mantis software tool allows the identification of off-target cut sites from Digenome-seq data with an associated ‘cleavage score’. While Mantis uses a similar core scoring function to the publicly available digenome toolkit2, Mantis improves the set of returned off-target sites by employing several additional features.

The first set of features affect how the Digenome-seq data is processed. By accounting for high levels of optical duplicates observed in Digenome-seq data and resolving multi-mapped reads with the publicly available samtools markdup and “MMR” bioinformatic tools respectively, the Mantis workflow greatly reduces sequencing artifacts not otherwise accounted for in the Digenome-seq workflow. Mantis additionally discards off-target cut sites at a user-customizable threshold level if there are insufficient reads at adjacent genomic positions. This expands the “cutoff” for the total number of reads present required to call a significant off-target cut site beyond the site of the cut itself, which was all that was previously considered. With Mantis, all nucleotides used to calculate the cleavage score must meet this minimum read coverage requirement.

The second set of features refine how the cleavage score is calculated within Mantis. Mantis only returns the best peak within a user-defined region of each sample, rather than returning all peaks that exceed a given threshold, thus collapsing signal noise into a single most-likely peak. Mantis further allows the user to require a particular shape of the signal peak, allowing adjustment for nucleases with overhanging cuts and varying rates of DNA degradation during library preparation. Finally, Mantis returns information about sequence features adjacent to the called cut sites, allowing the user to select biologically relevant sites according to PAM availability and gRNA sequence matches.

Together, these features reduce the number of off-target cut sites that are called from Digenome-seq data due to sequencing artifacts and other noise. The improved set of off-target cut site candidates reduce the burden of down-stream validation experiments and produce a more reproducible set of nominated off-target sites from Digenome-seq data.

### Cell-Based Methods

In certain cases, cell-based off-target prediction or validation may be used. Exemplary cell-based techniques include Hybrid capture, Amplicon-seq, Kromatid dGH assay, rhAmp-seq, and ddPCR: both indel and translocation detection and quantification.

### Functional Categories and Databases

In certain embodiments, one or more databases are queried for information related to an off-target site. The one or more databases can comprise information regarding potential function related to one or more functional categories. A given database may be queried with a information e.g., genomic position, for an off-target site to determine whether or not the off-target site falls within one or more functional categories. Any suitable database or set of databases may be used so long as it/they provide information that can be used to determine a hazard level, and can be queried with information obtained from determinations of potential off-target sites, e.g., genomic location of a particular off-target site. Functional categories can include any suitable functional category related to a potential hazard from an alteration at the off-target site; whether or not a particular database for a functional category, or a subset of information in a database for a functional category, is related to a potential hazard can depend on a process in which a gNA will be used, a product or products produced by the method, and/or the method in which the product or products are used.

In certain embodiments, one or more databases comprise information regarding cancer-associated genes. Any suitable database or databases may be used. Exemplary databases include COSMIC's published Tier 1 Cancer Census and the Human Protein Atlas. Additionally or alternatively, in certain embodiments, one or more databases comprise information regarding disease-associated genes. Exemplary databases include Human Protein Atlas (for diseases other than cancer), and ClinVar. Additionally or alternatively, in certain embodiments, one or more databases comprise information regarding genes associated with proliferation, development, cell differentiation, and/or metabolism. An exemplary database is Gene Ontology (GO). Additionally or alternatively, in certain embodiments, one or more databases include information regarding protein-coding exons. Exemplary databases include ENSEMBL and UniProt. Additionally or alternatively, in certain embodiments, one or more databases include information regarding one or more regulatory elements. An exemplary database is ENCODE Candidate cis-Regulatory Elements. Additionally or alternatively, in certain embodiments, one or more databases include information regarding functional non-coding nucleotide sequences. An exemplary database is MultiMir. Additionally or alternatively, one or more of the following databases may be used: Annotatr, CADD, geneHancer, NCBI BLAST, UCSC BLAT, Genome Magician, COSMIC gene annotations, DECIPHER, TumorPortal, NCBI RefSeq, GENCODE, REACTOME, KEGG, AmiGO 2, Gene2Function, HuVarBase, GENEMANIA, JASPAR, ChIP Base, MEME, Factorbook, and AUGUSTU.

Cell-Based Information Regarding gNAs

In certain embodiments, cell-based information regarding one or more gNAs is used in determining one or more hazard levels, a recommendation, or other process. Cell-based information is typically produced by introducing a CRISPR complex comprising a gNA and a CRISPR nuclease, and/or one or more polynucleotides coding for one or more components of the complex, into cells in a population of cells and assessing the cells in the population after introduction. Any suitable cell-based method may be used. Suitable cell-based methods include methods providing information regarding sequences at potential off-target sites and/or sequences affected by off-target events; translocations; off-target insertions; growth, proliferation, and or survival of cells into which the complex is introduced or their progeny; and expression levels of genes associated with a pathology.

Cell-based methods that that provide information regarding sequences at potential off-target sites and/or sequences affected by off-target events include rhAmpSeq and/or droplet digital (dd) PCR). In some cases, sequence information can be used to eliminate potential off-target sites for a given gNA based on low or no frequency of sequence changes found at the potential off-target sites and/or to increase resolution of genomic location for a particular off-target site. Either or both of these results may be used to refine determination of a hazard level for a gNA, querying one or more databases for functional effects, or both. In the former case, for example, hazard levels for a subset of potential off-target sites, rather than a hazard levels for all potential off-target sites from in silico and/or in vitro methods, may be used in determining a hazard level for a particular gNA. In the latter case, increasing resolution for a particular genomic location to be queried in one or more databases can result in elimination of some potential functional effects for the gNA that were included in earlier assessments using the less-resolved genomic location. That is, more functional effects will likely be indicated if the genomic location is resolved to a level of, e.g., 20 base pairs than will be indicated if the genomic location is resolved to a level of, e.g., one or two base pairs. In addition, in other cell-based assays the number of potential areas to be investigated may be reduced to only those for which actual effect at an off-target site was found.

Cell-based assays for translocations can include any suitable assays, for example one or both of assays of karyotype, e.g., G-banding or other suitable assay, and micro-translocation. “Micro-translocation,” as that term is used herein, includes translocations that do not produce a result visible by karyotyping. Exemplary assays for micro-translocations can include hybrid capture and suitable analysis, e.g, by ddPCR.

Cell-based assays for off-target insertions can include any suitable assays, such as hybridization, in some cases including ddPCR.

Cell-based assays for growth, proliferation, and/or viability are well-known in the art and any suitable assay or combination of assays may be used.

Cell-based assays for expression levels of one or more genes associated with pathology are well-known in the art. In certain cases, a pathology is cancer. One or more screening panels may be used, according to the pathology to be investigated. These assays can be orthogonal to other cell-based assays used in methods herein; that is, the results they detect are not dependent on knowledge of any particular off-target sites.

In certain cases, cell-based assays are used in one or more processes that determine an overall hazard level for a gNA. For example, sequencing, translocation, and/or gene insertion assays may be used to provide preliminary hazard levels for a gNA based on information from each respective assay, and the preliminary hazard levels combined to give an overall hazard level for the gNA. A preliminary hazard level determination can be based on information from a particular cell-based assay. Thus, for a given gNA there may be a preliminary hazard level based on a sequencing assay, a preliminary hazard level based on a translocation assay, a preliminary hazard level based on an insertion level, etc. The preliminary hazard levels may be combined, e.g., by summation, to determine an overall hazard level for the gNA. Determination of a preliminary hazard level may include, for a given off-target event produced at a given off-target site assayed by a particular assay, a loci hazard multiplier (Lⱼ) for the off-target site, a frequency of events at the off-target site (Fⱼ) (or derivative thereof) in the particular assay, and a performance assessment for the particular assay used (P_{A}). Lⱼ for a given off-target site may be based on, e.g., information obtained by querying one or more databases regarding the genomic location of the site, as described above. Lⱼ can be determined according to the hazard level assigned to the site, either as a value from continuous values (e.g., a numerical score from 0 to 1, 0 being no hazard, and 1 being highest hazard) or a value that corresponds to a discrete hazard level classification. An example of the latter is if an off-target site is classified as high hazard, an Lⱼ of 100 is assigned, if classified as moderate hazard, an Lⱼ of 1 is assigned, and if classified as low hazard an LJ of 0.1 is assigned. These values are merely exemplary, and there may be 2 hazard levels or more than 3, and each hazard level may be assigned a different multiplier than in this example. Fⱼ can be determined as frequency of event (e.g., proportion of cells in a population of cells in which the event is detected), such as a percentage. If a derivative of Fⱼ is used, any suitable derivative may be used. P_{A} is determined as a numerical value that reflects the reliability of the assay, e.g., as a regression coefficient for a line determined by evaluation of results of the assay and ideal and/or standardized results.

An exemplary calculation of a hazard level (also referred to herein as a hazard score, or risk score, or the like) for an off-target site, as evaluated by a particular assay is:

$${E}_{j} = {L}_{j} * {log}_{10} ( {F}_{j} + 1 ) * {P}_{A}$$

where Fⱼ is expressed as a percentage.

A hazard score for the off—

target site may then be obtained by summing the hazard scores for each assay used: R_{E}=Σⱼ₌ₙ Eⱼ, wherein Eⱼ is the hazard score for a given off-target event j.
In certain cases, Fj and/or PA may be set to a fixed value. For example, in assessment of in silico hazard levels (scores), Fj and PA may be fixed, so that the value of E is based solely on Lj for the site. For a plurality of gNAs being evaluated, overall hazard scores for each of the gNAs may be determined, and the gNAs ranked, or the overall hazard score for each of the gNAs may be combined with other information, to provide a recommendation, a report, or other output for a user to determine a gNA, or a set of gNAs, to be used in a CRISPR process. Other information can include further cell-based assay information. For example, cell-based assays for growth, proliferation, and/or viability may performed with certain of the plurality of gNAs; such information can indicate whether a given gNA will produce cells of sufficient robustness, ability to produce viable progeny, and/or other indicators, to determine the usefulness of the gNA in one or more processes in which it will be used—a gNA that produces few cells or progeny that are viable, and/or that cells proliferate poorly, or the like, may be passed over in favor of one or more gNAs producing more favorable results in the assays. Additionally or alternatively, a gNA that produces results in a cell-based assay of expression levels associated with pathology, e.g., associated with cancer, that indicate that such expression occurs in some portion, or all, of the cells into which it is introduced, may be passed over in favor of one or more gNAs that do not produce such results, or that produce a lower level of such results.

Further, at any point in the evaluation process for a given gNA, one or more factors that modulate, for a product to be produced by using the gNA, a process to be used to produce the product, and/or a desired use of the product, one or more effects for an off-target event or set of such events for a gNA may be used in a determination as to whether or not to recommend and/or use the gNA. For example, if one or more off-target events produce one or more markers that can be used, e.g., to identify and/or eliminate cells in which the event or events have occurred, the gNA may be useful so long as the cells are partially or completely eliminated. Alternatively or additionally, the process for which the gNA will be used may allow the ability to select for one or more populations of cells produced in the process, e.g. clonal populations, wherein the off-target events have not occurred. For example, clonal cell populations produced from a stem cell, e.g., an iPSC, can be tested using appropriate assays to determine if an off-target event has occurred in the cells, and, if so, the clonal population will not be used in the rest of the process. Additionally or alternatively, a level of risk of the use of a product produced in a method using the gNA may assessed and may affect a decision whether or not to use the gNA. For example, a particular off-target site may produce an effect only in tissues not related to the intended area of use, the population for which the product will be used will not be affected (e.g., if a product will be used in adults and an effect occurs only in pediatric patients, or a sex-linked risk, and the like).

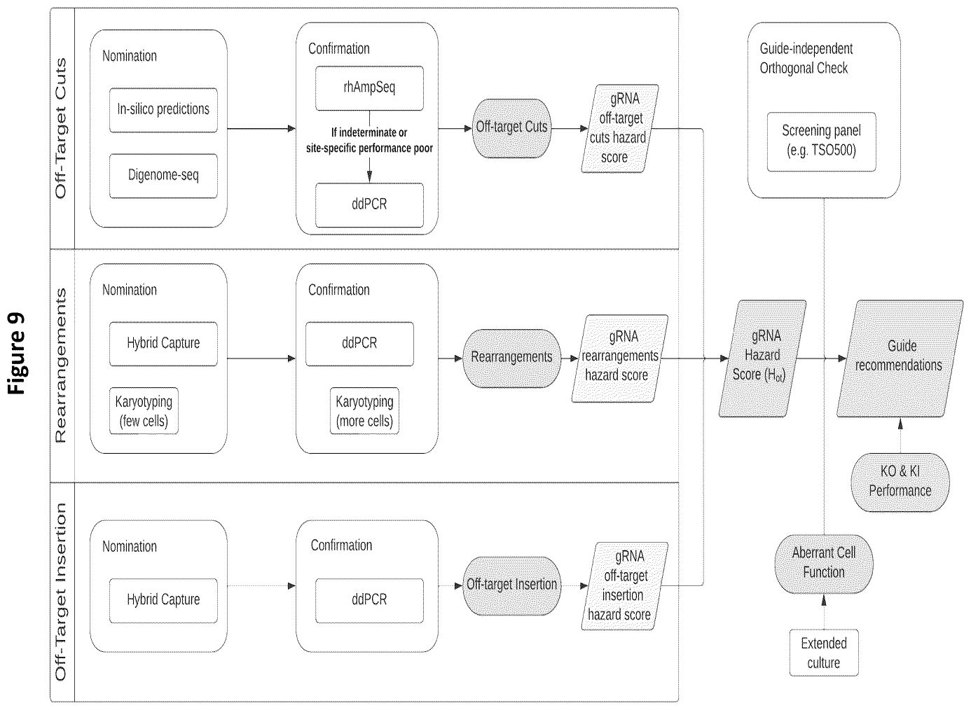

An exemplary process for evaluating gNAs is shown in FIG. 9. For off-target cuts, potential off-target sites from in silico predictions, or from in silico predictions that are used to select a subset of gNAs that are then tested in vitro, e.g., by Digenome-Seq, or in combination with in vitro testing, e.g., by Digenome-seq, are confirmed using rhAmp-Seq, and ddPCR if indeterminate or site-specific performance is poor to provide a selection of off-target cuts (sites) each of which is assigned a hazard score (level). For rearrangements, hybrid capture and karyotyping of a few cells can be confirmed by ddPCR and karyotyping providing a selection of off-target sites leading to rearrangements, each of which is assigned a hazard score (level). For off-target insertion, potential off-target sites are subject to hybrid capture followed by ddPCR, and off-target sites leading to insertion are each assigned a hazard score (level). The hazard scores are combined to determine an overall hazard score for a gRNA. Further testing can include cell-based assays for transcription of genes involved in one or more pathologies, e.g., cancer and/or cell-based assays to determine viability, growth, and/or proliferation. Some or all of these steps can be performed for a plurality of gRNAs, and can produce guide recommendations for one or more gRNAs to be used in CRISPR processes.

For any of the methods that include evaluating gNAs as described herein, part or all of the method may be computer implemented, and such computer-implemented methods are included herein, as well as apparatus, such as a data processing apparatus, to carry out some or all of the steps of the method; a computer program comprising instructions which, when the program is executed by a computer, cause the computer to carry out some or all of the steps of the method (or a computer-readable data carrier having stored thereon the program, or a data carrier signal carrying the program); or a computer-readable storage medium comprising instructions which, when executed by a computer, cause the computer to carry out some or all of the steps of the method. A “computer,” as that term is used herein, includes a general purpose computer modified (e.g., programmed or configured) by software to be a special-purpose computer to perform part or all of methods described herein. Computers can include a processor coupled to code and data memory and an input/output system (for example, comprising interfaces for a network and/or storage media and/or other communications. A computer may also comprise a user interface and a user display. A computer can be a single computing device or multiple computing devices connected in such a manner as to allow performance of some or all of the methods described herein. A computer may provide output at one or more stages of a method, for example output in a user-readable form, such as on a display, in a communication from the computer, and/or as hard copy. A computer can include a memory unit configured to receive and/or store information regarding potential off-target sites, information from which potential off-target sites may be derived (e.g., data for gNAs with various spacer sequences, or data allowing such sequences to be derived, data regarding one or more target polynucleotides, data regarding one or more genomes for an in silico determination of off-target sites, data from in vitro determination of target sites, and the like) and one or more processors that alone or in combination are programmed to carry out some or all of the steps of a method described herein. A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to methods and compositions described herein can be transmitted over such networks or connections (or any other suitable means for transmitting information, including but not limited to mailing a physical report, such as a print-out) for reception and/or for review by a receiver. The receiver can be but is not limited to an individual or group of individuals, and/or electronic system (e.g. one or more computers, and/or one or more servers).

Further provided herein are compositions wherein at least part of the composition is selected on the basis of methods for evaluating gNAs as described herein. In certain embodiments provided is a composition comprising a gNA, or one or more polynucleotides coding therefor, wherein the gNA is compatible with a CRISPR nuclease wherein the gNA comprises a spacer sequence partially or completely complementary to a target sequence in a target polynucleotide, and wherein the gNA is selected from a plurality of potential gNAs, each of which is complementary to a different target sequence in the target polynucleotide, by any one of the methods for evaluating gNAs described herein.

Thus, in certain embodiments provided herein is computer-implemented method for evaluating an off-target site, e.g., a potential off-target site for a guide nucleic acid (gNA), wherein the gNA comprises a spacer sequence partially or completely complementary to a target sequence in a target polynucleotide in a genome and is compatable with a CRISPR-associated nuclease, comprising providing to the computer a genomic position for the potential off-target site for the gNA; and, on the computer, determining a hazard level for the off-target site or potential off-target site. The hazard level may be determined by any suitable method such as a method based, at least in part, on the genomic position. In certain embodiments, the hazard level is determined by a method comprising querying one or more databases that comprise information regarding potential function with the genomic position of the off-target or potential off-target site to determine whether or not the site falls within one or more functional categories; and determining a hazard level for the potential off-target site based, at least in part, on the results of the querying. Any suitable databases may be used. In certain embodiments, one or more databases comprising information regarding cancer-associated genes is used. Alternatively or additionally, one or more databases comprising information regarding disease-associated genes is used. Alternatively or additionally, one or more databases comprising information regarding genes associated with proliferation, development, cell differentiation, and/or metabolism is used. Alternatively or additionally, one or more databases comprising information regarding protein-coding exons is used. Alternatively or additionally, one or more databases comprising information regarding one or more regulatory elements is used Alternatively or additionally, one or more databases comprising information regarding functional non-coding nucleotide sequences is used. Off-target site or potential off-target sites may be determined by any suitable method, such as a method described herein. In certain embodiments, off-target sites or potential off-target sites are determined for a Type V CRISPR nuclease, e.g., a Type VA nuclease, such as a nuclease that is partially or completely identical to SEQ ID NO: 37, e.g., as described in the section Determining Spacer Sequences and off-target or potential off-target sites. The method may further comprise evaluating a plurality of off-target or potential off-target sites for the gNA, where each off-target site or potential off-target site is different from other off-target sites or potential off-target sites, and where a hazard level for each off-target site or potential off-target site is determined as described above, and determining a hazard level for the gNA, based, at least in part, on the combining the hazard levels thus determined. The method can further comprise determining hazard levels for a plurality of gNAs, wherein each of the gNAs comprises a spacer sequence partially or completely complementary to a target sequence in the target polynucleotide, and wherein each target sequence is different from other target sequences, comprising performing the steps described above for each gNA. The method can further comprise ranking the plurality of gNAs based, at least in part, on the gNA hazard levels thus determined. In certain embodiments, the ranking is based also on editing efficiency for each gNA; in certain of these embodiments, potential off-target sites for each gNA are determined in silico, and gNAs ranked on the basis of hazard level combined with editing efficiency. In certain embodiments, in vitro methods are used to determine off-target sites or potential off-target sites. gNAs can be ranked based, at least in part, on hazard levels determined for potential off-target sites determined in silico, and a subset of the gNAs selected based, at least in part, on their rankings, for further testing in vitro, where in vitro testing is used to determine off-target or potential off-target sites for each of the gNAs in the subset, and hazard levels for each of the sites determined, then hazard level for each gNA determined, at least in part, by combining the hazard levels of the sites. At one or more steps in the above process, cell-based information regarding the one or more gNAs is provided to the computer, and the cell-based information is used in one or more steps relating to determining a hazard level for a gNA, ranking of gNAs, or both. In certain embodiments, cell-based information is obtained from cells into which have been introduced the CRISPR-associated nuclease, or one or more poynucleotides coding therefor, and the gNA, or one or more polynucleotides coding therefor, and the cell-based information comprises information regarding off-target events for each gNA. In certain embodiments the cell-based information comprises sequence information for the one or more potential off-target sites. In certain embodiments the sequence information for the one or more potential off-target sites is used to eliminate potential off-target sites from consideration in determining a hazard level for a gNA, to increase genome location resolution to determine a hazard level for a potential off-target site, or both. Additionally or alternatively cell-based information comprises translocation information, such as information regarding karyotype and/or micro-translocations Additionally or alternatively cell-based information comprises information regarding off-target insertions. Additionally or alternatively cell-based information comprises information regarding growth, proliferation, and/or viability of cells into which the gNA is introduced or their progeny. Additionally or alternatively cell-based information comprises information regarding information regarding expression levels of one or more genes associated with a pathology, such as cancer, of cells into which the gNA is introduced. In certain embodiments a preliminary hazard level for each cell-based assay is determined by assigning a numerical value for hazard level for the off-target event or events of each cell-based assay and multiplying by a frequency of the occurrence of the off-target event in the assay. The determination may further comprise assigning a numerical value to performance of each assay and multiplying the value obtained by multiplying hazard level and frequency by the numerical value. In certain embodiments, the method comprises combining the preliminary hazard levels for the cell-based assays a gNA comprises cell-based information regarding to determine an overall hazard level for the gNA. In certain embodiments, a preliminary hazard level is determined for a gNA from cell-based sequence information regarding off-target or potential off-target sites, translocations, and/or insertions is used in determining a hazard level for a gNA. The hazard level thus obtained may be modified by information regarding expression levels of one or more genes associated with pathology, e.g., cancer, in cells in which the gNA has been used in a CRISPR process and/or by information regarding growth, proliferation, and/or viability of cells into which the gNA is introduced or their progeny. At any stage of the method a report and/or recommendation may be generated based, at least in part, on the information obtained in the method to that point. Generating the report and/or recommendation can further comprise determining one or more factors that modulate one or more effects of one or more events for an off-target site for the one or more gNAs on a desired product to be produced in a method comprising introducing the gNA and its compatible CRISPR nuclease into cells, a process to produce the product, and/or desired use of the product. In certain embodiments the one or more factors comprise a presence of one or more cell markers directly or indirectly produced by the one or more off-target events for the off-target site, wherein the one or more cell markers can be used to selectively remove cells displaying the one or more cell markers from a population of cells used to produce the product. Additionally or alternatively the one or more factors comprise an ability to select for a population of cells, e.g., clonal populations, used in the process to produce the product, wherein the one or more events at the one or more off-target sites has not occurred in the cells. Additionally or alternatively the one or more factors comprises determining a level of acceptable risk for the occurrence of the one or more events at the one or more off-target sites in a subject or population of subjects for whom the product will be used in treatment. In certain embodiments provided is a data processing apparatus comprising a processor configured to perform one or more of the above methods (i.e., methods described in this paragraph). In certain embodiments provided is a computer program comprising instructions which, when the program is executed by a computer, causes the computer to carry out one or more of the above methods. In certain embodiments provided is data carrier signal carrying the computer program. In certain embodiments provided is a computer-readable storage medium comprising instructions which, when executed by a computer, cause the computer to carry out one or more of the above methods. In certain embodiments provide is a composition comprising a gNA, or one or more polynucleotides coding therefor, wherein the gNA is compatible with a CRISPR nuclease, such as a Type VA nuclease, wherein the gNA comprises a spacer sequence partially or completely complementary to a target sequence in a target polynucleotide, and wherein the gNA is selected from a plurality of potential gNAs, each of which is complementary to a different target sequence in the target polynucleotide, by one or more of the above methods. In certain embodiments the composition further comprises the CRISPR nuclease or one or more polynucleotides coding therefor. In certain embodiments provided is a cell comprising the composition, or a progeny thereof.

In certain embodiments, one or more guide nucleic acids (gNAs), each comprising a spacer sequence can be generated for a target gene. In certain embodiments, a spacer sequence can be cross-reference with a first set of databases to provide a list comprising a plurality of target and off-target sequences. Any suitable database can be used, such as a database comprising off-target sequences generated via in silico modeling, for example casOFFinder, genomic data, in vitro data, cell-free data, cell-based data, preclinical data, animal data, and/or clinical data. In certain embodiments, the set of databases comprise data generated by casOFFinder and sequencing data. In certain embodiments, the set of databases comprises a single database. In certain embodiments, the set of databases comprises two or more databases. Any suitable number of databases can be used, such as at least any of 1, 2, 3, 4, 5, 6, 7, 8, 9, 10, 11, 12, 13, 14, 15, 20, 25, 30, 35, 40, or 45 and/or not more than any of 2, 3, 4, 5, 6, 7, 8, 9, 10, 11, 12, 13, 14, 15, 20, 25, 30, 35, 40, 45, or 50 databases, for example 1-50 databases, preferably 1-20 databases, more preferably 1-10 databases, even more preferably 7 databases. In certain embodiments an algorithm or a computer-implemented method is used to cross-reference the spacer sequence with the one or more databases, wherein the output is a list of target and/or off-target sequence entries, each of which corresponds to a site in which the spacer sequence shows at least some complementary to and has the potential to bind and act when complexed with a nucleic acid-guided nuclease.

In certain embodiments, each target and/or off-target site entry in the list is cross-referenced with a second set of one or more databases related to the functional properties of the entry, wherein a plurality of risks are associated with each entry. Any suitable number of databases can be used, such as at least any of 1, 2, 3, 4, 5, 6, 7, 8, 9, 10, 11, 12, 13, 14, 15, 20, 25, 30, 35, 40, or 45 and/or not more than any of 2, 3, 4, 5, 6, 7, 8, 9, 10, 11, 12, 13, 14, 15, 20, 25, 30, 35, 40, 45, or 50 databases, for example 1-50 databases, preferably 1-20 databases, more preferably 1-10 databases, even more preferably 7 databases. In certain embodiments, an entry is classified as a high risk site if little to know information about the site is known. In certain embodiments, an entry is classified as a high risk site if it is associated with a site associated with a cancer and/or a known disease gene. In certain embodiments, an entry is classified as high risk site if it is associated with a gene involved in cell kinetics and/or cell growth/proliferation. In certain embodiments, an entry is classified as moderate risk if it is associated with a coding and/or transcribed region. In certain embodiments, an entry is classified as moderate risk if it is associated with a region involved in regulating the expression of one or more genes, such as a promoter and/or a transcription factor. In certain embodiments, an entry is classified as low risk if it is associated with a non-coding region, for example not in an ENCODE cis-Reg site. In certain embodiments, collated risks for each entry for a spacer sequence comprises the aggregate risk profile for the spacer sequence. In certain embodiments, the risk profile can be viewed as a histogram, wherein the x-axis represents the risk category (low, medium, high) and the y-axis represents the count of each risk category. Any suitable visualization and/or data storage method may be used for the risk profile.

In certain embodiments, the risk profile is manually assessed by one or more individuals. In certain embodiments, the risk profile can be updated by the assessment of the individual and inputted into the computer as necessary. In certain cases an individual can manually curate the moderate any of the entries in the risk profile with supplementary data, for example in vitro cell analytics data and/or in vitro/in vivo study data. In certain embodiments, the individual may assess a moderate risk entry for the following four criteria: (1) is detectable in drug substance, (2) has a known relevance, (3) demonstrates an acceptable level of risk, and/or (4) has a risk mitigation strategy available. In certain embodiments, an individual may promote a moderate risk entry to a high risk entry is any of the 4 criteria are not met. In certain embodiments, an individual may maintain an entry as moderate risk if all of the 4 criteria are met.

In certain embodiments, the first and/or second set of databases may contain clinical information from the use of the gNAs in one or more clinical programs. In certain embodiments, the clinical data comprises sequencing data from one or more subjects and/or outcomes from one or more subjects. Any suitable clinical data can be used.

In certain embodiments, provide herein is a computer-implemented method for identifying potential off-target sites and/or calculating risk profiles for one or more guide nucleic acids (gNAs) each comprising a spacer sequence. In certain embodiments, the computer-implemented method comprises providing to a computer one or more spacer sequences, wherein the spacer sequence is at least partially complementary to a target sequence, and, optionally, one or more off-target sequences. The one or more spacer sequences can be provided to the computer using any suitable method, for example a csv file and/or a graphic user interface. Any number of spacer sequences can be provided to the computer. In certain embodiments, the computer-implemented method comprises, for each spacer sequence, cross-referencing the spacer sequence with a first set of one or more databases to provided a list comprising a plurality of target and off-target sequence entries. Any suitable number of databases can be used, such as at least any of 1, 2, 3, 4, 5, 6, 7, 8, 9, 10, 11, 12, 13, 14, 15, 20, 25, 30, 35, 40, or 45 and/or not more than any of 2, 3, 4, 5, 6, 7, 8, 9, 10, 11, 12, 13, 14, 15, 20, 25, 30, 35, 40, 45, or 50 databases, for example 1-50 databases, preferably 1-20 databases, more preferably 1-10 databases, even more preferably 7 databases. In certain embodiments, the first set of databases comprises in silico data, for example casOFFinder, genomic data, in vitro data, cell-free data, cell-based data, preclinical data, and/or clinical data. In certain embodiments, the in vitro data comprises sequencing data, for example Amplicon-sesq and/or Digenome-seq, qPCR data, digital PCR data, isothermal amplification data, and/or microarray data. In certain embodiments, the cell-based data comprises karyotyping data, growth data, proliferation data, and/or survival data. In certain embodiments, the computer-implemented method comprises, for each spacer sequence and for each target and/or off-target sequence entry, cross-referencing the entry with a second set of one or more databases related to the functional properties of the entry to provide a plurality of risk associated with the entry. Any suitable number of databases can be used, such as at least any of 1, 2, 3, 4, 5, 6, 7, 8, 9, 10, 11, 12, 13, 14, 15, 20, 25, 30, 35, 40, or 45 and/or not more than any of 2, 3, 4, 5, 6, 7, 8, 9, 10, 11, 12, 13, 14, 15, 20, 25, 30, 35, 40, 45, or 50 databases, for example 1-50 databases, preferably 1-20 databases, more preferably 1-10 databases, even more preferably 7 databases. In certain embodiments, the computer-implemented method comprises for each spacer sequence, calculating a first risk profile comprising the plurality of risks for each spacer sequence. In certain embodiments, the risk profile is calculated from the plurality of risks comprises a set of categorized risk values obtained by binning the risks into low, medium, and high and subsequently summing the risks in each category to provide the categorized risk value. In certain embodiments, the computer-implemented method comprises a user reviewing the first risk profile and, optionally, providing to the computer a second risk profile, the computer-implemented method storing the second risk profile in memory. In certain embodiments, the computer-implemented method comprises a user entering clinical data relevant to the use of a gNA comprising the spacer sequence to the computer, the computer-implemented method storing the clinical data in memory and, optionally, calculating and storing a third risk profile. In certain embodiments, an output of the risk profile is provided to the user.

In certain embodiments, provided herein are a computer system for identifying potential off-target sites and/or calculating a risk profile for a guide nucleic acid. In certain embodiments, the at least one computing device, comprises at least one process, a memory, and a communication bus connecting the at least one processor with the memory. In certain embodiments, the processor is configured to perform the computer implement method as described in the paragraph above.

### II. ENGINEERED NON-NATURALLY-OCCURRING CRISPR-CAS SYSTEMS

A CRISPR-Cas system generally comprises a Cas protein and one or more guide nucleic acids (gNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence, also referred to herein as a target sequence, in the target strand of the target polynucleotide. Typically, both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called a spacer sequence that is at least partially complementary to and can hybridize with a target nucleotide sequence, where target nucleotide sequence is located adjacent to a PAM in an orientation operable with the Cas protein. It has been observed that not all CRISPR-Cas systems designed by these criteria are equally effective. The larger polynucleotide in which a target nucleotide sequence is located may be referred to as a target polynucleotide; e.g., a chromosome or other genomic DNA, or portion thereof, or any other suitable polynucleotide within which a target nucleotide sequence is located. The target polynucleotide in double stranded DNA comprises two strands. The strand of the DNA duplex to which the spacer sequence is complementary herein is called the “target strand,” while the strand to which the spacer sequence shares sequence identity herein is called the “non-target strand.”

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168:328). Among the types of class 2 CRISPR-Cas systems, type II and type V systems typically target DNA and type VI systems typically target RNA (id.). Naturally occurring type II effector complexes include Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85:227). Certain naturally occurring type V systems, such as type V-A, type V-C, and type V-D systems, do not require tracrRNA and use crRNA alone as the guide for cleavage of target DNA (see, Zetsche et al. (2015) CELL, 163:759; Makarova et al. (2017) CELL, 168:328.

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type II Cas nucleases have been developed, for example, by linking the crRNA and the tracrRNA (see, e.g., U.S. Pat. Nos. 10,266,850 and 8,906,616). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

Naturally occurring Type V-A, Type V-C, and Type V-D CRISPR-Cas systems lack a tracrRNA and rely on a single crRNA to guide the CRISPR-Cas complex to the target polynucleotide. Dual guide nucleic acids capable of activating type V-A, type V-C, or type V-D Cas nucleases have been developed, for example, by splitting the single crRNA into a targeter nucleic acid and a modulator nucleic acid (see, e.g., International (PCT) Application Publication No. WO 2021/067788). Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. These CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

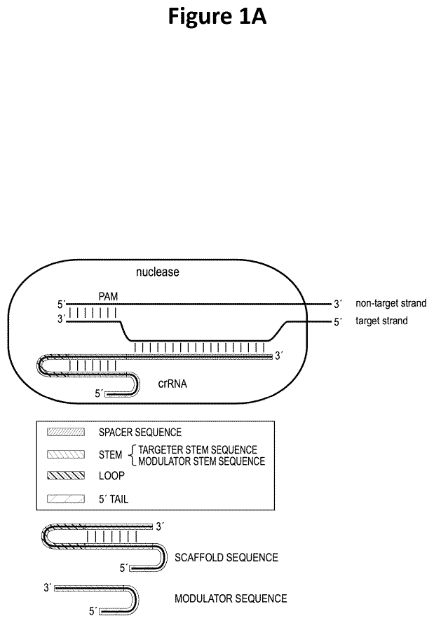

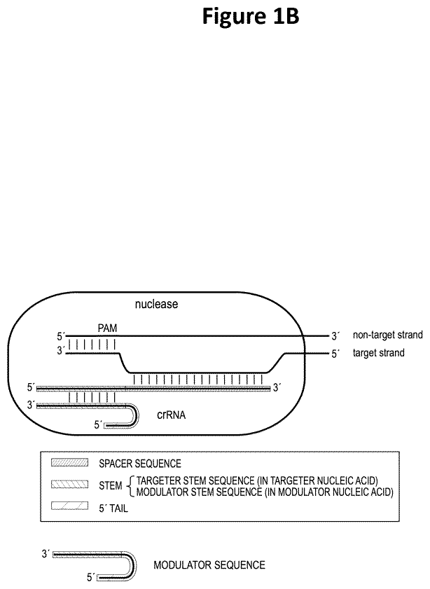

Elements in an exemplary single guide CRISPR Cas system, e.g., a type V-A CRISPR-Cas system, are shown in FIG. 1A. The single gNA can also be called a “crRNA” or “single gRNA” where it is present in the form of an RNA. It can comprise, from 5′ to 3′, an optional 5′ sequence, e.g., a tail, a modulator stem sequence, a loop, a targeter stem sequence complementary to the modulator stem sequence, and a spacer sequence that is at least partially complementary to and can hybridize with a target sequence in the target strand of the target polynucleotide. Where a 5′ tail is present, the sequence including the 5′ tail and the modulator stem sequence can also be called a “modulator sequence” herein. A fragment of the single guide nucleic acid from the optional 5′ tail to the targeter stem sequence, also called a “scaffold sequence” herein, bind the Cas protein. In addition, the PAM in the non-target strand of the target DNA binds the Cas protein.

Elements in an exemplary dual guide type CRISPR Cas system, e.g., a dual guide type V-A CRISPR-Cas system are shown in FIG. 1B. The first guide nucleic acid, which can be called a “modulator nucleic acid” herein, comprises, from 5′ to 3′, an optional 5′ tail and a modulator stem sequence. Where a 5′ tail is present, the sequence including the 5′ tail and the modulator stem sequence can also called a “modulator sequence” herein. The second guide nucleic acid, which can be called “targeter nucleic acid” herein, comprises, from 5′ to 3′, a targeter stem sequence complementary to the modulator stem sequence and a spacer sequence that is at least partially complementary to and can hybridize with the target sequence in the target strand of the target polynucleotide. The duplex between the modulator stem sequence and the targeter stem sequence, plus the optional 5′ tail, constitute a structure that binds the Cas protein. In addition, the PAM in the non-target strand of the target DNA binds the Cas protein. It is understood that, in a dual gNA, e.g., dual gRNA, the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

The terms “targeter stem sequence” and “modulator stem sequence,” as used herein, can refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

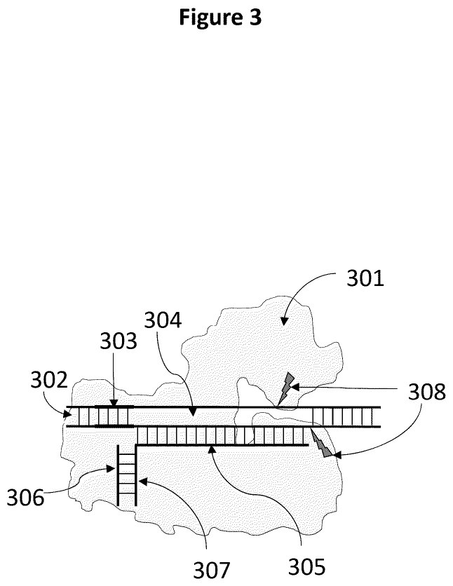

An illustrative example of a nucleic acid-guided nuclease complex is shown in FIG. 3. Specifically, FIG. 3 shows a Type V-A nucleic acid guided nuclease (301) complexed with a gual gNA comprising a modulator nucleic acid (306) and a targeter nucleic acid (307), wherein the modulator nucleic acid and targeter nucleic acid are hybridized through a stem. The targeter nucleic acid further comprises a spacer sequence (305) at least partially complementary to a target nucleotide sequence (304), i.e., a protospacer, in a target polynucleotide (302) adjacent to a suitable PAM (303). Upon binding to the target nucleotide sequence, the nucleic acid-guided nuclease complex can generate one or more strand breaks (308) in the target polynucleotide at or near the target nucleotide sequence.

### A. Cas Proteins

A guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of binding a CRISPR Associated (Cas) protein, e.g., a Cas nuclease. In certain embodiments, the guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of activating a Cas nuclease. A gNA capable of activating a particular Cas nuclease is said to be “compatible” with the Cas nuclease; a Cas nuclease capable of being activated by a particular gNA is said to be “compatible” with the gNA.

The terms “CRISPR-Associated protein,” “Cas protein,” and “Cas,” as used interchangeably herein, can refer to a naturally occurring Cas protein or an engineered Cas protein. Non-limiting examples of Cas protein engineering include but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of engineered Cas comprises altered ability (e.g., specificity or kinetics) to bind a naturally occurring gNA, e.g., gRNA or engineered gNA, e.g., gRNA, altered ability (e.g., specificity or kinetics) to bind a target nucleotide sequence, altered processivity of nucleic acid scanning, and/or altered effector (e.g., nuclease) activity. A Cas protein having nuclease activity can be referred to as a “CRISPR-Associated nuclease” or “Cas nuclease,” or simply “nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, a type V-A Cas nuclease comprises AsCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 3 of International (PCT) Application Publication No. WO 2021/158918. In certain embodiments, a type V-A Cas protein comprises the amino acid sequence set forth in SEQ ID NO: 3 of International (PCT) Application Publication No. WO 2021/158918.

In certain embodiments, a type V-A Cas nuclease comprises LbCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 4 of International (PCT) Application Publication No. WO 2021158918. In certain embodiments, a type V-A Cas protein comprises the amino acid sequence set forth in SEQ ID NO: 4 of International (PCT) Application Publication No. WO 2021/158918.

In certain embodiments, a type V-A Cas nuclease comprises FnCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 5 of International (PCT) Application Publication No. WO 2021158918. In certain embodiments, a type V-A Cas protein comprises the amino acid sequence set forth in SEQ ID NO: 5 of International (PCT) Application Publication No. WO 2021/158918.

In certain embodiments, a type V-A Cas nuclease comprises Prevotella bryantii Cpf1 (PbCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 6 of International (PCT) Application Publication No. WO 2021/158918. In certain embodiments, a type V-A Cas protein comprises the amino acid sequence set forth in SEQ ID NO: 6 of International (PCT) Application Publication No. WO 2021/158918.

In certain embodiments, a type V-A Cas nuclease comprises Proteocatella sphenisci Cpf1 (PsCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 7 of International (PCT) Application Publication No. WO 2021158918. In certain embodiments, a type V-A Cas protein comprises the amino acid sequence set forth in SEQ ID NO: 7 of International (PCT) Application Publication No. WO 2021/158918.

In certain embodiments, a type V-A Cas nuclease comprises Anaerovibrio sp. RM50 Cpf1 (As2Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 8 of International (PCT) Application Publication No. WO 2021158918. In certain embodiments, a type V-A Cas protein comprises the amino acid sequence set forth in SEQ ID NO: 8 of International (PCT) Application Publication No. WO 2021/158918.

In certain embodiments, a type V-A Cas nuclease comprises Moraxella caprae Cpf1 (McCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 9 of International (PCT) Application Publication No. WO 2021/158918. In certain embodiments, a type V-A Cas protein comprises the amino acid sequence set forth in SEQ ID NO: 9 of International (PCT) Application Publication No. WO 2021/158918.

In certain embodiments, a type V-A Cas nuclease comprises Lachnospiraceae bacterium COE1 Cpf1 (Lb3Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 10 of International (PCT) Application Publication No. WO 2021158918. In certain embodiments, a type V-A Cas protein comprises the amino acid sequence set forth in SEQ ID NO: 10 of International (PCT) Application Publication No. WO 2021/158918.

In certain embodiments, a type V-A Cas nuclease comprises Eubacterium coprostanoligenes Cpf1 (EcCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 11 of International (PCT) Application Publication No. WO 2021158918. In certain embodiments, a type V-A Cas protein comprises the amino acid sequence set forth in SEQ ID NO: 11 of International (PCT) Application Publication No. WO 2021/158918.

In certain embodiments, a type V-A Cas nuclease is not Cpf1. In certain embodiments, a type V-A Cas nuclease is not AsCpf1.

In certain embodiments, a type V-A Cas nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20, or variants thereof. MAD1-MAD20 are known in the art and are described in U.S. Pat. No. 9,982,279.

In certain embodiments, a type V-A Cas nuclease comprises MAD7 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 37. In certain embodiments, a type V-A Cas protein comprises the amino acid sequence set forth in SEQ ID NO: 37.

MAD7
(SEQ ID NO: 37)
MNNGTNNFQNFIGISSLQKTLRNALIPTETTQQFIVKNGIIKEDELRGENRQILKDIMDDYYRGF
ISETLSSIDDIDWTSLFEKMEIQLKNGDNKDTLIKEQTEYRKAIHKKFANDDRFKNMFSAKLISD
ILPEFVIHNNNYSASEKEEKTQVIKLFSRFATSFKDYFKNRANCFSADDISSSSCHRIVNDNAEI
FFSNALVYRRIVKSLSNDDINKISGDMKDSLKEMSLEEIYSYEKYGEFITQEGISFYNDICGKVN
SFMNLYCQKNKENKNLYKLQKLHKQILCIADTSYEVPYKFESDEEVYQSVNGFLDNISSKHIVER
LRKIGDNYNGYNLDKIYIVSKFYESVSQKTYRDWETINTALEIHYNNILPGNGKSKADKVKKAVK
NDLQKSITEINELVSNYKLCSDDNIKAETYIHEISHILNNFEAQELKYNPEIHLVESELKASELK
NVLDVIMNAFHWCSVFMTEELVDKDNNFYAELEEIYDEIYPVISLYNLVRNYVTQKPYSTKKIKL
NFGIPTLADGWSKSKEYSNNAIILMRDNLYYLGIFNAKNKPDKKIIEGNTSENKGDYKKMIYNLL
PGPNKMIPKVFLSSKTGVETYKPSAYILEGYKQNKHIKSSKDFDITFCHDLIDYFKNCIAIHPEW
KNFGFDFSDTSTYEDISGFYREVELQGYKIDWTYISEKDIDLLQEKGQLYLFQIYNKDFSKKSTG
NDNLHTMYLKNLFSEENLKDIVLKLNGEAEIFFRKSSIKNPIIHKKGSILVNRTYEAEEKDQFGN
IQIVRKNIPENIYQELYKYFNDKSDKELSDEAAKLKNVVGHHEAATNIVKDYRYTYDKYFLHMPI
TINFKANKTGFINDRILQYIAKEKDLHVIGIDRGERNLIYVSVIDTCGNIVEQKSFNIVNGYDYQ
IKLKQQEGARQIARKEWKEIGKIKEIKEGYLSLVIHEISKMVIKYNAIIAMEDLSYGFKKGRFKV
ERQVYQKFETMLINKLNYLVFKDISITENGGLLKGYQLTYIPDKLKNVGHQCGCIFYVPAAYTSK
IDPTTGFVNIFKFKDLTVDAKREFIKKFDSIRYDSEKNLFCFTFDYNNFITQNTVMSKSSWSVYT
YGVRIKRRFVNGRFSNESDTIDITKDMEKTLEMTDINWRDGHDLRQDIIDYEIVQHIFEIFRLTV
QMRNSLSELEDRDYDRLISPVLNENNIFYDSAKAGDALPKDADANGAYCIALKGLYEIKQITENW
KEDGKFSRDKLKISNKDWFDFIONKRYL

In certain embodiments, a type V-A Cas nuclease comprises MAD2 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 38. In certain embodiments, a type V-A Cas protein comprises the amino acid sequence set forth in SEQ ID NO: 38.

MAD2
(SEQ ID NO: 38)
MSSLTKFTNKYSKQLTIKNELIPVGKTLENIKENGLIDGDEQLNENYQKAKIIVDDFLRDFINKA
LNNTQIGNWRELADALNKEDEDNIEKLQDKIRGIIVSKFETFDLFSSYSIKKDEKIIDDDNDVEE
EELDLGKKTSSFKYIFKKNLFKLVLPSYLKTTNQDKLKIISSFDNFSTYFRGFFENRKNIFTKKP
ISTSIAYRIVHDNFPKFLDNIRCFNVWQTECPQLIVKADNYLKSKNVIAKDKSLANYFTVGAYDY
FLSQNGIDFYNNIIGGLPAFAGHEKIQGLNEFINQECQKDSELKSKLKNRHAFKMAVLFKQILSD
REKSFVIDEFESDAQVIDAVKNFYAEQCKDNNVIFNLLNLIKNIAFLSDDELDGIFIEGKYLSSV
SQKLYSDWSKLRNDIEDSANSKQGNKELAKKIKINKGDVEKAISKYEFSLSELNSIVHDNTKFSD
LLSCTLHKVASEKLVKVNEGDWPKHLKNNEEKQKIKEPLDALLEIYNTLLIFNCKSFNKNGNFYV
DYDRCINELSSVVYLYNKTRNYCTKKPYNTDKFKLNFNSPQLGEGFSKSKENDCLTLLFKKDDNY
YVGIIRKGAKINFDDTQAIADNTDNCIFKMNYFLLKDAKKFIPKCSIQLKEVKAHFKKSEDDYIL
SDKEKFASPLVIKKSTFLLATAHVKGKKGNIKKFQKEYSKENPTEYRNSLNEWIAFCKEFLKTYK
AATIFDITTLKKAEEYADIVEFYKDVDNLCYKLEFCPIKTSFIENLIDNGDLYLFRINNKDFSSK
STGTKNLHTLYLQAIFDERNLNNPTIMLNGGAELFYRKESIEQKNRITHKAGSILVNKVCKDGTS
LDDKIRNEIYQYENKFIDTLSDEAKKVLPNVIKKEATHDITKDKRFTSDKFFFHCPLTINYKEGD
TKQFNNEVLSFLRGNPDINIIGIDRGERNLIYVTVINQKGEILDSVSFNTVTNKSSKIEQTVDYE
EKLAVREKERIEAKRSWDSISKIATLKEGYLSAIVHEICLLMIKHNAIVVLENLNAGFKRIRGGL
SEKSVYQKFEKMLINKLNYFVSKKESDWNKPSGLLNGLQLSDQFESFEKLGIQSGFIFYVPAAYT
SKIDPTTGFANVLNLSKVRNVDAIKSFFSNFNEISYSKKEALFKFSFDLDSLSKKGFSSFVKFSK
SKWNVYTFGERIIKPKNKQGYREDKRINLTFEMKKLLNEYKVSFDLENNLIPNLTSANLKDTFWK
ELFFIFKTTLQLRNSVINGKEDVLISPVKNAKGEFFVSGTHNKTLPQDCDANGAYHIALKGLMIL
ERNNLVREEKDTKKIMAISNVDWFEYVQKRRGVL

In certain embodiments, a type V-A Cas nucleases comprises Csm1. Csm1 proteins are known in the art and are described in U.S. Pat. No. 9,896,696. Csm1 orthologs can be found in various bacterial and archaeal genomes. For example, in certain embodiments, a Csm1 protein is derived from Smithella sp. SCADC (Sm), Sulfuricurvum sp. (Ss), or Microgenomates (Roizmanbacteria) bacterium (Mb).

In certain embodiments, a type V-A Cas nuclease comprises SmCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 12 of International (PCT) Application Publication No. WO 2021/158918. In certain embodiments, a type V-A Cas protein comprises the amino acid sequence set forth in SEQ ID NO: 12 of International (PCT) Application Publication No. WO 2021/158918.

In certain embodiments, a type V-A Cas nuclease comprises SsCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 13 of International (PCT) Application Publication No. WO 2021/158918. In certain embodiments, a type V-A Cas protein comprises the amino acid sequence set forth in SEQ ID NO: 13 of International (PCT) Application Publication No. WO 2021/158918.

In certain embodiments, a type V-A Cas nuclease comprises MbCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 14 of International (PCT) Application Publication No. WO 2021/158918. In certain embodiments, a type V-A Cas protein comprises the amino acid sequence set forth in SEQ ID NO: 14 of International (PCT) Application Publication No. WO 2021/158918.

In certain embodiments, the type V-A Cas nuclease comprises an ART nuclease or a variant thereof. In general, such nucleases sequences have <60% AA sequence similarity to Cas12a, <60% AA sequence similarity to a positive control nuclease, and >80% query cover. In certain embodiments, the Type V-A nuclease comprises an ART1, ART2, ART3, ART4, ART5, ART6, ART7, ART8, ART9, ART10, ART11, ART12, ART13, ART14, ART15, ART16, ART17, ART18, ART19, ART20, ART21, ART22, ART23, ART24, ART25, ART26, ART27, ART28, ART28, ART30, ART31, ART32, ART33, ART34, ART35, or ART11* (i.e., ART11_L679F, i.e., ART11 wherein leucine (L) at amino acid position 679 is replaced with phenylalanine (F)) nuclease, as shown in Table 1. In certain embodiments, the type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence designated for the individual ART nuclease as shown in Table 1. In certain embodiments, provided is a nucleic acid-guided nuclease comprising a nucleic acid-guided nuclease polypeptide having at least 85% identity to an amino acid sequence represented by SEQ ID NOs: 1-36 or a nucleic acid encoding a nucleic acid-guided nuclease polypeptide comprising at least 85% identity with the polynucleotide represented by SEQ ID NOs: 1-36. In certain embodiments, provided is a nucleic acid-guided nuclease comprising a polypeptide having at least 90% identity to the amino acid sequence represented by SEQ ID NOs: 1-36, wherein the polypeptide does not contain a peptide motif of YLFQIYNKDF (SEQ ID NO: 39). In certain embodiments, provided is a nucleic acid-guided nuclease comprising a nucleic acid encoding a polypeptide having at least 90% identity to nucleic acids represented by SEQ ID NOs: 808-845 wherein an encoded polypeptide does not contain a peptide motif of YLFQIYNKDF (SEQ ID NO: 39). In certain embodiments, provided is a nucleic acid-guided nuclease wherein the polypeptide comprises at least 90% identity with the amino acid sequence represented by SEQ ID NOs: 1-9. In certain embodiments, provided is a nucleic acid-guided nuclease, wherein the polypeptide comprises a polypeptide comprising at least 90% identity with the amino acid sequence represented by SEQ ID NO: 2, 11, or 36.

TABLE 1
ART nucleases

| | SEQ | |
|---|---|---|
| Name | ID NO | Amino Acid Sequence |
| ART1 | 1 | METFSGFTNLYPLSKTLRFRLIPVGETLKHFIDSGILEEDQHRAESYVK |
| | | VKAIIDDYHRAYIENSLSGFELPLESTKENSLEEYYLYHNIRNKTEEIQ |
| | | NLSSKVRTNLRKQVVAQLTKNEIFKRIDKKELIQSDLIDFVKNEPDANE |
| | | KIALISEFRNFTVYFKGFHENRRNMYSDEEKSTSIAFRLIHENLPKFID |
| | | NMEVFAKIQNTSISENFDAIQKELCPELVTLCEMEKLGYFNKTLSQKQI |
| | | DAYNTVIGGKTTSEGKKIKGLNEYINLYNQQHKQEKLPKMKLLFKQILS |
| | | DRESASWLPEKFENDSQVVGAIVNFWNTIHDTVLAEGGLKTIIASLGSY |
| | | GLEGIFLKNDLQLTDISQKATGSWGKISSEIKQKIEVMNPQKKKESYET |
| | | YQERIDKIFKSYKSFSLAFINECLRGEYKIEDYFLKLGAVNSSSLQKEN |
| | | HFSHILNTYTDVKEVIGLYSESTDTKLIQDNDSIQKIKQFLDAVKDLQA |
| | | YVKPLLGNGDETGKDERFYGDLIEYWSLLDLITPLYNMVRNYVTQKPYS |
| | | VDKIKINFQNPTLLNGWDLNKETDNTSVILRRDGKYYLAIMNNKSRKVF |
| | | LKYPSGTDRNCYEKMEYKLLPGANKMLPKVFFSKSRINEFMPNERLLSN |
| | | YEKGTHKKSGTCFSLDDCHTLIDFFKKSLDKHEDWKNFGFKFSDTSTYE |
| | | DMSGFYKEVENQGYKLSFKPIDATYVDQLVDEGKIFLFQIYNKDFSEHS |
| | | KGTPNMHTLYWKMLFDETNLGDVVYKLNGEAEVFFRKASINVSHPTHPA |
| | | NIPIKKKNLKHKDEERILKYDLIKDKRYTVDQFQFHVPITMNFKADGNG |
| | | NINQKAIDYLRSASDTHIIGIDRGERNLLYLVVIDGNGKICEQFSLNEI |
| | | EVEYNGEKYSTNYHDLLNVKENERKQARQSWQSIANIKDLKEGYLSQVI |
| | | HKISELMVKYNAIVVLEDLNAGFMRGRQKVEKQVYQKFEKKLIEKLNYL |
| | | VFKKQSSDLPGGLMHAYQLANKFESFNTLGKQSGFLFYIPAWNTSKMDP |
| | | VTGFVNLFDVKYESVDKAKSFFSKEDSIRYNVERDMFEWKFNYGEFTKK |
| | | AEGTKTDWTVCSYGNRIITFRNPDKNSQWDNKEINLTENIKLLFERFGI |
| | | DLSSNLKDEIMQRTEKEFFIELISLFKLVLQMRNSWTGTDIDYLVSPVC |
| | | NENGEFFDSRNVDETLPQNADANGAYNIARKGMILLDKIKKSNGEKKLA |
| | | LSITNREWLSFAQGCCKNG |
| ART2 | 2 | MISNFTNQYQLSKTERFELKPVGDTLKHIEKSGLIAQDEIRSQEYQEVK |
| | | TIIDKYHKAFIDEALQNVVLSNLEEYEALFFERNRDEKAFEKLQAVIRK |
| | | EIVAHFKQHPQYKTLFKKELIKADLKNWQELSDAEKELVSHFDNFTTYF |
| | | TGFHENRANMYIDEAKHSSIAYRIIHENLPIFLINKKLFETIKQKAPHL |
| | | AQETQDALLEYLSGAIVEDMFELSYFNHLLSQTHIDLYNQMIGGVKQDS |
| | | IKIQGINEKINLYRQANGLSKRELPNLKPLHKQILSDRETLSWLPESFE |
| | | SDEELMQGVQAYFESEVLAFECCDGKVNLLEKLPELLHQTQDYDESKVY |
| | | FKNDLALTAASQAIFKDYRIIKEALWEVNKPKKSKDLVADEEKFENKKN |
| | | SYFSIEQIDGALNSAQLSANMMHYFQSESTKVIEQIQLTYNDWKRNSSN |
| | | KELLKAFLDALLSYQRLLKPLNAPNDLEKDVAFYAYFDAYFTSLCGVVK |
| | | LYDKVRNEMTKKPYSLEKFKLNFENSTLLDGWDVNKESDNTAILFRKEG |
| | | LYYLGIMNKKYNKVERNISSSQDEGYQKIDYKLLPGANKMLPKVFESDK |
| | | NKEYFKPNAKLLERYKAGEHKKGDNEDLDFCHELIDFEKTSIEKHQDWK |
| | | HFAYQFSPTESYEDLSGFYREVEQQGYKISYKNIAASFIDTLVAEGKLY |
| | | FFQIYNKDFSPYSKGTPNMHTLYWRALFDEKNLADVIYKINGQAEIFFR |
| | | KKSIEYSQEKLQKGHHHEMLKDKFAYPIIKDRRFAFDKFQFHVPITLNE |
| | | KAEGNENITPKTFEYIRSNPDNIKVIGIDRGERHLLYLSLIDAEGKIVE |
| | | QFTLNQIINSYNGKDHVIDYHAKLDAKEKDRDKARKEWGTVENIKELKE |
| | | GYLSHVIHKIATLIIEHGAVVAMEDLNFGFKRGRFKVEKQVYQKFEKAL |
| | | IDKLNYLVDKKKEPHKLGGLLNALQLTSKFQSFEKMGKQNGFLFYVPAW |
| | | NTSKIDPVTGFVNLEDTRYASVEKSKAFFTKFQSICYNEAKDYFELVED |
| | | YNDFTEKAKETRSEWTLCTYGERIVSERNAEKNHQWDSKTIHLITEFKN |
| | | LEGELHGNDVKEYILEQNSVEFFKSLIYLLKITLQMRNSITGTDIDYLV |
| | | SPVADEAGNFYDSRKADTSLPKDADANGAYNIARKGIMIMHRIQNAEDL |
| | | KKVNLAISNRDWLRNAQGLDK |
| ART3 | 3 | MIDLKQFIGIYPVSKTLRFELRPVGKTQEWIEKNRVLEGDEQKAADYPV |
| | | VKKLIDDYHKVCIHDSLNHVHFDWEPLKDAIEIFQKTKSDEAKKRLEAE |
| | | QAMMRKKIAAAIKDFKHFKELTAATPSDLITSVLPEFSDDGSLKSFRGF |
| | | ATYFSGFQENRNNIYSQEAISTGVPYRLVHDNFPKELSDLEVFERIKST |
| | | CPEVINQASAELQPFLEGVMIDDIFSLDFYNSLLTQNGIDFFNQVIGGV |
| | | SEKDKQKYRGINEFSNLYRQQHKEIAASKKAMTMIPLFKQILSDRDTLS |
| | | YIPAQIRTEDELVSSITQFYDHITHFEHDGKTINVLSEIVALLGKLDTY |
| | | DPNGICITARKLTDISQKVYGKWSVIEEKMKEKAIQQYGDISVAKNKKK |
| | | VDAFLSRKAYSLSDLCFDEEISFSRYYSELPQTLNAISGYWLQFNEWCK |
| | | SDEKQKFLNNQTGTEVVKSLLDAMMELFHKCSVLVMPEEYEVDKSFYNE |
| | | FLPLYEELDTLFLLYNKVRNYLTQKPSDVKKFKLNFESPSLASGWDQNK |
| | | EMKNNAILLFKDGKSYLGVLNAKNKAKIKDAKGDVSSSSYKKMIYKLLS |
| | | DPSKDLPHKIFAKGNLDFYKPSEYILEGRELGKYKKGPNFDKKFLHDFI |
| | | DFYKAAISIDPDWSKENFQYSPTESYDDIGMFFSEIKKQAYKIRFTDIS |
| | | EAQVNEWVDNGQLYLFQLYNKDYAEGAHGRKNLHTLYWENLFTDENLSN |
| | | LVLKLNGQAELFCRPQSIKKPVSHKIGSKMLNRRDKSGMPIPESIYRSL |
| | | YQYYNGKKKESELTVAEKQYIDQVIVKDVTHEIIKDRRYTRQEYFFHVP |
| | | LTFNANADGNEYINEHVLNYLKDNPDVNIIGIDRGERHLIYLTLINQRG |
| | | EILKQKTFNVVNSYNYQAKLEQREKERDEARKSWDSVGKIKDLKEGELS |
| | | AVIHEITNMMIENNAIVVLEDLNFGFKRGRFKVERQVYQKFEKMLIDKL |
| | | NYLSFKDREAGEEGGILRGYQMAQKFISFQRLGKQSGFLFYIPAAYTSK |
| | | IDPVSGFVNHFNFSDITNAEKRKDFLMKMDRIEMKNGNIEFTFDYRKEK |
| | | TFQTDYQNVWTVSTFGKRIVMRIDEKGYKKMVDYEPTNDIIKAFKNKGI |
| | | LLSEGSDLKALIAEIEANATNAGFYSTLLYAFQKTLQMRNSNAVTEEDY |
| | | ILSPVAKDGHQFCSTDEANKGKDAQGNWVSKLPVDADANGAYHIALKGL |
| | | YLLRNPETKKIENEKWLQFMVEKPYLE |
| ART4 | 4 | MSYNREKMEEKELGKNQNFQEFIGVSPLQKTLRNELIPTETTKKNIAQL |
| | | DLLTEDEVRAQNREKLKEMMDDYYRDVIDSTLRGELLIDWSYLFSCMRN |
| | | HLSENSKESKRELERTQDSVRSQIHDKFAERADFKDMFGASIITKLLPT |
| | | YIKQNSKYSERYDESVKIMKLYGKFTTSLTDYFETRKNIFSKEKISSAV |
| | | GYRIVEENAEIFLQNQNAYDRICKIAGLDLHGLDNEITAYVDGKTLKEV |
| | | CSDEGFAKVITQGGIDRYNEAIGAVNQYMNLLCQKNKALKPGQFKMKRL |
| | | HKQILCKGTTSFDIPKKFENDKQVYDAVNSFTEIVTKNNDLKRLLNITQ |
| | | NANDYDMNKIYVVADAYSMISQFISKKWNLIEECLLDYYSDNLPGKGNA |
| | | KENKVKKAVKEETYRSVSQLNEVIEKYYVEKTGQSVWKVESYISSLAEM |
| | | IKLELCHEIDNDEKHNLIEDDEKISEIKELLDMYMDVFHIIKVERVNEV |
| | | LNFDETFYSEMDEIYQDMQEIVPLYNHVRNYVTQKPYKQEKYRLYFHTP |
| | | TLANGWSKSKEYDNNAIILVREDKYYLGILNAKKKPSKEIMAGKEDCSE |
| | | HAYAKMNYYLLPGANKMLPKVFLSKKGIQDYHPSSYIVEGYNEKKHIKG |
| | | SKNFDIRFCRDLIDYFKECIKKHPDWNKENFEFSATETYEDISVFYREV |
| | | EKQGYRVEWTYINSEDIQKLEEDGQLFLFQIYNKDFAVGSTGKPNLHTL |
| | | YLKNLFSEENLRDIVLKLNGEAEIFFRKSSVQKPVIHKCGSILVNRTYE |
| | | ITESGTTRVQSIPESEYMELYRYFNSEKQIELSDEAKKYLDKVQCNKAK |
| | | TDIVKDYRYTMDKFFIHLPITINFKVDKGNNVNAIAQQYIAEQEDLHVI |
| | | GIDRGERNLIYVSVIDMYGRILEQKSFNLVEQVSSQGTKRYYDYKEKLQ |
| | | NREEERDKARKSWKTIGKIKELKEGYLSSVIHEIAQMVVKYNAIIAMED |
| | | LNYGFKRGRFKVERQVYQKFETMLISKLNYLADKSQAVDEPGGILRGYQ |
| | | MTYVPDNIKNVGRQCGIIFYVPAAYTSKIDPTTGFINAFKRDVVSTNDA |
| | | KENFLMKFDSIQYDIEKGLFKFSFDYKNFATHKLTLAKTKWDVYTNGTR |
| | | IQNMKVEGHWLSMEVELTTKMKELLDDSHIPYEEGQNILDDLREMKDIT |
| | | TIVNGILEIFWLTVQLRNSRIDNPDYDRIISPVLNNDGEFFDSDEYNSY |
| | | IDAQKAPLPIDADANGAFCIALKGMYTANQIKENWVEGEKLPADCLKIE |
| | | HASWLAFMQGERG |
| ART5 | 5 | MSAVFKIKESTMKDFTHQYSLSKTLRFELKPVGETAERIEDFKNQGLKS |
| | | IVEEDRQRAEDYKKMKRILDDYHKEFIEEVLNDDIFTANEMESAFEVYR |
| | | KYMASKNDDKLKKEITEIFTDLRKKIAKAFENKSKEYCLYKGDFSKLIN |
| | | EKKTGKDKGPGKLWYWLKAKADAGVNEFGDGQTFEQAEEALAKENNEST |
| | | YFTGFNQNRDNIYTDAEQQTAISYRVINENMTRYFDNCIRYSSIENKYP |
| | | ELVKQLEPLSGKFAPGNYKDYLSQTAIDIYNEAVGHKSDDINAKGINQF |
| | | INEYRQRNSIKGRELPIMSVLYKQILSDINKDLIIDKFENAGELLDAVK |
| | | TLHRELTDKKILLKIKQTLNEFLTEDNSEDIYIKSGTDLTAVSNAIWGE |
| | | WSVIPKALEMYAENITDMNAKAREKWLKREAYHLKTVQEAIEAYLKDNE |
| | | EFETRNISEYFTNFKSGENDLIQVVQSAYAKMESIFGIEDFHKDRRPVT |
| | | ESGEPGEGFRQVELVREYLDSLINVEHFIKPLHMERSGKPIELEDCNSN |
| | | FYDPLNEAYKELDVVFGIYNKVRNYVTQKPYSKDKFKINFQNSTLLDGW |
| | | DVNKESANSSVLLLKNGKYYLGVMKQGASNILNYRPEPSDSKNKINAKK |
| | | QLSEIALAGATDDYYEKMIYKLLPDPAKMLPKVFFSAKNIEFYNPSQEI |
| | | IYIRENGLFKKDAGDKESLKKWIGFMKTSLLKHPEWGSYENFEFEPAED |
| | | YQDISIFYKQVAEQGYSVTFDKIKTSYIEEKVASGELYLFEIYNKDFSP |
| | | HSKGRPNLHTMYWKSLFEKENLQNLVTKLNGEAEVFFRQHSIKRNEKVV |
| | | HRANRPIQNKNPLTEKKQSIFEYDLVKDRRFTKDKFFLHCPITLNFKEA |
| | | GPGRFNDKVNKYIAGNPDIRIIGIDRGERHLLYYSLIDQSGRIVEQGTL |
| | | NQITSTLNSGGREIPKTTDYRGLLDTKEKERDKARKSWSMIENIKELKS |
| | | GYLSHIVHKLAKLMVKNNAVVVLEDLNFGFKRGRFKVEKQVYQKFEKAL |
| | | IEKLNYLVFKDARPAEPGHYLNAYQLTAPLESFKKLGKQSGFIYYVPAW |
| | | NTSKIDPVTGFVNQFYIEKNSMQYLKNFFGKEDSIRFNPDKNYFEFGED |
| | | YKNFHNKAAKSKWTICTHGDKRSWYNRKQRKLEIHNVTENLASLLSGKG |
| | | INFADGGSIKDKILSVDDASFFKSLAFNFKLTAQLRHTFEDNGEEIDCI |
| | | ISPVAAADGTFFCSETAKKLNMELPHDADANGAYNIARKGLMVLRQIRE |
| | | SGKPKPISNADWLDFAQQNED |
| ART6 | 6 | MQERKKISHLTHRNSVQKTIRMQLNPVGKTMDYFQAKQILENDEKLKEN |
| | | YQKIKEIADRFYRNLNEDVLSKTGLDKLKDYAEIYYHCNTDAERKRLDE |
| | | CASELRKEIVKNFKNRDEYNKLFNKKMIEIVLPQHLKNEDEKEVVASFK |
| | | NFTTYFTGFFTNRKNMYSDGEESTAIAYRCINENLPKHLDNVKAFEKAI |
| | | SKLSKNAIDDLDATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVNYYKTSLMQLTDN |
| | | LSDKYNEAAPLLNKSYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCRDEKYYLAIIDKSNNSILENIDE |
| | | QDCDENDCYEKIIYKLLPGPNKMLPKVFFSEKCKKLLSPSDEILKIRKN |
| | | GTFKKGDKFSLDDCHKLIDFYKESFKKYPNWLIYNFKEKNTNEYNDIRE |
| | | FYNDVASQGYNISKMKIPTSFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKKTSTFPYDLIKDKRYTKWQFSLHFPITMNFKAPDRAMIND |
| | | DVRNLLKSCNNNFIIGIDRGERNLLYVSVIDSNGAIIYQHSLNIIGNKF |
| | | KGKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEGGGLLHAYQLTNKLESFDKLGMQSGFIFYVRPDFTSKIDPVTGF |
| | | VNLLYPRYENIDKAKDMISREDDIGYNAGEDFFEFDIDYDKEPKTASDY |
| | | RKRWTICTNGERIEAFRNPAKNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKSDNVSTVG |
| | | PVIHNDKWLKFVQENDMANN |
| ART7 | 7 | MNILKENYMKEIKELTGLYSLTKTIGVELKPVGKTQELIEAKKLIEQDD |
| | | QRAEDYKIVKDIIDRYHKDFIDKCLNCVKIKKDDLEKYVSLAENSNRDA |
| | | EDFDKIKTKMRNQITEAFRKNSLFTNLFKKNLIKEYLPAFVSEEEKSVV |
| | | NKFSKFTTYFDAFNDNRKNLYSGDAKSGTIAYRLIHENLPMFLDNIASF |
| | | NAISGIGVNEYFSSIETEFTDTLEGKRLTEFFQIDFENNTLTQKKIGNY |
| | | NYIVGAVNKAVNLYKQQHKTVRVPLLKPLYKMILSDRVTPSWLPERFES |
| | | DEEMLTAIKAAYESLREVLVGDNDESLRNLLLNIEHYDLEHIYIANDSG |
| | | LTSISQKIFGCYDTYTLAIKDQLQRDYPATKKQREAPDLYDERIDKLYK |
| | | KVGSFSIAYLNRLVDAKGHFTINEYYKQLGAYCREEGKEKDDFFKRIDG |
| | | AYCAISHLFFGEHGEIAQSDSDVELIQKLLEAYKGLQRFIKPLLGHGDE |
| | | ADKDNEFDAKLRKVWDELDIITPLYDKVRNWLSRKIYNPEKIKLCFENN |
| | | GKLLSGWVDSRTKSDNGTQYGGYIFRKKNEIGEYDFYLGISADTKLERR |
| | | DAAISYDDGMYERLDYYQLKSKTLLGNSYVGDYGLDSMNLLSAFKNAAV |
| | | KFQFEKEVVPKDKENVPKYLKRLKLDYAGFYQILMNDDKVVDAYKIMKQ |
| | | HILATLTSSIRVPAAIELATQKELGIDELIDEIMNLPSKSFGYFPIVTA |
| | | AIEEANKRENKPLFLFKMSNKDLSYAATASKGLRKGRGTENLHSMYLKA |
| | | LLGMTQSVFDIGSGMVFFRHQTKGLAETTARHKANEFVANKNKLNDKKK |
| | | SIFGYEIVKNKRFTVDKYLFKLSMNLNYSQPNNNKIDVNSKVREIISNG |
| | | GIKNIIGIDRGERNLLYLSLIDLKGNIVMQKSLNILKDDHNAKETDYKG |
| | | LLTEREGENKEARRNWKKIANIKDLKRGYLSQVVHIISKMMVEYNAIVV |
| | | LEDLNPGFIRGRQKIERNVYEQFERMLIDKLNFYVDKHKGANETGGLLH |
| | | ALQLTSEFKNFKKSEHQNGCLFYIPAWNTSKIDPATGFVNLENTKYTNA |
| | | VEAQEFFSKFDEIRYNEEKDWFEFEFDYDKFTQKAHGTRTKWTLCTYGM |
| | | RLRSFKNSAKQYNWDSEVVALTEEFKRILGEAGIDIHENLKDAICNLEG |
| | | KSQKYLEPLMQFMKLLLQLRNSKAGTDEDYILSPVADENGIFYDSRSCG |
| | | DQLPENADANGAYNIARKGLMLIEQIKNAEDLNNVKFDISNKAWINFAQ |
| | | QKPYKNG |
| ART8 | 8 | MAKENIFNELTGKYQLSKTLRLELKPVGNTQQMLKDEDVFEKDRIIREK |
| | | YRETRPHFDRLHREFIEQALKNQKLSDLGKYFQCLAKLQNNKKDKEAQE |
| | | EFKRISQNLRKEVNDLFKIDPLFGEGVFALLKEKYGEKDDAFLREQDGQ |
| | | YVLDENKKKISIFDSWKGFTGYFTKFQETRKNFYKDDGTATAVATRIID |
| | | QNLKRFCENIQIFKSIQKKVDFKEVEDNFSVDLEDIFSLGFYSSCELQE |
| | | GIDVYNKILGGEPKTTGEKLRGLNELINRYRQDHKGEKLPFFKMLDKQI |
| | | LSEKEKFIESIEDDEELLKTLKEFYSSAEEKTTVLKELENDFIKNNENY |
| | | DLSEIYISREALNTISHRWVSAATLPEFEKSVYEVMKKDKPSGLSFDKD |
| | | DNSYKFPDFIALSYIKGSFEKLSGEKLWKDGYFRDETRNGDKGFLIGNE |
| | | SLWTQFIKIFEFEFNSLFEAKNTERSVGYYHFKKDFEKIITNDESVNPE |
| | | DKVIIREFADNVLAIYQMAKYFAIEKKRKWMDQYDTGDFYNHPDFGYKT |
| | | KFYDNAYEKIVKARMLLQSYLTKKPFSTDKWKLNFECGYLLNGWSSSEN |
| | | TYGSLLFRTGNEYYLGVVNGSALRTEKIKRLIGNITEANSCHKMVYDFQ |
| | | KPDNKNVPRIFIRSKGDKFAPAVSELNLPVDSILEIYDKGLFKTENKNS |
| | | PFFKPSLKKLIDYFKLGFSRHASYKHYQFKWKDSSEYKNISEFYNDTIR |
| | | SCYQIKWEELNFEEVKKLTNSKDLFLFQIYNKDFSEKSTGNKNLHSIYF |
| | | DGLFLDNNINAQDGVILKLSGGGEIFFRPKTDVKKLGSRTDTKGKLVIK |
| | | NKRYSQDKIFLHFPIELNYSNTQESNFNKLVRNFLADNPDINIIGVDRG |
| | | EKHLIYYAGIDQKGNTLKDKDDKDVLGSLNEINGVNYYKLLEERAKARE |
| | | KARQDWQNIQGIKDLKMGYISLVVRKLADLIIEYNAILVLEDLNMRFKQ |
| | | IHGGIEKSVYQQLEKALIEKLNFLVNKGEKDPERAGHLLRAYQLTAPES |
| | | TFKDMGKQTGVLFYTQASYTSKTCPQCGFRPNIKLHEDNLENAKKMLEK |
| | | INIVYKDNHFEIGYKVSDFTKTEKTSRGNILYGDRQGKDTFVISSKAAI |
| | | RYKWFARNIKNNELNRGESLKEHTEKGVTIQYDITECLKILYEKNGIDH |
| | | SGDITKQSIRSELPAKFYKDLLFYLYLLTNTRSSISGTEIDYINCPDCG |
| | | FHSEKGFNGCIFNGDANGAYNIARKGMLILKKINQYKDQHHTMDKMGWG |
| | | DLFIGIEEWDKYTQVVSRS |
| ART9 | 9 | MKEIKELTGLYSLTKTIGVELKPVGKTQELIEAKKLIEQDDQRAEDYKI |
| | | VKDIIDRYHKDFIDKCLNCVKIKKDDLEKYVSLAENSNRDAEDFDKIKT |
| | | KMRNQITEAFRKNSLFTNLFKKNLIKEYLPAFVSEEEKSVVNKFSKETT |
| | | YFDAFNDNRKNLYSGDAKSGTIAYRLIHENLPMFLDNIASFNAISGIGV |
| | | NEYFSSIETEFTDTLEGKRLTEFFQIDFFNNTLTQKKIGNYNYIVGAVN |
| | | KAVNLYKQQHKTVRVPLLKPLYKMILSDRVTPSWLPERFESDEEMLTAI |
| | | KAAYESLREVLVGDNDESLRNLLLNIEHYDLEHIYIANDSGLTSISQKI |
| | | FGCYDTYTLAIKDQLQRDYPATKKQREAPDLYDERIDKLYKKVGSFSIA |
| | | YLNRLVDAKGHFTINEYYKQLGAYCREEGKEKDDFFKRIDGAYCAISHL |
| | | FFGEHGEIAQSDSDVELIQKLLEAYKGLQRFIKPLLGHGDEADKDNEED |
| | | AKLRKVWDELDIITPLYDKVRNWLSRKIYNPEKIKLCFENNGKLLSGWV |
| | | DSRTKSDNGTQYGGYIFRKKNEIGEYDFYLGISADTKLFRRDAAISYDD |
| | | GMYERLDYYQLKSKTLLGNSYVGDYGLDSMNLLSAFKNAAVKFQFEKEV |
| | | VPKDKENVPKYLKRLKLDYAGFYQILMNDDKVVDAYKIMKQHILATLTS |
| | | SIRVPAAIELATQKELGIDELIDEIMNLPSKSFGYFPIVTAAIEEANKR |
| | | ENKPLFLFKMSNKDLSYAATASKGLRKGRGTENLHSMYLKALLGMTQSV |
| | | FDIGSGMVFFRHQTKGLAETTARHKANEFVANKNKLNDKKKSIFGYEIV |
| | | KNKRFTVDKYLFKLSMNLNYSQPNNNKIDVNSKVREIISNGGIKNIIGI |
| | | DRGERNLLYLSLIDLKGNIVMQKSLNILKDDHNAKETDYKGLLTEREGE |
| | | NKEARRNWKKIANIKDLKRGYLSQVVHIISKMMVEYNAIVVLEDLNPGF |
| | | IRGRQKIERNVYEQFERMLIDKLNFYVDKHKGANETGGLLHALQLTSEF |
| | | KNFKKSEHQNGCLFYIPAWNTSKIDPATGFVNLFNTKYTNAVEAQEFFS |
| | | KFDEIRYNEEKDWFEFEFDYDKFTQKAHGTRTKWTLCTYGMRLRSFKNS |
| | | AKQYNWDSEVVALTEEFKRILGEAGIDIHENLKDAICNLEGKSQKYLEP |
| | | LMQFMKLLLQLRNSKAGTDEDYILSPVADENGIFYDSRSCGDQLPENAD |
| | | ANGAYNIARKGLMLIEQIKNAEDLNNVKFDISNKAWLNFAQQKPYKNG |
| ART10 | 10 | MNFQPFFQKFVHLYPISKTLRFELIPQGATQKFISEKQVLLQDEIRARK |
| | | YPEMKQAIDGYHKDFIQRALSNIDSQVFEQALNTFEDLFLRSQAERATD |
| | | AYKKDFETAQTKLRELIVHSFEKGEFKQEYKSLEDKNLITNLLKPWVEQ |
| | | QNQIGDSNYTYHEDFNKFTTYFLGFHENRKNIYSKDPHKTALAYRLIHE |
| | | NLPKFLENNKILLKIQNDHPSLWEQLQTLNQTMPQLFDGWDFSQLMQVS |
| | | FFSNTLTQTGIDQYNTIIGGISEGENRQKIQGINELINLYNQKQDKKNR |
| | | VAKLKQLYKQILSDRSTLSFLPEKFVDDTELYHAINMFYLEHLHHQSMI |
| | | NGHSYTLLERVQLLINELANYDLSKVYLAPNQLSTVSHQMFGDFGYIGR |
| | | ALNYYYMQVIQPDYEQLLASAKTTKKIEATEKLKTIFLDTPQSLVVIQA |
| | | AIDEYIQLQPSTKPHTQLTDFIISLLKQYETVADDQSIKVINVESDIEG |
| | | KYSCIKGLVNTKSESKREVLQDEKLATDIKAFMDAVNNVIKLLKPFSLN |
| | | EKLVASVEKDARFYSDFEEIYQSLLIFVPLYNKVRNYITQKPYSTEKFK |
| | | LNFNKPTLLSGWDANKEADNLSILLRKNGNYYLAIMDTAKGANKAFEPK |
| | | TLNQLKVDDTTDCYEKMVYKLLSGPSKMFPKAFKAKNNEGNYYPTPELL |
| | | TSYNNNEHLKNDKNFTLASLHAYIDWCKEYINRNPSWHQFNFKFSPTQS |
| | | FQDISQFYSEVSSQSYKVHFQTIPSDYIDQLVAEGKLYLFQIYNKDESP |
| | | NAKGKENLHTLYFKALFSDENLKQPVFKLSGEAEMFYRPASLQLANTTI |
| | | HKAGEPMAAKNPLTPNATRTLAYDIIKDRRFTTDKYLLHVPISLNFHAQ |
| | | ESMSIKKHNDLVRQMIKHNHQDLHVIGIDRGEKHLLYVSVIDLKGNIVY |
| | | QESLNSIKSEAQNFETPYHQLLQHREEGRAQARTAWGKIENIKELKDGY |
| | | LSQVVHRIQQLILKYNAIVMLEDLNFGFKRGRFKIEKQIYQKFEKALIH |
| | | KLNYVVDKSTQADELGGVRKAYQLTAPFESFEKLGKQSGVLFYVPAWNT |
| | | SKIDPVTGFVDLLKPKYENLDKAQAFFNAFDSIHYNAQKNYFEFKVNLK |
| | | QFAGLKAQAAQAEWTICSYGDERHVYQKKNAQQGETVIVNVTEELKVLF |
| | | AKNNIEVAQSVELKETICTQTQVDFFKRLMWLLQVLLALRYSSSKDKLD |
| | | YILSPVANAQGEFFDSRHASVQLPQDSDANGAYHIALKGLWVIEQLKAA |
| | | DNLDKVKLAISNDDWLHFAQQKPYLA |
| ART11 | 11 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKFSKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKELDNIKAYSIAKSAGV |
| | | RAKELTEEEQDCLEMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVEGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNENRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYLFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNRFDSICENKVAGYFEF |
| | | SFDYKNFTDRACGMRSKWKVCINGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |
| ART12 | 12 | MAKNFEDFKRLYPLSKTLRFEAKPIGATLDNIVKSGLLEEDEHRAASYV |
| | | KVKKLIDEYHKVFIDRVLDNGCLPLDDKGDNNSLAEYYESYVSKAQDED |
| | | AIKKFKEIQQNLLSIIAKKLTDDKAYANLFGNKLIESYKDKADKTKLID |
| | | SDLIQFINTAESTQLVSMSQDEAKELVKEFWGFTTYFEGFFKNRKNMYT |
| | | PEEKSTGIAYRLINENLPKFIDNMEAFKKAIARPEIQANMEELYSNFSE |
| | | YLNVESIQEMFLLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMEGN |
| | | WGVIQNAIMQNIKHVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNRPLTITKEVEDL |
| | | NNVLYGKYKKFQKGYLTATGDNVGYTHAVNVWIKFCMDFLDSYDSTCIY |
| | | DFSSLKPESYLSLDSFYQDVNLLLYKLSFTDVSASFIDQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFEYDLIKDRRYTVDKFMFHVP |
| | | ITMNFKSSGSENINQDVKAYLRHADDMHIIGIDRGERHLLYLVVIDLQG |
| | | NIKEQFSLNEIVNDYNGNTYHTNYHDLLDVREDERLKARQSWQTIENIK |
| | | ELKEGYLSQVIHKITQLMVRYHAIVVLEDLSKGFMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTERNKEKNSQWDNQEVDLT |
| | | TEMKSLLEHYYIDIHGNLKDAISTQTDKAFFTGLLHILKLTLQMRNSIT |
| | | GTETDYLVSPVADENGIFYDSRSCGDQLPENADANGAYNIARKGLMLVE |
| | | QIKDAEDLDNVKFDISNKAWLNFAQQKPYKNG |
| ART13 | 13 | MAKNFEDFKRLYSLSKTLRFEAKPIGATLDNIVKSGLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDESE |
| | | YLNVESIQEMFQLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMEGN |
| | | WGVIQNAIMQNIKRVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNKPLTITKEVEDL |
| | | NNVLYGKYKKFQKGYLTATGDNVGYTHAVNVWIKFCMDELNSYDSTCIY |
| | | DFSSLKPESYLSLDAFYQDANLLLYKLSFARASVSYINQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFDYDLIKDRRYTVDKFMFHVP |
| | | ITMNFKSVGSENINQDVKAYLRHADDMHIIGIDRGERHLLYLVVIDLQG |
| | | NIKEQYSLNEIVNEYNGNTYHTNYHDLLDVREEERLKARQSWQTIENIK |
| | | ELKEGYLSQVIHKITQLMVRYHAIVVLEDLSKGEMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTFRNKEKNSQWDNQEVDLT |
| | | TEMKSLLEHYYIDIHGNLKDAISAQTDKAFFTGLLHILKLTLQMRNSIT |
| | | GTETDYLVSPVADENGIFYDSRSCGNQLPENADANGAYNIARKGLMLIE |
| | | QIKNAEDLNNVKFDISNKAWINFAQQKPYKNG |
| ART14 | 14 | MAKNFEDFKRLYSLSKTLRFEAKPIGATLDNIVKSDLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDESE |
| | | YLNVESIQEMFQLDYYNMLLTQKQIDVYNAIIGGKTDDEHDVKIKGIND |
| | | YINLYNQKHKDDKLPKLKALFKQILSDRNAISWLPEEENSDQEVLNAIK |
| | | DCYERLSENVLGDKVLKSMLGSLADYSLDGIFIRNDLQLTDISQKMEGN |
| | | WSVIQNAIMQNIKHVAPARKHKESEEEYENRIAGIFKKADSFSISYIDA |
| | | CLNETDPNNAYFVENYFATLGAVDTPTMQRENLFALVQNAYTEITALLH |
| | | SDYPTEKNLAQDKANVAKIKALLDAIKSLQHFVKPLLGKGDESDKDERF |
| | | YGELASLWAELDTMTPLYNMIRNYMTRKPYSQKKIKLNFENPQLLGGWD |
| | | ANKEKDYATIILRRNGLYYLAIMNKDSKKLLGKAMPSDGECYEKMVYKL |
| | | LPGANKMLPKVFFAKSRMEDFKPSKELVEKYYNGTHKKGKNENIQDCHN |
| | | LIDYFKQSIDKHEDWSKFGFKFSDTSTYEDLSGFYREVEQQGYKLSFAR |
| | | VSVSYINQLVEEGKMYLFQIYNKDFSEYSKGTPNMHTLYWKALFDERNL |
| | | ADVVYKLNGQAEMFYRKKSIENTHPTHPANHPILNKNKDNKKKESLFGY |
| | | DLIKDRRYTVDKFLFHVPITMNFKSSGSENINQDVKAYLRHADDMHIIG |
| | | IDRGERHLLYLVVIDLQGNIKEQFSLNEIVNDYNGNTYHTNYHDLLDVR |
| | | EDERLKARQSWQTIENIKELKEGYLSQVIHKITQLMVKYHAIVVLEDLN |
| | | MGFMRGRQKVEKQVYQKFEKMLIEKLNYLVDKKADASVSGGLLNAYQLT |
| | | SKEDSFQKLGKQSGFLFYIPAWNTSKIDPVTGFVNLLDTRYQNVEKAKS |
| | | FFSKFDAIRYNKDKEWFEFNLDYDKFGKKAEGTRTKWTLCTRGMRIDTF |
| | | RNKEKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPENA |
| | | DANGAYNIARKGLMMIEQIKDAKDLDNLKFDISNKAWLNFAQQKPYKNG |
| ART15 | 15 | MLFQDFTHLYPLSKTVRFELKPIGRTLEHIHAKNFLSQDETMADMYQKV |
| | | KVILDDYHRDFIADMMGEVKLTKLAEFYDVYLKFRKNPKDDELQKQLKD |
| | | LQAVLRKESVKPIGNGGKYKAGHDRLFGAKLFKDGKELGDLAKFVIAQE |
| | | GKSSPKLAHLAHFEKFSTYFTGFHDNRKNMYSDEDKHTAIAYRLIHENL |
| | | PRFIDNLQILTTIKQKHSALYDQIINELTASGLDVSLASHLDGYHKLLT |
| | | QEGITAYNRIIGEVNGYTNKHNQICHKSERIAKLRPLHKQILSDGMGVS |
| | | FLPSKFADDSEMCQAVNEFYRHYADVFAKVQSLEDGEDDHQKDGIYVEH |
| | | KNLNELSKQAFGDFALLGRVLDGYYVDVVNPEFNERFAKAKTDNAKAKL |
| | | TKEKDKFIKGVHSLASLEQAIKHHTARHDDESVQAGKLGQYFKHGLAGV |
| | | DNPIQKIHNNHSTIKGFLERERPAGERALPKIKSGKNPEMTQLRQLKEL |
| | | LDNALNVAHFAKLLMTKTTLDNQDGNFYGEFGVLYDELAKIPTLYNKVR |
| | | DYLSQKPFSTEKYKLNFGNPTLLNGWDLNKEKDNFGVILQKDGCYYLAL |
| | | LDKAHKKVFDNAPNTGKNVYQKMIYKLLPGPNKMLPRVFFAKSNLDYYN |
| | | PSAELLDKYAQGTHKKGDNFNLKDCHALIDFFKAGINKHPEWQNFGFKE |
| | | SPTSSYRDLSDFYREVEPQGYQVKFVDINADYIDELVEQGQLYLFQIYN |
| | | KDFSPKAHGKPNLHTLYFRALFSEDNLANPIYKLNGEAQIFYRKASLGM |
| | | NETTIHRAGEILENKNPDNPKERVFTYDIIKDRRYTQDKEMLHVPITMN |
| | | FGVQGMTIKEFNKKVNQSIRQYDDVNVIGIDRGERHLLYLTVINSKGEI |
| | | LEQRSLNDITTASANGTQMTTPYHKILDKREIERLNARVGWGEIETIKE |
| | | LKSGYLSHVVHQVSQLMLKYNAIVVLEDLNFGFKRGRFKVEKQIYQNFE |
| | | NALIKKLNHLELKDKADDEIGSYKNALQLTNNFTDLKNIGKQTGFLFYV |
| | | PAWNTSKIDPETGFVDLLKPRYENIAQSQAFFGKEDKICYNADKDYFEF |
| | | HIDYAKFTDKAKNSRQTWTICSHGDKRYVYDKTANQNKGATKGINVNDE |
| | | LKSLFARYHINEKQPNLVMDICQNNDKEFHKSLMYLLKTLLALRYSNAS |
| | | SDEDFILSPVANDEGVFENSALADDTQPQNADANGAYHIALKGLWLLNE |
| | | LKNSDDLNKVKLAIDNQTWLNFAQNR |
| ART16 | 16 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKFSTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLFDREDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGPNKMLPKVFFAKSNLDYYN |
| | | PSAELLDKYAQGTHKKGDNFNLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKEMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKFDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART17 | 17 | MLFQDFTHLYPLSKTVRFELKPIGKTLEHIHAKNELSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKESTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLFDREDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGSNKMLPKVFFAKSNLDYYN |
| | | PSAELLDKYAQGTHKKGDNENLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKFMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKFDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART18 | 18 | MKYTDFTGIYPVSKTLRFELIPQGSTVENMKREGILNNDMHRADSYKEM |
| | | KKLIDEYHKVFIERCLSDFSLKYDDTGKHDSLEEYFFYYEQKRNDKTKK |
| | | IFEDIQVALRKQISKRFTGDTAFKRLFKKELIKEDLPSFVKNDPVKTEL |
| | | IKEFSDFTTYFQEFHKNRKNMYTSDAKSTAIAYRIINENLPKFIDNINA |
| | | FHIVAKVPEMQEHFKTIADELRSHLQVGDDIDKMENLQFFNKVLTQSQL |
| | | AVYNAVIGGKSEGNKKIQGINEYVNLYNQQHKKARLPMLKLLYKQILSD |
| | | RVAISWLQDEFDNDQDMLDTIEAFYNKLDSNETGVLGEGKLKQILMGLD |
| | | GYNLDGVFLRNDLQLSEVSQRLCGGWNIIKDAMISDLKRSVQKKKKETG |
| | | ADFEERVSKLFSAQNSFSIAYINQCLGQAGIRCKIQDYFACLGAKEGEN |
| | | EAETTPDIFDQIAEAYHGAAPILNARPSSHNLAQDIEKVKAIKALLDAL |
| | | KRLQRFVKPLLGRGDEGDKDSFFYGDFMPIWEVLDQLTPLYNKVRNRMT |
| | | RKPYSQEKIKLNFENSTLLNGWDLNKEHDNTSVILRREGLYYLGIMNKN |
| | | YNKIFDANNVETIGDCYEKMIYKLLPGPNKMLPKVFFSKSRVQEFSPSK |
| | | KILEIWESKSFKKGDNENLDDCHALIDFYKDSIAKHPDWNKENFKESDT |
| | | QSYTNISDFYRDVNQQGYSLSFTKVSVDYVNRMVDEGKLYLFQIYNKDF |
| | | SPQSKGTPNMHTLYWRMLFDERNLHNVIYKLNGEAEVFYRKASLRCDRP |
| | | THPAHQPITCKNENDSKRVCVEDYDIIKNRRYTVDKEMFHVPITINYKC |
| | | TGSDNINQQVCDYLRSAGDDTHIIGIDRGERNLLYLVIIDQHGTIKEQF |
| | | SLNEIVNEYKGNTYCTNYHTLLEEKEAGNKKARQDWQTIESIKELKEGY |
| | | LSQVIHKISMLMQRYHAIVVLEDLNGSFMRSRQKVEKQVYQKFEHMLIN |
| | | KLNYLVNKQYDAAEPGGLLHALQLTSRMDSFKKLGKQSGFLFYIPAWNT |
| | | SKIDPVTGFVNLEDTRYCNEAKAKEFFEKFDDISYNDERDWFEFSFDYR |
| | | HFTNKPTGTRTQWTLCTQGTRVRTFRNPEKSNHWDNEEFDLTQAFKDLF |
| | | NKYGIDIASGLKARIVNGQLTKETSAVKDFYESLLKLLKLTLQMRNSVT |
| | | GTDIDYLVSPVADKDGIFFDSRTCGSLLPANADANGAFNIARKGLMLLR |
| | | QIQQSSIDAEKIQLAPIKNEDWLEFAQEKPYL |
| ART19 | 19 | METFSGFTNLYPLSKTLRFRLIPVGETLKYFIGSGILEEDQHRAESYVK |
| | | VKAIIDDYHRAYIENSLSGFELPLESTGKENSLEEYYLYHNIRNKTEEI |
| | | QNLSSKVRTNLRKQVVAQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAIVNFWNTIHDTVLAEGGLKTIIASLGS |
| | | YGLEGIFLKNDLQLTDISQKATGSWGKISSEIKQKIEVMNPQKKKESYE |
| | | TYQERIDKIFKSYKSFSLAFINECLRGEYKIEDYFLKLGAVNSSSLQKE |
| | | NHFSHILNTYTDVKEVIGFYSESTDTKLIRDNGSIQKIKLFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLDKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNTLGKQSGELFYIPAWNTSKMD |
| | | PVTGFVNLFDVKYESVDKAKSFFSKEDSIRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART20 | 20 | METFSGFTNLYPLSKTLRFRLIPVGETLKHFIDSGILEEDQHRAESYVK |
| | | VKAIIDDYHRAYIENSLSGFELPLESTGKENSLEEYYLYHNIRNKTEEI |
| | | QNLSSKVRTNLRKQVVVQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLPEKFENDSQVVGAMVNFWNTIHDTVLAEGGLKTIIASLGS |
| | | YGLEGIFLKNDLQLTDISQKATGSWSKISSEIKQKIEVMNPQKKKESYE |
| | | SYQERIDKLFKSYKSFSLAFINECLRGEYKIEDYFLKLGAVNSSSLQKE |
| | | NHFSHILNAYTDVKEAIGFYSESTDTKLIQDNDSIQKIKQFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDGNCYEKMEYKLLPGANKMLPKVFFSKSRINEFMPNERLLS |
| | | NYEKGTHKKSGICFSLDDCHTLIDFFKKSLDKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLCSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESFNALGKQSGELFYIPAWNTSKMD |
| | | PVTGFVNLFDVKYESVDKAKSFFSKFDSMRYNVERDMFEWKENYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART21 | 21 | METFSGFTNLYPLSKTLRFRLIPVGETLKHFIGSGILEEDQHRAESYVK |
| | | VKAIIDDYHRTYIENSLSGFELPLESTGKENSLEEYYLYHNIRNKTEEI |
| | | QNLSSKVRTNLRKQVVTQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYENKTLSQKQ |
| | | IDAYNTVIGGKTTSEGKKIKGLNEYINLYNQQHKQEKLPKMKLLFKQIL |
| | | SDRESASWLLEKFENDSQVVGAMVNFWNTIHDTVLAEGGLKTIIASLGS |
| | | YGLEGIFLKNDLQLTDISQKATGSWSKISSEIKQKIEAMNPQKKKESYE |
| | | SYQERIDKLFKSYKSFSLAFVNECLRGEYKIEDYFLKLGAVNSSLLQKE |
| | | NHFSHILNTYTDVKEVIGFYSESTDTKLIQDNDSIQKIKQFLDAVKDLQ |
| | | AYVKPLLGNSDETGKDERFYGDLIEYWSLLDLITPLYNMVRNYVTQKPY |
| | | SVDKIKINFQNPTLLNGWDLNKEMDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLNKHEDWKNFGFKFSDTSTY |
| | | EDMSGFYKEVENQGYKLSFKPIDATYVDQLVDEGKIFLFQIYNKDESEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKANGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGEMRGRQKVEKQVYQKFEKKLIEKLNY |
| | | LVFKKQSSDLPGGLMHAYQLANKFESFNTLGKQSGFLFYIPAWNTSKMD |
| | | PVTGFVNLFDVKYESVDKAKSFFSKFDSIRYNVERDMFEWKENYDEFTK |
| | | KAEGTKTDWTVCSYGNRIITFRNPDKNSQWDNKEINLTENIKLLFERFG |
| | | IDLSSNLKDEIMERTEKEFFIELISLFKLVLQMRNSWTGTDIDYLVSPV |
| | | CNENGEFFDSRNVDETLPQNADANGAYNIARKGMILLDKIKKNNGEKKL |
| | | TLSITNREWLSFAQGCCKNG |
| ART22 | 22 | MLFQDFTHLYPLSKTVRFELKPIGKTLEHIHAKNFLSQDKTMADMYQKV |
| | | KAILDDYHRDFIADMMGEVKLTKLAEFCDVYLKERKNPKDDGLQKQLKD |
| | | LQAVLRKEIVKPIGNGGKYKVGYDRLFGAKLFKDGKELGDLAKFVIAQE |
| | | SESSPKLPQIAHFEKFSTYFTGFHDNRKNMYSSDDKHTAIAYRLIHENL |
| | | PRFIDNLQILATIKQKHSALYDQIASELTASGLDVSLASHLGGYHKLLT |
| | | QEGITAYNRIIGEVNSYTNKHNQICHKSERIAKLRPLHKQILSDGMGVS |
| | | FLPSKFADDSEMCQAVNEFYRHYADVFAKVQSLEDREDDYQKDGIYVEH |
| | | KNLNELSKRAFGDFGFLKRFLEEYYADVIDPEFNEKFAKTEPDSDEQKK |
| | | LAGEKDKFVKGVHSLASLEQVIEYYTAGYDDESVQADKLGQYFKHRLAG |
| | | VDNPIQKIHNSHSTIKGFLERERPAGERALPKIKSDKSPEMTQLRQLKE |
| | | LLDNALNVVHFAKLVSTETVLDTRSDKFYGEFRPLYVELAKITTLYNKV |
| | | RDYLSQKPFSTEKYKLNFGNPTLLNGWDLNKEKDNFGVILQKDGCYYLA |
| | | LLDKAHKKVEDNAPNTGKSVYQKMVYKQIANARRDLACLLIINGKVVRK |
| | | TKGLDDLREKYLPYDIYKIYQSESYKVLSPNFNHQDLVKYIDYNKILAS |
| | | GYFEYFDFRFKESSEYKSYKEFLDDVDNCGYKISFCNINADYIDELVEQ |
| | | GQLYLFQIYNKDFSPKAHGKPNLHTLYFKALFSEDNLANPIYKLNGEAQ |
| | | IFYRKASLDMNETTIHRAGEVLENKNPDNPKQRQFVYDIIKDKRYTQDK |
| | | FMLHVPITMNFGVQGMTIEGENKKVNQSIQQYDDVNVIGIDRGERHLLY |
| | | LTVINSKGEILEQRSLNDIITTSANGTQMTTPYHKILNKKKEGRLQARK |
| | | DWGEIETIKELKAGYLSHVVHQISQLMLKYNAIVVLEDLNFGFKRGRLK |
| | | VENQVYQNFENALIKKLNHLVLKDKTDDEIGSYKNALQLTNNFTDLKSI |
| | | GKQTGFLFYVPARNTSKIDPETGFVDLLKPRYENITQSQAFFGKEDKIC |
| | | YNTDKGYFEFHIDYAKFTDEAKNSRQTWVICSHGDKRYVYNKTANQNKG |
| | | ATKGINVNDELKSLFACHHINDKQPNLVMDICQNNDKEFHKSLMYLLKA |
| | | LLALRYSNANSDEDFILSPVANDEGVFFNSALADDTQPQNADANGAYHI |
| | | ALKGLWVLEQIKNSDDLDKVDLEIKDDEWRNFAQNR |
| ART23 | 23 | MGKNQNFQEFIGVSPLQKTLRNELIPTETTKKNITQLDLLTEDEIRAQN |
| | | REKLKEMMDDYYRDVIDSTLHAGIAVDWSYLFSCMRNHLRENSKESKRE |
| | | LERTQDSIRSQIYNKFAERADFKDMFGASIITKLLPTYIKQNPEYSERY |
| | | DESMEILKLYGKFTTSLTDYFETRKNIFSKEKISSAVGYRIVEENAEIF |
| | | LQNQNAYDRICKIAGLDLHGLDNEITAYVDGKTLKEVCSDEGFAKAITQ |
| | | EGIDRYNEAIGAVNQYMNLLCQKNKALKPGQFKMKRLHKQILCKGTTSF |
| | | DIPKKFENDKQVYDAVNSFTEIVMKNNDLKRLLNITQNVNDYDMNKIYV |
| | | AADAYSTISQFISKKWNLIEECLLDYYSDNLPGKGNAKENKVKKAVKEE |
| | | TYRSVSQLNELIEKYYVEKTGQSVWKVESYISRLAETITLELCHEIEND |
| | | EKHNLIEDDDKISKIKELLDMYMDAFHIIKVFRVNEVLNFDETFYSEMD |
| | | EIYQDMQEIVPLYNHVRNYVTQKPYKQEKYRLYFNTPTLANGWSKNKEY |
| | | DNNAIILMRDDKYYLGILNAKKKPSKQTMAGKEDCLEHAYAKMNYYLLP |
| | | GANKMLPKVFLSKKGIQDYHPSSYIVEGYNEKKHIKGSKNFDIRFCRDL |
| | | IDYFKECIKKHPDWNKENFEFSATETYEDISVFYREVEKQGYRVEWTYI |
| | | NSEDIQKLEEDGQLFLFQIYNKDFAVGSTGKPNLHTLYLKNLFSEENLR |
| | | DIVLKLNGEAEIFFRKSSVQKPVIHKCGSILVNRTYEITESGTTRVQSI |
| | | PESEYMELYRYFNSEKQIELSDEAKKYLDKVQCNKAKTDIVKDYRYTMD |
| | | KFFIHLPITINFKVDKGNNVNAIAQQYIAEQEDLHVIGIDRGERNLIYV |
| | | SVIDMYGRILEQKSFNLVEQVSSQGTKRYYDYKEKLQNREEERDKARKS |
| | | WKTIGKIKELKEGYLSSVIHEIAQMVVKYNAIIAMEDLNYGFKRGRFKV |
| | | ERQVYQKFETMLISKLNYLADKSQAVDEPGGILRGYQMTYVPDNIKNVG |
| | | RQCGIIFYVPAAYTSKIDPTTGFINAFKRDVVSTNDAKENFLMKEDSIQ |
| | | YDIEKGLFKFSFDYKNFATHKLTLAKTKWDVYINGTRIQNMKVEGHWLS |
| | | MEVELTTKMKELLDDSHIPYEEGQNILDDLREMKDITTIVNGILEIFWL |
| | | TVQLRNSRIDNPDYDRIISPVLNNDGEFFDSDEYNSYIDAQKAPLPIDA |
| | | DANGAFCIALKGMYTANQIKENWVEGEKLPADCLKIEHASWLAFMQGER |
| | | G |
| ART24 | 24 | MNTSLFSSFTRQYPVTKTLRFELKPMGATLGHIQQKGFLHKDEELAKIY |
| | | KKIKELLDEYHRAFIADTLGDAQLVGLDDFYADYQALKQDSKNSHLKDK |
| | | LTKTQDNLRKQITKNFEKTPQLKERYKRLFTKELFKAGKDKGDLEKWLI |
| | | NHDSEPNKAEKISWIHQFENFTTYFQGFYENRKNMYSDEVKHTAIAYRL |
| | | IHENLPRFVDNIQVLSKIKSDYPDLYHELNHLDSRTIDFADEKEDDMLQ |
| | | MDFYHHLLIQSGITAYNTLLGGKVLEGGKKLQGINELINLYGQKHKIKI |
| | | AKLKPLHKQILSDGQSVSFLPKKFDNDYELCQTVNHFYREYVAIFDELV |
| | | VLFQKFYDYDKDNIYINHQQLNQLSHELFADERLLSRALDFYYCQIIDG |
| | | DENNKINNAKSQNAKEKLLKEKERYTKSNHSINELQKAINHYASHHEDT |
| | | EVKVISDYFSATNIRNMIDGIHHHESTIKGFLEKDNNQGESYLPKQKNS |
| | | NDVKNLKLFLDGVLRLIHFIKPLALKSDDTLEKEEHFYGEFMPLYDKLV |
| | | MFTLLYNKVRDYISQKPYNDEKIKLNFGNSTLLNGWDVNKEKDNFGVIL |
| | | CKEGLYYLAILDKSHKKVFDNAPKATSSHTYQKMVYKLLPGPNKMLPKV |
| | | FFAKSNIGYYQPSAQLLENYEKGTHKKGSNFSLTDCHHLIDFFKSSIAK |
| | | HPEWKEFGFRFSDTHTYQDLSDFYKEIEPQSYKVKFIDIDADYIDDLVE |
| | | KGQLYLFQLYNKDFSKQSYGKPNLHTLYFKSLFSDDNLKNPIYKLNGEA |
| | | EIFYRRASLSVSDTTIHQAGEILTPKNPNNTHNRTLSYDVIKNKRYTTD |
| | | KFFLHIPITMNFGIENTGFKAFNHQVNTTLKNADKKDVHIIGIDRGERH |
| | | LLYVSVIDGDGRIVEQRTLNDIVSISNNGMSMSTPYHQILDNREKERLA |
| | | ARTDWGDIKNIKELKAGYLSHVVHEVVQMMLKYNAMIVLEDLNFGFKHG |
| | | RFKVEKQVYQNFENALIKKLNYLVLKNADNHQLGSVRKALQLTNNETDI |
| | | KSIGKQTGFIFYVPAWNTSKIDPTTGFVDLLKPRYENMAQAQSFISRFK |
| | | KIAYNHQLDYFEFEFDYADFYQKTIDKKRIWTLCTYGDVRYYYDHKTKE |
| | | TKTVNITKELKSLLDKHDLSYQNGHNLVDELANSHDKSLLSGVMYLLKV |
| | | LLALRYSHAQKNEDFILSPVMNKDGVFFDSRFADDVLPNNADANGAYHI |
| | | ALKGLWVLNQIQSADNMDKIDLSISNEQWLHFTQSR |
| ART25 | 25 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKFSQDPEYKVMLSAGMITKILPKYILEKYETD |
| | | REDRLEAIKRFYGFTVYFKEFWASRQNVFSDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSFNALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNEDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWFDFIQNRRF |
| ART26 | 26 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKFSQDPEYKVMLSAGMITKILPKYILEKYETD |
| | | REDRLEAIKRFYGFTVYFKEFWASRQNVESDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSENALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDEKEN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNFDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWFDFIQNRRFQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSENMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVENFREYSNFETKLD |
| | | FVRSLDSIRYDTEKRLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDEKGRFYDSEN |
| | | YIFNEETELPKDADANGAYCIALKGLYNVIAIKNNWKEGEKFNRKLLSL |
| | | NNYNWEDFIQNRRF |
| ART27 | 27 | MQEHKKISHLTHRNSVQKTIRMQLNPVGKTMDYFQAKQILENDEKLKED |
| | | YQKIKEIADRFYRNLNEDVLSKTGLDKLKDYAEIYYHCNTDADRKRLDE |
| | | CASELRKEIVKNFKNRDEYNKLENKKMIEIVLPQHLKNEDEKEVVASFK |
| | | NFTTYFTGFFTNRKNMYSDGEESTAIAYRCINENLPKHLDNVKAFEKAI |
| | | SKLSKNAVDDLDTTYSGLCGTNLYDVFTVDYFNFLLPQSGITEYNKIIG |
| | | GYTTSDGTKVKGINEYINLYNQQVSKRYKIPNLKILYKQILSESEKVSF |
| | | IPPKFEDDNELLSAVSEFYANDETFDGMPLKKAIDETKLLFGNLDNSSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFNESYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDF |
| | | QDCDESDCYEKIIYKLLPGPNKMLPKVFFSEKCKKLLSPSDEILKIRKN |
| | | GTFKKGDKESLDDCHKLIDFYKESFKKYPNWLIYNFKFKKTNEYNDISE |
| | | FYNDVASQGYNISKMKIPTSFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKRASTFPYDLIKDKRYTKWQFSLHFPITMNFKAPDRAMIND |
| | | DVRNLLKSCNNNFIIGIDRGERNLLYVSIIDSNGAIIYQHSLNIIGNKF |
| | | KGKTYETNYREKLETREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPRYENIDKAKDMISREDDIRYNAGEDFFEFDIDYDKFPKTASDY |
| | | RKKWTICTNGERIEAFRNPASNNEWSYRTIILAEKFKELFDNNSINYRD |
| | | SDNLKAEILSQTKGKFFEDFFKLLRLTLQMRNSNPETGEDRILSPVKDK |
| | | NGNFYDSSKYDEKSNLPCDADANGAYNIARKGLWIVEQFKKSDNVSTVE |
| | | PVIHNDKWLKFVQENDMANN |
| ART28 | 28 | MKNLANFTNLYSLQKTLRFELKPIGKTLDWIIKKDLLKQDEILAEDYKI |
| | | VKKIIDRYHKDFIDLAFESAYLQKKSSDSFTAIMEASIQSYSELYFIKE |
| | | KSDRDKKAMEEISGIMRKEIVECFTGKYSEVVKKKFGNLFKKELIKEDL |
| | | LNFCEPDELPIIQKFADFTTYFTGFHENRENMYSNEEKATAIANRLIRE |
| | | NLPRYLDNLRIIRSIQGRYKDFGWKDLESNLKRIDKNLQYSDFLTENGF |
| | | VYTFSQKGIDRYNLILGGQSVESGEKIQGLNELINLYRQKNQLDRRQLP |
| | | NLKELYKQILSDRTRHSFVPEKFSSDKALLRSLLDFHKEVIQNKNLFEE |
| | | KQVSLLQAIRETLTDLKSEDLDRIYLTNDTSLTQISNFVFGDWSKVKTI |
| | | LAIYFDENIANPKDRQRQSNSYLKAKENWLKKNYYSIHELNEAISVYGK |
| | | HSDEELPNTKIEDYFSGLQTKDETKKPIDVLDAIVSKYADLESLLTKEY |
| | | PEDKNLKSDKGSIEKIKNYLDSIKLLQNFLKPLKPKKVQDEKDLGFYND |
| | | LELYLESLESANSLYNKVRNYLTGKEYSDEKIKLNFKNSTLLDGWDENK |
| | | ETSNLSVIFRDTNNYYLGILDKQNNRIFESIPEIQSGEETIQKMVYKLL |
| | | PGANNMLPKVFFSEKGLLKENPSDEITSLYSEGRFKKGDKESINSLHTL |
| | | IDFYKKSLAVHEDWSVENFKFDETSHYEDISQFYRQVESQGYKITFKPI |
| | | SKKYIDTLVEDGKLYLFQIYNKDFSQNKKGGGKPNLHTIYFKSLFEKEN |
| | | LKDVIVKLNGQAEVFFRKKSIHYDENITRYGHHSELLKGRFSYPILKDK |
| | | RFTEDKFQFHFPITLNFKSGEIKQFNARVNSYLKHNKDVKIIGIDRGER |
| | | HLLYLSLIDQDGKILRQESLNLIKNDQNFKAINYQEKLHKKEIERDQAR |
| | | KSWGSIENIKELKEGYLSQVVHTISKLMVEHNAIVVLEDLNFGFKRGRQ |
| | | KVERQVYQKFEKMLIEKLNFLVFKDKEMDEPGGILKAYQLTDNFVSFEK |
| | | MGKQTGFVFYVPAWNTSKIDPKTGFVNFLHLNYENVNQAKELIGKFDQI |
| | | RYNQDRDWFEFQVTTDQFFTKENAPDTRTWIICSTPTKRFYSKRTVNGS |
| | | VSTIEIDVNQKLKELFNDCNYQDGEDLVDRILEKDSKDFFSKLIAYLRI |
| | | LTSLRQNNGEQGFEERDFILSPVVGSDGKFFNSLDASSQEPKDADANGA |
| | | YHIALKGLMNLHVINETDDESLGKPSWKISNKDWLNFVWQRPSLKA |
| ART29 | 29 | MQEHKKISHLTHRNSVQKTIRMQLNPVGKTMDYFQAKQILENDEKLKEN |
| | | YQKIKEIADRFYRNLNEDVLSKTRLDKLKDYTDIYYHCNTDADRKRLDE |
| | | CASELRKEIVKNFKNRDEYNKLFNKKMIEIVLPKHLKNEDEKEVVTSFK |
| | | NFTTYFTGFFTNRKNMYSDGEESTAIAYRCINENLPKHLDNVKAFEKAI |
| | | SKLSKNAIDDLDTTYSGLCGTNLYDVFTVDYFNFLLPQSGITEYNKIIG |
| | | GYTTNDGTKVKGINEYINLYNQQVSKRDKIPNLKILYKQILSESEKVSF |
| | | IPPKFEDDNELLSAVSEFYANDETFDGMPLKKAIDETKLLFGNLDNPSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYNEAAPLLNENYSNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDF |
| | | QDCDESDCYEKIIYKLLPGPNKMLPKVFFSEKCKKLLSPSDEILKIYKS |
| | | GTFKTGDKFSLDDCHKLIDFYKESFKKYPNWLIYNFKFKKTNEYNDIRE |
| | | FYNDVALQGYNISKMKIPTSFIDKLVDEGKIYLFQLYNKDESPHSKGTP |
| | | NLHTLYFKMLFDERNLEDVVYRLNGEAEMFYRPASIKYDKPTHPKNTPI |
| | | KNKNTLNDKKTSTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KEKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPQYENIDKAKDMISRFDEIRYNAGEDFFEFDIDYDEFPKTASDY |
| | | RKKWTICTNGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDQWLKFVQENDMANN |
| ART30 | 30 | MQEHKKISHLTHRNSVQKTIRMQLNPVGKTMDYFQAKQILENDEKLKED |
| | | YQKIKEIADRFYRNLNEDVLSKTGLDKLKDYADIYYHCNTDADRKRLNE |
| | | CASELRKEIVKNFKNRDEYNKLFNKKMIEIVLPKHLKNEDEKEVVASFK |
| | | NFTTYFTGFFTNRKNMYSDGEESTAIAYRCINENLPKHLDNVKVFEKAI |
| | | SKLSKNAIDDLGATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFSENYDNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDKDKEREYGAVLLCKDEKYYLAIIDKSNNSILENIDF |
| | | QDCNESDYYEKIVYKLLTKINGNLPRVFFSEKRKKLLSPSDEILKIYKS |
| | | GTFKKGDKFSLDDCHKLIDFYKESFKKYPNWLIYNFKFKNTNEYNDISE |
| | | FYNDVASQGYNISKMKIPTTFIDKLVDEGKIYLFQLYNKDESPHSKGTP |
| | | NLHTLYFKMLFDERNLEDVVYKLNGEAEMFYRPASIKYDKPTHPKNTPI |
| | | KNKNTLNDKKASTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KGKTYETNYREKLATREKDRTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGTQSGFIFYVRPDFTSKIDPVTGF |
| | | VNLLYPRYENIDKAKDMISREDDIRYNAGEDFFEFDIDYDKFPKTASDY |
| | | RKKWTICINGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDKWLKFVQENDMANN |
| ART31 | 31 | MQERKKISHLTHRNSVKKTIRMQLNPVGKTMDYFQAKQILENDEKLKEN |
| | | YQKIKEIADRFYRNLNEDVLSKTGLDKLKDYAEIYYHCNTDADRKRLNK |
| | | CASELRKEIVKNFKNRDEYNKLFDKRMIEIVLPKHLKNEDEKEVVASEK |
| | | NFTTYFTGFFTNRKNMYSDGEESTAIAYRCINENLPKHLDNVKAFEKAI |
| | | SKLSKNAIDDLDAYSGLCGTNLYDVFTVDYFNFLLPQSGITEYNKIIGG |
| | | YTTNDGTKVKGINEYINLYNQQVSKRDKIPNLQILYKQILSESEKVSFI |
| | | PPKFEDDNELLSAVSEFYANDETFDGMPLKKAIDETKLLFGNLDNSSLN |
| | | GIYIQNDRSVINLSNSMFGSWSVIEDLWNKNYDSVNSNSRIKDIQKRED |
| | | KRKKAYKAEKKLSLSFLQVLISNSENDEIRKKSIVDYYKTSLMQLTDNL |
| | | SDKYNEAAPLLNENYSNEKGLKNDDKSISLIKNFLDAIKEIEKFIKPLS |
| | | ETNITGEKNDLFYSQFTPLLDNISRIDILYDKVRNYVTQKPFSTDKIKL |
| | | NFGNYQLLNGWDKDKEREYGAVLLCKDEKYYLAIIDKSNNRILENIDFQ |
| | | DCDESDCYEKIIYKLLPTPNKMLPKVFFAKKHKKLLSPSDEILKIYKNG |
| | | TFKKGDKFSLDDCHKLIDFYKESFKKYPKWLIYNFKFKKTNGYNDIREF |
| | | YNDVALQGYNISKMKIPTSFIDKLVDEGKIYLFQLYNKDFSPHSKGTPN |
| | | LHTLYFKMLFDERNLEDVVYRLNGEAEMFYRPASIKYDKPTHPKNTPIK |
| | | NKNTLNDKRASTFPYDLIKDKRYTKWQFSLHFPITMNFKDPDKAMINDD |
| | | VRNLLKSCNNNFIIGIDRGERNLLYVSVINSNGAIIYQHSLNIIGNKFK |
| | | GKTYETNYREKLATREKDRTEQRRNWKAIESIKELKEGYISQAVHVICQ |
| | | LVVKYDAIIVMEKLTDGFKRGRTKFEKQVYQKFEKMLIDKLNYYVDKKL |
| | | DPDEEGGLLHAYQLTNKLESFDKLGTQSGFIFYVRPDFTSKIDPVTGFV |
| | | NLLYPRYEKIDKAKDMISREDDIRYNAGEDFFEFDIDYDKFPKTASDYR |
| | | KKWTICTNGERIEAFRNPANNNEWSYRTIILAEKFKELFDNNSINYRDS |
| | | DDLKAEILSQTKGKFFEDFFKLLRLTLQMRNSNPETGEDRILSPVKDKN |
| | | GNFYDSSKYDEKSKLPCDADANGAYNIARKGLWIVEQFKKADNVSTVEP |
| | | VIHNDKWLKFVQENDMANN |
| ART32 | 32 | KTGLDKLKDYAEIYYHCNTDADRKRLNKCASELRKEIVKNFKNRDEYNK |
| | | LFDKRMIEIVLPKHLKNEDEKEVVASFKNFTTYFTGFFTNRKNMYSDGE |
| | | ESTAIAYRCINENLPKHLDNVKAFEKAISKLSKNAIDDLDATYSGLCGT |
| | | NLYDVFTVDYFNFLLPQSGITEYNKIIGGYTTSDGTKVKGINEYINLYN |
| | | QQVSKRDKIPNLQILYKQILSESEKVSFIPPKFEDDNELLSAVSEFYAN |
| | | DETFDEMPLKKAIDETKLLFGNLDNSSLNGIYIQNDRSVTNLSNSMFGS |
| | | WSVIEDLWNKNYDSVNSNSRIKDIQKREDKRKKAYKAEKKLSLSFLQVL |
| | | ISNSENNEIREKSIVDYYKTSLMQLTDNLSDKYNEVAPLLNENYSNEKG |
| | | LKNDDKSISLIKNFLDAIKEIEKFIKPLSETNITGEKNDLFYSQFTPLL |
| | | DNISRIDILYDKVRNYVTQKPFSTDKIKLNFGNYQLLNGWDKDKEREYG |
| | | AVLLCRDEKYYLAIIDKSNNRILENIDFQDCDESDCYEKIIYKLLPTPN |
| | | KMLPKVFFAKKHKKLLSPSDEILKIRKNGTFKKGDKFSLDDCHKLIDFY |
| | | KESFKKYPNWLIYNFKFKKTNEYNDIREFYNDVALQGYNISKMKIPTSF |
| | | IDKLVDEGKIYLFQLYNKDFSPHSKGTPNLHTLYFKMLFDERNLEDVVY |
| | | KLNGEAKMFYRPASIKYDKPTHPKNTPIKNKNTLNDKKASTFPYDLIKD |
| | | KRYTKWQFSLHFSITMNFKAPDKAMINDDVRNLLKSCNNNFIIGIDRGE |
| | | RNLLYVSVIDSNGAIIYQHSLNIIGNKFKGKTYETNYREKLATREKERT |
| | | EQRRNWKAIESIKELKEGYISQAVHVICQLVVKYDAIIVMEKLTDGFKR |
| | | GRTKFEKQVYQKFEKMLIDKLNYYVDKKLDPDEEGGLLHAYQLINKLES |
| | | FDKLGTQSGFIFYVRPDFTSKIDPVTGFVNLLYPRYENIDKAKDMISRF |
| | | DDIRYNAGEDFFEFDIDYDKFPKTASDYRKKWTICTNGERIEAFRNPAN |
| | | NNEWSYRTIILAEKFKELFDNNSINYRDSDDLKAEILSQTKGKFFEDFF |
| | | KLLRLTLQMRNSNPETGEDRILSPVKDKNGNFYDSSKYDEKSKLPCDAD |
| | | ANGAYNIARKGLWIVEQFKKSDNVSTVEPVIHNDKWLKFVQENDMANN |
| ART33 | 33 | MSININKESDECRKIDFFTDLYNIQKTLRFSLIPIGATADNFEFKGRLS |
| | | KEKDLLDSAKRIKEYISKYLADESDICLSQPVKLKHLDEYYELYITKDR |
| | | DEQKFKSVEEKLRKELADLLKEILKRLNKKILSDYLPEYLEDDEKALED |
| | | IANLSSFSTYFNSYYDNCKNMYTDKEQSTAIPYRCINDNLPKFIDNMKA |
| | | YEKALEELKPSDLEELRNNFKGVYDTTVDDMFTLDYFNCVLSQSGIDSY |
| | | NAIIGNDKVKGINEYINLHNQTAEQGHKVPNLKRLYKQIGSQKKTISFL |
| | | PSKFESDNELLKAVYDFYNTGDAEKNFTALKDTITEFEKIFDNLSEYNL |
| | | DGVFVRNDISLTNLSQSMENDWSVERNLWNDQYDKVNNPEKAKDIDKYN |
| | | DKRHKVYKKSESFSINQLQELIATTLEEDINSKKITDYFSCDFHRVTTE |
| | | VENKYQLVKDLLSSDYPKNKNLKTSEEDVALIKDFLDSVKSLESFVKIL |
| | | TGTGKESGKDELFYGSFTKWFDQLRYIDKLYDKVRNYITEKPYSLDKIK |
| | | LSFDNPQFLGGWQHSKETDYSAQLFMKDGLYYLGVMDKETKREFKTQYN |
| | | TPENDSDTMVKIEYNQIPNPGRVIQNLMLVDGKIVKKNGRKNADGVNAV |
| | | LEELKNQYLPENINRIRKTESYKTTSNNENKDDLKAYLEYYIARTKEYY |
| | | CKYNFVFKSADEYGSFNEFVDDVNNQAYQITKVKVSEKQLLSLVEQGKL |
| | | YLFKIYNKDFSEYSKGKKNLHTMYFQMLFDDRNLENLVYKLQGGAEMFY |
| | | RPASIKKDSEFKHDANVEIIKRTCEDKVNDKDNPTDDEKAKYYSKFDYD |
| | | IVKNKRFTKDQFSLHLTLAMNCNQPDHYWLNNDVRELLKKSNKNHIIGI |
| | | DRGERNLIYVTIINSDGVIVDQINFNIIENSYNGKKYKTDYQKKLNQRE |
| | | EDRQKARKTWKTIETIKELKDGYISQVVHQICKLIVQYDAIVVMENLNG |
| | | GFKRGRTKVEKQVYQKFETMLINKLNYYVDKGTDYKECGGLLKAYQLTN |
| | | KFETFERIGKQSGIIFYVDPYLTSKIDPVTGFANLLYPKYETIPKTHNF |
| | | ISNIDDIRYNQSEDYFEFDIDYDKFPQGSYNYRKKWTICSYGNRIKYYK |
| | | DSRNKTASVVVDITEKFKETFTNAGIDFVNDNIKEKLLLVNSKELLKSF |
| | | MDTLKLTVQLRNSEINSDVDYIISPIKDRNGNFYYSENYKKSNNEVPSQ |
| | | PQDGDANGAYNIARKGLMIINKLKKADDVINNELLKISKKEWLEFAQKG |
| | | DLGE |
| ART34 | 34 | MKATSIWDNFTRKYSVSKTLRFELRPVGKTEENIVKKEIIDAEWISGKN |
| | | IPKGTDADRARDYKIVKKLLNQLHILFINQALSSENVKEFEKEDKKSKT |
| | | FVAWSDLLATHFDNWIQYTRDKSNSTVLKSLEKSKKDLYSKLGKLLNSK |
| | | ANAWKAEFISYHKIKSPDNIKIRLSASNVQILFGNTSDPIQLLKYQIEL |
| | | DNIKFLKDDGSEYTTKELADLLSTFEKFGTYFSGFNQNRANVYDIDGEI |
| | | STSIAYRLFNQNIEFFFQNIKRWEQFTSSIGHKEAKENLKLVQWDIQSK |
| | | LKELDMEIVQPRFNLKFEKLLTPQSFIYLLNQEGIDAFNTVLGGIPAEV |
| | | KAEKKQGVNELINLTRQKLNEDKRKFPSLQIMYKQIMSERKINFIDQYE |
| | | DDVEMLKEIQEFSNDWNEKKKRHSASSKEIKESAIAYIQREFHETFDSL |
| | | EERATVKEDFYLSEKSIQNLSIDIFGGYNTIHNLWYTEVEGMLKSGERP |
| | | LTRVEKEKLKKQEYISFAQIERLISKHSQQYLDSTPKEANDRSLFKEKW |
| | | KKTFKNGFKVSEYTNLKLNELISEGETFQKIDQETGKETTIKIPGLFES |
| | | YENAILVESIKNQSLGTNKKESVPSIKEYLDSCLRLSKFIESFLVNSKD |
| | | LKEDQSLDGCSDFQNTLTQWLNEEFDVFILYNKVRNHVTKKPGNTDKIK |
| | | INFDNATLLDGWDVDKEAANFGFLLKKADNYYLGIADSSFNQDLKYFNE |
| | | GERLDEIEKNRKNLEKEESKNISKIDQEKVKKYKEVIDDLKAISNLNKG |
| | | RYSKAFYKQSKFTTLIPKCTTQLNEVIEHFKKEDTDYRIENKKFAKPFI |
| | | ITKEVFLLNNTVYDTATKKFTLKIGEDEDTKGLKKFQIGYYRATDDKKG |
| | | YESALRNWITFCIEFTKSYKSCLNYNYSSLKSVSEYKSLDEFYKDLNGI |
| | | GYTIDFVDISEEYINKKINEGKLYLFQIYNKDFSEKSKGKENLHTTYWK |
| | | LLFDSKNLEDVVIKLNGQAEVFFRPASIHEKEKITHFKNQEIQNKNPNA |
| | | VKKTSKFEYDIIKDNRFTKNKFLFHCPITLNFKADGNPYVNNEVQENIA |
| | | KNPNVNIIGIDRGEKHLLYFTVINQQGQILDAGSLNSIKSEYKDKNQQS |
| | | VSFETPYHKILDKKESERKEARESWQEIENIKELKAGYLSHVVHQLSNL |
| | | IVKYNAIVVLEDLNKGFKRGRFKVEKQVYQKFEKSLIEKLNYLVFKDRK |
| | | ESNEPGHHLNAYQLTNKELSFERLGKQSGVLFYATASYTSKVDPVTGFM |
| | | QNIYDPYHKEKTREFYKNFTKIVYNGNYFEFNYDLNSVKPDSEEKRYRT |
| | | NWTVCSCVIRSEYDSNSKTQKTYNVNDQLVKLFEDAKIKIENGNDLKST |
| | | ILEQDDKFIRDLHFYFIAIQKMRVVDSKIEKGEDSNDYIQSPVYPFYCS |
| | | KEIQPNKKGFYELPSNGDSNGAYNIARKGIVILDKIRLRVQIEKLFEDG |
| | | TKIDWQKLPNLISKVKDKKLLMTVFEEWAELTHQGEVQQGDLLGKKMSK |
| | | KGEQFAEFIKGLNVTKEDWEIYTQNEKVVQKQIKTWKLESNST |
| ART35 | 35 | MKAINEYYKQLGAYCREEGKEKDDFFKRIDGAYCAISHLFFGEHGEIAQ |
| | | SDSDVELIQKLLEAYKGLQRFIKPLLGHGDEADKDNEFDAKLRKVWDEL |
| | | DIITPLYDKVRNWLSRKIYNPEKIKLCFENNGKLLSGWVDSRTKSDNGT |
| | | QYGGYIFRKKNEIGEYDFYLGISADTKLFRRDAAISYDDGMYERLDYYQ |
| | | LKSKTLLGNSYVGDYGLDSMNLLSAFKNAAVKFQFEKEVVPKDKENVPK |
| | | YLKRLKLDYAGFYQILMNDDKVVDAYKIMKQHILATLTSSIRVPAAIEL |
| | | ATQKELGIDELIDEIMNLPSKSFGYFPIVTAAIEEANKRENKPLFLFKM |
| | | SNKDLSYAATASKGLRKGRGTENLHSMYLKALLGMTQSVEDIGSGMVFF |
| | | RHQTKGLAETTARHKANEFVANKNKLNDKKKSIFGYEIVKNKRFTVDKY |
| | | LFKLSMNLNYSQPNNNKIDVNSKVREIISNGGIKNIIGIDRGERNLLYL |
| | | SLIDLKGNIVMQKSLNILKDDHNAKETDYKGLLTEREGENKEARRNWKK |
| | | IANIKDLKRGYLSQVVHIISKMMVEYNAIVVLEDLNPGFIRGRQKIERN |
| | | VYEQFERMLIDKLNFYVDKHKGANETGGLLHALQLTSEFKNFKKSEHQN |
| | | GCLFYIPAWNTSKIDPATGFVNLENTKYTNAVEAQEFFSKFDEIRYNEE |
| | | KDWFEFEFDYDKFTQKAHGTRTKWTLCTYGMRLRSFKNSAKQYNWDSEV |
| | | VALTEEFKRILGEAGIDIHENLKDAICNLEGKSQKYLEPLMQFMKLLLQ |
| | | LRNSKAGTDEDYILSPVADENGIFYDSRSCGDQLPENADANGAYNIARK |
| | | GLMLIEQIKNAEDLNNVKFDISNKAWLNFAQQKPYKNGMKAINEYYKQL |
| | | GAYCREEGKEKDDFFKRIDGAYCAISHLFFGEHGEIAQSDSDVELIQKL |
| | | LEAYKGLQRFIKPLLGHGDEADKDNEFDAKLRKVWDELDIITPLYDKVR |
| | | NWLSRKIYNPEKIKLCFENNGKLLSGWVDSRTKSDNGTQYGGYIFRKKN |
| | | EIGEYDFYLGISADTKLFRRDAAISYDDGMYERLDYYQLKSKTLLGNSY |
| | | VGDYGLDSMNLLSAFKNAAVKFQFEKEVVPKDKENVPKYLKRLKLDYAG |
| | | FYQILMNDDKVVDAYKIMKQHILATLTSSIRVPAAIELATQKELGIDEL |
| | | IDEIMNLPSKSFGYFPIVTAAIEEANKRENKPLFLFKMSNKDLSYAATA |
| | | SKGLRKGRGTENLHSMYLKALLGMTQSVFDIGSGMVFFRHQTKGLAETT |
| | | ARHKANEFVANKNKLNDKKKSIFGYEIVKNKRFTVDKYLFKLSMNLNYS |
| | | QPNNNKIDVNSKVREIISNGGIKNIIGIDRGERNLLYLSLIDLKGNIVM |
| | | QKSLNILKDDHNAKETDYKGLLTEREGENKEARRNWKKIANIKDLKRGY |
| | | LSQVVHIISKMMVEYNAIVVLEDLNPGFIRGRQKIERNVYEQFERMLID |
| | | KLNFYVDKHKGANETGGLLHALQLTSEFKNFKKSEHQNGCLFYIPAWNT |
| | | SKIDPATGFVNLFNTKYTNAVEAQEFFSKFDEIRYNEEKDWFEFEFDYD |
| | | KFTQKAHGTRTKWTLCTYGMRLRSFKNSAKQYNWDSEVVALTEEFKRIL |
| | | GEAGIDIHENLKDAICNLEGKSQKYLEPLMQFMKLLLQLRNSKAGTDED |
| | | YILSPVADENGIFYDSRSCGDQLPENADANGAYNIARKGLMLIEQIKNA |
| | | EDLNNVKFDISNKAWLNFAQQKPYKNG |
| ART11* | 36 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKESKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKFLDNIKAYSIAKSAGV |
| | | RAKELTEEEQDCLEMTETFERTLTQDGIDNYNELIGKLNFAINLYNQQN |
| | | NKLKGFRKVPKMKELYKQILSEREASFVDEFVDDEALLINVESFSAHIK |
| | | EFLESDSLSRFAEVLEESGGEMVYIKNDTSKTTFSNIVFGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYFFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNREDSICENKVAGYFEF |
| | | SFDYKNFTDRACGMRSKWKVCTNGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |

In certain embodiments, a Cas nuclease comprises ABW1 (SEQ ID NO: 3), ABW2 (SEQ ID NO: 16), ABW3 (SEQ ID NO: 29), ABW4 (SEQ ID NO: 42), ABW5 (SEQ ID NO: 55), ABW6 (SEQ ID NO: 68), ABW7 (SEQ ID NO: 81), ABW8 (SEQ ID NO: 94), or ABW9 (SEQ ID NO: 107) (all SEQ ID NOs for ABW1-9 and variants thereof from International (PCT) Application Publication No. WO 2021/108324), or variants thereof, such as any one of variants 1-10 of ABW1 (SEQ ID NOs: 4-13, respectively), any one of variants 1-10 of ABW2 (SEQ ID NOs: 17-26, respectively), any one of variants 1-10 of ABW3 (SEQ ID NOs: 30-39, respectively), any one of variants 1-10 of ABW4 (SEQ ID NOs: 43-52, respectively), any one of variants 1-10 of ABW5 (SEQ ID NOs: 56-65, respectively), any one of variants 1-10 of ABW6 (SEQ ID NOs: 69-78, respectively), any one of variants 1-10 of ABW7 (SEQ ID NOs: 82-91, respectively), any one of variants 1-10 of ABW8 (SEQ ID NOs: 95-104, respectively), any one of variants 1-10 of ABW9 (SEQ ID NOs: 108-117, respectively). ABW1-ABW9, and variants thereof are known in the art and are described in International (PCT) Application Publication No. WO 2021/108324.

More type V-A Cas nucleases and their corresponding naturally occurring CRISPR-Cas systems can be identified by computational and experimental methods known in the art, e.g., as described in U.S. Pat. No. 9,790,490 and Shmakov et al. (2015) MOL. CELL, 60:385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163:759.

In certain embodiments, the Cas protein is a Cas nuclease that directs cleavage of one or both strands at the target locus, such as the target strand (i.e., the strand having the target nucleotide sequence that is at least partially complementary to and can hybridize with a single guide nucleic acid or dual guide nucleic acids) and/or the non-target strand. In certain embodiments, the Cas nuclease directs cleavage of one or both strands within about 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e. generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, a composition provided herein comprises a Cas nuclease that a compatible guide nucleic acid (gNA), e.g., a gRNA, is capable of activating. In certain embodiments, a composition provided herein further comprises a Cas protein that is related to the Cas nuclease that a compatible guide nucleic acid (gNA), e.g., a gRNA, is capable of activating. For example, in certain embodiments, a Cas protein comprises an amino acid sequence at least 80% (e.g., at least 85%, at least 90%, at least 91%, at least 92%, at least 93%, at least 94%, at least 95%, at least 96%, at least 97%, at least 98%, or at least 99%) identical to the Cas nuclease amino acid sequence. In certain embodiments, a Cas protein comprises a nuclease-inactive mutant of the Cas nuclease. In certain embodiments, a Cas protein further comprises an effector domain.

In certain embodiments, a Cas nuclease has the activity to cleave a double-stranded DNA and result in a double-strand break.

In certain embodiments, a protospacer adjacent motif (PAM) or PAM-like motif directs binding of a Cas protein complex to a target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using any suitable method, such as testing cleavage, targeting, or modification of oligonucleotides having the target nucleotide sequence and different PAM sequences.

Exemplary PAM sequences are provided in Tables 2 and 3. In certain embodiments, a Cas protein comprises MAD7 and the PAM is TTTN, wherein N is A, C, G, or T. In certain embodiments, a Cas protein comprises MAD7 and the PAM is CTTN, wherein N is A, C, G, or T. In certain embodiments, a Cas protein comprises AsCpf1 and the PAM is TTTN, wherein N is A, C, G, or T. In certain embodiments, a Cas protein comprises FnCpf1 and the PAM is 5′ TTN, wherein N is A, C, G, or T. PAM sequences for certain other type V-A Cas proteins are disclosed in Zetsche et al. (2015) CELL, 163:759 and U.S. Pat. No. 9,982,279. Further, engineering of the PAM Interacting (PI) domain of a Cas protein may allow programing of PAM specificity, improve target site recognition fidelity, and/or increase the versatility of an engineered, non-naturally occurring system. Exemplary approaches to alter the PAM specificity of Cpf1 are described in Gao et al. (2017) NAT. BIOTECHNOL., 35:789.

In certain embodiments, an engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the PI domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, an engineered Cas protein comprises one or more nuclear localization signal (NLS) motifs. In certain embodiments, an engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 40); the NLS from nucleoplasmin, e.g., the nucleoplasmin bipartite NLS having the amino acid sequence of KRPAATKKAGQAKKKK (SEQ ID NO: 41); the c-myc NLS, having the amino acid sequence of PAAKRVKLD (SEQ ID NO: 42) or RQRRNELKRSP (SEQ ID NO: 43); the hRNPA1 M9 NLS, having the amino acid sequence of NQSSNFGPMKGGNFGGRSSGPYGGGGQYFAKPRNQGGY (SEQ ID NO: 44); the importin-α IBB domain NLS, having the amino acid sequence of RMRIZFKNKGKDTAELRRRRVEVSVELRKAKKDEQILKRRNV (SEQ ID NO: 45); the myoma T protein NLS, having the amino acid sequence of VSRKRPRP (SEQ ID NO: 46) or PPKKARED (SEQ ID NO: 47); the human p53 NLS, having the amino acid sequence of PQPKKKPL (SEQ ID NO: 48); the mouse c-abl IV NLS, having the amino acid sequence of SALIKKKKKMAP (SEQ ID NO: 49); the influenza virus NS1 NLS, having the amino acid sequence of DRLRR (SEQ ID NO: 50) or PKQKKRK (SEQ ID NO: 51); the hepatitis virus 8 antigen NLS, having the amino acid sequence of RKLKKKIKKL (SEQ ID NO: 52); the mouse Mx1 protein NLS, having the amino acid sequence of REKKKFLKRR (SEQ ID NO: 53); the human poly(ADP-ribose) polymerase NLS, having the amino acid sequence of KRKGDEVDGVDEVAKKKSKK (SEQ ID NO: 54); the human glucocorticoid receptor NLS, having the amino acid sequence of RKCLQAGMNLEARKTKK (SEQ ID NO: 55), and synthetic NLS motifs such as PAAKKKKLD (SEQ ID NO: 56).

In general, the one or more NLS motifs are of sufficient strength to drive accumulation of the Cas protein in a detectable amount in the nucleus of a eukaryotic cell. The strength of nuclear localization activity may derive from the number of NLS motif(s) in the Cas protein, the particular NLS motif(s) used, the position(s) of the NLS motif(s), or a combination of these and/or other factors. In certain embodiments, an engineered Cas protein comprises at least 1 (e.g., at least 2, at least 3, at least 4, at least 5, at least 6, at least 7, at least 8, at least 9, or at least 10) NLS motif(s) at or near the N-terminus (e.g., within about 1, 2, 3, 4, 5, 10, 15, 20, 25, 30, 40, 50, or more amino acids along the polypeptide chain from the N-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within about 1, 2, 3, 4, 5, 10, 15, 20, 25, 30, 40, 50, or more amino acids along the polypeptide chain from the C-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to a nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs . . .

### B. Guide Nucleic Acids

A guide nucleic acid can be a single gNA (sgNA, e.g., sgRNA), in which the gNA is a single polynucleotide, or a dual gNA (e.g., dual gRNA), in which the gNA comprises two separate polynucleotides (these can in some cases be covalently linked, but not via a conventional internucleotide linkage). In certain embodiments, a single guide nucleic acid is capable of activating a Cas nuclease alone (e.g., in the absence of a tracrRNA).

In general, a gNA comprises a modulator nucleic acid and a targeter nucleic acid. In a sgNA the modulator and targeter nucleic acids are part of a single polynucleotide. In a dual gNA the modulator and targeter nucleic acids are separate, e.g., not joined by a conventional nucleotide linkage, such as not joined at all. The targeter nucleic acid comprises a spacer sequence and a targeter stem sequence. The modulator nucleic acid comprises a modulator stem sequence and, generally, further nucleotides, such as nucleotides comprising a 5′ tail. The modulator stem sequence and targeter stem sequence can each comprise any suitable number of nucleotides and are of sufficient complementarity that they can hybridize. In a single gNA there may be additional NTs between the targeter stem sequence and the modulator stem sequence; these can, in certain cases, form secondary structure, such as a loop.

In certain embodiments, the guide nucleic acid comprises a targeter nucleic acid that, in combination with a modulator nucleic acid, is capable of binding a Cas protein. In certain embodiments, the guide nucleic acid comprises a targeter nucleic acid that, in combination with a modulator nucleic acid, is capable of activating a Cas nuclease. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA. Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. It is understood that these sequences are merely illustrative, and other guide nucleic acid sequences may also be used with these Cas proteins.

TABLE 2
Type V-A Cas Protein and Corresponding Single Guide Nucleic Acid Sequences

| Cas Protein | Scaffold Sequence¹ | PAM² |
|---|---|---|
| MAD7 (SEQ ID | UAAUUUCUACUCUUGUAGA (SEQ ID NO: 57), | 5′ TTTN |
| NO: 37) | AUCUACAACAGUAGA (SEQ ID NO: 58), | or 5′ |
| | AUCUACAAAAGUAGA (SEQ ID NO: 59), | CTTN |
| | GGAAUUUCUACUCUUGUAGA (SEQ ID NO: 60), | |
| | UAAUUCCCACUCUUGUGGG (SEQ ID NO: 61) | |
| MAD2 (SEQ ID | AUCUACAAGAGUAGA (SEQ ID NO: 62), | 5′ TTTN |
| NO: 38) | AUCUACAACAGUAGA (SEQ ID NO: 58), | |
| | AUCUACAAAAGUAGA (SEQ ID NO: 59), | |
| | AUCUACACUAGUAGA (SEQ ID NO: 63) | |
| AsCpf1 (SEQ | UAAUUUCUACUCUUGUAGA (SEQ ID NO: 57) | 5′ TTTN |
| ID NO: 3 of | | |
| WO | | |
| 2021/158918) | | |
| LbCpf1 (SEQ | UAAUUUCUACUAAGUGUAGA (SEQ ID NO: 64) | 5′ TTTN |
| ID NO: 4 of | | |
| WO | | |
| 2021/158918) | | |
| FnCpf1 (SEQ | UAAUUUUCUACUUGUUGUAGA (SEQ ID NO: 65) | 5′ TTN |
| ID NO: 5 of | | |
| WO | | |
| 2021/158918) | | |
| PbCpf1 (SEQ | AAUUUCUACUGUUGUAGA (SEQ ID NO: 66) | 5′ TTTC |
| ID NO: 6 of | | |
| WO | | |
| 2021/158918) | | |
| PsCpf1 (SEQ | AAUUUCUACUGUUGUAGA (SEQ ID NO: 66) | 5′ TTTC |
| ID NO: 7 of | | |
| WO | | |
| 2021/158918) | | |
| As2Cpf1 (SEQ | AAUUUCUACUGUUGUAGA (SEQ ID NO: 66) | 5′ TTTC |
| ID NO: 8 of | | |
| WO | | |
| 2021/158918) | | |
| McCpf1 (SEQ | GAAUUUCUACUGUUGUAGA (SEQ ID NO: 67) | 5′ TTTC |
| ID NO: 9 of | | |
| WO | | |
| 2021/158918) | | |
| Lb3Cpf1 (SEQ | GAAUUUCUACUGUUGUAGA (SEQ ID NO: 67) | 5′ TTTC |
| ID NO: 10 of | | |
| WO | | |
| 2021/158918) | | |
| EcCpf1 (SEQ | GAAUUUCUACUGUUGUAGA (SEQ ID NO: 67) | 5′ TTTC |
| ID NO: 11 of | | |
| WO | | |
| 2021/158918) | | |
| SmCsm1 (SEQ | GAAUUUCUACUGUUGUAGA (SEQ ID NO: 67) | 5′ TTTC |
| ID NO: 12 of | | |
| WO | | |
| 2021/158918) | | |
| SsCsm1 (SEQ | GAAUUUCUACUGUUGUAGA (SEQ ID NO: 67) | 5′ TTTC |
| ID NO: 13 of | | |
| WO | | |
| 2021/158918) | | |
| MbCsm1 (SEQ | GAAUUUCUACUGUUGUAGA (SEQ ID NO: 67) | 5′ TTTC |
| ID NO: 14 of | | |
| WO | | |
| 2021/158918) | | |
| ART2 (SEQ ID | GUCUAAAGGUACCACCAAAUUUCUACUGUUGUAGAU | 5′ TTTN |
| NO: 2 | (SEQ ID NO: 68) | or 5′ |
| | | NTTN |
| ART11 (SEQ ID | GCUUAGAACCUUUAAAUAAUUUCUACUAUUGUAGAU | 5′ TTTN |
| NO: 11 | (SEQ ID NO: 69) | or 5′ |
| | | NTTN |
| ART11* (SEQ | GCUUAGAACCUUUAAAUAAUUUCUACUAUUGUAGAU | 5′ TTTN |
| ID NO: 36 | (SEQ ID NO: 69) | or 5′ |
| | | NTTN |

¹The modulator sequence in the scaffold sequence is underlined; the targeter stem sequence in the scaffold sequence is bold-underlined. It is understood that a “scaffold sequence” listed herein constitutes a portion of a single guide nucleic acid. Additional nucleotide sequences, other than the spacer sequence, can be comprised in the single guide nucleic acid.
² In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 3
Type V-A Cas Protein and Corresponding Dual Guide Nucleic Acid Sequences

| | | Targeter | |
|---|---|---|---|
| | | Stem | |
| Cas Protein | Modulator Sequence¹ | Sequence | PAM² |
| MAD7 (SEQ ID NO: | UAAUUUCUAC (SEQ ID NO: | GUAGA | 5′ TTTN |
| 37) | 70) | | or 5′ |
| | AUCUAC (SEQ ID NO: 71) | GUAGA | CTTN |
| | GGAAUUUCUAC (SEQ ID NO: | GUAGA | |
| | 72) | | |
| | UAAUUCCCAC (SEQ ID NO: | GUGGG | |
| | 73) | | |
| MAD2 (SEQ ID NO: | AUCUAC (SEQ ID NO: 71) | GUAGA | 5′ TTTN |
| 38) | | | |
| AsCpf1 (SEQ ID NO: | UAAUUUCUAC (SEQ ID NO: | GUAGA | 5′ TTTN |
| 3 of WO | 70) | | |
| 2021/158918) | | | |
| LbCpf1 (SEQ ID NO: | UAAUUUCUAC (SEQ ID NO: | GUAGA | 5′ TTTN |
| 4 of WO 2021/158918) | 70) | | |
| FnCpf1 (SEQ ID NO: | UAAUUUUCUACU (SEQ ID NO: | GUAGA | 5′ TTN |
| 5 of WO | 74) | | |
| 2021/158918) | | | |
| PbCpf1 (SEQ ID NO: | AAUUUCUAC (SEQ ID NO: 75) | GUAGA | 5′ TTTC |
| 6 of WO | | | |
| 2021/158918) | | | |
| PsCpf1 (SEQ ID NO: | AAUUUCUAC (SEQ ID NO: 75) | GUAGA | 5′ TTTC |
| 7 of WO | | | |
| 2021/158918) | | | |
| As2Cpf1 (SEQ ID | AAUUUCUAC (SEQ ID NO: 75) | GUAGA | 5′ TTTC |
| NO: 8 of WO | | | |
| 2021/158918) | | | |
| McCpf1 (SEQ ID NO: | GAAUUUCUAC (SEQ ID NO: | GUAGA | 5′ TTTC |
| 9 of WO | 76) | | |
| 2021/158918) | | | |
| Lb3Cpf1 (SEQ ID | GAAUUUCUAC (SEQ ID NO: | GUAGA | 5′ TTTC |
| NO: 10 of WO | 76) | | |
| 2021/158918) | | | |
| EcCpf1 (SEQ ID NO: | GAAUUUCUAC (SEQ ID NO: | GUAGA | 5′ TTTC |
| 11 of WO | 76) | | |
| 2021/158918) | | | |
| SmCsm1 (SEQ ID NO: | GAAUUUCUAC (SEQ ID NO: | GUAGA | 5′ TTTC |
| 12 of WO | 76) | | |
| 2021/158918) | | | |
| SsCsm1 (SEQ ID NO: | GAAUUUCUAC (SEQ ID NO: | GUAGA | 5′ TTTC |
| 13 of WO | 76) | | |
| 2021/158918) | | | |
| MbCsm1 (SEQ ID NO: | GAAUUUCUAC (SEQ ID NO: | GUAGA | 5′ TTTC |
| 14 of WO | 76) | | |
| 2021/158918) | | | |
| ART2 (SEQ ID NO: 2) | AAAUUUCUAC (SEQ ID NO: | GUAGA | 5′ TTTN |
| | 77) | | or 5′ |
| | | | NTTN |
| ART11 (SEQ ID NO: | UAAUUUCUAC (SEQ ID NO: | GUAGA | 5′ TTTN |
| 11) | 70) | | or 5′ |
| | | | NTTN |
| ART11* (SEQ ID NO: | UAAUUUCUAC (SEQ ID NO: | GUAGA | 5′ TTTN |
| 36) | 70) | | or 5′ |
| | | | NTTN |

¹It is understood that a “modulator sequence” listed herein may constitute the nucleotide sequence of a modulator nucleic acid. Alternatively, additional nucleotide sequences can be comprised in the modulator nucleic acid 5′ and/or 3′ to a “modulator sequence” listed herein.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

In certain embodiments, a guide nucleic acid, in the context of a type V-A CRISPR-Cas system, comprises a targeter stem sequence listed in Table 3. The same targeter stem sequences, as a portion of scaffold sequences, are bold-underlined in Table 2.

In certain embodiments, a guide nucleic acid is a single guide nucleic acid that comprises, from 5′ to 3′, a modulator stem sequence, a loop sequence, a targeter stem sequence, and a spacer sequence. In certain embodiments, the targeter stem sequence in the single guide nucleic acid is listed in Table 2 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 2 as an underlined portion of a scaffold sequence, a loop sequence, a targeter stem sequence a bold-underlined portion of the same scaffold sequence, and a spacer sequence. In certain embodiments, an engineered, non-naturally occurring system comprises a single guide nucleic acid comprising a scaffold sequence listed in Table 2. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 2. In certain embodiments, the system further comprises a Cas protein (e.g., Cas nuclease) comprising the amino acid sequence set forth in the SEQ ID NO listed in the same line of Table 2. In certain embodiments, the system is useful for targeting, editing, or modifying a nucleic acid comprising a target nucleotide sequence close or adjacent to (e.g., immediately downstream of) a PAM listed in the same line of Table 2 when using the non-target strand (i.e., the strand not hybridized with the spacer sequence) as the coordinate.

In certain embodiments, a guide nucleic acid, e.g, dual gNA, comprises a targeter guide nucleic acid that comprises, from 5′ to 3′, a targeter stem sequence and a spacer sequence. In certain embodiments, the targeter stem sequence in the targeter nucleic acid is listed in Table 3. In certain embodiments, an engineered, non-naturally occurring system comprises the targeter nucleic acid and a modulator stem sequence complementary (e.g., 100% complementary) to the targeter stem sequence. In certain embodiments, the modulator nucleic acid comprises a modulator sequence listed in the same line of Table 3. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 3. In certain embodiments, the system further comprises a Cas protein (e.g., Cas nuclease) comprising the amino acid sequence set forth in the SEQ ID NO listed in the same line of Table 3. In certain embodiments, the system is useful for targeting, editing, or modifying a nucleic acid comprising a target nucleotide sequence close or adjacent to (e.g., immediately downstream of) a PAM listed in the same line of Table 3 when using the non-target strand (i.e., the strand not hybridized with the spacer sequence) as the coordinate.

A single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and/or modulator nucleic acid. In certain embodiments, a single guide nucleic acid is no more than 100, 90, 80, 70, 60, 50, 40, 30, or 25 nucleotides in length. In certain embodiments, a single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a targeter nucleic acid is no more than 100, 90, 80, 70, 60, 50, 40, 30, or 25 nucleotides in length. In certain embodiments, a targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a modulator nucleic acid is no more than 100, 90, 80, 70, 60, 50, 40, 30, or 20 nucleotides in length. In certain embodiments, a modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

It is contemplated that the length of the duplex formed within the single guide nuclei acid or formed between the targeter nucleic acid and the modulator nucleic acid, e.g. in a dual gNA, may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at the 3′ end of the loop) in a crRNA of a corresponding single guide CRISPR-Cas system. It is understood that an additional nucleotide sequence 5′ to the targeter stem sequence can be dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see, Kocak et al. (2019) Nat. Biotech. 37:657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at the 5′ end of the loop) in a crRNA of a corresponding single guide CRISPR-Cas system. It is understood that an additional nucleotide sequence 3′ to the modulator stem sequence can be dispensable. Accordingly, in certain embodiments, the modulator nucleic acid does not comprise any additional nucleotide 3′ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of a complex comprising the targeter nucleic acid and the modulator nucleic acid.

The stability of a complex comprising a targeter nucleic acid and a modulator nucleic acid can be assessed by the Gibbs free energy change (AG) during the formation of the complex, either calculated or actually measured. Where all the predicted base pairing in the complex occurs between a base in the targeter nucleic acid and a base in the modulator nucleic acid, i.e., there is no intra-strand secondary structure, the AG during the formation of the complex correlates generally with the AG during the formation of a secondary structure within the corresponding single guide nucleic acid. Methods of calculating or measuring the AG are known in the art. An exemplary method is RNAfold (rna.tbi.univie.ac.at/cgi-bin/RNAWebSuite/RNAfold.cgi) as disclosed in Gruber et al. (2008) Nucleic Acids Res., 36 (Web Server issue): W70-W74. Unless indicated otherwise, the AG values in the present disclosure are calculated by RNAfold for the formation of a secondary structure within a corresponding single guide nucleic acid. In certain embodiments, the AG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the AG is greater than or equal to −10 kcal/mol, e.g., greater than or equal to −9 kcal/mol, greater than or equal to −8.5 kcal/mol, or greater than or equal to −8 kcal/mol. In certain embodiments, the AG is in the range of −10 to −4 kcal/mol. In certain embodiments, the AG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol. In certain embodiments, the AG is about-8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the AG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the AG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ tail” positioned 5′ to the modulator stem sequence. In a naturally occurring type V-A CRISPR-Cas system, the 5′ tail is a nucleotide sequence positioned 5′ to the stem-loop structure of the crRNA. A 5′ tail in an engineered type V-A CRISPR-Cas system, whether single guide or dual guide, can be reminiscent to the 5′ tail in a corresponding naturally occurring type V-A CRISPR-Cas system.

Without being bound by theory, it is contemplated that the 5′ tail may participate in the formation of the CRISPR-Cas complex. For example, in certain embodiments, the 5′ tail forms a pseudoknot structure with the modulator stem sequence, which is recognized by the Cas protein (see, Yamano et al. (2016) Cell, 165:949). In certain embodiments, the 5′ tail is at least 3 (e.g., at least 4 or at least 5) nucleotides in length. In certain embodiments, the 5′ tail is 3, 4, or 5 nucleotides in length. In certain embodiments, the nucleotide at the 3′ end of the 5′ tail comprises a uracil or is a uridine. In certain embodiments, the second nucleotide in the 5′ tail, the position counted from the 3′ end, comprises a uracil or is a uridine. In certain embodiments, the third nucleotide in the 5′ tail, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ tail comprises the nucleotide sequence of 5′-AUU-3′. In certain embodiments, the 5′ tail comprises the nucleotide sequence of 5′-AAUU-3′. In certain embodiments, the 5′ tail comprises the nucleotide sequence of 5′-UAAUU-3′. In certain embodiments, the 5′ tail is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than about 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than about 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and P A Carr and G M Church, 2009, Nature Biotechnology 27 (12): 1151-62).

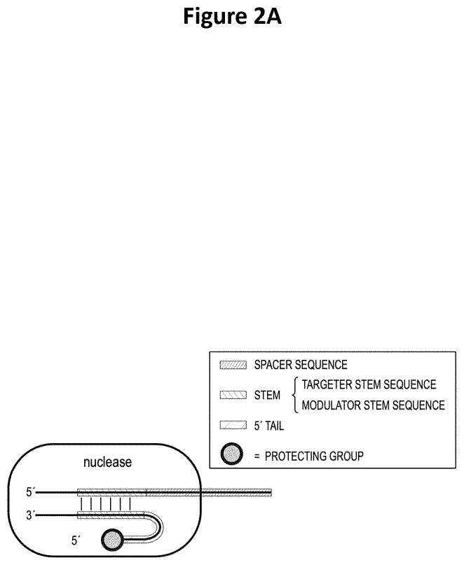

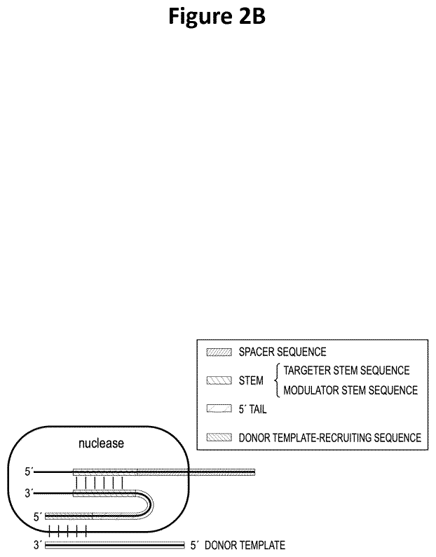

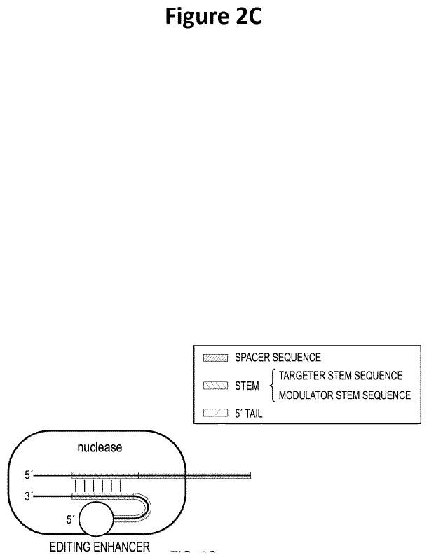

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ tail, if present, or to the modulator stem sequence, of the single guide nucleic acid or the modulator nucleic acid through an internucleotide bond or a nucleotide linker.

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) Nat. Commun. 9:3313. In certain embodiments, the editing enhancer sequence is positioned 5′ to the 5′ tail, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is about 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. (2018) Cell. Mol. Life Sci., 75 (19): 3593-3607). Secondary structures can be predicted by methods known in the art, such as the online webserver RNAfold developed at University of Vienna using the centroid structure prediction algorithm (see, Gruber et al. (2008) Nucleic Acids Res., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at the 5′ or 3′ end of the single guide nucleic acid, the modulator nucleic acid, and/or the targeter nucleic acid. In certain embodiments, the single guide nucleic acid comprises a protective nucleotide sequence at the 5′ end, at the 3′ end, or at both ends, optionally through a nucleotide linker. In certain embodiments, the modulator nucleic acid comprises a protective nucleotide sequence at the 5′ end, at the 3′ end, or at both ends, optionally through a nucleotide linker. In particular embodiments, the modulator nucleic acid comprises a protective nucleotide sequence at the 5′ end (see FIG. 2A). In certain embodiments, the targeter nucleic acid comprises a protective nucleotide sequence at the 5′ end, at the 3′ end, or at both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ tail, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ tail, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, an engineered, non-naturally occurring system further comprises one or more compounds (e.g., small molecule compounds) that enhance HDR and/or inhibit NHEJ. Exemplary compounds having such functions are described in Maruyama et al. (2015) Nat Biotechnol. 33 (5): 538-42; Chu et al. (2015) Nat Biotechnol. 33 (5): 543-48; Yu et al. (2015) Cell Stem Cell 16 (2): 142-47; Pinder et al. (2015) Nucleic Acids Res. 43 (19): 9379-92; and Yagiz et al. (2019) Commun. Biol. 2:198. In certain embodiments, an engineered, non-naturally occurring system further comprises one or more compounds selected from the group consisting of DNA ligase IV antagonists (e.g., SCR7 compound, Ad4 E1B55K protein, and Ad4 E4orf6 protein), RAD51 agonists (e.g., RS-1), DNA-dependent protein kinase (DNA-PK) antagonists (e.g., NU7441 and KU0060648), B3-adrenergic receptor agonists (e.g., L755507), inhibitors of intracellular protein transport from the ER to the Golgi apparatus (e.g., brefeldin A), and any combinations thereof.

In certain embodiments, an engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### C. gNA Modifications

Guide nucleic acids, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. Spacer sequences can be presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments engineered, non-naturally occurring systems comprising a targeter nucleic acid comprising: a spacer sequence designed to hybridize with a target nucleotide sequence and a targeter stem sequence; and a modulator nucleic acid comprising a modulator stem sequence complementary to the targeter stem sequence, and, optionally, a 5′ sequence, e.g., a tail sequence, wherein, in a single guide nucleic acid the targeter nucleic acid and the modulator nucleic acid are part of a single polynucleotide, and in a dual guide nucleic acid, the targeter nucleic acid and the modulator nucleic acid are separate nucleic acids; modifications can include one or more chemical modifications to one or more nucleotides or internucleotide linkages at or near the 3′ end of the targeter nucleic acid (dual and single gNA), at or near the 5′ end of the targeter nucleic acid (dual gNA), at or near the 3′ end of the modulator nucleic acid (dual gNA), at or near the 5′ end of the modulator nucleic acid (single and dual gNA), or combinations thereof as appropriate for single or dual gNA. In certain embodiments, the Cas nuclease is a type V-A Cas nuclease. Modulator and/or targeter nucleic sequences can include further sequences, as detailed in the Guide Nucleic Acids section, and modifications can be in these further sequences, as appropriate and apparent to one of skill in the art. In embodiments described in this section, below, in certain embodiments, guide nucleic acid is oriented from 5′ at the modulator nucleic acid to 3′ at the modulator stem sequence, and 5′ at the targeter stem sequence to 3′ at the targeter sequence (see, e.g., FIGS. 1A and 1B); in certain embodiments, as appropriate, guide nucleic acid is oriented from 3′ at the modulator nucleic acid to 5′ at the modulator stem sequence, and 3′ at the targeter stem sequence to 5′ at the targeter sequence.

The targeter nucleic acid may comprise a DNA (e.g., modified DNA), an RNA (e.g., modified RNA), or a combination thereof. The modulator nucleic acid may comprise a DNA (e.g., modified DNA), an RNA (e.g., modified RNA), or a combination thereof. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA. The nucleotide sequences disclosed herein are presented as DNA sequences by including thymidines (T) and/or RNA sequences including uridines (U). It is understood that corresponding DNA sequences, RNA sequences, and DNA/RNA chimeric sequences are also contemplated. For example, where a spacer sequence is presented as a DNA sequence, a nucleic acid comprising this spacer sequence as an RNA can be derived from the DNA sequence disclosed herein by replacing each T with U. As a result, for the purpose of describing a nucleotide sequence, T and U are used interchangeably herein.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA. In further embodiments, the remaining portion of the gNA that is not RNA comprises a modified ribonucleotide, a deoxyribonucleotide, a modified deoxyribonucleotide, or a synthetic, e.g., unnatural nucleotide, for example, not intended to be limiting, threose nucleic acid, locked nucleic acid, peptide nucleic acid, arabinonucleic acid, hexose nucleic acid, among others.

In certain embodiments, the targeter nucleic acid and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Pat. Nos. 10,900,034 and 10,767,175, U.S. Patent Application Publication No. 2018/0119140, Watts et al. (2008) Drug Discov. Today 13:842-55, and Hendel et al. (2015) NAT. BIOTECHNOL. 33:985.

In certain embodiments, a targeter nucleic acid, e.g., RNA, comprises at least one nucleotide at or near the 3′ end comprising a modification to a ribose, phosphate group, nucleobase, or terminal modification. In certain embodiments, the 3′ end of the targeter nucleic acid comprises the spacer sequence. In certain embodiments, the 3′ end of the targeter nucleic acid comprises the targeter stem sequence. Exemplary modifications are disclosed in Dang et al. (2015) Genome Biol. 16:280, Kocaz et al. (2019) Nature Biotech. 37:657-66, Liu et al. (2019) Nucleic Acids Res. 47 (8): 4169-4180, Schubert et al. (2018) J. Cytokine Biol. 3 (1): 121, Teng et al. (2019) Genome Biol. 20 (1): 15, Watts et al. (2008) Drug Discov. Today 13 (19-20): 842-55, and Wu et al. (2018) Cell Mol. Life. Sci. 75 (19): 3593-607.

Modifications in a ribose group include but are not limited to modifications at the 2′ position or modifications at the 4′ position. For example, in certain embodiments, the ribose comprises 2′-O—C1-4alkyl, such as 2′-O-methyl (2′-OMe, or M). In certain embodiments, the ribose comprises 2′-O—C1-3alkyl-O-C1-3alkyl, such as 2′-methoxyethoxy (2′-O-CH₂CH₂OCH₃) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′—NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications can also include a deoxy group, for example a 2′-deoxy-3′-phosphonoacetate (DP), a 2′-deoxy-3′-thiophosphonoacetate (DSP).

Internucleotide linkage modifications in a phosphate group include but are not limited to a phosphorothioate(S), a chiral phosphorothioate, a phosphorodithioate, a boranophosphonate, a C₁₋₄alkyl phosphonate such as a methylphosphonate, a boranophosphonate, a phosphonocarboxylate such as a phosphonoacetate (P), a phosphonocarboxylate ester such as a phosphonoacetate ester, an amide, a thiophosphonocarboxylate such as a thiophosphonoacetate (SP), a thiophosphonocarboxylate ester such as a thiophosphonoacetate ester, and a 2′,5′-linkage having a phosphodiester or any of the modified phosphates above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see, Piccirilli et al. (1990) NATURE, 343:33), 5-methyl-2-pyrimidine (see, Rappaport (1993) BIOCHEMISTRY, 32:3047), x(A,G,C,T), and y(A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers, propanediol), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein) propane-1,3-diol bis(phosphodiester) linker, which is incorporated as a phosphodiester linkage and can be incorporated anywhere between two nucleotides in the RNA.

The modifications disclosed above can be combined in the targeter nucleic acid and/or the modulator nucleic acid that are in the form of RNA. In certain embodiments, the modification in the RNA is selected from the group consisting of incorporation of 2′-O-methyl-3′phosphorothioate (MS), 2′-O-methyl-3′-phosphonoacetate (MP), 2′-O-methyl-3′-thiophosphonoacetate (MSP), 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, modifications can include 2′-O-methyl (M), a phosphorothioate(S), a phosphonoacetate (P), a thiophosphonoacetate (SP), a 2′-O-methyl-3′-phosphorothioate (MS), a 2′-O-methyl-3′-phosphonoacetate (MP), a 2′-O-methyl-3′-thiophosphonoacetate (MSP), a 2′-deoxy-3′-phosphonoacetate (DP), a 2′-deoxy-3′-thiophosphonoacetate (DSP), or a combination thereof, at or near either the 3′ or 5′ end of either the targeter or modulator nucleic acid, as appropriate for single or dual gNA. In certain embodiments, modifications can include either a 5′ or a 3′ propanediol or C3 linker modification.

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C1-4alkyl, 2′-halo (e.g., 2′-F, 2′-Br, 2′-Cl, or 2′-I), 2′MOE, a 2′-O—C1-3alkyl-O-C1-3alkyl, 2′—NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ sequence, e.g., a tail sequence, modulator stem sequence (dual guide nucleic acids), targeter stem sequence (dual guide nucleic acids), and/or spacer sequence (see, the “Targeter and Modulator nucleic acids” subsection).

In certain embodiments, the modification alters the specificity of the engineered, non-naturally occurring system. In certain embodiments, the modification enhances the specificity of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil. Within 10, 5, 4, 3, 2, or 1 nucleotide of the 3′ end, for example the 3′ end nucleotide, is modified

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the targeter nucleic acid and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides or internucleotide linkages. The modification can be made at one or more positions in the targeter nucleic acid and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide or internucleotide linkage at the position. For example, a specificity-enhancing modification may be suitable for a nucleotide or internucleotide linkage in the spacer sequence, the targeter stem sequence, or the modulator stem sequence. A stability-enhancing modification may be suitable for one or more terminal nucleotides or internucleotide linkages in the targeter nucleic acid and/or the modulator nucleic acid. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the targeter nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the targeter nucleic acid are modified. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the modulator nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the modulator nucleic acid are modified. Selection of positions for modifications is described in U.S. Pat. Nos. 10,900,034 and 10,767,175. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase.

It is understood that, in dual guide nucleic acid systems the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

### III. COMPOSITIONS AND METHODS FOR TARGETING, EDITING, AND/OR MODIFYING GENOMIC DNA

An engineered, non-naturally occurring system, such as disclosed herein, can be useful for targeting, editing, and/or modifying a target nucleic acid, such as a DNA (e.g., genomic DNA) in a cell or organism.

The present invention provides a method of cleaving a target nucleic acid (e.g., DNA) comprising the sequence of a preselected target sequence or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target sequence or a portion thereof, the method comprising contacting the target DNA with an engineered, non-naturally occurring system disclosed herein, thereby resulting in binding of the system to the target DNA. This method can be useful, e.g., for detecting the presence and/or location of the a preselected target gene, for example, if a component of the system (e.g., the Cas protein) comprises a detectable marker.

In addition, provided are methods of modifying a target nucleic acid (e.g., DNA) comprising the sequence of a preselected target sequence or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section I supra are applicable hereto.

An engineered, non-naturally occurring system can be contacted with the target nucleic acid as a complex. Accordingly, in certain embodiments, a method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

In certain embodiments, provided is a method of editing a human genomic sequence at one of a group of preselected target gene loci, the method comprising delivering an engineered, non-naturally occurring system disclosed herein into a human cell, thereby resulting in editing of the genomic sequence at the target gene locus in the human cell. In certain embodiments, provided herein is a method of detecting a human genomic sequence at one of a group of preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In certain embodiments, provided herein is a method of modifying a human chromosome at one of a group of preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell. The CRISPR-Cas complex may be delivered to a cell by introducing a pre-formed

ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 8,697,359, 10,113,167, 10,570,418, 10,829,787, 11,118,194, and 11,125,739 and U.S. Patent Application Publication Nos. 2015/0344912, 2018/0119140, and 2018/0282763.

It is understood that contacting a DNA (e.g., genomic DNA) in a cell with a CRISPR-Cas complex does not require delivery of all components of the complex into the cell. For example, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell.

Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell (e.g., E coli), an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, or the like, a fungal cell (e.g., a yeast cell, such as S. cervisiae), an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8+T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell; stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### A. Ribonucleoprotein (RNP) Delivery and “Cas RNA” Delivery

An engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, can refer to a complex comprising a nucleoprotein and a ribonucleic acid. A “nucleoprotein” as provided herein can refer to a protein capable of binding a nucleic acid (e.g., RNA, DNA). Where the nucleoprotein binds a ribonucleic acid it can be referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, or the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., at least 2 fold, at least 3 fold, at least 4 fold, or at least 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Pat. No. 10,829,787) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, microvesicles (e.g., exosomes and ARMMs), polycations, lipid: nucleic acid conjugates, electroporation, cell permeable peptides (see, U.S. Pat. No. 11,118,194), nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Where the target cell is a proliferating cell, the efficiency of RNP delivery can be enhanced by cell cycle synchronization (see, U.S. Pat. No. 10,570,418). In certain embodiments, an RNP is delivered into a cell by electroporation.

In certain embodiments, a CRISPR-Cas system is delivered into a cell in a “approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the single guide nucleic acid, or the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Pat. No. 10,829,787) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, polycations, lipid: nucleic acid conjugates, electroporation, nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Specific examples of the “nucleic acid only” approach by electroporation are described in International (PCT) Publication No. WO 2016/164356.

In certain embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### B. CRISPR Expression Systems

Also provided herein is a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid; this nucleic acid alone can constitute a CRISPR expression system. In certain embodiments, the nucleic acid comprises a regulatory element operably linked to a nucleotide sequence encoding a targeter nucleic acid. In certain embodiments, the nucleic acid further comprises a nucleotide sequence encoding a modulator nucleic acid, wherein the nucleotide sequence encoding the modulator nucleic acid is operably linked to the same regulatory element as the nucleotide sequence encoding the targeter nucleic acid or a different regulatory element; this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid and (b) a nucleic acid comprising a second regulatory element operably linked to a nucleotide sequence encoding a modulator nucleic acid.

In certain embodiments, a CRISPR expression system further comprises a nucleic acid comprising a third regulatory element operably linked to a nucleotide sequence encoding a Cas protein, such as a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” can mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of a CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

Nucleic acids of a CRISPR expression system can be provided in one or more vectors. The term “vector,” as used herein, can refer to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6:1149; Anderson (1992) SCIENCE, 256:808; Nabel & Feigner (1993) TIBTECH, 11:211; Mitani & Caskey(1993) TIBTECH, 11:162; Dillon (1993) TIBTECH, 11:167; Miller (1992) NATURE, 357:455; Vigne, (1995) RESTORATIVE NEUROLOGY AND NEUROSCIENCE, 8:35; Kremer & Perricaudet (1995) BRITISH MEDICAL BULLETIN, 51:31; Haddada et al. (1995) CURRENT TOPICS IN MICROBIOLOGY AND IMMUNOLOGY, 199:297; Yu et al. (1994) GENE THERAPY, 1:13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element,” as used herein, can refer to a transcriptional and/or translational control sequence, such as a promoter, enhancer, transcription termination signal (e.g., polyadenylation signal), internal ribosomal entry sites (IRES), protein degradation signal, or the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (see, Takebe et al. (1988) MOL. CELL. BIOL., 8:466); SV40 enhancer; and the intron sequence between exons 2 and 3 of rabbit β-globin (see, O'Hare et al. (1981) PROC. NATL. ACAD. SCI. USA., 78:1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a prokaryotic cell, e.g., E coli, eukaryotic host cell, e.g., a yeast cell (e.g., S. cerevisiae), a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/ and these tables can be adapted in a number of ways (see, Nakamura et al. (2000) NUCL. ACIDS RES., 28:292). Computer algorithms for codon optimizing a particular sequence for expression in a particular host cell, such as Gene Forge (Aptagen; Jacobus, Pa.), are also available. In certain embodiments, the codon optimization facilitates or improves expression of the Cas protein in the host cell.

### C. Donor Templates

Cleavage of a target nucleotide sequence in the genome of a cell by a CRISPR-Cas system or complex can activate DNA damage pathways, which may rejoin the cleaved DNA fragments by NHEJ or HDR. HDR requires a repair template, either endogenous or exogenous, to transfer the sequence information from the repair template to the target.

In certain embodiments, an engineered, non-naturally occurring system or CRISPR expression system further comprises a donor template. As used herein, the term “donor template” can refer to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g. about or more than about 1, 5, 10, 15, 20, 25, 30, 35, 40, or more nucleotides). The nucleotide sequence of the donor template is typically not identical to the genomic sequence that it replaces. Rather, the donor template may contain one or more substitutions, insertions, deletions, inversions or rearrangements with respect to the genomic sequence, so long as sufficient homology is present to support homology-directed repair. In certain embodiments, the donor template comprises a non-homologous sequence flanked by two regions of homology(i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within about 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. It is understood that a CRISPR-Cas system, such as a system disclosed herein, may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SCI USA, 84:4959; Nehls et al. (1996) SCIENCE, 272:886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as about or at least about 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8⁺ T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO 2017/053729). A skilled person in the art will be able to choose proper timing based upon the form of delivery(consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to a modulator nucleic acid. Covalent linkages suitable for this conjugation are known in the art and are described, for example, in U.S. Pat. No. 9,982,278 and Savic et al. (2018) ELIFE 7: e33761. In certain embodiments, the donor template is covalently linked to a modulator nucleic acid (e.g., the 5′ end of the modulator nucleic acid) through an internucleotide bond. In certain embodiments, the donor template is covalently linked to a modulator nucleic acid (e.g., the 5′ end of the modulator nucleic acid) through a linker.

In certain embodiments, the donor template can comprise any nucleic acid chemistry. In certain embodiments, the donor template can comprise DNA and/or RNA nucleotides. In certain embodiments, the donor template can comprise single-stranded DNA, linear single-stranded RNA, linear double-stranded DNA, linear double-stranded RNA, circular single-stranded DNA, circular single-stranded RNA, circular double-stranded DNA, or circular double-stranded RNA. In certain embodiments, the donor template comprises a mutation in a PAM sequence to partially or completely abolish binding of the RNP to the DNA. In certain embodiments, the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 ug μL⁻¹, for example 0.01-5 μg μL⁻¹. In certain embodiments, the donor template comprises one or more promoters. In certain embodiments, the donor template comprises a promoter that shares at least 70%, at least 75%, at least 80%, at least 85%, at least 90%, at least 95%, at least 99.5% sequence identity with any one of SEQ ID NOS: 78-85 of Table 4.

TABLE 4
Promoter sequences

| | SEQ | |
|---|---|---|
| | ID | |
| Name | NO | Sequence |
| CMV | 78 | CGTTACATAACTTACGGTAAATGGCCCGCCTGGCTGACCGCCCAACGACCCCC |
| | | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCT |
| SCP | 79 | GTACTTATATAAGGGGGTGGGGGCGCGTTCGTCCTCAGTCGCGATCGAACACT |
| | | CGAGCCGAGCAGACGTGCCTACGGACCG |
| CMVe- | 80 | CGTTACATAACTTACGGTAAATGGCCCGCCTGGCTGACCGCCCAACGACCCCC |
| SCP | | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTACTTATATAAGG |
| | | GGGTGGGGGCGCGTTCGTCCTCAGTCGCGATCGAACACTCGAGCCGAGCAGAC |
| | | GTGCCTACGGACCG |
| CMV | 81 | TCAATATTGGCCATTAGCCATATTATTCATTGGTTATATAGCATAAATCAATA |
| max | | TTGGCTATTGGCCATTGCATACGTTGTATCTATATCATAATATGTACATTTAT |
| | | ATTGGCTCATGTCCAATATGACCGCCATGTTGGCATTGATTATTGACTAGTTA |
| | | TTAATAGTAATCAATTACGGGGTCATTAGTTCATAGCCCATATATGGAGTTCC |
| | | GCGTTACATAACTTACGGTAAATGGCCCGCCTGGCTGACCGCCCAACGACCCC |
| | | CGCCCATTGACGTCAATAATGACGTATGTTCCCATAGTAACGCCAATAGGGAC |
| | | TTTCCATTGACGTCAATGGGTGGAGTATTTACGGTAAACTGCCCACTTGGCAG |
| | | TACATCAAGTGTATCATATGCCAAGTCCGCCCCCTATTGACGTCAATGACGGT |
| | | AAATGGCCCGCCTGGCATTATGCCCAGTACATGACCTTACGGGACTTTCCTAC |
| | | TTGGCAGTACATCTACGTATTAGTCATCGCTATTACCATGGTGATGCGGTTTT |
| | | GGCAGTACACCAATGGGCGTGGATAGCGGTTTGACTCACGGGGATTTCCAAGT |
| | | CTCCACCCCATTGACGTCAATGGGAGTTTGTTTTGGCACCAAAATCAACGGGA |
| | | CTTTCCAAAATGTCGTAATAACCCCGCCCCGTTGACGCAAATGGGCGGTAGGC |
| | | GTGTACGGTGGGAGGTCTATATAAGCAGAGGTCGTTTAGTGAACCGTCAGATC |
| | | ACTAGTAGCTTTATTGCGGTAGTTTATCACAGTTAAATTGCTAACGCAGTCAG |
| | | TGCTCGACTGATCACAGGTAAGTATCAAGGTTACAAGACAGGTTTAAGGAGGC |
| | | CAATAGAAACTGGGCTTGTCGAGACAGAGAAGATTCTTGCGTTTCTGATAGGC |
| | | ACCTATTGGTCTTACTGACATCCACTTTGCCTTTCTCTCCACAGGG |
| JET | 82 | GAATTCGGGCGGAGTTAGGGCGGAGCCAATCAGCGTGCGCCGTTCCGAAAGTT |
| | | GCCTTTTATGGCTGGGCGGAGAATGGGCGGTGAACGCCGATGATTATATAAGG |
| | | ACGCGCCGGGTGTGGCACAGCTAGTTCCGTCGCAGCCGGGATTTGGGTCGCGG |
| | | TTCTTGTTTGTGGATCCCTGTGATCGTCACTTGACA |
| CAG | 83 | ATCTCGACTAGTTATTAATAGTAATCAATTACGGGGTCATTAGTTCATAGCCC |
| | | ATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTG |
| | | ACGTCAATGACGGTAAATGGCCCGCCTGGCATTATGCCCAGTACATGACCTTA |
| | | TGGGACTTTCCTACTTGGCAGTACATCTACGTATTAGTCATCGCTATTACCAT |
| | | GGTCGAGGTGAGCCCCACGTTCTGCTTCACTCTCCCCATCTCCCCCCCCTCCC |
| | | CACCCCCAATTTTGTATTTATTTATTTTTTAATTATTTTGTGCAGCGATGGGG |
| | | GCGGGGGGGGGGGGGGGGCGCGCGCCAGGCGGGGCGGGGCGGGGCGAGGGGCG |
| | | GGGCGGGGCGAGGCGGAGAGGTGCGGCGGCAGCCAATCAGAGCGGCGCGCTCC |
| | | GAAAGTTTCCTTTTATGGCGAGGCGGCGGCGGCGGCGGCCCTATAAAAAGCGA |
| | | AGCGCGCGGCGGGCGGGGAGTCGCTGCGACGCTGCCTTCGCCCCGTGCCCCGC |
| | | TCCGCCGCCGCCTCGCGCCGCCCGCCCCGGCTCTGACTGACCGCGTTACTCCC |
| | | ACAGGTGAGCGGGCGGGACGGCCCTTCTCCTCCGGGCTGTAATTAGCGCTTGG |
| | | TTTAATGACGGCTTGTTTCTTTTCTGTGGCTGCGTGAAAGCCTTGAGGGGCTC |
| | | CGGGAGGGCCCTTTGTGCGGGGGGAGCGGCTCGGGGGGTGCGTGCGTGTGTGT |
| | | GTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCG |
| | | GCCGGGGGCGGTGCCCCGCGGTGCGGGGGGGGCTGCGAGGGGAACAAAGGCTG |
| | | CGTGCGGGGTGTGTGCGTGGGGGGGTGAGCAGGGGGTGTGGGCGCGTCGGTCG |
| | | GGCTGCAACCCCCCCTGCACCCCCCTCCCCGAGTTGCTGAGCACGGCCCGGCT |
| | | TCGGGTGCGGGGCTCCGTACGGGGCGTGGCGCGGGGCTCGCCGTGCCGGGCGG |
| | | GGGGTGGCGGCAGGTGGGGGTGCCGGGCGGGGGGGGGCCGCCTCGGGCCGGGG |
| | | AGGGCTCGGGGGAGGGGCGCGGCGGCCCCCGGAGCGCCGGCGGCTGTCGAGGC |
| | | GCGGCGAGCCGCAGCCATTGCCTTTTATGGTAATCGTGCGAGAGGGCGCAGGG |
| | | ACTTCCTTTGTCCCAAATCTGTGCGGAGCCGAAATCTGGGAGGCGCCGCCGCA |
| | | CCCCCTCTAGCGGGCGCGGGGCGAAGCGGTGCGGCGCCGGCAGGAAGGAAATG |
| | | GGCGGGGAGGGCCTTCGTGCGTCGCCGCGCCGCCGTCCCCTTCTCCCTCTCCA |
| | | GCCTCGGGGCTGTCCGCGGGGGGACGGCTGCCTTCGGGGGGGACGGGGCAGGG |
| | | CGGGGTTCGGCTTCTGGCGTGTGACCGGCGGCTCTAGAGCCTCTGCTAACCAT |
| | | GTTCATGCCTTCTTCTTTTTCCTACAGCTCCTGGGCAACGTGCTGGTTATTGT |
| | | GCTGTCTCATCATTTTGGCAAAGAATT |
| PGK | 84 | GGGGTTGGGGTTGCGCCTTTTCCAAGGCAGCCCTGGGTTTGCGCAGGGACGCG |
| | | GCTGCTCTGGGCGTGGTTCCGGGAAACGCAGCGGCGCCGACCCTGGGTCTCGC |
| | | ACATTCTTCACGTCCGTTCGCAGCGTCACCCGGATCTTCGCCGCTACCCTTGT |
| | | GGGCCCCCCGGCGACGCTTCCTGCTCCGCCCCTAAGTCGGGAAGGTTCCTTGC |
| | | GGTTCGCGGCGTGCCGGACGTGACAAACGGAAGCCGCACGTCTCACTAGTACC |
| | | CTCGCAGACGGACAGCGCCAGGGAGCAATGGCAGCGCGCCGACCGCGATGGGC |
| | | TGTGGCCAATAGCGGCTGCTCAGCAGGGCGCGCCGAGAGCAGCGGCCGGGAAG |
| | | GGGCGGTGCGGGAGGCGGGGTGTGGGGCGGTAGTGTGGGCCCTGTTCCTGCCC |
| | | GCGCGGTGTTCCGCATTCTGCAAGCCTCCGGAGCGCACGTCGGCAGTCGGCTC |
| | | CCTCGTTGACCGAATCACCGACCTCTCTCCCCAG |
| EF- | 85 | GAATTCAGGCTCCGGTGCCCGTCAGTGGGCAGAGCGCACATCGCCCACAGTCC |
| 1a | | CCGAGAAGTTGGGGGGAGGGGTCGGCAATTGAACCGGTGCCTAGAGAAGGTGG |
| | | CGCGGGGTAAACTGGGAAAGTGATGTCGTGTACTGGCTCCGCCTTTTTCCCGA |
| | | GGGTGGGGGAGAACCGTATATAAGTGCAGTAGTCGCCGTGAACGTTCTTTTTC |
| | | GCAACGGGTTTGCCGCCAGAACACAGGTAAGTGCCGTGTGTGGTTCCCGCGGG |
| | | CCTGGCCTCTTTACGGGTTATGGCCCTTGCGTGCCTTGAATTACTTCCACCTG |
| | | GCTGCAGTACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGTTGAGGCC |
| | | TGGCCTGGGCGCTGGGGCCGCCGCGTGCGAATCTGGTGGCACCTTCGCGCCTG |
| | | TCTCGCTGCTTTCGATAAGTCTCTAGCCATTTAAAATTTTTGATGACCTGCTG |
| | | CGACGCTTTTTTTCTGGCAAGATAGTCTTGTAAATGCGGGCCAAGATCTGCAC |
| | | ACTGGTATTTCGGTTTTTGGGGCCGCGGGCGGCGACGGGGCCCGTGCGTCCCA |
| | | GCGCACATGTTCGGCGAGGCGGGGCCTGCGAGCGCGGCCACCGAGAATCGGAC |
| | | GGGGGTAGTCTCAAGCTGGCCGGCCTGCTCTGGTGCCTGGTCTCGCGCCGCCG |
| | | TGTATCGCCCCGCCCTGGGCGGCAAGGCTGGCCCGGTCGGCACCAGTTGCGTG |
| | | AGCGGAAAGATGGCCGCTTCCCGGCCCTGCTGCAGGGAGCTCAAAATGGAGGA |
| | | CGCGGCGCTCGGGAGAGCGGGCGGGTGAGTCACCCACACAAAGGAAAAGGGCC |
| | | TTTCCGTCCTCAGCCGTCGCTTCATGTGACTCCACGGAGTACCGGGCGCCGTC |
| | | CAGGCACCTCGATTAGTTCTCGAGCTTTTGGAGTACGTCGTCTTTAGGTTGGG |
| | | GGGAGGGGTTTTATGCGATGGAGTTTCCCCACACTGAGTGGGTGGAGACTGAA |
| | | GTTAGGCCAGCTTGGCACTTGATGTAATTCTCCTTGGAATTTGCCCTTTTTGA |
| | | GTTTGGATCTTGGTTCATTCTCAAGCCTCAGACAGTGGTTCAAAGTTTTTTTC |
| | | TTCCATTTCAGGTGTCGTGACATCATTTT |

### D. Efficiency and Specificity

An engineered, non-naturally occurring system can be evaluated in terms of efficiency and/or specificity in nucleic acid targeting, cleavage, or modification.

In certain embodiments, an engineered, non-naturally occurring system has high efficiency. For example, in certain embodiments, at least 1, 1.5, 2, 2.5, 3, 4, 5, 10, 15, 20, 25, 30, 35, 40, 45, 50, 55, 60, 65, 70, 75, 80, 85, 90, 95, 96, 97, 98, 99, or 100% of a population of nucleic acids having the target nucleotide sequence and a cognate PAM, when contacted with the engineered, non-naturally occurring system, is targeted, cleaved, or modified. In certain embodiments, the genomes of at least 5, 10, 15, 20, 25, 30, 35, 40, 45, 50, 55, 60, 65, 70, 75, 80, 85, 90, 95, 96, 97, 98, 99, or 100% of a population of cells, when the engineered, non-naturally occurring system is delivered into the cells, are targeted, cleaved, or modified.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate in vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency may need to meet a certain standard to be suitable for therapeutic use. High editing efficiency in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto et al. (2018) Nat Protoc. 13 (11): 2615-42, and include discovery of in situ Cas off-targets and verification by sequencing (DISCOVER-seq) as disclosed in Wienert et al. (2019) Science 364 (6437): 286-89; genome-wide unbiased identification of double-stranded breaks (DSBs) enabled by sequencing (GUIDE-seq) as disclosed in Kleinstiver et al. (2016) Nat. Biotech. 34:869-74; circularization for in vitro reporting of cleavage effects by sequencing (CIRCLE-seq) as described in Kocak et al. (2019) Nat. Biotech. 37:657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### E. Multiplexing

The method of targeting, editing, and/or modifying a genomic DNA disclosed herein can be conducted in multiplicity. For example, a library of targeter nucleic acids can be used to target multiple genomic loci; a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described herein, can be used for constitutively or inducibly expressing one or more elements. For example, the specificity of CRISPR nucleases is at least partially dictated by the uniqueness of the spacer (in combination with spacer sequence's proximity to a requisite PAM) and its off-target score can be calculated with algorithms, such as crispr.mit.edu (Hsu et al. (2013) Nat. Biotech. 31:827-832). The highest possible score is 100, which shows probability for high specificity and few off targets. Because our SHS library targets intergenic regions, the algorithm for gRNA prediction should be able to make alignments with repeated regions and low-complexity sequences.

It is further understood that despite the need to introduce multiple elements—the single guide nucleic acid and the Cas protein; or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids, such as a plurality of guide nucleic acids disclosed herein. In another aspect, the present invention provides a library comprising a plurality of nucleic acids each comprising a regulatory element operably linked to a different guide nucleic acid such as a different guide nucleic acid disclosed herein. These libraries can be used in combination with one or more Cas proteins or Cas-coding nucleic acids, such as disclosed herein, and/or one or more donor templates, such as disclosed herein, for a screening or selection method.

Expression of exogenous genes, e.g., transgenes, in desired cell types and/or developmental/differentiation stages relies on integration into suitable target polynucleotide comprising a target nucleotide sequence that results in sufficient expression, to a degree sufficient for the intended purpose, from the candidate locus. Expression from a specific genomic site can be affected by many factors including but not limited to cell type and differentiation stage, as one or more components of the target polynucleotide get activated during differentiation while others get silenced, and changes in chromatin architecture. Therefore, the identification of suitable target polynucleotides comprising a target nucleotide sequence in the human genome wherein insertion of exogenous DNA, e.g., a transgene, leads to sufficient expression in the target human cell, and, in the case of stem cells, the expression is maintained at a sufficient level through (1) differentiation and (2) through clonal expansion is desired.

Provided herein are compositions and methods for genome engineering. Certain embodiments comprise composition for editing genomes. embodiments disclosed herein concern novel guide nucleic acids (gNAs), e.g., gRNAs, that are complementary to a target nucleotide sequence in a target polynucleotide. As used herein, a “target polynucleotide,” includes a polynucleotide in which a target nucleotide sequence is located. As used herein, a “target nucleotide sequence” includes a sequence to which a guide sequence can bind, e.g., has complementarity to, where binding between a target nucleotide sequence and a guide sequence may allow the activity of a nucleic acid-guided nuclease complex. Further embodiments disclosed herein concern novel gNAs, e.g., gRNAs, that are complementary to a target nucleotide sequence in a target polynucleotide into which insertion of exogenous DNA, e.g., a transgene, doesn't negatively affect the cell, e.g., significantly affect the expression of one or more endogenous genes or result in a malignant transformation of the cell. In further embodiments disclosed herein, gene expression demonstrated in the human target cell is maintained through differentiation of the human target cell and/or through proliferation in the one or more progeny cells at a level sufficient for the ultimate use of the cells. Certain embodiments disclosed herein concern novel nucleic acid-guided nuclease complexes, e.g., RNPs, such as Cas bound to a gNA, that are complementary to a target nucleotide sequence within a target polynucleotide and hydrolyze the phosphodiester back bone (also referred as cleave or cut) in at least one position on at least one strand of the target polynucleotide. Certain embodiments disclosed herein concern methods for selecting and using gNAs, e.g., gRNAs, for genome engineering. Certain embodiments concern methods for using gNAs that are complementary to a target nucleotide sequence within a target polynucleotide, synthesizing the gNA and nucleic-acid-guided nuclease, and/or combining the nucleic guided nuclease with the gNA to form a nucleic acid-guided nuclease complex, e.g., RNP. Certain embodiments disclosed herein concern methods. Certain embodiments disclosed herein concern methods for engineering genomes. Certain embodiments disclosed herein concern methods where a nucleic acid-guided nuclease complex, e.g., RNP, is introduced, e.g., transfected, into a human target cell along with a donor template, e.g., an exogenous DNA, e.g., a transgene, in which the nucleic-acid guided nuclease cleaves the backbone at a least one position in at least one of the strands of the target polynucleotide and the donor template is used to repair the cleaved target polynucleotide, introducing at least a portion of the donor template into the target polynucleotide. As used herein, “exogenous DNA” or a “transgene” includes any gene, natural or synthetic, which is introduced into the genome of an organism or cell to which it is not endogenous. The transgene may or may not retain the ability to be expressed and/or produce RNA or protein in the human target cell. The transgene may or may not alter the resulting phenotype of the human target cell. Certain embodiments include human target cells, e.g., a eukaryotic cell, e.g., a mammalian cell, such as a human cell, for example a stem cell or an immune cell, generated through a method where the nucleic acid-guided nuclease complex, e.g., RNP, is introduced, e.g., transfected, into a human target cell along with a donor template, e.g., as an exogenous DNA or a transgene, such as a chimeric antigen receptor (CAR), in which the nucleic-acid guided nuclease cleaves at or near a targets sequence in a target polynucleotide and the donor template is used to repair the cleaved target polynucleotide introducing at least a portion of the donor template into the target polynucleotide. Certain embodiments disclosed herein include promoter sequences adjacent to an exogenous gene, e.g., a transgene; in certain cases, constructs including the promoter, when introduced into a target polynucleotide of a human target cell, e.g., an immune cell or a stem cell, maintain sufficient gene expression in the edited human target cell for the intended purpose of the cell or its progeny. In certain embodiments, the human target cell is viable after introduction of the exogenous DNA.

As used herein, a “human target cell” includes a cell into which an exogenous product, e.g., a protein, a nucleic acid, or a combination thereof, has been introduced. In certain cases, a human target cell may be used to produce a gene product from an exogenous DNA, e.g., a transgene, such as an exogenous protein, e.g., a CAR. In certain cases, a human target cell may comprise a target nucleotide sequence within target polynucleotide wherein a nucleic acid-guided nuclease hybridizes and cleaves at a site of cleavage at one or more positions on one or more strands of the target polynucleotide at or near the target nucleotide sequence.

As used herein, a “site of cleavage” includes the location or locations at which a nucleic acid-guided nuclease complex will hydrolyze the phosphodiester backbone of a single-stranded or double-stranded target polynucleotide, after binding at a target nucleotide sequence in the target polynucleotide. In certain cases in which the target polynucleotide of a nucleic acid-guided nuclease complex is double stranded, binding of the nucleic acid-guided nuclease complex to a target nucleotide sequence within the target polynucleotide can result in hydrolysis of one of the strands of the target polynucleotide at or near the target nucleotide sequence, resulting in strand cleavage. In such a case, the nucleic acid-guided nuclease complex can cleave either strand of the target polynucleotide. In certain cases, binding of the nucleic acid-guided nuclease complex to a target nucleotide sequence within a target polynucleotide can result in hydrolysis of both strands of the target polynucleotide at or near the target nucleotide sequence, resulting in cleavage of both strands. The sites of cleavage can be the same for both strands, resulting in a blunt end, or the sites of cleavage for each strand can be offset resulting in single strand overhangs, e.g., sticky ends. In certain cases, mismatches at or near the site of cleavage may or may not affect the cleavage efficiency of the nucleic acid-guided nuclease complex.

In certain cases, uncontrolled gene integration next to regulatory elements of proto-oncogenes has been shown to cause oncogenic transformation, which is particularly important
when engineering cells for therapeutic applications. Therefore, it is desired to identify suitable target polynucleotides comprising target nucleotide sequences that result in safe, stable integration of exogenous DNA with sufficient expression in a human target cell and its resultant progeny.

Exemplary characteristics of a target nucleotide sequence that can demonstrate predictable function without potentially harmful alterations in human target cell genomic activity include one or more of (1)>150 kb, for example, >200, such as >250, and in some cases >300 kb away from a known cancer-related gene, (2)>150 kb, for example, >200, such as >250, and in some cases >300 kb away from any miRNA/other functional small RNA, (3)>10 kb, for example, >20, such as >30, and in some cases >50 kb away from any 5′ gene end, (4)>10 kb, for example, >20, such as >30, and in some cases >50 kb away from any replication origin, (5)>10 kb, for example, >20, such as >30, and in some cases >50 kb away from any ultra-conserved element, (6) demonstrating low transcriptional activity, (7) outside of a copy number variable region, (8) located in open chromatin, and (9) unique, i.e., 1 copy per genome.

In certain embodiments, provided herein are compositions. In certain embodiments, provided herein are compositions for engineering a human target cell at suitable target nucleotide sequences within a target polynucleotide of the human target cell.

In certain embodiments, a suitable target polynucleotide that comprises a target nucleotide sequence has at least one of the exemplary characteristics. In certain embodiments, a suitable target polynucleotide that comprises a target nucleotide sequence has at least two of the exemplary characteristics. In certain embodiments, a suitable target polynucleotide that comprises a target nucleotide sequence has at least three of the exemplary characteristics. In certain embodiments, a suitable target polynucleotide that comprises a target nucleotide sequence has at least four of the exemplary characteristics. In certain embodiments, a suitable target polynucleotide that comprises a target nucleotide sequence has at least five of the exemplary characteristics. In certain embodiments, a suitable target polynucleotide that comprises a target nucleotide sequence has at least six of the exemplary characteristics. In certain embodiments, a suitable target polynucleotide that comprises a target nucleotide sequence has at least seven of the exemplary characteristics. In certain embodiments, a suitable target polynucleotide that comprises a target nucleotide sequence has at least eight of the exemplary characteristics. In certain embodiments, a suitable target polynucleotide that comprises a target nucleotide sequence has all the exemplary characteristics.

In certain embodiments, a suitable target polynucleotide is >10 kb, for example, >20, such as >30, and in some cases >50 kb away from any 5′ gene end. In certain embodiments, a suitable target polynucleotide is >10 kb, for example, >20, such as >30, and in some cases >50 kb away from any 5′ gene end and further comprises at one additional exemplary characteristic. In certain embodiments, a suitable target polynucleotide is >10 kb, for example, >20, such as >30, and in some cases >50 kb away from any 5′ gene end and further comprises at least two additional exemplary characteristics. In certain embodiments, a suitable target polynucleotide is >10 kb, for example, >20, such as >30, and in some cases >50 kb away from any 5′ gene end and further comprises at least three additional exemplary characteristics. In certain embodiments, a suitable target polynucleotide is >10 kb, for example, >20, such as >30, and in some cases >50 kb away from any 5′ gene end and further comprises at least four additional exemplary characteristics. In certain embodiments, a suitable target polynucleotide is >10 kb, for example, >20, such as >30, and in some cases >50 kb away from any 5′ gene end and further comprises at least five additional exemplary characteristics. In certain embodiments, a suitable target polynucleotide is >10 kb, for example, >20, such as >30, and in some cases >50 kb away from any 5′ gene end and further comprises at least six additional exemplary characteristics. In certain embodiments, a suitable target polynucleotide is >10 kb, for example, >20, such as >30, and in some cases >50 kb away from any 5′ gene end and further comprises at least seven additional exemplary characteristics. In certain embodiments, a suitable target polynucleotide is >10 kb, for example, >20, such as >30, and in some cases >50 kb away from any 5′ gene end and further comprises all eight additional exemplary characteristics.

In certain embodiments, a suitable target polynucleotide is >150 kb, for example, >200, such as >250, and in some cases >300 kb away from a known cancer-related gene. In certain embodiments, a suitable target polynucleotide is >150 kb, for example, >200, such as >250, and in some cases >300 kb away from a known cancer-related gene and further comprises at one additional exemplary characteristic. In certain embodiments, a suitable target polynucleotide is >150 kb, for example, >200, such as >250, and in some cases >300 kb away from a known cancer-related gene and further comprises at least two additional exemplary characteristics. In certain embodiments, a suitable target polynucleotide is >150 kb, for example, >200, such as >250, and in some cases >300 kb away from a known cancer-related gene and further comprises at least three additional exemplary characteristics. In certain embodiments, a suitable target polynucleotide is >150 kb, for example, >200, such as >250, and in some cases >300 kb away from a known cancer-related gene and further comprises at least four additional exemplary characteristics. In certain embodiments, a suitable target polynucleotide is >150 kb, for example, >200, such as >250, and in some cases >300 kb away from a known cancer-related gene and further comprises at least five additional exemplary characteristics. In certain embodiments, a suitable target polynucleotide is >150 kb, for example, >200, such as >250, and in some cases >300 kb away from a known cancer-related gene and further comprises at least six additional exemplary characteristics. In certain embodiments, a suitable target polynucleotide is >150 kb, for example, >200, such as >250, and in some cases >300 kb away from a known cancer-related gene and further comprises at least seven additional exemplary characteristics. In certain embodiments, a suitable target polynucleotide is >150 kb, for example, >200, such as >250, and in some cases >300 kb away from a known cancer-related gene and further comprises all eight additional exemplary characteristics.

In certain embodiments, a suitable target polynucleotide is >150 kb, for example, >200, such as >250, and in some cases >300 kb away from a known cancer-related gene, and >10 kb, for example, >20, such as >30, and in some cases >50 kb away from any 5′ gene end. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least one additional exemplary characteristic. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least two additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least three additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least four additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least five additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least six additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises all seven additional exemplary characteristics.

In a preferred embodiment, a suitable target polynucleotide is >10 kb, for example, >20, such as >30, and in some cases >50 kb away from any 5′ gene end and >150, for example, >200, such as >250, and in some cases >300 kb away from a known cancer-related gene.

In certain embodiments, a suitable target polynucleotide comprising a target nucleotide sequence, e.g., for transgene insertion, may comprise any one of SEQ ID NOs: 2020-2043 of Table 5. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2043. In a preferred embodiment, a suitable target polynucleotide comprising a target nucleotide sequence is at least 98% identical to any one of SEQ ID NOs: 2020-2043. In a more preferred embodiment, a suitable target polynucleotide comprising a target nucleotide sequence is at least 99% identical to any one of SEQ ID NOs: 2020-2043.

In certain embodiments, a suitable target polynucleotide comprising a target nucleotide sequence, e.g., for transgene insertion, may comprise any one of SEQ ID NOs: 2020-2042 of Table 5. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2042. In a preferred embodiment, a suitable target polynucleotide comprising a target nucleotide sequence is at least 98% identical to any one of SEQ ID NOs: 2020-2042. In a more preferred embodiment, a suitable target polynucleotide comprising a target nucleotide sequence is at least 99% identical to any one of SEQ ID NOs: 2020-2042.

In certain embodiments, a suitable target polynucleotide comprising a target nucleotide sequence, e.g., for transgene insertion, may comprise any one of SEQ ID NOs: 2020-2041 and 2043 of Table 5. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2041 and 2043. In a preferred embodiment, a suitable target polynucleotide comprising a target nucleotide sequence is at least 98% identical to any one of SEQ ID NOs: 2020-2041 and 2043. In a more preferred embodiment, a suitable target polynucleotide comprising a target nucleotide sequence is at least 99% identical to any one of SEQ ID NOs: 2020-2041 and 2043.

In certain embodiments, a suitable target polynucleotide comprising a target nucleotide sequence, e.g., for transgene insertion, may comprise any one of SEQ ID NOs: 2020-2041 of Table 5. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2041. In a preferred embodiment, a suitable target polynucleotide comprising a target nucleotide sequence is at least 98% identical to any one of SEQ ID NOs: 2020-2041. In a more preferred embodiment, a suitable target polynucleotide comprising a target nucleotide sequence is at least 99% identical to any one of SEQ ID NOs: 2020-2041.

In certain embodiments, a suitable target polynucleotide comprising a target nucleotide sequence, e.g., for transgene insertion, may comprise at least a portion of, for example, nucleotides 1-495, 1-490, 1-485, 1-480, 1-475, 1-470, 1-465, 1-460, 1-455, 1-450, 1-445, 1-440, 1-435, 1-430, 1-425, 1-420, 1-415, 1-410, 1-405, or 1-400, of any one of SEQ ID NOs: 2020-2030 of Table 5. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to the portion of any one of SEQ ID NOs: 2020-2030.

In certain embodiments, a suitable target polynucleotide comprising a target nucleotide sequence, e.g., for transgene insertion, may comprise at least a portion of, for example, nucleotides 5-500, 10-500, 15-500, 20-500, 25-500, 30-500, 35-500, 40-500, 45-500, 50-500, 55-500, 60-500, 65-500, 70-500, 75-500, 80-500, 85-500, 90-500, 95-500, or 100-500, of any one of SEQ ID NOs: 2031-2041 of Table 5. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to the portion of any one of SEQ ID NOs: 2031-2041.

TABLE 5
suitable target polynucleotides comprising a target nucleotide sequence
for transgene insertion

| SEQ ID NO | Sequence |
|---|---|
| 2020 | GCCTCCCAAAGTGCTGAGATTATGGGCATGAGCCACCGCACCTGGCCCTGAC |
| | AAGAACCTTTGAGTTAGGTATAATGGTTCACCCCAATTTATAGATAATGAAC |
| | CCAAGTCACAGGGGAAGTGAAGTCAGTTGCCTAAGGTCAGACAGCAGTAAAT |
| | GGTTCTCTGACCCTAACTCCACTGCCTCCCTCTCATAAAAACACTGGGTGGT |
| | TACAGTGGGCCCACCTGGAGAAGTCAAGCTATTCTCTCCATCTCAAGAACAT |
| | TAATTTAATCATCCTTTTTACCATATAAGATAACATCTTCACAGGTTCTGAG |
| | GATGAGAATGTTGACATCTTTGGGTGGTCGTTATTCAGCCTATCACAGGTAT |
| | CCAGGGAAGAAAAAAGGAATTTCCAAAAAGAGAAAATACGAACATTGGGAAG |
| | GCTAATTACAGATGGTGACTACTGAAGGGTTAGTCAGAAGCATAATGGAGGC |
| | AGTGATGAGATGACAGCACAGATGCATGACTCTAGTCCCAGCAACTCCTAAA |
| | AGGTAAAGAAATGTATCCTGCCACCCTCAGCTTCTTTGGGGTGTCCTCATAA |
| | AAGAGAGGCAGTAAAGCAGAATCAGAGTCAGATAGAGAGGTTGTAAGAAGAG |
| | AAGCAGAGGTGAGTAAGCTGTGTTTCAAACCCAGAGTCAAGGCTCTTGCCCC |
| | TCTGCGGTGCTGCCGAAGCCCAGGGTGGGTGGGGACTGACATGCAACTCAGG |
| | TACTGTGTGGCAGACTTTGTGCCTTGGCATGAAACTATGCCTGCCCACAGGA |
| | AGGGGCACCATTTTCTCATTAGCTCAAAGAGACTTCTGCTGGCCAATTCCTG |
| | TCTTCTCAATACTGCAGCTCTCCAGAGACAACACTGTTCTCTATTCTCCTGT |
| | AAGTGAGGCAGAGCCTGGCAGTACCCTCTATGCCACCTCTCACTAGTACAGG |
| | TTAGCACTCAGGGTGGCCCACTGGTGTGTGTCTCAGCTGCTGGTGTGCGTGC |
| | TGGTGCAGGTAC |
| 2021 | TGGGCTGAGGGTTGTGGCTGGATCTCTTTGCATTGCCACATCCACAACAGAA |
| | TTTTGAGAAGTCCGAGAATTCTAAATTGGAGCCTGACCTTCTTCATAATAGT |
| | ATATTTGTCAAGGTAGGAGGATAAAACATTTTATTGAACAGTTTGCTAAGCT |
| | GATTTAAAATTTTCCAGCATTTAGCTATATGGTATATGGACCTCCACATGTA |
| | TGATTTCATTTATATTAAATGTCCAGAATAGACAAATCTATAATGACAATAA |
| | AGAGATTAGTAGTTGCCAGAGGCTGGGAGGAGGGGAGAAACAATGAGTGATT |
| | GCGGACGGGTGTGGGGTTTCTTTCTGGGGCGATAACAGTGTCCTGGAATTCA |
| | ATAGTGATAATGGATGCACACTGTGAATATACTAAAAGCCACTCACACTTTA |
| | AAAGTGTGGGTTTTATGGTAATTTGAATGATATATCAAGCTATCACCAAAAA |
| | TACACAATGGGAGTTCAGAAATGCCACCCCAAACTATGATGATTTGACATGC |
| | TCATTACTTTGAACTGATGTCACTTGGGGAAAAACAGATGCAGGCAGAGACT |
| | TTCTCTGAGATCTGCTTATCTGCCTAAGACAGATCAAGGGATCCTCCAAAAG |
| | GAACTCAATTGTCATGAATCCCCTCCCCTGGAACCTTATCAACCAGGGACAA |
| | TGAACTTAGATCAGAGAGGGGGAGACTGGAGGTTGACATCATGCCTAGACAG |
| | CCACCTCTTCTTCTGAGGGCTGCTCCAAGAGAACCTTTATTACTTGAGAGGC |
| | TTCTCATTTGCATAACAAGAAACCTTTGTTCACCATACACTTCCTCCCCTCA |
| | TATTCTCATAACTGGTGTCACCACCACCCACGCAGAAGTCCAAAGCCTCTAT |
| | TCCCTTCTGTACCTCAGGGTGCTATATAAGCTTCAATCATCTGACCCTTCTT |
| | TGAATCTCATATTTTGTGGGCTTGCATGGGTATGTACATAATTAAAAATGGA |
| | TTTCCTCTTGTT |
| 2022 | ATTTACACACATGCCACAGACAGAAACATTTTAATAGACCTTTGCTTATGGA |
| | AAAGTAAAGCAAAAATGTAATTCTAGAAGGGAGAAATTTTAGTCAATTAGAA |
| | AATAAGATGGTCAGGCATTGTAGCTCTCATGTGTAATCCCAGTGCTTTGGAA |
| | GGTTGAGGCGAGAGGATTGCTTGAGACCAGGAGTTTGCGACCAGCCTAGACA |
| | ACATAGCTGGTCATATAAAAAAACTTCAAAAAAATTAGCTAGCTGTAGAGCT |
| | TTCTGCCTATATTTCCAGCTACTCAAGGATGAGGCAAAAGAATCCCTTAAGC |
| | CCAGGAGGTTGAGGTTGCAGTGAACTGTAATTGCACCACCACACTCTAGCCT |
| | GGGTAACAGAGCAAGGTCCCATCTCCTAAAAAAAAAAGAAAGGAAAATAAAA |
| | AGAAAATAAACTATTCTCCATAATAATGTAGACAGCAATCCTCACTGTGAAC |
| | CAGAAGGAACCTCGGCAAATTTTTTAGACATCAATGGGATTTCACTATCAGC |
| | TGAGAGTGTTCCCTTTTTAGCATGGCAAGCTGTTTCCTGAAGCAATAGAGAG |
| | AAGCAAGACCAAGGAAAAATCTAGAAAGAGCCTCTCTGTAGAAAAGCAGAGC |
| | AATGATCTCTAATCACAATGCTATCAAATATTCCAGGCTAAATTTTCCTTTA |
| | TAGCATTAAAATTTTCCTCACATCCACAAGATTCCAATAGTTTTCTTAATGC |
| | CATAGCCTGGTGTCTATTCTGCCTTGTGGATTCCCATAATGCAAAATGCCAT |
| | TAAAAAAGGAACAGACCATGAGAAGTGGGCCTCCGAAGCACATGAAGCTTGG |
| | TATCATCAGAAAGATAAGGGGCAACAGTCAGGAATAATTGTTGGGACATTTA |
| | ATAAGTCCCTGGAAATTCCTAGAAACATAATTTTTTTTTGAGTCTAAGATGC |
| | TATCATTTTAAGGTGCACCATTATTTTATTTGCTACAATGTAGAAAACAATA |
| | ACACTGCCAATT |
| 2023 | TGATTAGGTAAAATATCAGAGACACAAATCAGGTTAAATTGATTTTTTATTG |
| | TAATTACATTTAAAATTTTAGAATTCATCAGTAGGTATGAACAAACATATAC |
| | ATACATATATATAATTTATATTATAAGTTTATTATTTATACTATACATTATA |
| | AAAATAACTGAGAGATAAACTTTCGTTTATCCTTAATGCTAAAATAATTCAT |
| | TTACCTTGGAGAGATCAGAACTCTGTCCATTTCCCCTACATAAAAACTAGAG |
| | AGTACTATTGCTTTCTCTTTCTCGGGCTTACTCTGGTCTCATAGAATATGCA |
| | TTTTCATTTTTTTTCAACAGAATATCCGTGGATAGCTAAAATTTCTGCTTCC |
| | TTTGTCAACATTTGTATTTCCCCAGTGGACATTTCTGCAAAATTTATTTTCA |
| | TTTCTTTGTTACCAGAGAAACTCTGTTGGTCAAGTTCAATAGCATCCTCAGC |
| | ATAATTTCAGAAGGAAATTACAGGGAGCAATTGAAGTCCATCACTTTCTTGG |
| | AGGGGAAATATTAACACCCTCACCTCTTGCTCCCAATATTAGGTGGTAGGCA |
| | GGAGTGAGTTACTCATTTTCTGAAGGAGCAGTAACTCTTTGGACCCCTCGAG |
| | TCACTTGGTAAATAAACTCTAGCACTGCCCCGAAGAGTGCCTCAGAGATTTC |
| | AAGGAATAAATGCTTTAAAGGTAGGAAAATGCTAAGAAACACCATCATATAA |
| | GTGAGTTATTTCCAATTTTATTTTAAATACAGCCATATATTATTACATACAG |
| | CCACACATTATTAAATAATGTATTAATACATTATTATTAAATACAGCCATAT |
| | ATATGTATATATGTGTGTGTGTATATATATACATATATATGTAAGTATGTAG |
| | CTGCTATACCCTCCTGAAGCAATGAATGTAGCTGCTATACCCTCCAGAAGCA |
| | ATGATACCCTCCAGAGGTGATAACAGATACAAGTAACAACCACACTCTCTGG |
| | TTTTGACAACCA |
| 2024 | CAGAGAGCTTCCAAGGCATTATCCCATCCAAAGGGTAAAGAGGCTGGGATAT |
| | TTATCGACTAGCTCCCATTCTTCACTGGCTGTAACTTGTCCACGTCTCACAG |
| | CTGTAACTCCCTTGTATTCCCTACCTATCTGGTGTGAGGACCAAGCTTGTGT |
| | CTGTGGATAGAGAAAGCCCTAAAGCAGAAAGTCTAGGTGCTTGCACAAAAAG |
| | ATCATCTGCACAGAATGATGATCAAGAGATGTGAGTGGGGCACCACAACATT |
| | TACCTCAGGAATCTCTGTTCAGGACTCAGCTTTGGTCTCAAACCTTGGGAAG |
| | CTTATACACTGAGGCAGTGTTAGGATCTCTTTTCTCTGCCTTCCTGTGCTTT |
| | TAAGTGTATTTCACTGTTTTTGATCCCTTGTCTGCCCCTTATATTTGACTAT |
| | CAGGCTCTTGAAGGTCTATTACACTTACTCATTGTTTTTACCCCCTGTTCCT |
| | ATCTCAGTGCCCAACACAGAGCTGACAGTTAATATATGTTGGTTGGATGCAT |
| | GTGTGGGTATCTTATCTTTTTATCCTTTAAAAGACCTCACACGTAGATGAAA |
| | ATTTTAAAATCATTAATTCAATCATCAATTCAATTCAATCATCTTTTTATCC |
| | TTTAAAAGACCTCACACATTGATGAAAATTTTAAAATCATTAATTCAATTGA |
| | AGAGGCCTTGTGATTGACATGAGTATAAATTGGACCATTATTAACTTCAAAC |
| | TAATTCTACTATGCCAGAAACCATGCCTGAAGTATTAAAACATCACGTTAAA |
| | AAACAAAAGACAAAAAAAAAACTTATCTAAAAAATTACATTAAATAAAATAG |
| | ACCAAAGGTAAATCTTACTCAAGTTTTCAGGAAAAAAAAATTGTTTTCTATA |
| | CTCTTTTCTCACCTATTCTTCCTTGTCACAGAGAAGCAATTATTATATTAGA |
| | CTTTCCTTTTTCAATGTGTAGATGACATCATATGATTTAAATTTTTTATGTA |
| | TTTCTCTTGCAA |
| 2025 | ATCAGCAGCAGAGGCTGCAGAACAGCGGATATTAGTGAAAAGCAAATGTTGC |
| | TGTCTGATCGTTCCTGTGGAAGTTTTGTCTCAGAGGAGTACCCGGCCGTGTG |
| | AGGTGTCAGTCTGCCCCTACTCGGGGGTGCCTCCCAGTTAGGCTACTCAGGG |
| | GTCAGGGACCCACTTGAGGAGGCAGTCTGTCTGTTCTCAGATCTCAAGCTGT |
| | GTGCTGGGAGAACCACTACTCTCTTCAAAGCTGTCAGACAGGGACATTTAAG |
| | TCTGCAGAGGTTTCTGCTGCCTTTTGTTGGGCTATGCCCTGCCCCCAGAGGT |
| | GGAGTCTACAGAGACAGGCAGGCCTTGAGCTGCAGTGGGCTCCACCCAGTTC |
| | GAGCTTCCTGGCTGCTTTGTTTACCTACAATGGTGGGCTCCCCTCCCCCAGC |
| | CTTGCTGCTGCCTTGCAGTTTGATCTCAGACTGCTGTGCTAGCAATGAGCGA |
| | GGCTCCATGGGCGTAGGACCCTCCGAGCCAGGTGGGATACAATCTTCTAGTT |
| | TGCTGTTTGCTAGGACCATTGGAAAAGCACAGTATTAGGGTGGGAGTGACCC |
| | GATTTTCCAGGTGCTGTCTGTCACCCCTTTCCTTGGCTAGGAAAGGGAATTC |
| | CCTGACCCCTTGCGCTTCCTGGGTGAGGTGATGCCTTGCCCTGCTTCGGCTC |
| | ATGCTCAGTGCACTGCACCCACTGTCTTGCACCCACTGTCCGACAATCCCCA |
| | GTGTGATGAACCCGGTACCTCAGTTGGAAATGCAGAAATCATTCATCTTCTG |
| | AGTCACTCACGCTGGGAGCTGTAGACTGGAGCTGTTCCTATTCGGCCATCTA |
| | CATGTTCTTTCTTCCCTCATCATCACTTCTTTACTTCTTTTATTTCACTTCT |
| | GGCTTTCTGTCCTCCCACGCTGAGGAAGACTGATTTGGTGGACATGTATTTA |
| | TTCTGCTGAGTACCAGTTGATGTGGAAGTAGTTGTTTTATAGTCAACATGTT |
| | TTTATGACTAAT |
| 2026 | GAGTGATGTCTAATCACAATCTGTGATAGGTATTTGCTTTAAGGTGCATCTA |
| | ATAACATGACAGTGATTTTCATCTCATATAACCTTCATTAACTCTGGTTCCC |
| | TGCTAAGATAAAGCCTTCCCTATAAGCCAACTGAGAATACTGTAGTCAGAAT |
| | TTACAGGTACTTCCCATTGTGGTTGTTCACCTTATTTGTGCCAGTTTTTCTT |
| | CTTCTTTATTCATACCTTTTGCCATGTGAATTTGCATTTCTTCTGGGTTGGA |
| | GTCAAGTATATATTTATCCTTTTTACCTTTGACTCTGAGGCTGGCCAAAGGA |
| | ATAAGGTGGATGTGACAAGGTACAATTTCTGAGCCTAGCCCTTAGAGGCCTT |
| | CCATGTTTCCACTTGTTCTCTTGCACTTGCGACGTTGCTGTCAAAAGAACAT |
| | GCAATGGCTAGCTAGCAGCCTGTGCACCTGCAGTGAGAACCAGAGCCACCCA |
| | GTTGCTGCAGCCTGAGACCAAGCTGCTCAGCTAAGCATAGCTTAGATCACCA |
| | TTGAGTTCTGAGGTGGTTTGTCATACAGCAATGGCAATCAGATATATCCACA |
| | CAAATATAATTTTAGTTTATATTTTTGTTACTGCAGTTCTCATCTTATTCTG |
| | AGGATACGTGACAAAATAATTCTTTCAAAAATATTGATGCTGTGCCAGATTA |
| | CTATTTTGAATGAATTATTAGACAAATACTTCATATGTATCTTATTATGTGG |
| | GTTTACACATTATTTATCTTATTGATTTAACTTCAAAACTAAACTTTAGTTT |
| | AGCTCTTGGGCCCTATCTGGGAAAGGGTCATCTTTTAATCACCATTAAATCA |
| | CTGAAGTCATCAGTTTATTCAAAGTACTCTGCACAAAATTAGCATTCTTTAG |
| | TGGTTGTGAAATAAATAGACTTTAAACTTATCATTAATATTCCCAATGGTAC |
| | TATGGGGGAGGCAAAATTTTCTATCTTCTTAGTGGTTTTTTTTTTTTTGGCT |
| | AGGGCTAAGGAT |
| 2027 | ACGCACCTGAGAAATGTGTTAAGGATTAAGATGCTAGTGCTAGATGTTTGAT |
| | TTTCTGAATCGAACCACTATTGGTGAGATCCAGAAGCTCAAAGACATGATAT |
| | ACCCACCTTCAAATAATGTTTATGTAGGTAATCTATTCTCAGGATTTATAGA |
| | CACTGCTGTTAAGACCTATTGTCATTGGGGTAAAAAAAAATCCTTATTATAT |
| | TATACAAATTATTATATACTATTATATTATAGAAATTATATTTCTATTAAAT |
| | AGCTTGTGTAGAAAGTAACCATATATAGTTAGAAAAACACTGATCTCAAGAA |
| | CAGGATTTTAGATTTGACTCTGACAATTTCTGTTCGGTCTTGTATAAATGTA |
| | TCAATTTAGATTTAGGGCTTTATTTTCTAATCCATAAAATGTGTAGCATACT |
| | TCTGCTAGCTATACATTTACTGAAGTTATTATTTTAAACTATTTTTATTTTC |
| | ATTTTTTTGTTTTGAGTTATAATCATAATTAATGGATTCAAGTGACAGAGAA |
| | AAGAAAGTAATTAGTCATCTTTTTTCAGAATACAGTCTTTGTTCTGAAGGTA |
| | TTTCGTATGAATCAAGTTTCAAATCTTCAGATAAATTTTCACCTTGCCAATG |
| | TGCTTTCTGCTCTAAATCATTCCTGAATTTTGCTATGATTTTTCTTTCTTAT |
| | AAAATCTTGACACTAAATTGTCAGGAGATATACATATATGTATATATGTAAA |
| | ATATATATATCATATATAAATATATATAAATTTTGAGTTAAAGTACTATTAC |
| | AGTATTCAATTCTACCAGTAATTCTAATAGTATGAAAATAAAGTCACCAGTT |
| | GAAGTAAGACCTACTGACACCTTCTATTATATTTCGATAATTCTATTTGAAA |
| | CTAATTATATAGTAGGACATTTTCATTGTTTTCAGTATTAACTGGCACTCAT |
| | GTAGATATTGCAGGCCAAATTTTACCTCTACCTTTTGGAATTTTCTGGGGTA |
| | GACTTGAGAATT |
| 2028 | TACATGTGTAAACAGTTTTAGCGTAGATTTCCTCGCACTTTTAAATTTTGGA |
| | TTCTTAATTTCCCTGTCCCCCCTGCCCCCCCCCCAAAAAAAACCTGCTAACG |
| | TTTAAACGAACACAGTTTGGGAAATCTGCGTTAAGTCCTTCGTGGGAGTGGG |
| | GTTGCTCAGCTCACAGTAGGCCACGAACCTGAATTTTCTCTTGTCTGCTGCC |
| | CCCTTTTGATAGATGGAGGGAAGAGCAGGCTTCCAGTGCAATGGACAGAAGA |
| | GGGAGCCTGCAAGTTGGTAACAGAGTCTATTAGGGAAAGAGAGAGTCACTTG |
| | AATCCTCAGAGCTGCTCCTGTCAACTGCTTTGTGCAGTTTTTGTGACTTATT |
| | AGCTGCTTGTTTGCACTCTATCTACGCCTGCCCAGGTGTGTTTGGGCCCTAG |
| | AGCGAAGGGAGCACAGGCGTTCATTTAGAAACTTATCCCTCCGTCCAAATAT |
| | TGGATGCTTACCATGTGCCTGGTGCAATGCAGGGTGATACAAAGAGGAAGAT |
| | AAGTGAGGCATTCTTATCGAAGGACCAGACACTCTTCCAGCCTGACTATATT |
| | CATTACACTCGTGCCTGACCTTTCTTTGACTCTAAGATTCTTCCTTTCTAAA |
| | TGTGAATCTTAAAGACTGAAGTCTTTGATCTAAGACTGCTTTCTTATCACAT |
| | CACATCCAACAACCAACTTTTCACAGCTTCCCAGATCCCAAATTCTGTTTAG |
| | CAAGGACACTTGGATTTTTTTGTTTTTTGTTATAAATGACCTCTTCAGGTTC |
| | ATATTTTCACTATGTCCAGAATTCTTATTTTATTCTGTTTTGTGCTGACATT |
| | GGAGGCAGAGTCTGTGTCACAGAATACACCACTAGGGGTTACCCTGGACATG |
| | GAAGGGTATTCACTCGGGGAAGAAATTTTAATGGAATTTTTAATATCTAGAG |
| | CTGTCATTATCCTGTGATGGTTCACAAGAAATGGAACACTTAAAAATTTCTA |
| | CAGAAAAAAAGG |
| 2029 | GCCACAAATTTGTTTTCTGTATCTGTAGATTTGCATTTTTTTCCGAACATCT |
| | CATATGAATAGAATCACAAAATTTGTGTATTTTGTGCCAAACTTCTTTCACT |
| | TAGCATACTGATTTCAAAATTGATCCAACTTATAGCATATATCAGTACTTTA |
| | TTCCTTTTTAGGGCAAAGAAATCTTCCATTACACGGATACCCCACATTTTAT |
| | TTCTCTACCCATCGCTTGCTGGGCATGAGTTGTTTGTGACAAATATTCATAT |
| | ACATATTCTTGTGTGGACATATGTTTTCGCTTCTCTTGGGTATATATCTAGG |
| | AGTAGGATTGCTGGGTCATATGGTAAGTCTCTATTTAATGGTTTAGACTCAG |
| | TACTTTGTTTTCTGCCTTTCCACAGCTCAGTTTCATAAAGAGGCAGGAGCCT |
| | TTTGTTCAGGGCTCCTTGGCAGTAAGGTAATTTCTTCTTCTGCATTGTATCC |
| | AGCTGACCCTTGCTCAGTGCTGTTCTTTGGGGGAAAGATGGAATGCTGGGAA |
| | GCCAGCACCTCTTATTCCTTCTAGCTAACACTTTTACAGTGACGGATATAAT |
| | AGATATCTTCAACTAGTATTGTTGAATTATCTCCCTGATGCTGTCCAATTTT |
| | GCTTCATATATTTTGGGGCTCTGTTATTAGGTATGCATATATAGTCATTATT |
| | GTTATATCTTTGTGGTGGTGTGGCCTTTTTATTATTTTAGCACTTTTATATC |
| | TTTACCTCTAATAACGTTTTTAAAAATTGAACGTTGATTTTGTCTGATGTTA |
| | GTACAACCACTTCAGCTTCTTTGTAGTTGCTGTTTGCATGACATATCTTTCT |
| | CCATTCTTTTACTTTCAATCTATTTGTATCTCTGGGTCTAAAATGTGTAGAT |
| | AGCACATAGTTGAATCTTTTAAAAAATACATTTTACAATCTCTGATTTTTAT |
| | TGGAATGTTTAATCCATCCACATTTAATGTTACGATTGATGGAGCTGGACTT |
| | ATTTCTGCCATA |
| 2030 | AACACAGAGCTAAAACCAAGTAAGAGGCGATTCTCCAAAAGCACTTCCTCAG |
| | CAAACAGCATATCTATTGTGTGTGGGTTCTTTAATTGGCTGAGAACTGAATT |
| | TCACCTTTGGCATTAAAGAGAAGTGTTTATTTTTACTGTCTTCACTGTTTTA |
| | ATGTTTAAACAAAATCTAAATACTGAGGTGAACTCTATCATAAAACAAGTGA |
| | AACGGCAACATAGGTTGATCCAGAAAGAAGCAAATTCCAGCATGGCGGGCAC |
| | TACATGTTTCAGCTCATCAGTTATCTGAATCTTATGGCTCTAAAGATGGATG |
| | GATGAGAATACATAGGCAGAAGCTTCCTGGTGAGGCTGGTATGATTCTGTTG |
| | TCCTATCTTCAACACTATCCTTCTACCTTCAGGGTTGCTGTTGTAGGTTTTA |
| | TTTCTTTGGCTTCTGTTGCCAGTAATGGAAAAGGACCACATGGAAGACTGTA |
| | TTTATGTACATCATGTCCAAACAGAATATCCTATAATAGTGAATCTTGGAAG |
| | AAAGCTTGAGAGATGTGGCCCAGCGCGGTGGCTCACACCTGTAATCCCAGCA |
| | CTTTGGGAGACTGAGGTGGGCTGATCACGAGGTCAGGAGTTCGAGACCAGTG |
| | TGACCAACATGGTGAAACCCCATCTCTACTAAAAAGACAAAAATTAGCCGGG |
| | CCTGGTGGTGTTGCACCCGTAATCCCAGCTACCCAGGAGGCTGAGGCAGGAG |
| | AATTGCTTGAACCCAGGAGGCAGAGGTTGCAGTGAGCCAAGATCGTACCACT |
| | GCACTCCAGCCCTCCAGCCTGGACAACAGAGCAAGACTCTGTCTCAAAAGAA |
| | AAAAAAAATACCAGTTTGAGAGATGTATGTGAGGACTGATTACCGAAAGCGA |
| | AAGGGTTTAGTACATCTCATGAGAACAGAGCAGTCACAAGTGATATAAACCA |
| | AACTCCCTTGGAAATTTGTAATCTATCAACTTCTTTATTTAAAGAGAATAGG |
| | AGGTTTACTGTG |
| 2031 | ACTCCCACTCCTACTAATTACAGCTTGTGTGTCCTTCAGTCATTCACTTCCC |
| | TTCACATGACCAGCCCAGCAGAAATGAACTACCAGGAACATGAGCTCAGAGC |
| | GATGGGCTGGCCACCTGCCAAGCACCTCTGAATGGAAAGAGCAGAATTTTGC |
| | ATTGCCTGCCATGCCACGTGGAGCAGGCCCTGGGTGGCTCTTTAGGGGATGG |
| | GTGTGGACTCCCACAACAAAACCAAGGGCCATATTCAAAGTTAAAAGCTCTG |
| | CCATAGATGGTATTTGTTGAGGCTGTGTGTGGTAGCTCATGCATGTATGCCC |
| | AACACTTTAGGAGGCTGAGGTGGGAAGATCACTTGAGGCTGGGAGTTCAAGT |
| | CTAGCCTAGGCAAGATAGTGAGATCCCTTCTCTAAAAAAGATAAAATATTAA |
| | CTGGGCATCATGGACGTGCCTGTAGCCCCAGCTACTGGGGAGGCTGAGGCAG |
| | GAGGATGGCTTGAGTCCAGGAGTTTGAGACTGCAGTGAGCTGTGATTGCACC |
| | ATTGCTCCCTAGCCCGGGTGACAGAACAAGACTTTTATTTCTTTAAAAAAAA |
| | AAAAAAAAAAGAAGGTGTTTACTGCAGTTGCTTTATTAAAAAAAAAGTAAAT |
| | GAATGTTCTGACTGTTCTACTTTTGAAAATAAGTGGCAAGGAATTAGAACTG |
| | TATCTTTCAGCAACAAAATGTACACTGTGGTTCCATGTCACAGCCAGGAATG |
| | GAGTCAGATGTCTCAGACCAGAATCACAGCTCTGCCACCTCCTGTGACATGG |
| | ACTTGCTAAGCTACCTTGACTCTCTGGAGCTCACTATGCCCATCAATAACAA |
| | GAAATAAATAAATCCGTCCTGTAAGGTTGTCAGGAGAAACAAATGAGGCACT |
| | ATATGTGGAAGTTCCTGGAATAGTGACCAGCACAGAGGACGTCTCAAAGAAA |
| | GATTTGCTGAACCCCAAAAGACAGGAGGACTGGAGGAACAACAAAGAGACAG |
| | GAAAGCTAGCAT |
| 2032 | AATTCATAGCCCAGCCAAGGAACTTAGAAGAGTAGAGGGAAGTCATTTTTCA |
| | CTCCCCTACAAGAACATTCTGCTGTAAAGAGGAGCTAGAAATAATTTTTGTT |
| | TTAAATTCAACCAAACATAGGGATAATTCTGAAATTTGGAACCAAAAGAATT |
| | ATAAGTACACTACTGGTGAATTTGTGCTTATCTGAAATCTACACATGTAGCT |
| | GTCTTTATGTATCTCTGTATATCGATGTTTTTCTATATATATAATCAGTGAA |
| | GTAAGATATCTAGTCATTCATTTACTCACCAAGTGATTGCAGTGGGGTGACA |
| | GGGACAGTGGGGGGTGTGGTGGCGGGTTGCCAGAGCATGAGGAGTATGCAAT |
| | AGAATCTAAGAAATCATACCTACCTGGCCAGGCACAGTTGCTCATGCCTGTA |
| | ATCCCAGCACTTTGGGAGGCAGAGGCAGGCGGATCACTTGAGGTCAGGAGTT |
| | CCAGACCAGCCTGGCCAACATGGTGAAATCCCATCTCTACTAAAAATACAAA |
| | AAATACAAAAAATTAGCTGGGTGTGGTGGCACATGCCTGTAATCCTGGCTAC |
| | TCTGGAGGCTGAGGCAGGAGAATGGCTTGAACCTGGGAGGCAGAGGCTGCAG |
| | TGAGCTGAAATTGTACTACTGCACTCCAGCCTGGGTGACAGAGTGAGACTCC |
| | ATCTCAAAAAAAAAAAAAAAAAAAAAAAATCAGACCTGCCTTCCATGAGCTC |
| | ATGGTATACTTGAATCTCCATAGGCTAGTTATTCAGGAGGGTATGTAATGTA |
| | ACTCAACAATGCACAATTACTTAAATTCGCTCAGGAGAATTACCTCATTTTG |
| | CCCAACTTGTTACTGTGAAAAAAAAAAAAGAAAGAAAATTTCAGGACCTTCC |
| | AAATTTATTATGCCAAAGGGAAAAGTCAAGCCCTGGAAACCAAGTCATGTAA |
| | CACGGCTGTTTTTCTTCTCTGGTGCATGACTGTTGCTTCCTGATCTTTTTGT |
| | TGATGTTATACA |
| 2033 | CATATAAATTAAATATTTATGTTATATTGAAGGAATACTTTTAGACTTGTTT |
| | AAACACAAATCTTTAAAAATTACATATCACTCTTGCATGTACATAAAAAATG |
| | AAAATATAGGCAATTAAATTAAGAGAGGTCTACAGTGTCTTTACATCAAGTC |
| | TGACTCTACTGAGTCCCTTTTTGACTCAGAGTCATTAATATATTGTTTTTTT |
| | CCAGTAATAATGTAGTGATGCAGCCTGTCTTCAAAGACTGCTCTACTATTGA |
| | CTCAGATTTTCTCCCAAGCCATTGATACTAGTTTTGAAGCTGATGCTTTTTA |
| | AATCTTGCTGTCAGACTTACGGGAAGGTTTTCATACAACAGGGCTCATATTC |
| | TTTCCTCAAATTATCCTTACATGTAAATGTTCAGAATGTCGAGATGATACAT |
| | AGGCCAGTTATGCCACTGTGAATATCTACCAAGGTCACATGTGTAATGAACA |
| | AAGACAGCTATTTCTGCTGCTGGCTGGCAGTGATTTGCAAGATTTTGTTGAC |
| | TGTAGGACATATCCTACTTCAATGATGTTAAAATGTGAACAAATATGCACTT |
| | CAGACTTTGTAAAATGTAGCACAGCACTTACAGAGCACACTAGGCTTCTGGC |
| | ACTCGCATAAAATGAAGACTTGGAGTTTTAGCTGAGTACTAAAGGAGGACCA |
| | TCCTCCCACCGAAGGATGAAGAATTTAAGGATATGTAAGTTGAGCTGTACTT |
| | ATGTTCATCTGTGATTTTTACAAGTCACTTATTGCTACATGTATCCTTTAAA |
| | TATGCGTTGTCCTTCCTCCTAAAATGGTTTCACCATAATAAGTGAAATGTCA |
| | GCTTGTCACATTAAATTATAAATTATAAATTACCATCACCTTAGTCCTCTAC |
| | ATATCCTTCAACTTCATTATGACACTGTCCTTCAGAGATAAGGAACAGAAAG |
| | GCTTTAATGAAAACTTCAGCTAATGTAATAATTAGGGAAGGATGAGCTAATT |
| | AAGAAACATACA |
| 2034 | CAAAGTCTCCCTAGAGGGCAAAATTGTCCCCATTGAAGACCACTGGGTTAGA |
| | TAGAAACTTACATCTCACACATGGAGAGTCCAGGCTGGCATGGTCGCTCTGC |
| | TGTGCACTGGGAGCCCAGGTTCCTCCTCGCTTTGCAAATTGTACAAGCTGCC |
| | CTCATCACCTGGATGCCTACATCTCACTTAAGAGTCTCAGTTCTAGGAGGGC |
| | ACAGACAATGGTGTACTGGTAAACAGACTCTGTTAAAAAAAAAAAAAAAAAA |
| | AACCAACACAATCAGGAACATTTTTTAAAAGCCCAGATTTGTAGTGTTTGCA |
| | GATTCTTATGTTTTAAATACTCCTGCCATGGCTGATGTGAAACTACCAACAG |
| | TTTAACAACTGGCTTACTAAATTTCTGAATATTTACCATTTGTCCCTTGTAA |
| | GACAGTATTAGTGGGCTGCAGTATATCAACAGAGAAAGGGAAGGAAAAGATA |
| | CAACCTTTTGTTGAAGGACAAAATGACATTTCACTTTTCTTCAGCCCCACTG |
| | GCCAAAACTTAGTCCCATGTTCACCTTAGCTGCAGGGGAGGCTGAAATGCAG |
| | TGTTTATTCTAAACAACCATGTATCCAGCCACAATACCAGGGGAATTTATCA |
| | CCAAGAGAAAGAGAGAGAGAATATCTAGTGCTTGAAAATTATCAGTCTCTGC |
| | CACAATTTTATTTAAAAAATAACCAGAAAAATGAGAGTGAATTTTATCTGAG |
| | AGGATCTTAGAAATCTCAGCATCGAGAAGGTAATAAATAAAGAGAGATAAGT |
| | CACAGACTTCCTGCGACAGTCAAGAATTCCCCATGCAGATGACACCCCAGGA |
| | GATGCCGGGTGATTGTTCTTACAATTTCTTCAGTTGAAGGTAAATGTGGCAC |
| | TAGCCATTTATTCTTTTAGCTCACGTTGTTTGAAGTGCATCGCCTATGTACT |
| | TCACCCTTTGGACTCACTAGAAAACAAAGAGAATTTTGGAATTAGAAGAGGC |
| | TTAATAATGTTA |
| 2035 | AAATATAAATAAAACATTTCTTTTGGAAATTTTATAATTCAAGCTAATTTAA |
| | AATTATGTAAACCTCTATCTTTCATGTAATCTTCTTCCTTCTTTTAAAACAA |
| | CATTTTTTTGGTGGTCATCTGTTCGGGAGAAAATGAAATTTTCTGTGGATAA |
| | GCAGATATTCTTCACGGAGAAAGCTAACATTCTGCATTCCTCTATTTTAAAA |
| | GTGGAAAACATAGTCCTGTTATTTGTATTTAGATGTATTTCTCACCAAAGAG |
| | TGCCAGGCTGGATTACAGAAGATCTATATTCTGATCTTGTCCTTTTTCTTTG |
| | CAAGCCTGAGGAATTGTCCAGACACAGAATTCCCTAGATCCCCAGATTTCTC |
| | ACCTATAATATGAAGGGTTGAAAGAGAGGTCTCAATCGGCTTTGAATTTTCT |
| | GTTCTATACTTCTGCACCACCACTGTAGCACTGACAATTGCATGAAAATATT |
| | AAGCTCTATTATGTTTTCAGTACTATCCTTAGCTTCTTTAAAAAATTAGTCT |
| | AGCTGTGTTTGTAAATAAATGATGTCACTGGAAAAATGGTTTCATACCATTG |
| | TTGTCAATAGTTGAATGTGGCTTGCCCTCAGGAACAATGCATTCTTCAATAA |
| | TATGGAGGATGGAAGGTGTATAAGGACTCAGATAGCTATTATTCTCATTTGC |
| | CCATGATCCTTTCATATCCCCGCCTCTGGTTTAGCATTCTCTTTCTTCCAGG |
| | GGAATTTCTCCCCCATTCCATGCATTCTAGTAGAATTTTTTATCACAGTAGA |
| | TTGTCCTGCCCTGCCACAGAAATGGGCATTTGACACAGTGGCCACAAAGATT |
| | GGTCTAAGCAGTAGGCCTGTGACCCAAGGTAGGCCAATTAGAGTTTTCTGTA |
| | GAATTTTTTAGATTCAAAGTGTATGTGTGTGGGGGGGATGACTCTTCTTGAA |
| | TTTTATATTAGGATGCATGCCAGAAATTGTTGAAAGGTCTTTAATGTACCAT |
| | GTACAGGAAGCT |
| 2036 | CACCTATAAGAGGAAATATACTTATGTCTAGGTGGACTCCAATGTGTCTGTT |
| | TACTGATACTTATTTATTCATTATTTTCAAGTAAAATGTAGAAGTGAATAAC |
| | TTAAGAGAATAACTATTTTTATGAGAGAAAAATACCCACTTTCTTTTTTATT |
| | ACTTTGTTCCTCTAGAGGTTCATGAATAATATATTGAACATGTGAGGAGTGA |
| | GGCCTGTCTAGCTCTTTTCCTAACATCTTCCACTCCTGTGGCCTCTTATTAG |
| | GTACCTTTCTCAGTGAAGATATACAATAAGAATTTTGCATGCTTATTGGGAA |
| | TTTATCTGTGAAAAATCACTCAAATGTCATTAAGTCTTTTCTGATAAACCTT |
| | AATCATCCAACAACCAGAGTTTTTCTTAAAATAGCTGTTGCTCTAGAAGAAT |
| | ACCATAGAATGAAGTTGCTTCCTAGCATGGCAGTCAAGGATCCTGGTTCCAA |
| | GTATGAGCTCTGAAGAAGATAGACTATGTTCACCGCTTACTATAGCTGAGTG |
| | CCCTTGGACAATTCATTTAAACTGCCCCTAATTTTCTTCCATCATCTGTAAA |
| | ATGAATGTAATAATAGCTCTTAATGAGTATTAAATTAGATAATAAGGGCACT |
| | GGCATTTATTAAGAACTTAATAAATGTTAGCTTTTGTTATTTCACATTTTTC |
| | CTTGATCACTCCTACCAGGAATAAAATTCTGGGAGGGTATAAGTAGGTAGTG |
| | AAGTGCTAACTGGTCTGGTTAATTGTTAGAGTTCTGTTAAAAAAAAGTTATT |
| | TGAAAAAAGTATTTTGGAGCTAGGATCTAATTTATTAATATATCTGGATTTT |
| | CTTTTTCAATTTTGGTGTCCATTATTCACATAAGTAATTGTGGTTTTGCTAT |
| | ATTTTTTCCTCCTGAAAAATTATGGCTATACAACTAACTTTATTGTATACTG |
| | AATTTTGGAATTTTTTAGGATTTGATGTTCTTACTGGGGAGAGGATTTTGAA |
| | TTATTTAACCAC |
| 2037 | AACAAGAGGAAAGCATACAAATTTATTTAATACATGTTTTATGTGGCACAGG |
| | AGCCCTCATAAAGTAATAAAAAATCCCCAAACACAGTTAGAGCTGAACATTT |
| | ATATACTAATCTGGACAAAACATTTATATACTGCGTGGACAAAGAGCAGTAA |
| | ATTGTGAAAATGGAACAAGGCAAGGGGGCTTAGACTACAGTAGTTAATCATC |
| | AAGAAGTGACAAAAAAAAATAAGGGTTAGTTAATAAGATTTGTTTAAGCAGA |
| | TTTCTCCCAGCTTTAGCTCTCTGTCTCTGGTGATCAGAATGCACTCCTTCCT |
| | TCAGACTCAGTGAGCACATATTCCACACGGAAGATTTCTTCCCTAGCTTTTA |
| | GGAAATCCAGAGAACCCTTTTTGTATCTGTTGTTTTTTTTTTTTTTTAAATG |
| | TCTTGTCTTTAACTCAAAACAATTTATGTGCCAGGATGACATATCTTTGGAT |
| | AATGTGTTCTGAACTCCTTCAGTACATACGTATATAAATTAAAGCAAATATT |
| | TTTTATGATAAGCTGGCATAATAGTTTCATAATTTAATCACTGATTTAAAAA |
| | TTTAATTAAAATTATTTTTTAATATTTTGTGTAATAATTTTTGAGGAGTATC |
| | TTTTGTGCTTAATGAGTGGCAGATGACACCCATGTTCTTAGCAGCATCATTC |
| | ACAATAGCTAAAAGATAGGAACAACTGCGTATTGATGGATGAATGGATAAGC |
| | AAAATGAGGTATATACATATAAGGGAATATTCTTCATCCTTAAAAAGGAAGG |
| | AAATTCTGACATATGCTACAACAAGGTTGAACCTCTAAGGACATTATGCTAA |
| | ATGAAATAAACCAGTCTCAAAAAGACAAATACTATGTGATTCCAGATACATA |
| | AGGCACCTAGAGACAAACTGATAGAGACAGAAAGTAGAATGAGTGATTACCA |
| | GGGGTTGTGAGAGGAAAAAAGAGAGGGTTGTTTGATACAGAGTTTCAGTTTT |
| | GCAAGATAAAAG |
| 2038 | AACAGGAGAAAAGCGTACAAGTTTATTAAATAGAAGTTTTGCAGCCGGGCGC |
| | GCTGGCTCACGCTTGTAATCCTGGCACTTTGGGAGGCCGAGGCGGGCAGATC |
| | ACGAGGTCAGGAGATCGAGACCACGGTGAAACCCCGTCTCTACTAAAAATAC |
| | AACAAATTAGCCAGGCGTGGTAGCGAGGCAGGAGAATGGTGTGAACCCGGGA |
| | GGCGGAGCTTGCCTCTGCACTCCAGATCATGCCACTGCACTCCAGCCTGGGT |
| | GACAGACCAAGACTCCGTCTCAAAAAAAAAAAAAAAAAAAAAAAAAAAAAAG |
| | AAGTTTTGCATGACATGGGAACCCTCATAAAAAAAGTGAAGTCCCAAAAAAG |
| | TGGCAAAATCTAAATGCTTTTATATTATGTTGACGAAAGAGGGGCAATTGTG |
| | GAAAAGTAACTAAATTATGAGGGTTAGGCTAACAGAAGATAAAAATTATTTT |
| | AACAAGTTCTGTTTGTATAAAATTTTCTCAATTTCAGCTACCCATCCTTGAT |
| | GATTAGAATGTTGCATTCCTTCTGGTATACAAGGAACATCTTCCATATGGGG |
| | GTTTTATCTTCTGCTTTCAGAAAAAAAAAAATAACTCTGTGTGTGGTAGGAT |
| | GAAGGTGTCAGAACATTCTTCTTGCACCTGCTGGTTGCTATCTTTTTAAACT |
| | GCATTTGTTTCAAAACAATCCTTATGACAAAGGGGTGTATTTTGGGGTGGCA |
| | TATTCTGTCACCCGTCAATATCAAAGTGGTTTTTGAGTTTGTGCTCATCTTC |
| | TTTCCTTATTCTGTTCCTTGTAAGGTAAGACTAAATATAATGGAATTTGCCG |
| | TCACATGTCTCTTATATGTAGTGAGTTTTAACAGGCATTCCAGGAAATGCCA |
| | TATGGTCTTTTAGCTTGGAAATTATTTTAGAAAACGATAAAATCTTTAGTGT |
| | GAAGTTATTTCCCAGATATGTATCGCTAAAATTATCATTACAGGTGCTCTAG |
| | GTAATATGTTTG |
| 2039 | TTAGTACTTCCATCCCTTTCCTGGCTGCTCTAACTTTACAGGTACTTGTAAG |
| | TGGCAATTAAGCACTTTTTCCTAATTCCAGAGTCTTGCCCCACTTCAGAGCA |
| | ACATAGAGTGGCCTAGACAGGCTGAGGTACTTTGCCGCTTCAGTCATCATTA |
| | ATCTATGGTATTTACTGATGAGAAGTAAAGTGGTAGAAGAAAAAAAAATTTT |
| | CTGTTATCCTGGGCACTTGGAAATGAATGTATTCTCACAATCTGTTCTCAAA |
| | ACAACTTACTGATTCTGGGGTTCTGGAAGCTCTGATGTGCAGGTGAGCCTTT |
| | TAAATTCCTCACTGTTGGAGCTCCTATCTAGGACTCACTGGCTGGATGAAAA |
| | CGGTTCTTTTTATTGCTTTCTGAATGTCTGCTAGACAGGCGTAAGCAACACC |
| | TTATATCTGCCTTCTGAAAAAGGTAAAAGAACTGGGACCCATCCACCATGCT |
| | GGACAGCTCGGCAGTGGCAGTGGCCTCCCCCAGACCCTGTTCCGAGTGCTCC |
| | ACCAACAAACTCACCAGCAGTCAGAGTCTAGCCTCTCCCCAAACTTCACCTT |
| | CATCACAATTCATTTTAAGCCCTTCCACAACCCAATCAACTCTAGATCTACT |
| | TAATGGATAATAATTTGATCTCATGCAAACTGCACTTTCCTCTTCTCAGAAT |
| | GATCCTTCTACCCCTTAATTAAACATTTGAGAGTGAAAGAAGAGAAAATTCG |
| | GGTTCAAAGATTGGTAAGTCTAAGAAACCTAAGGAAAAGGAGTTAGTAAACA |
| | TGTTAATCAAAGAGTGAGCACTTTTCGGAAGCGCAACATTCAGATACCTTTC |
| | TTGATTGGATTCCAGAAGACTATTTCTGGGAAGAGGAGATTTGCATTTTTCT |
| | AAAGTCTTCTACCCACAGCCTAACCACCCTAGGGCTTTGAAATATTTTTTTT |
| | CTGATGTGCAGTCATAATTGAATAAATAAAATGATTCCTGATCATTTCTTCT |
| | CTTCAGCTTTAT |
| 2040 | TATTCCTGTATTTCTATTGTACTTTTTTGCATTAAGAAACATTTTCCAATGT |
| | AACATTTTAATAGATTTTTCACTATTTGTTGAGTTATTTTTGAGTGGTTGTA |
| | CTTGAGCTTGCCATCTATGTCTTAACTTCAGATTTGTACTAACTTAATTCCA |
| | GGGAGATATAGAAGCATTATTCCTACATAGCTCTATATCAACCCCCTTTTCC |
| | TGTGGTATTATTGTTATACAAGGTACACCATATATGTTACAAATACAATTAT |
| | TTATAGTTATAATTATTACTTTAAATATATCATTTATGTCTATTAAAGAAGC |
| | TGAGAGCAGAGAGGAGATAAAGTATATATTTATAGAATTTGTTATATTAAGC |
| | TTCTTATTTGTCATTCTGATTCTCTTTGTTCTGGTGGACTTGAGTAAATATG |
| | TGATGTTATTTCATTATGCACACACAGCTTTGCTCCTTGTCATTTTATTTAT |
| | GCTGTCTTTCTCAAGTATATTGCATTTAAATACATTATAGGACCAACAATTC |
| | AAATATATTTATGTTGTGTTATACAATTGCTTTTTAAAATCAGTTAAGATAG |
| | ATGGGATATGCACTGATAGTATGGTTTTTAAAATTATACTTTAAGTTCTGGG |
| | TTACATATGCAGAACATGCTGTTTGGTTACATAGGTATACACGTGCCATGGT |
| | GGTTTGCTGCACCCATCAACCCACCACCTACATTAGGTATTTCTCCTAATGT |
| | TATCTGTCCTCTGGCCTCCAACCCCCCGACATGCCCCAGTGTGTGATGTTCC |
| | CCTCCCTGTGTCCATGTGTTCTCATTGTTCAACTCCCACTTATGAGTGAGAA |
| | CATGTGGTGTTTGGTTTTCTGATCTTGTGATAGTTTCCTGAGAATGATGGTT |
| | TCCAGCTTCATCCATGTCCCTGAAAAAGATATGAACTCATCCTAGACAATAA |
| | TTCAAACACACACACACACACACACACACACACACACACACACACGCAAATG |
| | GCACTAGTATCT |
| 2041 | TCCAGAAAACATAACAATTCAGAACATATATTTAATCCCTCCTCAATCCAGA |
| | TCCTTGTTGAAACAATGAAAGAGTACAATATACTGCCATGAAAAGTACTGAG |
| | AAAAGTCTACAGATAGTGACATGGAAGAAAAGAAAAAATATTAAATAGATCA |
| | AACTAGTTATATAATTTGTATCTCATTTCTGTAAAATAAATTTAACATTTAT |
| | AAGTGTATTAGTTTGTTCTCACATTGCTATAATAAAATACCTGAGACTGGGT |
| | AATTAAAAAAAAAAACAGATTTAATTGGCACACAGTTCTATAGGCTGTACAG |
| | AGAAAACAGTGGCTTCTGCTTCTGGGGAGGTTTCAGGAAACTTCCAATCATG |
| | ATGGAAGCCGAAGGGGAAGCAGACACATCTTACGTGGCCAGAGCAGGAGCAC |
| | AAGTGTGAAGGGAAGTGTCTGTTCATATTCTTCACTCACTTTTTAATGGGGT |
| | TGTTTGTTTTTTTCTTAGAAATTTAAGTTCCTTGTAGATTCTGGATATTAGG |
| | CCTTTGTCAGATGGATAGATTGCAAAAATGTTCTCCCATTCTGCAGGTTGCC |
| | TGTTCACTTTGATGATAGTTTCTTTTGCTGAGCAGAAGCTCTTTAGTTTAAT |
| | TTTGCAGGGACATGGATGAAGCTGGAAACCATTATCTTCAGTAGACTAACTG |
| | TTAACAGGAACAGAAAACCAAAAACAAACAAAAGCATGAAGAGGGAAGTGTC |
| | ACCCACATGAGAACTCACTATTGTGATGACAACACCAAGGGGAATGGTGTTA |
| | AACCATGAGAACCGGCCCCCATGATCCAATCACTTCCCACCAGGCCCCACCT |
| | CCAATACTGGATATTACAATTCAACAAGAGATTTGGGCAGGAATACAGATCC |
| | AAACCATATCAGTAAATATAATAAATATATATTAATAAATATGTAAATATAT |
| | GTATGCAAGTTAACAAATGAACCAGTTGGTATGTAAGTATGTATATAAAGGA |
| | CCATAGCAGTTA |
| 2042 | CTGAATACTAGAGGAGCAAGTACAACAAATGGAAAATGGGATCAAGTATGAG |
| | TGAGAGTTGCTAAGATGCCTGGTAGGGATGCAAAGGGGTAGAGAGCCTGGGG |
| | AGAGAGGGTGAGGGAGGGAAGCACTGGTTTCTCAAGCAAAAGCTAAAATTTT |
| | TCTATTAAGATTTAACCTGATGCTACACTTTGGTGGTGCAGCAAGGGTCTCA |
| | AATGGTATAAAACTCAGGTGATCATGCTTTATGTCTGTCTCTAGAAAAATGC |
| | TCCAAAAATGATAAGTAGTGATAATCCGCAGTCTCGTTGCATAAAATCAGCC |
| | CCAGGTGAATGACTAAGCTCCATTTCCCTACCCCACCCTTATTACAATAACC |
| | TCGACACCAACTCTAGTCCGTGGGAAGATAAACTAATCGGAGTCGCCCCTCA |
| | AATCTTACAGCTGCTCACTCCCCTGCAGGGCAACGCCCAGGGACCAAGTTAG |
| | CCCCTTAAGCCTAGGCAAAAGAATCCCGCCCATAATCGAGAAGCGACTCGAC |
| | ATGGAGGCGATGACGAGATCACGCGAGGAGGAAAGGAGGGAGGGCTTCTTCC |
| | AGGCCCAGGGCGGTCCTTACAAGACGGGAGGCAGCAGAGAACTCCCATAAAG |
| | GTATTGCGGCACTCCCCTCCCCCTGCCCAGAAGGGTGCGGCCTTCTCTCCAC |
| | CTCCTCCACCGCAGCTCCCTCAGGATTGCAGCTCGCGCCGGTTTTTGGAGAA |
| | CAAGCGCCTCCCACCCACAAACCAGCCGGACCGACCCCCGCTCCTCCCCCAC |
| | CCCCACGAGTGCCTGTAGCAGGTCGGGCTTGTCTCGCCCTTCAGGCGGTGGG |
| | AACCCGGGGCGGAGCCGCGGCCGCCGCCATCCAGAAGTCTCGGCCGGCAGCC |
| | CGCCCCCGCCTCCAGCGCGCGCTTCCTGCCACGTTGCGCAGGGGCGCGGGGC |
| | CAGACACTGCGGCGCTCGGCCTCGGGGAGGACCGTACCAACGCCCGCCTCCC |
| | CGCCACCCCCGCGCCCCGCGCAGTGGTTTCGCTCATGTGAGACTCGAGCCAG |
| | TAGCA |
| 2043 | GCCCTGCCAGGACGGGGCTGGCTACTGGCCTTATCTCACAGGTAAAACTGAC |
| | GCACGGAGGAACAATATAAATTGGGGACTAGAAAGGTGAAGAGCCAAAGTTA |
| | GAACTCAGGACCAACTTATTCTGATTTTGTTTTTCCAAACTGCTTCTCCTCT |
| | TGGGAAGTGTAAGGAAGCTGCAGCACCAGGATCAGTGAAACGCACCAGACGG |
| | CCGCGTCAGAGCAGCTCAGGTTCTGGGAGAGGGTAGCGCAGGGTGGCCACTG |
| | AGAACCGGGCAGGTCACGCATCCCCCCCTTCCCTCCCACCCCCTGCCAAGCT |
| | CTCCCTCCCAGGATCCTCTCTGGCTCCATCGTAAGCAAACCTTAGAGGTTCT |
| | GGCAAGGAGAGAGATGGCTCCAGGAAATGGGGGTGTGTCACCAGATAAGGAA |
| | TCTGCCTAACAGGAGGTGGGGGTTAGACCCAATATCAGGAGACTAGGAAGGA |
| | GGAGGCCTAAGGATGGGGCTTTTCTGTCACCAATCCTGTCCCTAGTGGCCCC |
| | ACTGTGGGGTGGAGGGGACAGATAAAAGTACCCAGAACCAGAGCCACATTAA |
| | CCGGCCCTGGGAATATAAGGTGGTCCCAGCTCGGGGACACAGGATCCCTGGA |
| | GGCAGCAAACATGCTGTCCTGAAGTGGACATAGGGGCCCGGGTTGGAGGAAG |
| | AAGACTAGCTGAGCTCTCGGACCCCTGGAAGATGCCATGACAGGGGGCTGGA |
| | AGAGCTAGCACAGACTAGAGAGGTAAGGGGGGTAGGGGAGCTGCCCAAATGA |
| | AAGGAGTGAGAGGTGACCCGAATCCACAGGAGAACGGGGTGTCCAGGCAAAG |
| | AAAGCAAGAGGATGGAGAGGTGGCTAAAGCCAGGGAGACGGGGTACTTTGGG |
| | GTTGTCCAGAAAAACGGTGATGATGCAGGCCTACAAGAAGGGGAGGCGGGAC |
| | GCAAGGGAGACATCCGTCGGAGAAGGCCATCCTAAGAAACGAGAGATGGCAC |
| | AGGCCCCAGAAGGAGAAGGAAAAGGGAACCCA |

In certain cases, expression of an exogenous DNA, e.g., transgene, inserted in a target polynucleotide at or near a target nucleotide sequence may depend on cell type and differentiation stage, as one or more components of a target polynucleotide get activated during differentiation while others get silenced, which may or may not be correlated with 5 rearrangements of the chromatin architecture reorganization during differentiation. To overcome this, in certain embodiments, additional to the exemplary characteristics described above, a suitable target polynucleotide comprising a target nucleotide sequence demonstrates suitable expression of an inserted exogenous DNA, e.g., transgene, throughout differentiation and clonal expansion.

### IV. EXAMPLES

### Example 1: Calculating Risk Profiles for Three gNAs Comprising Spacer Sequences Complementary to a Target Sequence in CIITA, TRAC, or B2M Genes

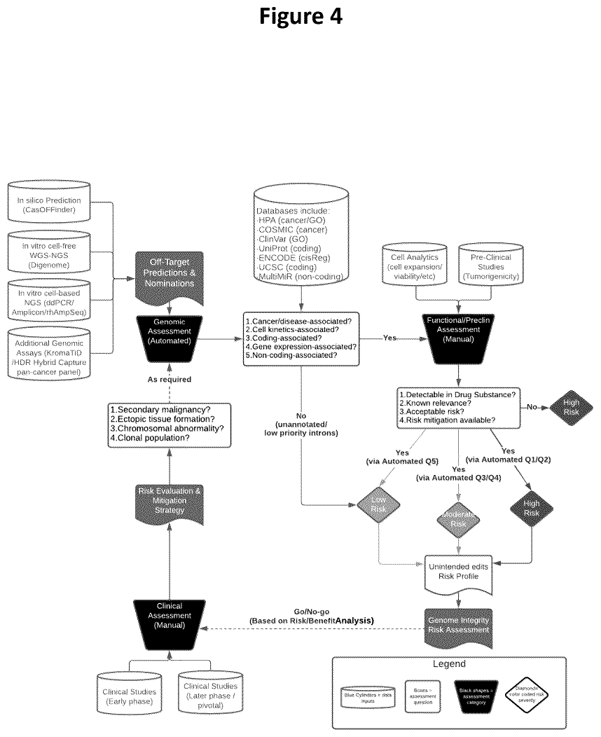

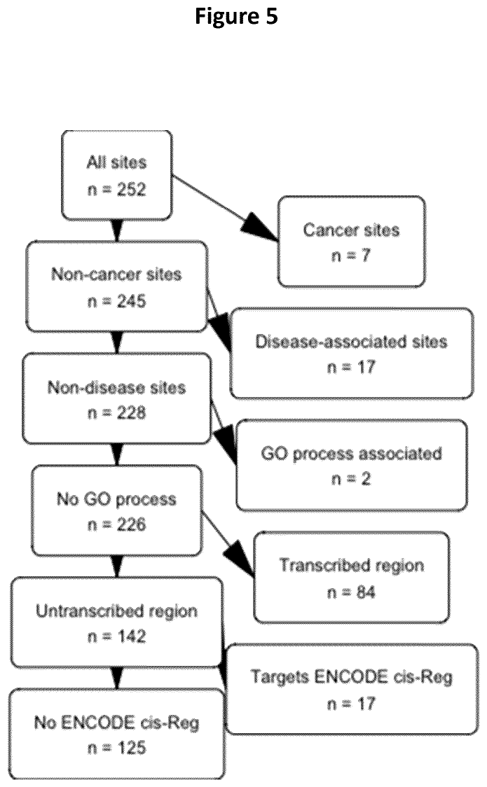

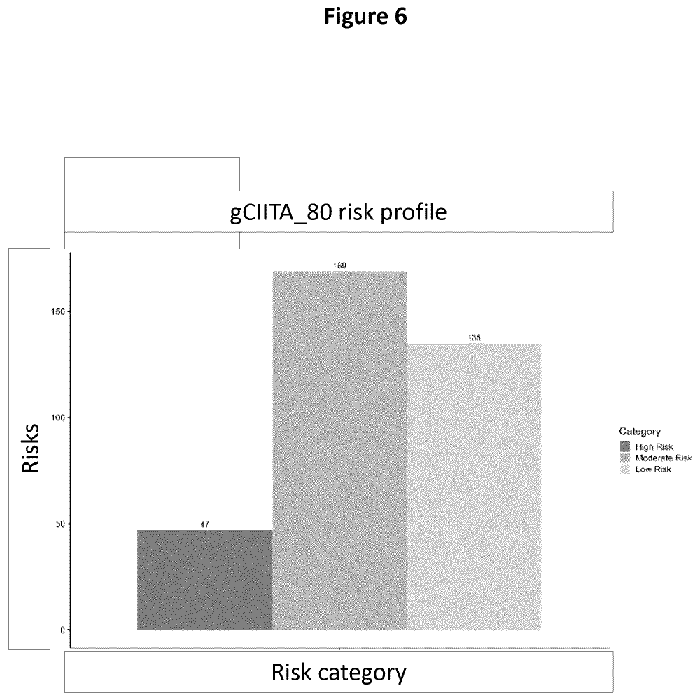

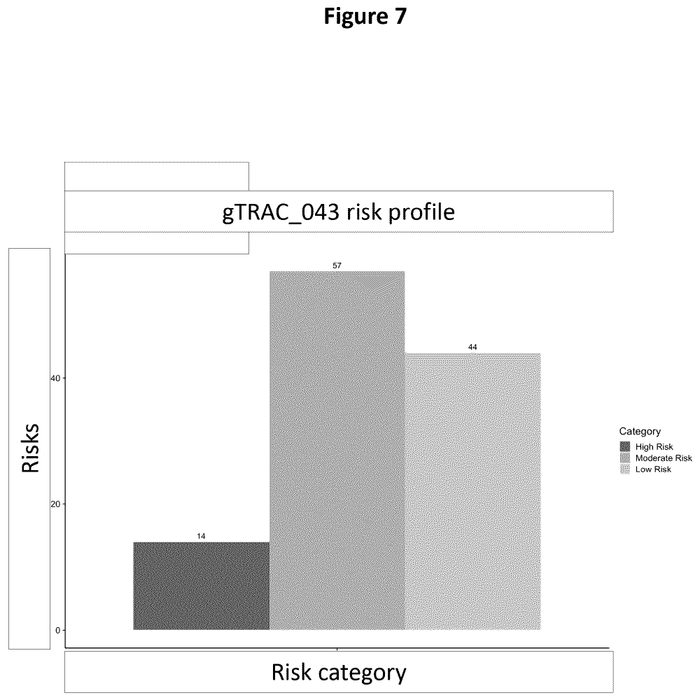

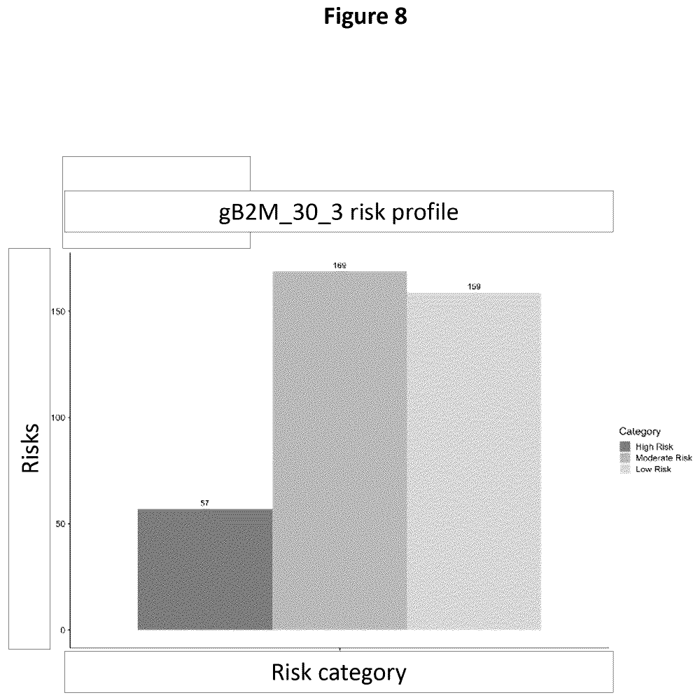

This example demonstrates the ability to calculate a risk profile for multiple gRNAs. Three gRNAs were selected comprising spacer sequences complementary to a target sequence in a CIITA (gCIITA_80), TRAC (gTRAC_043), or B2M (gB2M_30_3) gene. Each spacer sequence was examined using an exemplary decision-making framework (FIG. 4) and a risk profile was generated for each spacer sequence (FIGS. 6-8). First, a preliminary in silico off-target assessment was performed using CasOFFinder. Second, each gRNA complexed with MAD and combined with human genomic DNA, wherein the human genomic DNA was cleaved and the resulting cleavage products were analyzed by sequencing. The in silico and in vitro data were used to generated a list of off-target sites and each site was analyzed for its relative functional risk using the following risk ranking criteria: (1) if the site is associated with a cancer/disease-associated gene then the site is categorized as a high risk site; (2) if the site is associated with a cell kinetic/growth-associated gene then the site is categorized as a high risk site; (3) if the site is associated with a coding region then the site is categorized as a moderate risk site; (4) if the site is associated with a regulator of gene expression (such as a promoter or a transcription factor) then the site is categorized as a moderate risk site; (5) if the site is associated with a non-coding region then the site is categorized as a low risk site. Each off-target site was categorized as low, moderate, or high risk and the risk profile was generated as illustrated using a histogram of the count of each category for each spacer sequence (FIGS. 6-8). The site in the moderate risk category were than manually curated by assessing whether the off-target site match any of the four following criteria: (1) detectable in drug substance; (2) has a known relevance; (3) comprises an acceptable risk; (4) known risk mitigation available. If the site didn't meet each of the four criteria, then the site was elevated to high risk. If the site met each of the four criteria then the site remained as moderate risk. FIG. 5 shows the results from assessing in silico data categorizing risk for the three gNAs. Specifically, FIG. 5 shows the 3 gRNAs were associated with 252 off-target sites, of which 7 were sites associated with cancer and 245 were sites not associated with cancer. Of the 245 sites not associated with cancer, 17 site were associated with a known disease and 228 were not associated with a known disease. Of the 228 sites not associated with a known diseases, 2 sites were associated with a GO process and 226 sites were not associated with a GO process. Of the 226 sites not associated with a GO process, 84 were in a transcribed region and 142 sites were not in a transcribed region. Of the 142 sites not in a transcribed region, 17 of the sites were classified as ENCODE cis-Reg sites, and the remained 125 site were no ENCODE cis-Reg sites. The risks were the categorized as low, moderate, or high risk and a risk profile was generated for each spacer sequence as shown in FIGS. 6-8. Specifically, FIG. 6 shows the risk profile for the spacer sequence in gCIITA_80, wherein the risk profile comprises 47 high risk sites, 169 moderate risk sites, and 135 low risk sites. Specifically, FIG. 7 shows the risk profile for the spacer sequence in gTRAC_043, wherein the risk profile comprises 14 high risk sites, 57 moderate risk sites, and 44 low risk sites Specifically, FIG. 8 shows the risk profile for the spacer sequence in gB2M_30_3, wherein the risk profile comprises 57 high risk sites, 169 moderate risk sites, and 159 low risk sites.

This example demonstrates the ability to assess the relative risk of any number of gNAs comprising spacer sequences to any target site, and the utility in generating risk profiles to understand the associated risk with gNAs that enables genome editing companies to assess (and re-assess) in an actionable way any data about unintended edits in a consistent manner to inform benefit-risk decisions.

### Example 2

This example demonstrates the ability to calculate a hazard levels for multiple gRNAs targeting a single gene, and the ability to refine the set of gNA candidates for additional evaluation using these hazard levels at multiple stages of development.

gNAs were designed using the high-activity YTTV PAM preference of the ART STAR nuclease (nuclease comprising an amino acid sequence of MAD7) and the nucleotide sequence of the TRAC gene exons. The resulting 90 gNAs were checked against hg38 for sequence homology with potential off-target sites using the publicly available tool CasOFFinder v3.0, using the more permissive PAM sequence YTTN and allowing up to four sequence mismatches. Each off-target site produced was categorized as high, moderate, or low hazard as follows:

Several databases were queried for information related to these identified off-target sites. In the hazard levels shown in the accompanying figures, each site is assigned a hazard level according to whether the site falls within a series of functional categories: CANCER, DISEASE, BIO. FUNCTION, PROTEIN-CODING, REG. ELEMENT, and FUNCTIONAL NONCODING. Several different databases were queried for relevant information to determine potential off-target site function.

### Gene Annotation

The resolution of all subsequent features depends on the relevant underlying biology. For the first three categories, each predicted off-target site was first checked against the transcripts in the UCSC known gene database, as defined by the transcript start and end points from the ‘best-transcript’ tracks for hg38: the ‘knownCanonical’ gene tracks.

NCBI/EBI generated these annotations at UCSC as a subset of the GENCODE v29 track. As opposed to the hg19 knownCanonical table, which used computationally generated gene clusters and generally chose the longest isoform as the canonical isoform, the hg38 table uses ENSEMBL gene IDs to define clusters (that is to say, one canonical isoform per ENSEMBL gene ID), and the method of choosing the isoform is described as follows: “knownCanonical identifies the canonical isoform of each cluster ID or gene using the ENSEMBL gene IDs to define each cluster. The canonical transcript is chosen using the APPRIS principal transcript when available. If no APPRIS tag exists for any transcript associated with the cluster, then a transcript in the BASIC set is chosen. If no BASIC transcript exists, then the longest isoform is used.” If the off-target site lies within a gene, the entire gene is used for queries in the first three categories.

### Cancer Category

The Human Protein Atlas was queried for oncological annotations, as well as COSMIC's published Tier 1 Cancer Census set of cancer-associated genes. If the site was within a gene thus associated with cancer, it was marked as a “High Hazard’ off-target site, regardless of its location in exonic or intronic regions.

### Disease Category

The Clin Var database provided by the NCBI was also queried. To quote the website, “ClinVar is a freely accessible, public archive of reports of the relationships among human variations and phenotypes, with supporting evidence.” Once a site was determined to fall within a UCSC-annotated gene, ClinVar was for any known pathogenic variants within that gene associated with cancer. Specifically, the ‘clinSign’ annotation was used, which is the clinical significance value of reported variants. Variants annotated as ‘Likely pathogenic’, ‘Pathogenic’, ‘Likely pathogenic, low penetrance’, and ‘Pathogenic, low penetrance’ were used to identify genes associated with disease. These annotations have been established by Clin Var per the recommendation by ACMG/AMP or ClinGen. When multiple annotations for the same variant are present, the more severe phenotype annotation is used. Any annotations from the Human Protein Atlas for associations with diseases other than cancer are also used to flag the off-target sites as ‘High Hazard’.

### Biological Function Category

The Gene Ontology database was queried to check if the gene overlapping the off-target site is associated with proliferation (‘cell-division cycle’, GO: 007049; ‘cell population proliferation’, GO: 0008283), development (‘developmental process’, GO: 0032502), differentiation (‘cell differentiation’, CO: 0030154), or metabolism (‘metabolic process’, GO: 0008152). These sites are marked as ‘Moderate Hazard’

### Protein-Coding Category

Perturbation of protein domains was queried using the Uniprot database, and any overlapping transcripts. These sites were marked as ‘Moderate Hazard’

### Regulatory Element Category

The ENCODE Candidate cis-Regulatory Elements was queried for the noncoding features per site. These sites were marked as ‘Moderate Hazard’.

### Functional Noncoding Category

The MultiMir database is used to identify any noncoding RNA located at the predicted off-target site. These sites are marked as ‘Moderate Hazard’.

### Unannotated Category

All off-target sites which have no annotations in the above categories are marked as ‘Low Hazard’. The final number of ‘High Hazard’, ‘Moderate Hazard’, and ‘Low Hazard’ sites are reported for each set of off-target sites.

The off-target sites nominated by CasOFFinder for each of the TRAC-targeting gNAs were categorized as described above are summarized in the table below:

| | High | Moderate | Low |
|---|---|---|---|
| | Hazard | Hazard | Hazard |
| gNA | Sites | Sites | Sites |
| 4mm_gTRAC001_cutsite | 49 | 87 | 59 |
| 4mm_gTRAC002_cutsite | 23 | 43 | 37 |
| 4mm_gTRAC003_cutsite | 30 | 58 | 59 |
| 4mm_gTRAC004_cutsite | 24 | 47 | 51 |
| 4mm_gTRAC005_cutsite | 13 | 18 | 16 |
| 4mm_gTRAC006_cutsite | 9 | 7 | 17 |
| 4mm_gTRAC007_cutsite | 46 | 40 | 35 |
| 4mm_gTRAC008_cutsite | 78 | 118 | 103 |
| 4mm_gTRAC009_cutsite | 52 | 84 | 65 |
| 4mm_gTRAC010_cutsite | 73 | 99 | 97 |
| 4mm_gTRAC011_cutsite | 47 | 82 | 83 |
| 4mm_gTRAC012_cutsite | 31 | 49 | 62 |
| 4mm_gTRAC013_cutsite | 15 | 34 | 15 |
| 4mm_gTRAC014_cutsite | 8 | 13 | 16 |
| 4mm_gTRAC015_cutsite | 37 | 39 | 40 |
| 4mm_gTRAC016_cutsite | 11 | 13 | 11 |
| 4mm_gTRAC017_cutsite | 15 | 27 | 19 |
| 4mm_gTRAC018_cutsite | 10 | 20 | 11 |
| 4mm_gTRAC019_cutsite | 24 | 31 | 32 |
| 4mm_gTRAC020_cutsite | 13 | 19 | 11 |
| 4mm_gTRAC021_cutsite | 20 | 19 | 15 |
| 4mm_gTRAC022_cutsite | 16 | 25 | 16 |
| 4mm_gTRAC023_cutsite | 4 | 30 | 16 |
| 4mm_gTRAC024_cutsite | 41 | 60 | 56 |
| 4mm_gTRAC025_cutsite | 69 | 387 | 153 |
| 4mm_gTRAC026_cutsite | 19 | 30 | 28 |
| 4mm_gTRAC027_cutsite | 26 | 28 | 25 |
| 4mm_gTRAC028_cutsite | 16 | 49 | 33 |
| 4mm_gTRAC029_cutsite | 10 | 13 | 11 |
| 4mm_gTRAC030_cutsite | 8 | 17 | 16 |
| 4mm_gTRAC031_cutsite | 39 | 65 | 41 |
| 4mm_gTRAC032_cutsite | 52 | 115 | 63 |
| 4mm_gTRAC033_cutsite | 20 | 28 | 32 |
| 4mm_gTRAC034_cutsite | 63 | 85 | 72 |
| 4mm_gTRAC035_cutsite | 10 | 9 | 8 |
| 4mm_gTRAC036_cutsite | 3 | 9 | 11 |
| 4mm_gTRAC037_cutsite | 1 | 4 | 4 |
| 4mm_gTRAC038_cutsite | 5 | 10 | 6 |
| 4mm_gTRAC039_cutsite | 7 | 14 | 7 |
| 4mm_gTRAC040_cutsite | 2 | 1 | 5 |
| 4mm_gTRAC041_cutsite | 53 | 69 | 90 |
| 4mm_gTRAC042_cutsite | 20 | 22 | 23 |
| 4mm_gTRAC043_cutsite | 6 | 2 | 2 |
| 4mm_gTRAC044_cutsite | 6 | 14 | 8 |
| 4mm_gTRAC045_cutsite | 11 | 14 | 9 |
| 4mm_gTRAC046_cutsite | 16 | 31 | 23 |
| 4mm_gTRAC047_cutsite | 14 | 17 | 16 |
| 4mm_gTRAC048_cutsite | 20 | 37 | 23 |
| 4mm_gTRAC049_cutsite | 23 | 26 | 29 |
| 4mm_gTRAC050_cutsite | 5 | 17 | 7 |
| 4mm_gTRAC051_cutsite | 9 | 13 | 12 |
| 4mm_gTRAC055_cutsite | 15 | 20 | 12 |
| 4mm_gTRAC056_cutsite | 4 | 7 | 12 |
| 4mm_gTRAC057_cutsite | 4 | 4 | 9 |
| 4mm_gTRAC058_cutsite | 21 | 16 | 15 |
| 4mm_gTRAC059_cutsite | 8 | 12 | 3 |
| 4mm_gTRAC060_cutsite | 5 | 3 | 12 |
| 4mm_gTRAC061_cutsite | 0 | 2 | 3 |
| 4mm_gTRAC062_cutsite | 2 | 5 | 0 |
| 4mm_gTRAC063_cutsite | 34 | 49 | 35 |
| 4mm_gTRAC064_cutsite | 7 | 15 | 15 |
| 4mm_gTRAC065_cutsite | 9 | 10 | 7 |
| 4mm_gTRAC066_cutsite | 10 | 12 | 14 |
| 4mm_gTRAC067_cutsite | 11 | 4 | 5 |
| 4mm_gTRAC068_cutsite | 4 | 10 | 6 |
| 4mm_gTRAC069_cutsite | 1 | 8 | 10 |
| 4mm_gTRAC070_cutsite | 2 | 17 | 1 |
| 4mm_gTRAC071_cutsite | 7 | 11 | 18 |
| 4mm_gTRAC072_cutsite | 11 | 9 | 15 |
| 4mm_gTRAC073_cutsite | 12 | 22 | 21 |
| 4mm_gTRAC074_cutsite | 14 | 24 | 22 |
| 4mm_gTRAC075_cutsite | 11 | 23 | 20 |
| 4mm_gTRAC076_cutsite | 20 | 28 | 27 |
| 4mm_gTRAC077_cutsite | 19 | 40 | 33 |
| 4mm_gTRAC078_cutsite | 8 | 9 | 11 |
| 4mm_gTRAC079_cutsite | 13 | 14 | 19 |
| 4mm_gTRAC080_cutsite | 22 | 29 | 21 |
| 4mm_gTRAC081_cutsite | 11 | 15 | 24 |
| 4mm_gTRAC082_cutsite | 4 | 20 | 12 |
| 4mm_gTRAC083_cutsite | 12 | 25 | 15 |
| 4mm_gTRAC084_cutsite | 11 | 9 | 16 |
| 4mm_gTRAC085_cutsite | 10 | 23 | 12 |
| 4mm_gTRAC086_cutsite | 9 | 10 | 12 |
| 4mm_gTRAC087_cutsite | 25 | 26 | 23 |
| 4mm_gTRAC088_cutsite | 7 | 10 | 14 |
| 4mm_gTRAC089_cutsite | 23 | 25 | 20 |
| 4mm_gTRAC090_cutsite | 11 | 25 | 11 |
| 4mm_gTRAC091_cutsite | 104 | 140 | 144 |
| 4mm_gTRAC092_cutsite | 11 | 9 | 16 |
| 4mm_gTRAC093_cutsite | 4 | 4 | 4 |

These off-target cut site hazard profiles were used to compute a risk score R_{E} for each gNA:

$${R}_{E} ( g ) = ( \# high hazard * 100 ) + ( \# moderate hazard * 1 ) + ( \# low hazard * 0.1 )$$

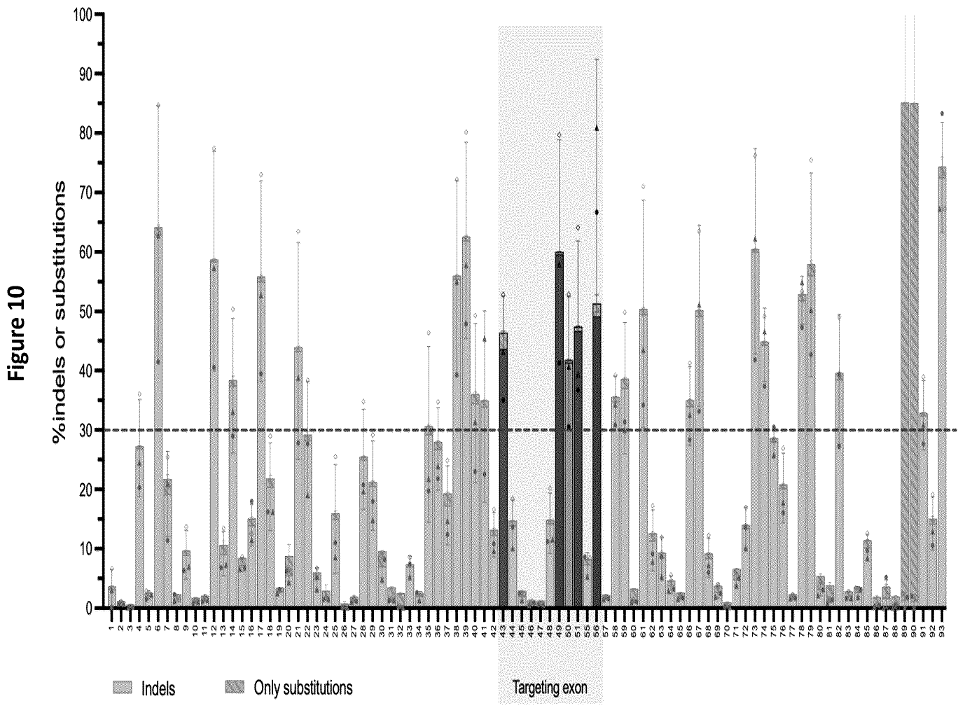

Separately, the on-target indel percentage introduced by each gNA was evaluated with Amplicon-seq. The results are shown in FIG. 10.

The top 5 gNA were selected based on the “guide score”, the ratio of % indel (on-target efficiency) to the R_{E} score of the gNA. These top-performing and lowest-risk gRNA were then further evaluated for additional on-target and off-target activity. For on-target performance, flow cytometry was performed to test for the presence of cell surface markers indicating a successful disruption of TRAC. Cell viability and proliferation were evaluated with cell count assays to ensure product requirements were met. For off-target activity, 20 cells were analyzed for abnormal karyotypes.

These results were used to update the “guide score”, with flow cytometry results replacing the % indel results for each gNA that passed the cell viability and proliferation criteria, and no updates to the R_{E} score due to the negative karyotyping results. The top 3 gNA were chosen based on this updated “guide score” for further off-target evaluation. Updated guide scores for the 5 gRNAs are shown below

STAR guide score = Functional score/Hazard score

| Guide name | STAR guide score |
|---|---|
| STAR guide 1 | 130.76 |
| STAR guide 2 | 10.62 |
| STAR guide 3 | 36.63 |
| STAR guide 4 | 19.73 |
| STAR guide 5 | 1.91 |

These 3 gNA were subjected to Digenome-seq analysis, which nominated a set of off-target cut sites observed in cell-free DNA upon treatment with the subject gNA. The Digenome-seq data was processed using Mantis software to modify the data, and the resulting candidate off-target cut sites categorized by further evaluation of off-target sites.

The Mantis software tool allows the identification of off-target cut sites from Digenome-seq data with an associated ‘cleavage score’. While Mantis uses a similar core scoring function to the publicly available digenome toolkit2, Mantis improves the set of returned off-target sites by employing several additional features.

The first set of features affect how the Digenome-seq data is processed. By accounting for high levels of optical duplicates observed in Digenome-seq data and resolving multi-mapped reads with the publicly available samtools markdup and “MMR” bioinformatic tools respectively, the Mantis workflow greatly reduces sequencing artifacts not otherwise accounted for in the Digenome-seq workflow. Mantis additionally discards off-target cut sites at a user-customizable threshold level if there are insufficient reads at adjacent genomic positions. This expands the “cutoff” for the total number of reads present required to call a significant off-target cut site beyond the site of the cut itself, which was all that was previously considered. With Mantis, all nucleotides used to calculate the cleavage score must meet this minimum read coverage requirement.

The second set of features refine how the cleavage score is calculated within Mantis. Mantis only returns the best peak within a user-defined region of each sample, rather than returning all peaks that exceed a given threshold, thus collapsing signal noise into a single most-likely peak. Mantis further allows the user to require a particular shape of the signal peak, allowing adjustment for nucleases with overhanging cuts and varying rates of DNA degradation during library preparation. Finally, Mantis returns information about sequence features adjacent to the called cut sites, allowing the user to select biologically relevant sites according to PAM availability and gRNA sequence matches.

Together, these features reduce the number of off-target cut sites that are called from Digenome-seq data due to sequencing artifacts and other noise. The improved set of off-target cut site candidates reduce the burden of down-stream validation experiments and produce a more reproducible set of nominated off-target sites from Digenome-seq data.

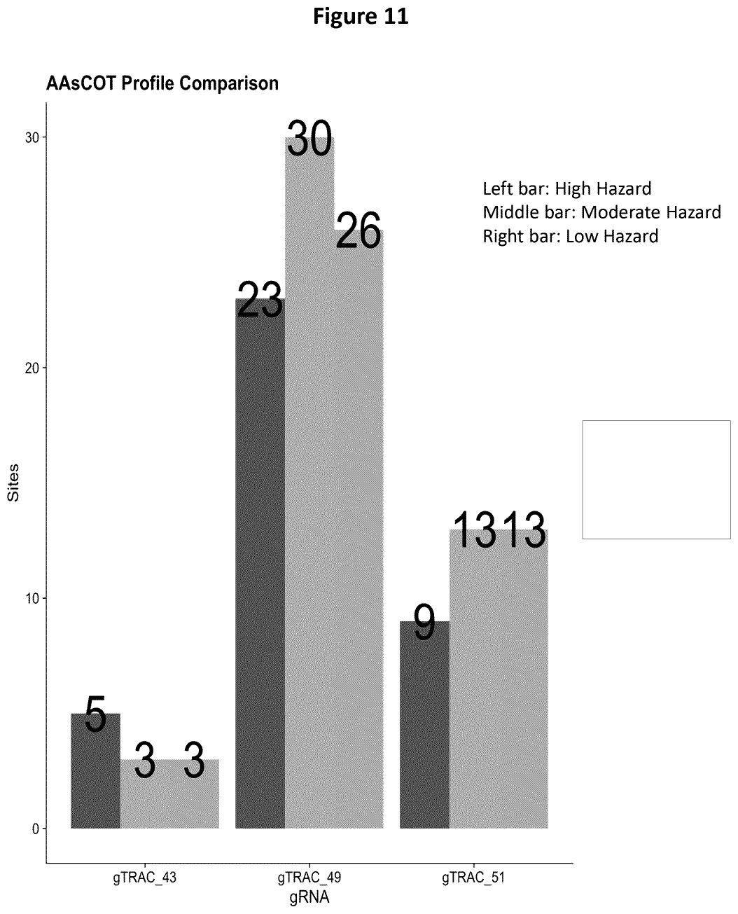

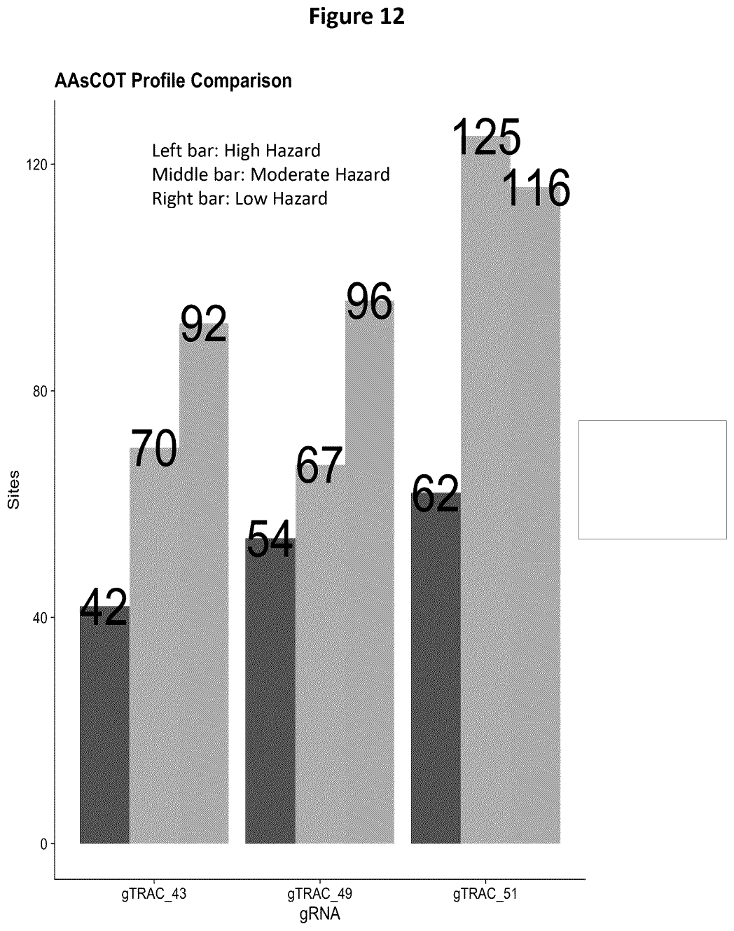

The hazard profiles of the three gNA targeting TRAC are shown in FIGS. 11 and 12 for off-target sites nominated with both CasOFFinder and Digenome-seq with Mantis.

CasOFFinder results are shown in FIG. 11. Results for Digenome-Seq sites called by Mantis are shown in FIG. 12.

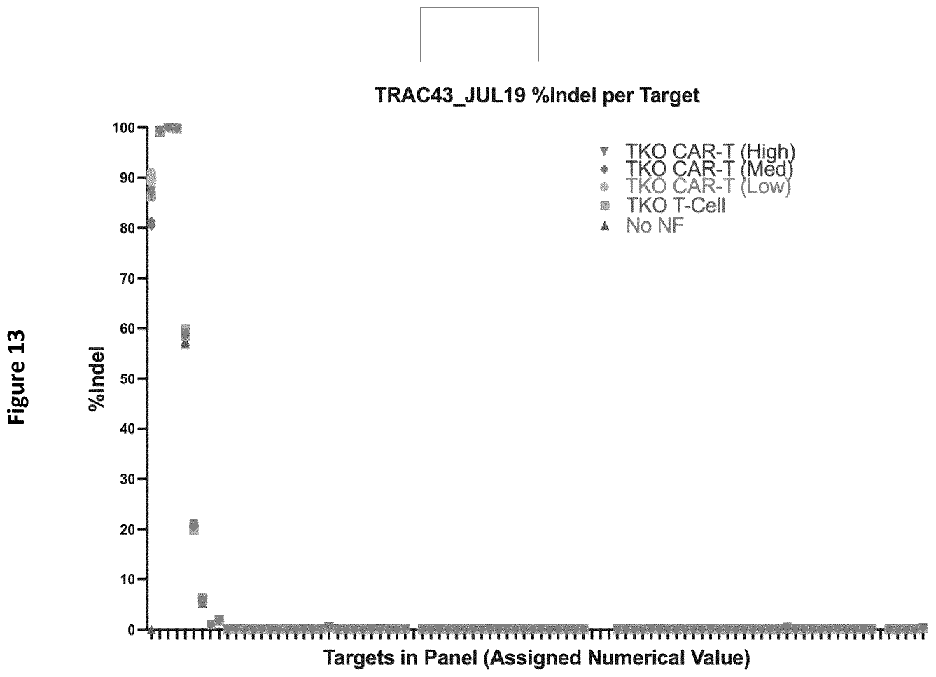

All sites categorized as High and Moderate hazard from either assay were selected for validation with rhAmp-seq. Results for TRAC43 are shown in FIG. 13. All sites confirmed with rhAmp-seq were then validated with ddPCR, and none were found to have detectable levels of gNA off-target activity for any of the three gNA evaluated. Because gTRAC43 had the best editing efficiency, gTRAC43 was recommended for further product development.

### V. EMBODIMENTS

Provided in embodiment 1 is a computer-implemented method for evaluating a potential off-target site for a guide nucleic acid (gNA), wherein the gNA comprises a spacer sequence partially or completely complementary to a target sequence in a target polynucleotide in a genome and is compatable with a CRISPR-associated nuclease, comprising (i) providing to the computer a genomic position for the potential off-target site for the gNA; and, (ii) on the computer, querying one or more databases that comprise information regarding potential function with the genomic position of the potential off-target site to determine whether or not the off-target site falls within one or more functional categories; and (iii) determining a hazard level for the potential off-target site based, at least in part, on the results of the querying of step (ii). In embodiment 2 provided is the computer-implemented method of embodiment 1 comprising evaluating a plurality of potential off-target sites for the gNA, wherein each potential off-target site is different from other potential off-target sites, comprising, for each potential off-target site performing steps (i)-(iii) and (iv) determining a hazard level for the gNA, based, at least in part, on the results of step (iii) for the plurality of potential off-target sites. In embodiment 3 provided is the computer-implemented method of embodiment 2 comprising determining hazard levels for a plurality of gNAs, wherein each of the gNAs comprises a spacer sequence partially or completely complementary to a target sequence in the target polynucleotide, and wherein each target sequence is different from other target sequences, comprising performing steps (i)-(iv) for each gNA. In embodiment 4 provided is the method of embodiment 3 further comprising (v) ranking the plurality of gNAs based, at least in part, on the results of step (iv) for each gNA. In embodiment 5 provided is the computer-implemented method of embodiment 4 further comprising outputting the ranking of the plurality of gNAs. In embodiment 6 provided is the method of any one of embodiments 1 through 4 wherein the one or more potential off-target sites are determined in silico, in vitro, or both. In embodiment 7 provided is the method of embodiment 6 wherein the potential off-target sites are determined both in silico and in vitro. In embodiment 8 provided is the method of embodiment 4 wherein the one or more potential off-target sites are determined in silico. In embodiment 9 provided is the method of embodiment 8 wherein the ranking of the plurality of gNAs is determined by a process that combines hazard ranking for each gNA with information regarding editing efficiency for each gNA. In embodiment 10 provided is the method of embodiment 9 wherein a subset of the plurality of gNAs is determined based, at least in part, by the ranking of the plurality of gNAs. In embodiment 11 provided is the method of embodiment 10 wherein the subset of gNAs is used in an in vitro method to identify potential off-target sites for each gNA. In embodiment 12 provided is the method of embodiment 11 wherein potential off-target sites determined in vitro for each gNA in the subset are used in step (iii) of analysis of potential off-target sites of the gNAs to determine a hazard level for each gNA in the subset. In embodiment 13 provided is the method of any one of embodiments 6, 7, or 11, wherein the in vitro method produces a plurality of signals related to potential off-target sites. In embodiment 14 provided is the method of embodiment 13 wherein the plurality of signals is processed by a method to eliminate likely false positive off-target sites, so that the information provided to the computer in step (i) does not include the likely false positive off-target sites. In embodiment 15 provided is the method of embodiment 14 wherein the method comprises evaluating the scores of flanking bases to call a peak in signal. In embodiment 16 provided is the method of embodiment 14 or embodiment 15 wherein the method comprises wherein peak assessment includes read coverage of adjacent bases within each scoring window. In embodiment 17 provided is the method of embodiment 16 wherein the method comprises adapting the size of the scoring window itself to individual nuclease signatures. In embodiment 18 provided is the method of any one of embodiments 14 through 17 wherein the method comprises evaluating position of adjacent PAMS. In embodiment 19 provided is the method of any previous embodiment wherein the one or more databases comprise a database comprising information regarding cancer-associated genes. In embodiment 20 provided is the method of any previous embodiment wherein the one or more databases comprise information regarding disease-associated genes. In embodiment 21 provided is the method of any previous embodiment wherein the one or more databases comprise information regarding genes associated with proliferation, development, cell differentiation, and/or metabolism. In embodiment 22 provided is the method of any previous embodiment wherein the one or more databases comprise information regarding protein-coding exons. In embodiment 23 provided is the method of any previous embodiment wherein the one or more databases comprise information regarding one or more regulatory elements. In embodiment 24 provided is the method of any previous embodiment wherein the one or more databases comprise information regarding functional non-coding nucleotide sequences. In embodiment 25 provided is the method of any previous embodiment further comprising providing the computer with cell-based information regarding the one or more gNAs, wherein the cell-based information is used in one or more steps relating to determining a hazard level for a gNA, ranking of gNAs, or both. In embodiment 26 provided is the method of embodiment 25 wherein the cell-based information is obtained from cells into which have been introduced the CRISPR-associated nuclease, or one or more poynucleotides coding therefor, and the gNA, or one or more polynucleotides coding therefor, and wherein the cell-based information comprises information regarding off-target events for each gNA. In embodiment 27 provided is the computer-implemented method of embodiment 25 or 26 wherein the cell-based information comprises sequence information for the one or more potential off-target sites. In embodiment 28 provided is the computer-implemented method of embodiment 27 wherein the sequence information for the one or more potential off-target sites is used to eliminate potential off-target sites from consideration in determining a hazard level for a gNA, to increase genome location resolution to determine a hazard level for a potential off-target site, or both. In embodiment 29 provided is the computer-implemented method of any one of embodiments 25 through 28 wherein the cell-based information comprises translocation information. In embodiment 30 provided is the computer-implemented method of embodiment 29 wherein the tranlocation information comprises information regarding karyotype and/or micro-translocation. In embodiment 31 provided is the computer-implemented method of any one of embodiments 25 through 30 wherein the sequence information for the one or more potential off-target sites comprises information regarding information regarding off-target insertions. In embodiment 32 provided is the method of any one of embodiments 25 through 31 wherein a preliminary hazard level for each cell-based assay is determined by assigning a numerical value for hazard level for the off-target event or events of each cell-based assay and multiplying by a frequency of the occurrence of the off-target event in the assay. In embodiment 33 provided is the method of embodiment 32 wherein determination of the preliminary hazard level further comprises assigning a numerical value to performance of each assay and multiplying the value obtained by multiplying hazard level and frequency by the numerical value. In embodiment 34 provided is the method of embodiment 33 comprising combining the preliminary hazard levels for the cell-based assays for each gNA to determine an overall hazard level for the gNA. In embodiment 35 provided is the method of embodiment 34 further comprising, for each gNA or for a subset of the gNAs, obtaining the cell-based information comprising information regarding growth, proliferation, and/or viability of cells into which the gNA is introduced or their progeny. In embodiment 36 provided is the method of embodiment 35 further comprising, for each gNA or a subset of the gNAs, obtaining cell-based information comprising information regarding expression levels of one or more genes associated with a pathology of cells into which the gNA is introduced. In embodiment 37 provided is the method of embodiment 36 wherein the pathology is cancer. In embodiment 38 provided is a method of generating a recommendation for use of one or more gNAs in a CRISPR process based, at least in part, on information obtained in any previous embodiment. In embodiment 39 provided is the method of embodiment 38 wherein generating the recommendation further comprises determining, at least in part one or more factors that modulate one or more effects of one or more events for an off-target site for the one or more gNAs on a desired product to be produced in a method comprising introducing the gNA and its compatible CRISPR nuclease into cells, a process to produce the product, and/or desired use of the product. In embodiment 40 provided is the method of embodiment 39 wherein the one or more factors comprise a presence of one or more cell markers directly or indirectly produced by the one or more off-target events for the off-target site, wherein the one or more cell markers can be used to selectively remove cells displaying the one or more cell markers from a population of cells used to produce the product. In embodiment 41 provided is the method of embodiment 39 or 40 wherein the one or more factors comprise an ability to select for a population of cells, e.g., clonal populations, used in the process to produce the product, wherein the one or more events at the one or more off-target sites has not occurred in the cells. In embodiment 42 provided is the method of any one of embodiments 39 through 41 wherein the one or more factors comprises determining a level of acceptable risk for the occurrence of the one or more events at the one or more off-target sites in a subject or population of subjects for whom the product will be used in treatment. In embodiment 43 provided is a data processing apparatus comprising a processor configured to perform the method of any previous embodiment. In embodiment 44 provided is a computer program comprising instructions which, when the program is executed by a computer, causes the computer to carry out the method of any one of embodiments 1 through 43. In embodiment 45 provided is a data carrier signal carrying the computer program of embodiment 45. In embodiment 46 provided is a computer-readable storage medium comprising instructions which, when executed by a computer, cause the computer to carry out the method of any one of embodiments 1 through 43. In embodiment 47 provided is a composition comprising a gNA, or one or more polynucleotides coding therefor, wherein the gNA is compatible with a CRISPR nuclease, wherein the gNA comprises a spacer sequence partially or completely complementary to a target sequence in a target polynucleotide, and wherein the gNA is selected from a plurality of potential gNAs, each of which is complementary to a different target sequence in the target polynucleotide, by the method of any one of embodiments 1 through 43. In embodiment 48 provided is the composition of embodiment 47 further comprising the CRISPR nuclease or one or more polynucleotides coding therefor. In embodiment 49 provided is a cell comprising the composition of embodiment 48, or a progeny thereof. In embodiment 50 provided is a method comprising introducing into a cell the composition of embodiment 48 and allowing the composition to bind to the target polynucleotide in the cell and produce a strand break in the polynucleotide.

In embodiment 51 provided is a method comprising providing information regarding potential off-target sites for a gNA, wherein the information is obtained by an in vitro method, wherein the in vitro method produces a plurality of signals related to potential off-target sites and processing the information by a method to eliminate likely false positive off-target sites. In embodiment 52 provided is the method of embodiment 51 comprising evaluating the scores of flanking bases to call a peak in signal. In embodiment 53 provided is the method of embodiment 51 or 52 wherein peak assessment includes read coverage of adjacent bases within each scoring window. In embodiment 54 provided is the method of embodiment 53 comprising adapting the size of the scoring window itself to individual nuclease signatures. In embodiment 55 provided is the method of any one of embodiments 51 through 54 wherein the method comprises evaluating position of adjacent PAMS.

In embodiment 56 provided is a method comprising introducing into a cell a CRISPR-associated nuclease, or one or more polynucleotides coding therefor, and a gNA, or one or more polynucleotides coding therefor, wherein the gNA comprises a spacer sequence partially or completely complementary to a target sequence in a target polynucleotide in the cell, and the gNA is selected from a plurality of gNAs, each of which comprises a spacer sequence that is complementary to a different target sequence in the polynucleotide, by a process comprising providing a plurality of potential off-target sites for each gNA, for each potential off-target site for each gNA, determining a hazard level for the off-target site, determining an overall hazard level for each gNA based, at least in part, on the results of (b), and selecting the gNA based, at least in part, on the overall hazard levels for each of the plurality of gNAs.

### VI. EQUIVALENTS

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A computer-implemented method for evaluating a potential off-target site for a guide nucleic acid (gNA), wherein the gNA comprises a spacer sequence partially or completely complementary to a target sequence in a target polynucleotide in a genome and is compatible with a CRISPR-associated nuclease, comprising
(i) providing to the computer a genomic position for the potential off-target site for the gNA; and, on the computer,
(ii) querying one or more databases that comprise information regarding potential function with the genomic position of the potential off-target site to determine whether or not the off-target site falls within one or more functional categories; and
(iii) determining a hazard level for the potential off-target site based, at least in part, on the results of the querying of step (ii).

2. The computer-implemented method of claim 1 comprising evaluating a plurality of potential off-target sites for the gNA, wherein each potential off-target site is different from other potential off-target sites, comprising, for each potential off-target site performing steps (i)-(iii) and
(iv) determining a hazard level for the gNA, based, at least in part, on the results of step (iii) for the plurality of potential off-target sites.

3. The computer-implemented method of claim 2 comprising determining hazard levels for a plurality of gNAs, wherein each of the gNAs comprises a spacer sequence partially or completely complementary to a target sequence in the target polynucleotide, and wherein each target sequence is different from other target sequences, comprising performing steps (i)-(iv) for each gNA.

4. The method of claim 3 further comprising
(v) ranking the plurality of gNAs based, at least in part, on the results of step (iv) for each gNA.

5. The computer-implemented method of claim 4 further comprising outputting the ranking of the plurality of gNAs.

6. The method of claim 1 wherein the one or more potential off-target sites are determined in silico, in vitro, or both.

7.-12. (canceled)

13. The method of claim 6, wherein the in vitro method produces a plurality of signals related to potential off-target sites.

14. (canceled)

15. The method of claim 14 wherein the method comprises evaluating the scores of flanking bases to call a peak in signal.

16.-17. (canceled)

18. The method of claim 14 wherein the method comprises evaluating position of adjacent PAMS.

19.-24. (canceled)

25. The method claim 1 further comprising providing the computer with cell-based information regarding the one or more gNAs, wherein the cell-based information is used in one or more steps relating to determining a hazard level for a gNA, ranking of gNAs, or both.

26. The method of claim 25 wherein the cell-based information is obtained from cells into which have been introduced the CRISPR-associated nuclease, or one or more polynucleotides coding therefor, and the gNA, or one or more polynucleotides coding therefor, and wherein the cell-based information comprises information regarding off-target events for each gNA.

27. The computer-implemented method of claim 25 wherein the cell-based information comprises sequence information for the one or more potential off-target sites or translocation information.

28. The computer-implemented method of claim 27 wherein the sequence information for the one or more potential off-target sites is used to eliminate potential off-target sites from consideration in determining a hazard level for a gNA, to increase genome location resolution to determine a hazard level for a potential off-target site, or both.

29. (canceled)

30. (canceled)

31. The computer-implemented method of claim 27 wherein the sequence information for the one or more potential off-target sites comprises information regarding off-target insertions.

32. The method of claim 25 wherein a preliminary hazard level for each cell-based assay is determined by assigning a numerical value for hazard level for the off-target event or events of each cell-based assay and multiplying by a frequency of the occurrence of the off-target event in the assay.

33. The method of claim 32 wherein determination of the preliminary hazard level further comprises assigning a numerical value to performance of each assay and multiplying the value obtained by multiplying hazard level and frequency by the numerical value.

34. The method of claim 33 comprising combining the preliminary hazard levels for the cell-based assays for each gNA to determine an overall hazard level for the gNA.

35. The method of claim 34 further comprising, for each gNA or for a subset of the gNAs, obtaining the cell-based information comprising information regarding growth, proliferation, and/or viability of cells into which the gNA is introduced or their progeny.

36. The method of claim 35 further comprising, for each gNA or a subset of the gNAs, obtaining cell-based information comprising information regarding expression levels of one or more genes associated with a pathology of cells into which the gNA is introduced.

37.-46. (canceled)

47. A composition comprising a gNA, or one or more polynucleotides coding therefor, wherein the gNA is compatible with a CRISPR nuclease, wherein the gNA comprises a spacer sequence partially or completely complementary to a target sequence in a target polynucleotide, and wherein the gNA is selected from a plurality of potential gNAs, each of which is complementary to a different target sequence in the target polynucleotide, wherein the gNA is selected by executing the steps of:
(i) providing an algorithm with a genomic position for a potential off-target site for the gNA; and,
(ii) querying, via the algorithm, one or more databases that comprise information regarding potential function with the genomic position of the potential off-target site to determine whether or not the potential off-target site falls within one or more functional categories; and
(iii) determining a hazard level for the potential off-target site based, at least in part, on results of the querying of step (ii).

48. The composition of claim 47 further comprising the CRISPR nuclease or one or more polynucleotides coding therefor.

49. (canceled)

50. A method comprising introducing into a cell the composition of claim 48 and allowing the composition to bind to the target polynucleotide in the cell and produce a strand break in the polynucleotide.

51.-55. (canceled)

56. A method comprising introducing into a cell a CRISPR-associated nuclease, or one or more polynucleotides coding therefor, and a gNA, or one or more polynucleotides coding therefor, wherein
(i) the gNA comprises a spacer sequence partially or completely complementary to a target sequence in a target polynucleotide in the cell, and
(ii) the gNA is selected from a plurality of gNAs, each of which comprises a spacer sequence that is complementary to a different target sequence in the polynucleotide, by a process comprising
(a) providing a plurality of potential off-target sites for each gNA,
(b) for each potential off-target site for each gNA, determining a hazard level for the off-target site,
(c) determining an overall hazard level for each gNA based, at least in part, on the results of (b), and
(d) selecting the gNA based, at least in part, on the overall hazard levels for each of the plurality of gNAs.
